(19) United States
(12) Patent Application Publication
(10) Pub. No.: US 2025/0197889 A1
(43) Pub. Date: Jun. 19, 2025

(54) Targeted CRISPR Delivery Platforms

(71) Applicant: University of Massachusetts, Westborough, MA (US)

(72) Inventors: Erik J. Sontheimer, Auburndale, MA (US); Raed Ibraheim, Shrewsbury, MA (US); Wen Xue, Natick, MA (US); Aamir Mir, Worcester, MA (US); Alireza Edraki, Worcester, MA (US); Ildar Gainetdinov, Worcester, MA (US)

(21) Appl. No.: 18/980,456

(22) Filed: Dec. 13, 2024

Related U.S. Application Data
(63) Continuation of application No. 18/612,111, filed on Mar. 21, 2024.
Application No. 18/612,111 is a continuation of application No. 16/186,352, filed on Nov. 9, 2018, now abandoned.
(60) Provisional application No. 62/667,084, filed on May 4, 2018.
(60) Provisional application No. 62/596,375, filed on Dec. 8, 2017.
(60) Provisional application No. 62/584,310, filed on Nov. 10, 2017.

(51) Int. Cl. C12N 15/86; A61K 48/00; C12N 15/11

(52) U.S. Cl. CPC C12N 15/86; A61K 48/0008; A61K 48/0066; A61K 48/0091; C12N 15/111; C12N 2310/20; C12N 2750/14141

(57) ABSTRACT

The present invention is related to compositions and methods for gene therapy. Several approaches described herein utilize the Neisseria meningitidis Cas9 system that provides a hyperaccurate CRISPR gene editing platform. Furthermore, the invention incorporates full length and truncated single guide RNA sequences that permit a complete sgRNA-Nme1Cas9 vector to be inserted into an adeno-associated viral plasmid that is compatible for in vivo administration. Furthermore, Type II-C Cas9 orthologs have been identified that target protospacer adjacent motif sequences limited to between one-four required nucleotides.

### STATEMENT OF RELATED CASES

This is a continuation of U.S. Non-Provisional application Ser. No. 18/612,111 filed on Mar. 21, 2024, having priority to Non-Provisional application Ser. No. 16/186,352 filed on Nov. 9, 2018, now Abandoned, having priority to U.S. Provisional Application No. 62/667,084 filed on May 4, 2018, U.S. Provisional Application No. 62/596,375 filed on Dec. 8, 2017 and U.S. Provisional Application No. 62/584,310 filed on Nov. 10, 2017.

### REFERENCE TO AN ELECTRONIC SEQUENCE LISTING

The contents of the electronic sequence listing (20677.xml; Size: 369,945 bytes; and Date of Creation: Mar. 4, 2025) is herein incorporated by reference in its entirety.

### FIELD OF THE INVENTION

The present invention is related to compositions and methods for gene therapy. Several approaches described herein utilize the Neisseria meningitidis Cas9 systems that provide hyperaccurate CRISPR gene editing platforms. Furthermore, the invention incorporates improvements of this Cas9 system: for example, truncating the single guide RNA sequences, and the packing of Nme1Cas9 or Nme2Ca9 with its guide RNA in an adeno-associated viral vector that is compatible for in vivo administration. Furthermore, Type II-C Cas9 orthologs have been identified that target protospacer adjacent motif sequences limited to between one-four required nucleotides.

### BACKGROUND

Clustered regularly interspaced short palindromic repeats (CRISPR)-CRISPR associated (Cas) is a unique RNA-guided adaptive immune system found in archaea and bacteria. These systems provide immunity by targeting and inactivating nucleic acids that originate from foreign genetic elements. Many different types of CRISPR-Cas systems have been identified to date and are categorized into two classes.

Within class II CRISPR systems, type II CRISPR-Cas systems are characterized by a single effector protein called Cas9, which forms a ribonucleoprotein (RNP) complex with CRISPR RNA (crRNA) and trans-activating RNA (tracrRNA) to target and cleave DNA. The crRNA contains a programmable guide sequence that can direct Cas9 to almost any DNA sequence in living organisms.

This programmability of Cas9 RNP complexes has been harnessed by many researchers for genome editing in eukaryotic systems. It has been used to edit the genomes of mammalian cells, human embryos, plants, rodents, and other living organisms. Cas9 RNPs have been used for precise (with donor template) and imprecise genome editing, both of which have found applications in gene therapy, agriculture, and elsewhere. In addition, the nuclease-dead versions of Cas9 orthologs are being used for transcription modulation, site-specific DNA labeling, and for proteome profiling at specific genomic loci. Several different Cas9s have been used for these applications. Central to the programmability of Cas9 and hence its applications is the ability to introduce any guide sequence in the crRNA. The crRNA and tracrRNA can be fused together to form a single-guide RNA (sgRNA), which is more stable and provides enhanced genome editing.

What is needed in the art are improved Cas9s and sgRNA sequences that can provide specific and accurate editing of a wider range of target sites, especially when combined with reliable nucleic acid delivery platforms.

### SUMMARY OF THE INVENTION

The present invention is related to compositions and methods for gene therapy. Several approaches described herein utilize Neisseria meningitidis Cas9 systems that provide hyperaccurate CRISPR gene editing platforms. Furthermore, the invention incorporates improvements of this Cas9 system: for example, truncating the single guide RNA sequences, and the packing of Nme1Cas9 or Nme2Cas9 with its guide RNA in an adeno-associated viral vector that is compatible for in vivo administration. Furthermore, Type II-C Cas9 orthologs have been identified that target protospacer adjacent motif sequences limited to between one-four required nucleotides.

In one embodiment, the present invention contemplates a single guide ribonucleic acid (sgRNA) sequence comprising a truncated repeat:anti-repeat region. In one embodiment, the sgRNA sequence further comprises a truncated Stem 2 region. In one embodiment, the sgRNA sequence further comprises a truncated spacer region. In one embodiment, said sgRNA sequence has a length of 121 nucleotides. In one embodiment, said sgRNA sequence length is selected from the group consisting of 111 nucleotides, 107 nucleotides, 105 nucleotides, 103 nucleotides, 102 nucleotides, 101 nucleotides, and 99 nucleotides. In one embodiment, said sgRNA sequence has a length of 100 nucleotides. In one embodiment, said sgRNA sequence is an Nme1Cas9 single guide ribonucleic acid sequence. In one embodiment, said sgRNA sequence is an Nme2Cas9 single guide ribonucleic acid sequence. In one embodiment, said sgRNA sequence is an Nme1Cas9 single guide ribonucleic acid sequence or an Nme2Cas9 single guide ribonucleic acid sequence.

In one embodiment, the present invention contemplates a single guide ribonucleic acid (sgRNA) sequence comprising a truncated Stem 2 region. In one embodiment, the sgRNA sequence further comprises a truncated repeat:anti-repeat region. In one embodiment, the sgRNA sequence further comprises a truncated spacer region. In one embodiment, said sgRNA sequence has a length is selected from the group consisting of 111 nucleotides, 107 nucleotides, 105 nucleotides, 103 nucleotides, 102 nucleotides, 101 nucleotides, and 99 nucleotides. In one embodiment, said sgRNA sequence has a length of 100 nucleotides.

In one embodiment, the present invention contemplates an adeno-associated viral (AAV) vector comprising a single guide ribonucleic acid-Neisseria meningitidis Cas9 (sgRNA-Nme1Cas9 or sgRNA-Nme2Cas9) nucleic acid vector. In one embodiment, said single guide ribonucleic acid-Neisseria meningitidis Cas9 nucleic acid vector comprises at least one promoter. In one embodiment, said at least one promoter is selected from the group consisting of a U6 promoter and a U1a promoter. In one embodiment, said single guide ribonucleic acid-Neisseria meningitidis Cas9 nucleic acid vector comprises a Kozak sequence. In one embodiment, said sgRNA comprises a nucleic acid sequence that is complementary to a gene-of-interest sequence. In one embodiment, said gene-of-interest sequence is selected from the group consisting of a PCSK9 sequence and a ROSA26 sequence. In one embodiment, said sgRNA comprises an untruncated sequence that has a length of 145 nucleotides. In one embodiment, said sgRNA comprises a truncated repeat-antirepeat sequence. In one embodiment, said sgRNA further comprises a truncated Stem 2 region. In one embodiment, said sgRNA further comprises a truncated spacer region. In one embodiment, said sgRNA sequence has a length of 121 nucleotides. In one embodiment, said sgRNA sequence has a length selected from the group consisting of 111 nucleotides, 107 nucleotides, 105 nucleotides, 103 nucleotides, 102 nucleotides, 101 nucleotides, and 99 nucleotides. In one embodiment, said sgRNA sequence has a length of 100 nucleotides. In one embodiment, said sgRNA comprises a truncated Stem 2 region. In one embodiment, said sgRNA further comprises a truncated repeat:antirepeat region. In one embodiment, said sgRNA further comprises a truncated spacer region. In one embodiment, said sgRNA sequence has a length selected from the group consisting of 111 nucleotides, 107 nucleotides, 105 nucleotides, 103 nucleotides, 101 nucleotides, and 99 nucleotides. In one embodiment, said sgRNA sequence has a length of 100 nucleotides. In one embodiment, said sgRNA comprises an untruncated sequence has a length of 145 nucleotides.

In one embodiment, the present invention contemplates a method, comprising: a) providing; i) a patient exhibiting at least one symptom of a medical condition, wherein said patient comprises a plurality of genes related to said medical condition; ii) a delivery platform comprising a single guide ribonucleic acid-Neisseria meningitidis Cas9 (sgRNA-Nme1Cas9 or sgRNA-Nme2Cas9) nucleic acid vector, wherein said sgRNA comprises a nucleic acid sequence that is complementary to a portion of at least one of said plurality of genes; and b) administering said AAV plasmid to said patient under conditions such that said at least one symptom of said medical condition is reduced. In one embodiment, the delivery platform comprises an adeno-associated viral (AAV) vector. In one embodiment, the delivery platform comprises a microparticle. In one embodiment, said medical condition comprises hypercholesterolemia. In one embodiment, said medical condition comprises tyrosinemia. In one embodiment, said at least one of said plurality of genes is a PCSK9 gene. In one embodiment, said sgRNA nucleic acid is complementary to a portion of said PCSK9 gene. In one embodiment, at least one of said plurality of genes is an FAH gene. In one embodiment, said sgRNA nucleic acid is complementary to a portion of said FAH gene. In one embodiment, said sgRNA comprises a truncated repeat-antirepeat sequence. In one embodiment, said sgRNA further comprises a truncated Stem 2 region. In one embodiment, said sgRNA further comprises a truncated spacer region. In one embodiment, said sgRNA sequence has a length of 121 nucleotides. In one embodiment, said sgRNA sequence has a length selected from the group consisting of 111 nucleotides, 107 nucleotides, 105 nucleotides, 103 nucleotides, 101 nucleotides, and 99 nucleotides. In one embodiment, said sgRNA sequence has a length of 100 nucleotides. In one embodiment, said sgRNA comprises a truncated Stem 2 region. In one embodiment, said sgRNA further comprises a truncated repeat:antirepeat region. In one embodiment, said sgRNA further comprises a truncated spacer region. In one embodiment, said sgRNA sequence has a length selected from the group consisting of 111 nucleotides, 107 nucleotides, 105 nucleotides, 103 nucleotides, 102 nucleotides, 101 nucleotides, and 99 nucleotides. In one embodiment, said sgRNA sequence has a length of 100 nucleotides. In one embodiment, said sgRNA comprises an untruncated sequence has a length of 145 nucleotides.

In one embodiment, the present invention contemplates an adeno-associated viral (AAV) plasmid encoding a Type II-C Cas9 nuclease protein wherein said protein comprises a protospacer adjacent motif recognition domain configured with a binding site to a protospacer adjacent motif sequence comprising between one to four required nucleotides. In one embodiment, said Type II-C Cas9 nuclease protein is selected from the group consisting of a Neisseria meningitidis strain De10444 Nme2Cas9 nuclease protein, a Haemophilus parainfluenzae HpaCas9 nuclease protein and a Simonsiella muelleri SmuCas9 nuclease protein. In one embodiment, said protospacer adjacent motif sequence comprising one to four required nucleotides is selected from the group consisting of N₄CN₃, N₄CT, N₄CCN, N₄CCA, and N₄GNT₃. In one embodiment, the one to four required nucleotides are selected from the group consisting of C, CT, CCN, CCA, CN₃ and GNT₂. In one embodiment, said Type II-C Cas9 nuclease protein is bound to a truncated sgRNA. In one embodiment, the adeno-associated viral plasmid encodes two sgRNA sequences. In one embodiment, the adeno-associated viral plasmid encodes a poly-adenosine sequence. In one embodiment, the adeno-associated viral plasmid encodes a homology-directed repair donor nucleotide template. In one embodiment, the adeno-associated viral plasmid is an all-in-one adeno-associated viral plasmid.

In one embodiment, the present invention contemplates, a method, comprising: a) providing; i) a patient exhibiting at least one symptom of a medical condition, wherein said patient comprises a plurality of genes related to said medical condition, wherein said plurality of genes comprise a protospacer adjacent motif comprising between one-four required nucleotides; ii) a delivery platform comprising at least one nucleic acid encoding a Type II-C Cas9 nuclease protein wherein said protein comprises a protospacer adjacent motif recognition domain configured with a binding site to said protospacer adjacent motif sequence comprising between two-four required nucleotides; and b) administering said delivery platform to said patient under conditions such that said at least one symptom of said medical condition is reduced. In one embodiment, said medical condition comprises hypercholesterolemia. In one embodiment, said medical condition comprises tyrosinemia. In one embodiment, said at least one of said plurality of genes is a PCSK9 gene. In one embodiment, said sgRNA nucleic acid is complementary to a portion of said PCSK9 gene. In one embodiment, at least one of said plurality of genes is an FAH gene. In one embodiment, said sgRNA nucleic acid is complementary to a portion of said FAH gene. In one embodiment, said delivery platform comprises an adeno-associated viral plasmid. In one embodiment, said delivery platform comprises a microparticle. In one embodiment, said Type II-C Cas9 nuclease protein is selected from the group consisting of a Neisseria meningitidis strain De10444 Nme2Cas9 nuclease protein, a Haemophilus parainfluenzae HpaCas9 nuclease protein and a Simonsiella muelleri SmuCas9 nuclease protein. In one embodiment, said protospacer adjacent motif sequence comprising one-four required nucleotides is selected from the group consisting of N₄CN₃, N₄CT, N₄CCN, N₄CCA, and N₄GNT₃. In one embodiment, the one to four required nucleotides are selected from the group consisting of C, CT, CCN, CCA, CN₃ and GNT₂. In one embodiment, said Type II-C Cas9 nuclease protein is bound to a truncated sgRNA. In one embodiment, the adeno-associated viral plasmid encodes two sgRNA sequences. In one embodiment, the adeno-associated viral plasmid encodes a poly-adenosine sequence. In one embodiment, the adeno-associated viral plasmid encodes a homology-directed repair donor nucleotide template. In one embodiment, the adeno-associated viral plasmid is an all-in-one adeno-associated viral plasmid.

In one embodiment, the present invention contemplates an adeno-associated viral (AAV) plasmid encoding a Type II-C Cas9 nuclease protein wherein said protein comprises a protospacer adjacent motif recognition domain (e.g., a PAM-Interacting Domain; PID) configured to bind with a protospacer adjacent motif (PAM) sequence, said PAM sequence comprising an adjacent cytosine dinucleotide pair. In one embodiment the adjacent cytosine dinucleotide pair is at the PAM positions five (5) and six (6). In one embodiment, said Type II-C Cas9 nuclease protein is derived from a Neisseria meningitidis strain. In one embodiment, the Neisseria meningitidis strain is De10444. In one embodiment, the Type II-C Cas9 nuclease protein is an Nme2Cas9 nuclease protein. In one embodiment, the Neisseria meningitidis strain is 98002. In one embodiment, the Type II-C Cas9 nuclease protein is an Nme3Cas9 nuclease protein. In one embodiment, said PAM sequence is selected from the group consisting of N₄CC, N₄CCN₃, N₄CCA, N₄CC(X), N₄CA₃ and N₁₀. In one embodiment, the PAM sequence is N₃CC. In one embodiment, the Type II-C Cas9 nuclease protein further comprises an sgRNA sequence. In one embodiment, the sgRNA sequence comprises a spacer ranging in length between approximately seventeen (17)-twenty four (24) nucleotides.

In one embodiment, the present invention contemplates a method, comprising: a) providing; i) a patient exhibiting at least one symptom of a medical condition, wherein said patient comprises a plurality of genes related to said medical condition, wherein said plurality of genes comprise a protospacer adjacent motif comprising an adjacent cytosine dinucleotide pair; ii) a delivery platform comprising at least one nucleic acid encoding a Type II-C Cas9 nuclease protein wherein said protein comprises a protospacer adjacent motif recognition domain (e.g., a PAM Interacting Domain; PID) configured to bind with said protospacer adjacent motif sequence comprising an adjacent cytosine dinucleotide pair; and b) administering said delivery platform to said patient under conditions such that said at least one symptom of said medical condition is reduced. In one embodiment, said delivery platform comprises an adeno-associated viral vector. In one embodiment, the adeno-associated viral vector is adeno-associated viral vector eight (AAV8). In one embodiment, said medical condition comprises hypercholesterolemia. In one embodiment, said medical condition comprises tyrosinemia. In one embodiment, the medical condition is x-linked chronic granulomatous disease. In one embodiment, the medical condition is aspartylglycosaminuria. In one embodiment, said at least one of said plurality of genes is a PCSK9 gene. In one embodiment, said sgRNA nucleic acid is complementary to a portion of said PCSK9 gene. In one embodiment, at least one of said plurality of genes is an FAH gene. In one embodiment, said sgRNA nucleic acid is complementary to a portion of said FAH gene. In one embodiment, the adeno-associated viral plasmid encodes at least one sgRNA sequence. In one embodiment, the adeno-associated viral plasmid encodes two sgRNA sequences. In one embodiment, the adeno-associated viral plasmid encodes a poly-adenosine sequence. In one embodiment, the adeno-associated viral plasmid encodes a homology-directed repair donor nucleotide template. In one embodiment, the adeno-associated viral plasmid is an all-in-one adeno-associated viral plasmid. In one embodiment, said delivery platform comprises a microparticle. In one embodiment the adjacent cytosine dinucleotide pair is at the PAM positions five (5) and six (6). In one embodiment, said Type II-C Cas9 nuclease protein is derived from a Neisseria meningitidis strain. In one embodiment, the Neisseria meningitidis strain is De10444. In one embodiment, the Type II-C Cas9 nuclease protein is an Nme2Cas9 nuclease protein. In one embodiment, the Neisseria meningitidis strain is 98002. In one embodiment, the Type II-C Cas9 nuclease protein is an Nme3Cas9 nuclease protein. In one embodiment, said PAM sequence is selected from the group consisting of N₄CC, N₄CCN₃, N₄CCA, N₄CC(X), N₄CA₃ and N₁₀. In one embodiment, the PAM sequence is N₃CC. In one embodiment, the Type II-C Cas9 nuclease protein further comprises an sgRNA sequence. In one embodiment, the sgRNA sequence comprises a spacer ranging in length between approximately seventeen (17)-twenty four (24) nucleotides.

### Definitions

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity but also plural entities and also includes the general class of which a specific example may be used for illustration. The terminology herein is used to describe specific embodiments of the invention, but their usage does not delimit the invention, except as outlined in the claims.

The term “about” or “approximately” as used herein, in the context of any of any assay measurements refers to +/−5% of a given measurement.

As used herein the “ROSA26 gene” or “Rosa26 gene” refers to a human or mouse (respectively) locus that is widely used for achieving generalized expression in the mouse. Targeting to the ROSA26 locus may be achieved by introducing a desired gene into the first intron of the locus, at a unique XbaI site approximately 248 bp upstream of the original gene trap line. A construct may be constructed using an adenovirus splice acceptor followed by a gene of interest and a polyadenylation site inserted at the unique XbaI site. A neomycin resistance cassette may also be included in the targeting vector.

As used herein the “PCSK9 gene” or “Pcsk9 gene” refers to a human or mouse (respectively) locus that encodes a PCSK9 protein. The PCSK9 gene resides on chromosome 1 at the band 1p32.3 and includes 13 exons. This gene may produce at least two isoforms through alternative splicing.

The term “proprotein convertase subtilisin/kexin type 9” and “PCSK9” refers to a protein encoded by a gene that modulates low density lipoprotein levels. Proprotein convertase subtilisin/kexin type 9, also known as PCSK9, is an enzyme that in humans is encoded by the PCSK9 gene. Seidah et al., “The secretory proprotein convertase neural apoptosis-regulated convertase 1 (NARC-1): liver regeneration and neuronal differentiation” Proc. Natl. Acad. Sci. U.S.A. 100 (3): 928-933 (2003). Similar genes (orthologs) are found across many species. Many enzymes, including PCSK9, are inactive when they are first synthesized, because they have a section of peptide chains that blocks their activity; proprotein convertases remove that section to activate the enzyme. PCSK9 is believed to play a regulatory role in cholesterol homeostasis. For example, PCSK9 can bind to the epidermal growth factor-like repeat A (EGF-A) domain of the low-density lipoprotein receptor (LDL-R) resulting in LDL-R internalization and degradation. Clearly, it would be expected that reduced LDL-R levels result in decreased metabolism of LDL-C, which could lead to hypercholesterolemia.

The term “hypercholesterolemia” as used herein, refers to any medical condition wherein blood cholesterol levels are elevated above the clinically recommended levels. For example, if cholesterol is measured using low density lipoproteins (LDLs), hypercholesterolemia may exist if the measured LDL levels are above, for example, approximately 70 mg/dl. Alternatively, if cholesterol is measured using free plasma cholesterol, hypercholesterolemia may exist if the measured free cholesterol levels are above, for example, approximately 200-220 mg/dl.

As used herein, the term “CRISPRs” or “Clustered Regularly Interspaced Short Palindromic Repeats” refers to an acronym for DNA loci that contain multiple, short, direct repetitions of base sequences. Each repetition contains a series of bases followed by 30 or so base pairs known as “spacer” sequence. The spacers are short segments of DNA from a virus and may serve as a ‘memory’ of past exposures to facilitate an adaptive defense against future invasions. Doudna et al. Genome editing. The new frontier of genome engineering with CRISPR-Cas9” Science 346(6213):1258096 (2014).

As used herein, the term “Cas” or “CRISPR-associated (cas)” refers to genes often associated with CRISPR repeat-spacer arrays.

As used herein, the term “Cas9” refers to a nuclease from type II CRISPR systems, an enzyme specialized for generating double-strand breaks in DNA, with two active cutting sites (the HNH and RuvC domains), one for each strand of the double helix. tracrRNA and spacer RNA may be combined into a “single-guide RNA” (sgRNA) molecule that, mixed with Cas9, could find and cleave DNA targets through Watson-Crick pairing between the guide sequence within the sgRNA and the target DNA sequence, Jinek et al. A programmable dual-RNA-guided DNA endonuclease in adaptive bacterial immunity” Science 337(6096):816-821 (2012).

As used herein, the term “catalytically active Cas9” refers to an unmodified Cas9 nuclease comprising full nuclease activity.

The term “nickase” as used herein, refers to a nuclease that cleaves only a single DNA strand, either due to its natural function or because it has been engineered to cleave only a single DNA strand. Cas9 nickase variants that have either the RuvC or the HNH domain mutated provide control over which DNA strand is cleaved and which remains intact. Jinek et al., “A programmable dual-RNA-guided DNA endonuclease in adaptive bacterial immunity” Science 337(6096):816-821 (2012) and Cong et al. Multiplex genome engineering using CRISPR/Cas systems” Science 339(6121):819-823 (2013).

The term, “trans-activating crRNA”, “tracrRNA” as used herein, refers to a small trans-encoded RNA. For example, CRISPR/Cas (clustered, regularly interspaced short palindromic repeats/CRISPR-associated proteins) constitutes an RNA-mediated defense system, which protects against viruses and plasmids. This defensive pathway has three steps. First a copy of the invading nucleic acid is integrated into the CRISPR locus. Next, CRISPR RNAs (crRNAs) are transcribed from this CRISPR locus. The crRNAs are then incorporated into effector complexes, where the crRNA guides the complex to the invading nucleic acid and the Cas proteins degrade this nucleic acid. There are several pathways of CRISPR activation, one of which requires a tracrRNA, which plays a role in the maturation of crRNA. TracrRNA is complementary to the repeat sequence of the pre-crRNA, forming an RNA duplex. This is cleaved by RNase III, an RNA-specific ribonuclease, to form a crRNA/tracrRNA hybrid. This hybrid acts as a guide for the endonuclease Cas9, which cleaves the invading nucleic acid.

The term “protospacer adjacent motif” (or PAM) as used herein, refers to a DNA sequence that may be required for a Cas9/sgRNA to form an R-loop to interrogate a specific DNA sequence through Watson-Crick pairing of its guide RNA with the genome. The PAM specificity may be a function of the DNA-binding specificity of the Cas9 protein (e.g., a “protospacer adjacent motif recognition domain” at the C-terminus of Cas9).

The terms “protospacer adjacent motif recognition domain”, “PAM Interacting Domain” or “PID” as used herein, refers to a Cas9 amino acid sequence that comprises a binding site to a DNA target PAM sequence.

The term “binding site” as used herein, refers to any molecular arrangement having a specific tertiary and/or quaternary structure that undergoes a physical attachment or close association with a binding component. For example, the molecular arrangement may comprise a sequence of amino acids. Alternatively, the molecular arrangement may comprise a sequence a nucleic acids. Furthermore, the molecular arrangement may comprise a lipid bilayer or other biological material.

As used herein, the term “sgRNA” refers to single guide RNA used in conjunction with CRISPR associated systems (Cas). sgRNAs are a fusion of crRNA and tracrRNA and contain nucleotides of sequence complementary to the desired target site. Jinek et al., “A programmable dual-RNA-guided DNA endonuclease in adaptive bacterial immunity” Science 337(6096):816-821 (2012) Watson-Crick pairing of the sgRNA with the target site permits R-loop formation, which in conjunction with a functional PAM permits DNA cleavage or in the case of nuclease-deficient Cas9 allows binds to the DNA at that locus.

As used herein, the term “orthogonal” refers to targets that are non-overlapping, uncorrelated, or independent. For example, if two orthogonal Cas9 isoforms were utilized, they would employ orthogonal sgRNAs that only program one of the Cas9 isoforms for DNA recognition and cleavage. Esvelt et al., “Orthogonal Cas9 proteins for RNA-guided gene regulation and editing” Nat Methods 10(11):1116-1121 (2013). For example, this would allow one Cas9 isoform (e.g. S. pyogenes Cas9 or SpyCas9) to function as a nuclease programmed by a sgRNA that may be specific to it, and another Cas9 isoform (e.g. N. meningitidis Cas9 or NmeCas9) to operate as a nuclease-dead Cas9 that provides DNA targeting to a binding site through its PAM specificity and orthogonal sgRNA. Other Cas9s include S. aureus Cas9 or SauCas9 and A. naeslundii Cas9 or AnaCas9.

The term “truncated” as used herein, when used in reference to either a polynucleotide sequence or an amino acid sequence means that at least a portion of the wild type sequence may be absent. In some cases, truncated guide sequences within the sgRNA or crRNA may improve the editing precision of Cas9. Fu, et al. “Improving CRISPR-Cas nuclease specificity using truncated guide RNAs” Nat Biotechnol. 2014 March; 32(3):279-284 (2014).

The term “base pairs” as used herein, refer to specific nucleobases (also termed nitrogenous bases), that are the building blocks of nucleotide sequences that form a primary structure of both DNA and RNA. Double-stranded DNA may be characterized by specific hydrogen bonding patterns. Base pairs may include, but are not limited to, guanine-cytosine and adenine-thymine base pairs.

The term “specific genomic target” as used herein, refers to any pre-determined nucleotide sequence capable of binding to a Cas9 protein contemplated herein. The target may include, but may be not limited to, a nucleotide sequence complementary to a programmable DNA binding domain or an orthogonal Cas9 protein programmed with its own guide RNA, a nucleotide sequence complementary to a single guide RNA, a protospacer adjacent motif recognition sequence, an on-target binding sequence and an off-target binding sequence.

The term “on-target binding sequence” as used herein, refers to a subsequence of a specific genomic target that may be completely complementary to a programmable DNA binding domain and/or a single guide RNA sequence.

The term “off-target binding sequence” as used herein, refers to a subsequence of a specific genomic target that may be partially complementary to a programmable DNA binding domain and/or a single guide RNA sequence.

The term “fails to bind” as used herein, refers to any nucleotide-nucleotide interaction or a nucleotide-amino acid interaction that exhibits partial complementarity, but has insufficient complementarity for recognition to trigger the cleavage of the target site by the Cas9 nuclease. Such binding failure may result in weak or partial binding of two molecules such that an expected biological function (e.g., nuclease activity) fails.

The term “cleavage” as used herein, may be defined as the generation of a break in the DNA. This could be either a single-stranded break or a double-stranded break depending on the type of nuclease that may be employed.

As used herein, the term “edit” “editing” or “edited” refers to a method of altering a nucleic acid sequence of a polynucleotide (e.g., for example, a wild type naturally occurring nucleic acid sequence or a mutated naturally occurring sequence) by selective deletion of a specific genomic target or the specific inclusion of new sequence through the use of an exogenously supplied DNA template. Such a specific genomic target includes, but may be not limited to, a chromosomal region, mitochondrial DNA, a gene, a promoter, an open reading frame or any nucleic acid sequence.

The term “delete”, “deleted”, “deleting” or “deletion” as used herein, may be defined as a change in either nucleotide or amino acid sequence in which one or more nucleotides or amino acid residues, respectively, are, or become, absent.

The term “gene of interest” as used herein, refers to any pre-determined gene for which deletion may be desired.

The term “allele” as used herein, refers to any one of a number of alternative forms of the same gene or same genetic locus.

The term “effective amount” as used herein, refers to a particular amount of a pharmaceutical composition comprising a therapeutic agent that achieves a clinically beneficial result (i.e., for example, a reduction of symptoms). Toxicity and therapeutic efficacy of such compositions can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index, and it can be expressed as the ratio LD₅₀/ED₅₀. Compounds that exhibit large therapeutic indices are preferred. The data obtained from these cell culture assays and additional animal studies can be used in formulating a range of dosage for human use. The dosage of such compounds lies preferably within a range of circulating concentrations that include the ED₅₀ with little or no toxicity. The dosage varies within this range depending upon the dosage form employed, sensitivity of the patient, and the route of administration.

The term “symptom”, as used herein, refers to any subjective or objective evidence of disease or physical disturbance observed by the patient. For example, subjective evidence is usually based upon patient self-reporting and may include, but is not limited to, pain, headache, visual disturbances, nausea and/or vomiting. Alternatively, objective evidence is usually a result of medical testing including, but not limited to, body temperature, complete blood count, lipid panels, thyroid panels, blood pressure, heart rate, electrocardiogram, tissue and/or body imaging scans.

The term “disease” or “medical condition”, as used herein, refers to any impairment of the normal state of the living animal or plant body or one of its parts that interrupts or modifies the performance of the vital functions. Typically manifested by distinguishing signs and symptoms, it is usually a response to: i) environmental factors (as malnutrition, industrial hazards, or climate); ii) specific infective agents (as worms, bacteria, or viruses); iii) inherent defects of the organism (as genetic anomalies); and/or iv) combinations of these factors.

The terms “reduce,” “inhibit,” “diminish,” “suppress,” “decrease,” “prevent” and grammatical equivalents (including “lower,” “smaller,” etc.) when in reference to the expression of any symptom in an untreated subject relative to a treated subject, mean that the quantity and/or magnitude of the symptoms in the treated subject is lower than in the untreated subject by any amount that is recognized as clinically relevant by any medically trained personnel. In one embodiment, the quantity and/or magnitude of the symptoms in the treated subject is at least 10% lower than, at least 25% lower than, at least 50% lower than, at least 75% lower than, and/or at least 90% lower than the quantity and/or magnitude of the symptoms in the untreated subject.

The term “attached” as used herein, refers to any interaction between a medium (or carrier) and a drug. Attachment may be reversible or irreversible. Such attachment includes, but is not limited to, covalent bonding, ionic bonding, Van der Waals forces or friction, and the like. A drug is attached to a medium (or carrier) if it is impregnated, incorporated, coated, in suspension with, in solution with, mixed with, etc.

The term “drug” or “compound” as used herein, refers to any pharmacologically active substance capable of being administered which achieves a desired effect. Drugs or compounds can be synthetic or naturally occurring, non-peptide, proteins or peptides, oligonucleotides or nucleotides, polysaccharides or sugars.

The term “administered” or “administering”, as used herein, refers to any method of providing a composition to a patient such that the composition has its intended effect on the patient. An exemplary method of administering is by a direct mechanism such as, local tissue administration (i.e., for example, extravascular placement), oral ingestion, transdermal patch, topical, inhalation, suppository etc.

The term “patient” or “subject”, as used herein, is a human or animal and need not be hospitalized. For example, out-patients, persons in nursing homes are “patients.” A patient may comprise any age of a human or non-human animal and therefore includes both adult and juveniles (i.e., children). It is not intended that the term “patient” connote a need for medical treatment, therefore, a patient may voluntarily or involuntarily be part of experimentation whether clinical or in support of basic science studies.

The term “affinity” as used herein, refers to any attractive force between substances or particles that causes them to enter into and remain in chemical combination. For example, an inhibitor compound that has a high affinity for a receptor will provide greater efficacy in preventing the receptor from interacting with its natural ligands, than an inhibitor with a low affinity.

The term “derived from” as used herein, refers to the source of a compound or sequence. In one respect, a compound or sequence may be derived from an organism or particular species. In another respect, a compound or sequence may be derived from a larger complex or sequence.

The term “protein” as used herein, refers to any of numerous naturally occurring extremely complex substances (as an enzyme or antibody) that consist of amino acid residues joined by peptide bonds, contain the elements carbon, hydrogen, nitrogen, oxygen, usually sulfur. In general, a protein comprises amino acids having an order of magnitude within the hundreds.

The term “peptide” as used herein, refers to any of various amides that are derived from two or more amino acids by combination of the amino group of one acid with the carboxyl group of another and are usually obtained by partial hydrolysis of proteins. In general, a peptide comprises amino acids having an order of magnitude with the tens.

The term “polypeptide”, refers to any of various amides that are derived from two or more amino acids by combination of the amino group of one acid with the carboxyl group of another and are usually obtained by partial hydrolysis of proteins. In general, a peptide comprises amino acids having an order of magnitude with the tens or larger.

The term “pharmaceutically” or “pharmacologically acceptable”, as used herein, refer to molecular entities and compositions that do not produce adverse, allergic, or other untoward reactions when administered to an animal or a human.

The term, “pharmaceutically acceptable carrier”, as used herein, includes any and all solvents, or a dispersion medium including, but not limited to, water, ethanol, polyol (for example, glycerol, propylene glycol, and liquid polyethylene glycol, and the like), suitable mixtures thereof, and vegetable oils, coatings, isotonic and absorption delaying agents, liposome, commercially available cleansers, and the like. Supplementary bioactive ingredients also can be incorporated into such carriers.

“Nucleic acid sequence” and “nucleotide sequence” as used herein refer to an oligonucleotide or polynucleotide, and fragments or portions thereof, and to DNA or RNA of genomic or synthetic origin which may be single- or double-stranded, and represent the sense or antisense strand.

The term “an isolated nucleic acid”, as used herein, refers to any nucleic acid molecule that has been removed from its natural state (e.g., removed from a cell and is, in a preferred embodiment, free of other genomic nucleic acid).

The terms “amino acid sequence” and “polypeptide sequence” as used herein, are interchangeable and to refer to a sequence of amino acids.

As used herein the term “portion” when in reference to a protein (as in “a portion of a given protein”) refers to fragments of that protein. The fragments may range in size from four amino acid residues to the entire amino acid sequence minus one amino acid.

The term “portion” when used in reference to a nucleotide sequence refers to fragments of that nucleotide sequence. The fragments may range in size from 5 nucleotide residues to the entire nucleotide sequence minus one nucleic acid residue.

The term “biologically active” refers to any molecule having structural, regulatory or biochemical functions. For example, biological activity may be determined, for example, by restoration of wild-type growth in cells lacking protein activity. Cells lacking protein activity may be produced by many methods (i.e., for example, point mutation and frame-shift mutation). Complementation is achieved by transfecting cells which lack protein activity with an expression vector which expresses the protein, a derivative thereof, or a portion thereof.

As used herein, the terms “complementary” or “complementarity” are used in reference to “polynucleotides” and “oligonucleotides” (which are interchangeable terms that refer to a sequence of nucleotides) related by the base-pairing rules. For example, the sequence “C-A-G-T,” is complementary to the sequence “G-T-C-A.” Complementarity can be “partial” or “total.” “Partial” complementarity is where one or more nucleic acid bases is not matched according to the base pairing rules. “Total” or “complete” complementarity between nucleic acids is where each and every nucleic acid base is matched with another base under the base pairing rules. The degree of complementarity between nucleic acid strands has significant effects on the efficiency and strength of hybridization between nucleic acid strands. This is of particular importance in amplification reactions, as well as detection methods which depend upon binding between nucleic acids.

As used herein, the term “hybridization” is used in reference to the pairing of complementary nucleic acids using any process by which a strand of nucleic acid joins with a complementary strand through base pairing to form a hybridization complex. Hybridization and the strength of hybridization (i.e., the strength of the association between the nucleic acids) is impacted by such factors as the degree of complementarity between the nucleic acids, stringency of the conditions involved, the Tₘ of the formed hybrid, and the G:C ratio within the nucleic acids.

As used herein the term “hybridization complex” refers to a complex formed between two nucleic acid sequences by virtue of the formation of hydrogen bounds between complementary G and C bases and between complementary A and T bases; these hydrogen bonds may be further stabilized by base stacking interactions. The two complementary nucleic acid sequences hydrogen bond in an antiparallel configuration. A hybridization complex may be formed in solution (e.g., C₀ t or R₀ t analysis) or between one nucleic acid sequence present in solution and another nucleic acid sequence immobilized to a solid support (e.g., a nylon membrane or a nitrocellulose filter as employed in Southern and Northern blotting, dot blotting or a glass slide as employed in in situ hybridization, including FISH (fluorescent in situ hybridization)).

Transcriptional control signals in eukaryotes comprise “promoter” and “enhancer” elements. Promoters and enhancers consist of short arrays of DNA sequences that interact specifically with cellular proteins involved in transcription. Maniatis, T. et al., Science 236:1237 (1987). Promoter and enhancer elements have been isolated from a variety of eukaryotic sources including genes in plant, yeast, insect and mammalian cells and viruses (analogous control elements, i.e., promoters, are also found in prokaryotes). The selection of a particular promoter and enhancer depends on what cell type is to be used to express the protein of interest.

The term “poly A site” or “poly A sequence” as used herein denotes a DNA sequence which directs both the termination and polyadenylation of the nascent RNA transcript. Efficient polyadenylation of the recombinant transcript is desirable as transcripts lacking a poly A tail are unstable and are rapidly degraded. The poly A signal utilized in an expression vector may be “heterologous” or “endogenous.” An endogenous poly A signal is one that is found naturally at the 3′ end of the coding region of a given gene in the genome. A heterologous poly A signal is one which is isolated from one gene and placed 3′ of another gene. Efficient expression of recombinant DNA sequences in eukaryotic cells involves expression of signals directing the efficient termination and polyadenylation of the resulting transcript. Transcription termination signals are generally found downstream of the polyadenylation signal and are a few hundred nucleotides in length.

The term “transfection” or “transfected” refers to the introduction of foreign DNA into a cell.

As used herein, the terms “nucleic acid molecule encoding”, “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

As used herein, the term “coding region” when used in reference to a structural gene refers to the nucleotide sequences which encode the amino acids found in the nascent polypeptide as a result of translation of a mRNA molecule. The coding region is bounded, in eukaryotes, on the 5′ side by the nucleotide triplet “ATG” which encodes the initiator methionine and on the 3′ side by one of the three triplets which specify stop codons (i.e., TAA, TAG, TGA).

As used herein, the term “structural gene” refers to a DNA sequence coding for RNA or a protein. In contrast, “regulatory genes” are structural genes which encode products which control the expression of other genes (e.g., transcription factors).

As used herein, the term “gene” means the deoxyribonucleotide sequences comprising the coding region of a structural gene and including sequences located adjacent to the coding region on both the 5′ and 3′ ends for a distance of about 1 kb on either end such that the gene corresponds to the length of the full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene which are transcribed into heterogeneous nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

In addition to containing introns, genomic forms of a gene may also include sequences located on both the 5′ and 3′ end of the sequences which are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers which control or influence the transcription of the gene. The 3′ flanking region may contain sequences which direct the termination of transcription, posttranscriptional cleavage and polyadenylation.

The term “viral vector” encompasses any nucleic acid construct derived from a virus genome capable of incorporating heterologous nucleic acid sequences for expression in a host organism. For example, such viral vectors may include, but are not limited to, adeno-associated viral vectors, lentiviral vectors, SV40 viral vectors, retroviral vectors, adenoviral vectors. Although viral vectors are occasionally created from pathogenic viruses, they may be modified in such a way as to minimize their overall health risk. This usually involves the deletion of a part of the viral genome involved with viral replication. Such a virus can efficiently infect cells but, once the infection has taken place, the virus may require a helper virus to provide the missing proteins for production of new virions. Preferably, viral vectors should have a minimal effect on the physiology of the cell it infects and exhibit genetically stable properties (e.g., do not undergo spontaneous genome rearrangement). Most viral vectors are engineered to infect as wide a range of cell types as possible. Even so, a viral receptor can be modified to target the virus to a specific kind of cell. Viruses modified in this manner are said to be pseudotyped. Viral vectors are often engineered to incorporate certain genes that help identify which cells took up the viral genes. These genes are called marker genes. For example, a common marker gene confers antibiotic resistance to a certain antibiotic.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1 presents representative sequence of a conventional, full-length, 145 nt Nme1Cas9 and Nme2Cas9 sgRNA.

FIG. 2 presents exemplary Nme1Cas9 sgRNA sequences and associated gene editing activity having a truncated repeat:anti-repeat region or a truncated Stem 2 region. Deletion/truncation series of Nme1Cas9 sgRNAs. Top: aligned sequences, color-coded as in FIG. 1. Bottom: T7E1 assays of editing at Nme1Cas9 target site 7 (NTS7), using the indicated sgRNAs as guides.

FIG. 3 presents exemplary Nme1Cas9 sgRNA sequences and associated gene editing activity having a truncated repeat:anti-repeat region or a truncated Stem 2 region. The shortest Nme1Cas9 sgRNAs (#10-101 nt; 24 nt guide sequence; and #11-100 nt; 23 nt guide sequence) efficiently edit three distinct target sites (NTS7, NTS27, and NTS55) in the human genome. Top: sequences of wild-type and minimized sgRNAs, using the same color scheme as in the previous figures. Bottom: T7E1 assays of editing efficiency at the three target sites in HEK293T cells.

FIG. 4A-D presents exemplary sequences (as secondary structures) of Nme1Cas9 wt sgRNA, and truncated sgRNAs 11 and 12 and associated gene editing by RNP delivery of Nme1Cas9 and sgRNAs. Three genomic sites (N-TS72, N-TS55 and N-TS40), and one traffic light reporter site was targeted in the human genome using HEK293T cells. Top: sequences shown as secondary structures of wild-type and minimized sgRNAs. Bottom: Editing efficiencies measured by T7E1 assay or flow cytometry are depicted as bar graphs.

FIG. 5 presents gene editing in PLB985 cells using minimized sgRNA 11, and in vitro transcribed wt sgRNA. Cells were transfected with RNP complexes of Nme1Cas9 and sgRNAs and gene editing at genomic site N-TS72 measured by TIDE.

FIG. 6A-C presents a schematic of one embodiment of an AAV vector comprising a complete CRISPR/Cas9 gene editing complex. Representative sequences of the various AAV vector regions are color coded in Appendix 1.

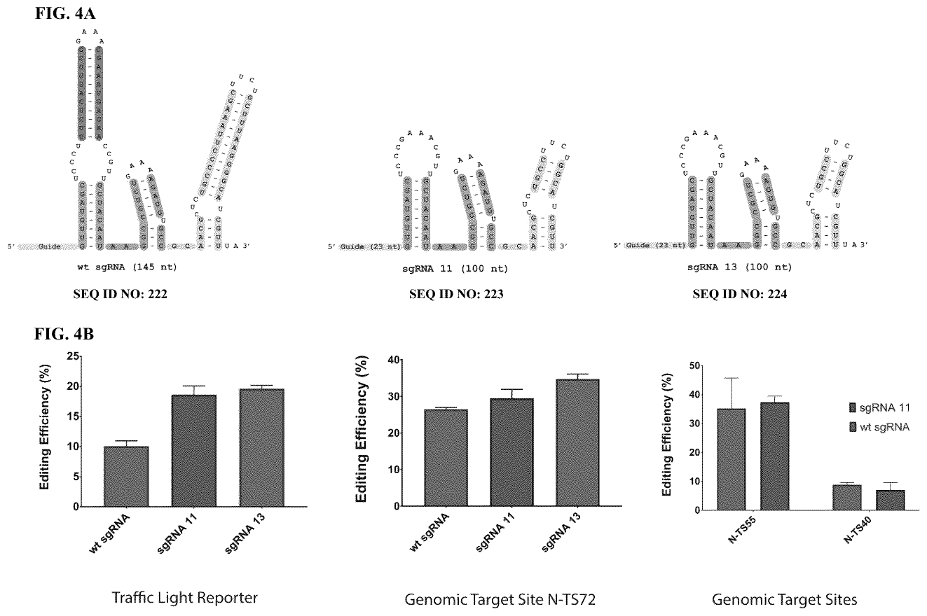

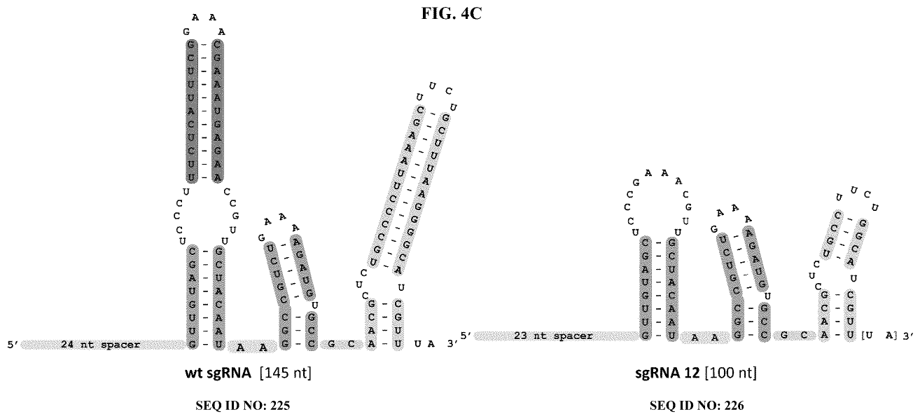

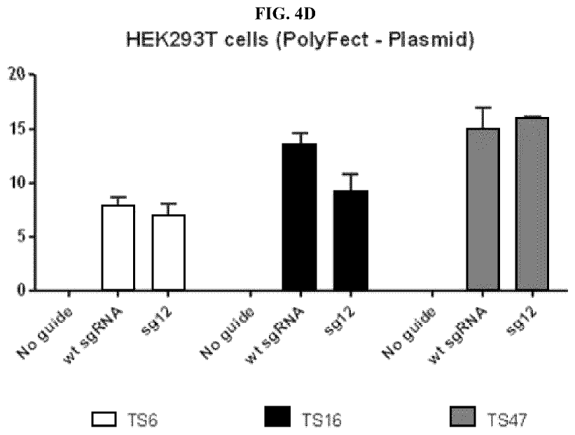

FIG. 7 presents one embodiment of a color-coded sequence of Nme single-guide RNA and a promoter as depicted in FIG. 4, wherein the backbone is linearized using SapI to insert a 24-nt target spacer.
U6 promoter: Turquoise.
Nme single guide RNA: Purple
SapI restriction sites: Bold

FIG. 8 presents one embodiment of a color-coded sequence of an Nme1Cas9 and promoter as depicted in FIG. 4, wherein Start and Stop codons underlined in bold.
U1a promoter: Blue
Kozak sequence: Grey
Humanized Nme1Cas9: Red
SV40 NLS: Green
Nucleoplasmin (NP) NLS: Yellow
HA Tags (3×): Bold Orange
Synthetic NLS: Turquoise
Beta-globin polyadenylation signal: Teal

FIG. 9 presents exemplary data showing editing efficiency of various target sites using AAV plasmids with sgRNA-Nme1Cas9 constructs guided to either the Pcsk9 gene or the Rosa26 gene (control).

FIG. 10 presents one embodiment of color-coded target site sequences for sgRNA-Nme1Cas9 constructs guided to either a Pcsk9 gene or a Rosa26 gene (control).
24-nt Nme1Cas9 target spacer, blue bold
Nme1Cas9 PAM underlined [NNNNGATT)
T7E1 primers binding sites: green italics
TIDE primers binding sites: purple italics

FIG. 11 presents exemplary data showing gene editing efficiency following in vivo hydrodynamic injection by mouse tail vein of 30 μg of endotoxin-free sgRNA-Nme1Cas9-AAV plasmid targeting Pcsk9.

FIG. 12A presents exemplary data showing gene editing efficiency in the liver at the Pcsk9 gene and the Rosa26 gene by Nme1-Cas9 vector packaged in hepatocyte-specific AAV8 serotype, at a dose of 4×10¹¹ genomic copies (gc) per mouse 14 days post vector administration. FIG. 12B presents exemplary data showing gene editing efficiency in the liver at a Pcsk9 gene and a Rosa26 gene by an Nme1-Cas9 vector packaged in hepatocyte-specific AAV8 serotype, at a dose of 4×10¹¹ genomic copies (gc) per mouse 50 days post vector administration.

FIG. 13 presents exemplary data showing reduction in mouse cholesterol levels following injection of sgRNA-Cas9-AAV vectors targeting a Pcsk9 gene, a Rosa26 gene and a PBS control group at 0, 25 and 50 days.

FIGS. 14A and 14B present exemplary data showing a genome-wide unbiased identification of double strand breaks (DSBs) enabled by sequencing (e.g., GUIDE-Seq) assay that searched for off-target editing sites for both the Pcsk9-sgRNA-Cas9-AAV (FIG. 14A) and the Rosa26-sgRNA-Cas9-AAV (FIG. 14B).

FIG. 15 presents exemplary data showing a targeted TIDE analyses in mice 14 days post-injection of both the Pcsk9-sgRNA-Cas9-AAV and the Rosa26-sgRNA-Cas9-AAV that revealed minimal cleavage. OnT, on-target site; OT1, OT2 etc.: off-target sites.

FIG. 16 presents exemplary data showing a hematoxylin and eosin stain assay in the liver sections of mice sacrificed at day 14 subsequent to injection of vectors targeting a Pcsk9 gene and a Rosa26 gene. No evidence for a host immune response is observed.

FIG. 17 illustrates one embodiment of an in vitro PAM library identification workflow. NGS, next-generation sequencing.

FIG. 18 presents putative sequence from an in vitro PAM discovery assay depicted in FIG. 17. Recombinantly purified Cas9 from each bacterium was incubated with an sgRNA and a target with randomized PAM. Nme1Cas9 was used as a control.

FIG. 19 presents exemplary data showing percent genome editing at a single site (top panel) in the human genome in HEK293T cells. Percentages show estimated indel formation using a T7E1 endonuclease assay (Nme2Cas9, HpaCas9) or a fluorescent assay (for SmuCas9) based on the “traffic light” reporter integrated into the genome of HEK293T cells.

FIG. 20 presents exemplary data showing genome editing in HEK293T cells of an integrated traffic light reporter with Nme2Cas9 targeting various protospacers with various PAMs (X-axis). The results suggest a preferred NNNNCC PAM for Nme2Cas9 in human cells.

FIG. 21 presents exemplary data showing genome editing in HEK293T cells in the presence of various anti-CRISPR (Acr) proteins. T7E1 digestion shows genome editing following plasmid transfection (to express Nme2Cas9 and its sgRNA) or RNA/protein delivery (HpaCas9 and its sgRNA). Nme2Cas9 is robustly inhibited by two Acr proteins (AcrIIC3_{Nme} and AcrIIC4_{Hpa}), while HpaCas9 is inhibited by four of the previously reported type II-C Acrs. These results show that these two Cas9 proteins are subject to off-switch control by anti-CRISPRs.

FIG. 22 presents exemplary data of traffic light reporter (TLR) gene editing using the Nme2Cas9-sgRNA complex on “CC” dinucleotide PAMs. FIG. 22. Black bars are the % of cells that exhibit fluorescence, whereas gray bars indicate % editing more accurately based on sequencing (“TIDE analysis”).

FIG. 23 presents exemplary data of gene editing by Nme2Cas9 using T7E1 assays at the AAVS1, Chromosome 14 NTS4, VEGF and CFTR loci.

FIG. 24 presents one embodiment for a wild type Nme2Cas9 bacterial open reading frame DNA sequence.

FIG. 25 presents one embodiment of a wild type Nme2Cas9 bacterial protein sequence.

FIG. 26 presents one embodiment of an Nme2Cas9 human-codon-optimized open reading frame DNA sequence. Yellow—SV40 NLS; Green—3×-HA-Tag; Blue: cMyc-like NLS.

FIG. 27 presents one embodiment of an Nme2Cas9 humanized protein sequence. Yellow—SV40 NLS; Green—3×-HA-Tag; Blue: cMyc-like NLS.

FIG. 28 presents one embodiment of an HpaCas9 bacterial protein sequence.

FIG. 29 presents one embodiment of an SmuCas9 native bacterial open reading frame DNA sequence.

FIG. 30 presents one embodiment of an SmuCas9 bacterial protein sequence.

FIG. 31 presents one embodiment of an SmuCas9 Human-codon-optimized open reading frame DNA sequence. Yellow—SV40 NLS; Green—3×-HA-Tag; Blue: cMyc-like NLS.

FIG. 32 presents one embodiment of an SmuCas9 humanized protein sequence. Yellow—SV40 NLS; Green—3×-HA-Tag; Blue: cMyc-like NLS.

FIG. 33 presents exemplary Type-II C Cas9 ortholog single guide RNA sequences compatible with short C-rich PAMs. Yellow—crRNA; Gray—Linker; Purple—tracrRNA.

FIG. 34A-E illustrates three closely related Neisseria meningitidis Cas9 orthologs that have distinct PAMs.

FIG. 34A: Schematic showing mutated residues (orange spheres) between Nme2Cas9 (left) and Nme3Cas9 (right) mapped onto the predicted structure of Nme1Cas9, revealing the cluster of mutations in the PID (black).

FIG. 34B: Experimental workflow of the in vitro PAM discovery assay with a 10 nt randomized PAM sequence downstream of a protospacer. Adapters were ligated to cleaved product and sequenced.

FIG. 34C: Sequence logos of the in vitro PAM discovery assay demonstrating an N₄GATT PAM for Nme1Cas9, as shown previously in cells.

FIG. 34D: Sequence logos showing Nme1Cas9 with its PID swapped with those of Nme2Cas9 (left) and Nme3Cas9 (right) recognize a C at position 5. The remaining nucleotides were determined with lower confidence due to the modest cleavage efficiency of the protein chimeras (FIG. 35C).

FIG. 34E: Sequence logo illustrating that full-length Nme2Cas9 recognizes an N₄CC PAM based on the PAM discovery assay with a fixed C at position 5, and PAM nts 1-4 and 6-8 randomized.

FIG. 35A-D shows a characterization of Neisseria meningitidis Cas9 orthologs with rapidly-evolving PIDs in accordance with FIG. 34.

FIG. 35A: Unrooted phylogenetic tree of NmeCas9 orthologs that are >80% identical to Nme1Cas9. Three distinct branches emerged, with the majority of mutations clustered in the PID. Group 1 (blue) PIDs with >98% identity to Nme1Cas9, group 2 (orange) with PIDs˜52% identical to Nme1Cas9, and group 3 (green) with PIDs˜86% identical to Nme1Cas9. Three representative Cas9 orthologs from each group (Nme1Cas9, Nme2Cas9 and Nme3Cas9) are marked.

FIG. 35B: Schematic showing the CRISPR loci of the strains encoding the three Cas9 orthologs (Nme1Cas9, Nme2Cas9, and Nme3Cas9) from (FIG. 35A). Percent identities of each CRISPR-Cas component to N. meningitidis 8013 (encoding Nme1Cas9) are shown.

FIG. 35C: Number of reads from cleaved DNAs from the in vitro assays for intact Nme1Cas9, and for chimeras with Nme1Cas9's PID swapped with those of Nme2Cas9 and Nme3Cas9. The reduced read counts indicate lower cleavage efficiencies in the chimeras.

FIG. 35D: Sequence logos from the in vitro PAM discovery assay on an NNNNCNNN randomized PAM by Nme1Cas9 with its PID swapped with those of Nme2Cas9 (left) or Nme3Cas9 (right).

FIG. 36A-D shows that the Nme2Cas9 uses a 22-24 nucleotide spacer to recognize and edit sites adjacent to an N₄CC PAM. All experiments were done in triplicate, and error bars represent standard error of mean (s.e.m.).

FIG. 36A: Schematic showing the transient transfection workflow on HEK293T TLR2.0 cells. Nme2Cas9 and sgRNA plasmids were transfected and mCherry+ cells were detected 72 hours after transfection.

FIG. 36B: Using Nme2Cas9 to target an array of PAMs in TLR2.0. All sites with N₄CC PAMs were targeted with varying degrees of efficiency, while no Nme2Cas9 targeting observed at an N₄GATT PAM or in the absence of sgRNA. SpyCas9 (targeting NGG) and Nme1Cas9 (targeting N₄GATT) were used as positive controls.

FIG. 36C: The effect of spacer length on the efficiency of Nme2Cas9 editing. An sgRNA targeting a TLR2.0 site (with an N₄CCA PAM) with spacer lengths varying from 24 to 20 nts (including a 5′-terminal G), showing highest editing efficiencies with 22-24 nucleotide spacers.

FIG. 36D: Nme2Cas9 nickases (HNH nickase=Nme2Cas9^{D16A}; RuvC nickase=Nme2Cas9^{H588A}) can be used in tandem to generate indels in TLR2.0. Targets with cleavage sites 32 base pairs and 64 base pairs apart were targeted using either nickase to generate indels. The HNH nickase shows efficient editing, particularly when the cleavage sites were close (32 bp). Wildtype Nme2Cas9 was used as a control. Green is GFP (HDR) and red is mCherry (NHEJ).

FIG. 37A-D presents exemplary data regarding PAM, spacer, and seed elements for Nme2Cas9 targeting in mammalian cells, in accordance with FIG. 36A-D. All experiments were done in triplicate and error bars represent s.e.m.

FIG. 37A: Nme2Cas9 targeting at N₄CD sites in TLR2.0. Four sites for each non-C nucleotide at the tested position (N₄CA, N₄CT and N₄CG) were examined, and an N₄CC site was used as a positive control.

FIG. 37B: Nme2Cas9 targeting at N₄DC sites in TLR2.0 [similar to (FIG. 37A)].

FIG. 37C: Guide truncations on another TLR2.0 site, revealing similar length requirements as those observed in FIG. 36C.

FIG. 37D: Nme2Cas9 targeting efficiency is differentially sensitive to single-nucleotide mismatches in the seed sequence. Data show the effects of walking single-nucleotide mismatches in the sgRNA along the 23-nt spacer in a TLR target site.

FIG. 38A-C presents exemplary data showing Nme2Cas9 genome editing efficiency at genomic loci in mammalian cells via multiple delivery methods. All results represent 3 independent biological replicates, and error bars represent s.e.m.

FIG. 38A: Nme2Cas9 genome editing using transient transfections with sgRNAs targeting loci throughout the human genome in HEK293T cells. 14 sites were selected based the initial screening of 38 sites to demonstrate the range of indels (as detected by TIDE) at different loci induced by Nme2Cas9. An Nme1Cas9 target site (with an N₄GATT PAM) was used as a negative control.

FIG. 38B: Left panel: Transient transfection of an all-in-one plasmid (Nme2Cas9+sgRNA) targeting the Pcsk9 and Rosa26 loci in Hepa1-6 mouse cells, as detected by TIDE. Right panel: Electroporation of sgRNA plasmids into K562 cells stably expressing Nme2Cas9 from a lentivector results in efficient indel formation at the intended loci.

FIG. 38C: Nme2Cas9 can be electroporated as an RNP complex for efficient genome editing. 40 picomoles Cas9 along with 50 picomoles of in vitro transcribed sgRNAs targeting three different loci were electroporated into HEK293T cells. Indels were measured using TIDE after 72 h.

FIG. 39A-B presents exemplary data showing dose dependence and block deletions by Nme2Cas9, in accordance with FIG. 38A-C.

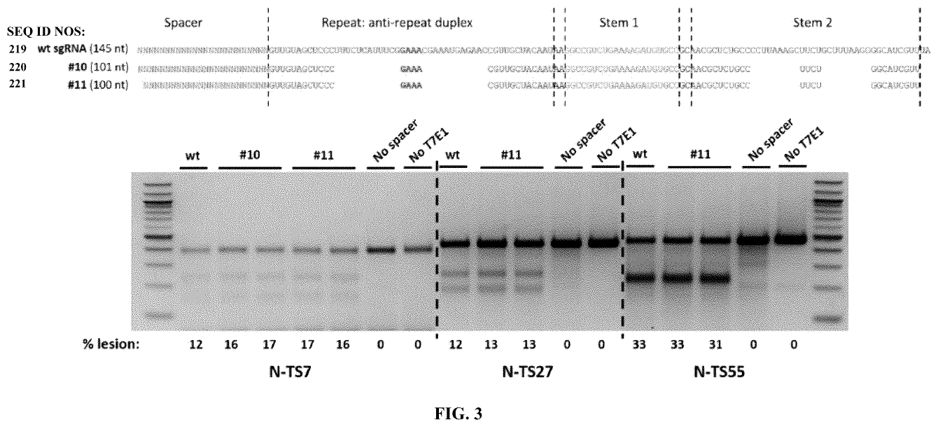

FIG. 39A: Increasing the dose of electroporated Nme2Cas9 plasmid (500 ng, vs. 200 ng in FIG. 3A) improves editing efficiency at two sites (TS16 and TS6).

FIG. 39B: Nme2Cas9 can be used to create block deletions. Two TLR2.0 targets with cleavage sites 32 bp apart were targeted simultaneously with Nme2Cas9. The majority of lesions created were exactly 32 bp deletions (green).

FIG. 40A-C presents exemplary data showing that Type II-C Anti-CRISPR proteins can be used to inhibit Nme2Cas9 gene editing activity (e.g., as an off-switch) in vitro and in vivo. All experiments were done in triplicate and error bars represent s.e.m.

FIG. 40A: In vitro cleavage assay of Nme1Cas9 and Nme2Cas9 in the presence of five previously characterized anti-CRISPR proteins (10:1 ratio of Acr:Cas9). Top: Nme1Cas9 efficiently cleaves a fragment containing a protospacer with an N₄GATT PAM in the absence of an Acr or in the presence of a control Acr (AcrE2). All other previously characterized Acrs inhibited Nme1Cas9, as expected. Bottom: Nme2Cas9 efficiently cleaves a target containing a protospacer with an N₄CC PAM in the presence of AcrE2 and AcrIIC5_{Smu}, suggesting that AcrIIC5_{Smu} is unable to inhibit Nme2Cas9 at a 10:1 molar ratio.

FIG. 40B: Genome editing in the presence of the five previously described anti-CRISPR proteins. Plasmids expressing Nme2Cas9, sgRNA and each respective Acr (200 ng Cas9, 100 ng sgRNA, 200 ng Acr) were co-transfected into HEK293T cells, and genome editing was measured using TIDE 72 hr post transfection. Except for AcrE2 and AcrIIC5_{Smu}, all other Acrs inhibited genome editing, albeit at different efficiencies.

FIG. 40C: Acr inhibition of Nme2Cas9 is dose-dependent with distinct apparent potencies. AcrIIC1_{Nme} and AcrIIC4_{Hpa} inhibit Nme2Cas9 completely at 2:1 and 1:1 ratios of cotransfected plasmids, respectively.

FIG. 41 presents exemplary data showing that a Nme2Cas9 PID swap renders Nme1Cas9 insensitive to AcrIIC5_{Smu} inhibition, in accordance with FIG. 40A-C. In vitro cleavage by the Nme1Cas9-Nme2Cas9PID chimera was performed in the presence of previously characterized Acr proteins (10 uM Cas9-sgRNA+100 uM Acr).

FIG. 42A-F presents exemplary data showing that Nme2Cas9 has no detectable off-targets in mammalian cells.

FIG. 42A: Schematic showing the dual sites (DS) targetable by both SpyCas9 and Nme2Cas9 by virtue of their non-overlapping PAMs. The Nme2Cas9 PAM (orange) and SpyCas9 PAM (blue) are highlighted.

FIG. 42B: Nme2Cas9 and SpyCas9 induce indels at dual sites. Six dual sites in VEGFA with GN₃GN₁₉NGGNCC sequences (SEQ ID NO: 206) were selected for direct comparisons between the two orthologs. Plasmids expressing each Cas9 (with same promoter and NLSs) were transfected along with each ortholog's cognate guide in HEK293T cells. Indel rates were determined by TIDE 72 hrs post transfection. Nme2Cas9 editing was detectable at all six sites and was more efficient than SpyCas9 on two sites (DS2 and 6). SpyCas9 edited four out of six sites (DS1, 2, 4 and 6), with two sites showing significantly higher editing rates than Nme2Cas9 (DS1 and 4). DS2, 4 and 6 were selected for GUIDE-Seq analysis as Nme2Cas9 was equally efficient, less efficient and more efficient than SpyCas9 at these sites, respectively.

FIG. 42C: Nme2Cas9 has a clean off-target profile in human cells. Numbers of off-target sites detected by GUIDE-Seq for each nuclease at individual target sites are shown. SpyCas9 off-target numbers are shown in black. In addition to dual sites, TS6 (because of its high efficiency and potential for off-targets) and two mouse sites (to test accuracy in another cell type) also showed zero or one off-target site per guide.

FIG. 42D: Targeted deep sequencing confirms the high Nme2Cas9 accuracy indicated by GUIDE-seq. Top off-target loci detected by GUIDE-seq were amplified and deep-sequenced. SpyCas9 showed off-targeting at most loci, while for Nme2Cas9, only one (the Rosa26 site) showed editing at the off-target locus at relatively low levels (˜40% on-target vs ˜1% off-target). Note the log scale on the y axis.

FIG. 42E: Nme2Cas9 and SpyCas9 efficiencies vary based on the locus and target site. Sites throughout the genome (with GN₃GN₁₉NGGNCC sequences) (SEQ ID NO: 206) were selected for direct comparisons of editing by the two orthologs. Plasmids expressing each Cas9 (with the same promoter, linkers, tags and NLSs) and its cognate guide were transfected into HEK293T cells. Indel efficiencies were determined by TIDE 72 hrs post-transfection. Box-and-whisker plots indicate editing efficiencies at twenty-eight (28) dual sites by Nme2Cas9 and SpyCas9 (left). The sites that showed no editing were excluded from the analysis. Relative efficiencies of Nme2Cas9 and SpyCas9 show that Nme2Cas9 is less efficient than SpyCas9 (right), on average. Editing efficiencies by both Cas9 orthologs at all twenty-eight (28) sites were included in the analysis of relative efficiencies in the right panel.

FIG. 42F presents nucleic acids sequences for the validated off-target site of the Rosa26 guide, showing the PAM region (underlined), the consensus CC PAM dinucleotide (bold), and three mismatches in the PAM-distal portion of the spacer (red).

FIG. 43A-E presents exemplary data showing the orthogonality and relative accuracy of Nme2Cas9 and SpyCas9 at dual target sites, in accordance with FIG. 42A-F.

FIG. 43A: Nme2Cas9 and SpyCas9 guides are orthogonal. TIDE results show the frequencies of indels created by both nucleases targeting DS12 with either their cognate sgRNAs, or with the sgRNAs of the other ortholog.

FIG. 43B: Nme2Cas9 and SpyCas9 exhibit comparable on-target editing efficiencies during GUIDE-seq. Bars indicate on-target read counts from GUIDE-Seq at the three dual sites targeted by each ortholog. Orange bars represent Nme2Cas9 and black bars represent SpyCas9.

FIG. 43C: SpyCas9's on-target vs. off-target reads for each site. Orange bars represent the on-target reads while black bars represent off-targets.

FIG. 43D: Nme2Cas9's on-target vs off-target reads for each site.

FIG. 43E: Bar graphs showing TIDE at expected off-target sites based on CRISPRseek, detecting no indels at off-target loci.

FIG. 44A-D presents exemplary data showing Nme2Cas9 genome editing in vivo via all-in-one AAV delivery.

FIG. 44A: Workflow for delivery of AAV8.Nme2Cas9+sgRNA to lower cholesterol levels in mice by targeting Pcsk9. Top: schematic of the all-in-one AAV vector expressing Nme2Cas9 and the sgRNA. Bottom: Timeline for AAV8.Nme2Cas9+sgRNA tail-vein injections, followed by cholesterol measurements at day 14 and indel, histology and cholesterol analyses at day 28.

FIG. 44B: Deep sequencing analysis to measure indels in DNA extracted from livers of mice injected with AAV8.Nme2Cas9+sgRNA targeting Pcsk9 and Rosa26 (control) loci.

FIG. 44C: Reduced serum cholesterol levels in mice injected with the Pcsk9-targeting guide compared to the Rosa26-targeting controls. P values are calculated by unpaired T-test.

FIG. 44D: H&E staining from livers of mice injected with AAV8.Nme2Cas9+sgRosa26 (left) or AAV8.Nme2Cas9+sgPcsk9 (right) vectors. Scale bar, 25 um.

FIG. 45 presents one embodiment of minimized AAV backbone and exemplary comparative TLR 2.0 data to the conventional sized AAV backbone.

FIG. 46 presents a comparison of Nme2Cas9 structures of truncated sgRNA 11 with truncated sgRNA 12.

FIG. 47 illustrates one embodiment of a minimized all-in-one AAV with a short polyA signal.

FIG. 48A-H illustrate embodiments of a minimized all-in-one AAV backbone.

FIG. 48A: Dual proximal sgRNAs in tandem.

FIG. 48B: Single proximal sgRNA and a distal donor template for homology directed repair.

FIG. 48C: Single proximal sgRNA and a distal donor template for homology directed repair.

FIG. 48D: Single proximal sgRNA and a proximal donor template for homology directed repair.

FIG. 48E: Dual proximal and distal sgRNAs.

FIG. 48F: Dual proximal and distal stRNAs.

FIG. 48G: Dual proximal sgRNAs in tandem.

FIG. 48H: Dual proximal sgRNAs in tandem.

FIG. 49A-D presents a validation of an all-in-one AAV-sgRNA-hNme1Cas9 construct.

FIG. 49A: Schematic representation of a single rAAV vector expressing human-codon optimized Nme1Cas9 and its sgRNA. The backbone is flanked by AAV inverted terminal repeats (ITR). The poly(a) signal is from rabbit beta-globin (BGH).

FIG. 49B: Schematic diagram of the Pcsk9 (top) and Rosa26 (bottom) mouse genes. Red bars represent exons. Zoomed-in views show the protospacer sequence (red) whereas the Nme1Cas9 PAM sequence is highlighted in green. Double-stranded break location sites are denoted (black arrowheads).

FIG. 49C: Stacked histogram showing a representative percentage distribution of insertions-deletions (indels) obtained by TIDE after AAV-sgRNA-hNme1Cas9 plasmid transfections in Hepa1-6 cells targeting Pcsk9 (sgPcsk9) and Rosa26 (sgRosa26) genes. Data are presented as mean values±SD from three biological replicates.

FIG. 49D: Stacked histogram showing a representative percentage distribution of indels at Pcsk9 in the liver of C57Bl/6 mice obtained by TIDE after hydrodynamic injection of AAV-sgRNA-hNme1Cas9 plasmid.

FIG. 50 presents exemplary data showing that many N₄GN₃ PAMs are inactive, and revealed no off-target sites with fewer than four mismatches in the mouse genome.

FIG. 51A-D presents exemplary data showing that Nme1Cas9-mediated knockout of Hpd rescues the lethal phenotype in hereditary tyrosinemia Type I mice.

FIG. 51A: Schematic diagram of the Hpd mouse gene. Red bars represent exons. Zoomed-in views show the protospacer sequences (red) for targeting exon 8 (sgHpd1) and exon 11 (sgHpd2). Nme1Cas9 PAM sequences are in green and double-stranded break locations are indicated (black arrowheads).

FIG. 51B: Experimental design. Three groups of Hereditary Tyrosinemia Type I Fah^{-/-} mice are injected with PBS or all-in-one AAV-sgRNA-hNme1Cas9 plasmids sgHpd1 or sgHpd2.

FIG. 51C: Weight of mice hydrodynamically injected with PBS (green), AAV-sgRNA-hNme1Cas9 plasmid sgHpd1 targeting Hpd exon 8 (red) or sgHpd2-targeting Hpd exon 11 (blue) were monitored after NTBC withdrawal. Error bars represent three mice for PBS and sgHpd1 groups and two mice for the sgHpd2 group. Data are presented as mean±SD.

FIG. 51D: Stacked histogram showing a representative percentage distribution of indels at Hpd in liver of Fah^{-/-} mice obtained by TIDE after hydrodynamic injection of PBS or sgHpd1 and sgHpd2 plasmids. Livers were harvested at the end of NTBC withdrawal (day 43).

FIG. 52 presents exemplary data showing average indel efficiencies of the guides presented in FIG. 51A-D.

FIG. 53 presents exemplary histological photomicrographs showing that liver damage is substantially less severe in the sgHpd1- and sgHpd2-treated mice compared to Fah^{mut/mut} mice injected with PBS, as indicated by the smaller numbers of multinucleated hepatocytes compared to PBS-injected mice.

FIG. 54A-D presents AAV-delivery of Nme1Cas9 for in vivo genome editing.

FIG. 54A: Experimental outline of AAV8-sgRNA-hNme1Cas9 vector tail-vein injections to target Pcsk9 (sgPcsk9) and Rosa26 (sgRosa26) in C57Bl/6 mice. Mice were sacrificed at 4 (n=1) or 50 days (n=5) post injection and liver tissues were harvested. Blood sera were collected at days 0, 25, and 50 post injection for cholesterol level measurement.

FIG. 54B: Serum cholesterol levels. p values are calculated by unpaired t test.

FIG. 54C: Stacked histogram showing a representative percentage distribution of indels at Pcsk9 or Rosa26 in livers of mice, as measured by targeted deep-sequencing analyses. Data are presented as mean±SD from five mice per cohort.

FIG. 54D: A representative anti-PCSK9 western blot using total protein collected from day 50 mouse liver homogenates. A total of 2 ng of recombinant mouse PCSK9 (r-PCSK9) was included as a mobility standard. The asterisk indicates a cross-reacting protein that is larger than the control recombinant protein.

FIG. 55A-B presents exemplary data showing that mice injected with AAV8-sgRNA-hNme1Cas9 generate anti-Nme1Cas9 antibodies.

FIG. 56A-C presents exemplary data showing GUIDE-seq genome-wide specificities of Nme1Cas9. Data are presented as mean±SD.

FIG. 56A: Number of GUIDE-seq reads for the on-target (OnT) and off-target (OT) sites.

FIG. 56B: Targeted deep sequencing to measure the lesion rates at each of the OT sites in Hepa1-6 cells. The mismatches of each OT site with the OnT protospacers is highlighted (blue). Data are presented as mean±SD from three biological replicates.

FIG. 56C: Targeted deep sequencing to measure the lesion rates at each of the OT sites using genomic DNA obtained from mice injected with all-in-one AAV8-sgRNA-hNme1Cas9sgPcsk9 and sgRosa26 and sacrificed at day 14 (D14) or day 50 (D50) post injection.

FIG. 57A-C presents exemplary data for Tyrosinase (Tyr) gene editing ex vivo by Nme2Cas9 in mouse zygotes, as related to FIG. 58A-C.

FIG. 57A: Two sites in Tyr gene, each with N₄CC PAMs, were tested for editing in Hepa1-6 cells. The sgTyr2 guide exhibited higher editing efficiency and was selected for further testing.

FIG. 57B: Seven mice survived post-natal development, and each exhibited coat color phenotypes as well as on-target editing, as assayed by TIDE.

FIG. 57C: Indel spectra from tail DNA of each mouse from FIG. 57B, as well as an unedited C57BL/6NJ mouse, as indicated by TIDE analysis. Efficiencies of insertions (positive) and deletions (negative) of various sizes are indicated.

FIG. 58A-C presents exemplary data of ex vivo Nme2Cas9 genome editing using an all-in-one AAV delivery.

FIG. 58A: Workflow for single-AAV Nme2Cas9 editing ex vivo to generate albino C57BL/6NJ mice by targeting the Tyr gene. Zygotes are cultured in KSOM containing AAV6.Nme2Cas9:sgTyr for 5-6 hours, rinsed in M2, and cultured for a day before being transferred to the oviduct of pseudo-pregnant recipients.

FIG. 58B: Albino (left) and chinchilla or variegated (middle) mice generated by 3×109 GCs, and chinchilla or variegated mice (right) generated by 3×108 GCs of zygotes with AAV6.Nme2Cas9:sgTyr.

FIG. 58C: Summary of Nme2Cas9.sgTyr single-AAV ex vivo Tyr editing experiments at two AAV doses.

FIG. 59 shows an alignment of Nme1Cas9 and Nme2Cas9 nucleotide sequences. Legend: Non-PID aa differences (turquoise shading); PID aa differences (yellow shading); active site residues (red letters).

FIG. 60 shows an alignment of Nme1Cas9 and Nme3Cas9 nucleotide sequences. Legend: Non-PID aa differences (turquoise shading); PID aa differences (yellow shading); active site residues (red letters).

FIG. 61 shows one embodiment of an Nme2Cas9 amino acid sequence. Legend: SV40 NLS (yellow shading); 3×-HA-Tag (green shading); cMyc-like NLS (turquoise shading); Linker (purple shading).

FIG. 62 shows one embodiment of an Nme2Cas9 amino acid sequence. Legend: SV40 NLS (yellow shading); 3×-HA-Tag (green shading); Nucleoplasmin-like NLS (red shading); c-myc NLS (turquoise shading); Linker (purple shading).

FIG. 63 shows one embodiment of a recombinant Nme2Cas9 (rNme2Cas9) amino acid sequence. Legend: SV40 NLS (yellow shading); Nucleoplasmin-like NLS (red shading); Linker (purple shading).

FIG. 64 shows one embodiment of an all-in-one AAV-sgRNA-hNmeCas9 plasmid Nucleotide sequence. Legend: sgRNA scaffold (brown letters); GUIDE sequence (black letters); U6 promoter (blue letters); U1a promoter (purple letters): NLS NLS (green letters); hNmeCas9 (red letters); NLS 3×-HA and NLS BGH-pA (alternating green/black letters).

### DETAILED DESCRIPTION OF THE INVENTION

The present invention is related to compositions and methods for gene therapy. Several approaches described herein utilize the Neisseria meningitidis Cas9 system that provides a hyperaccurate CRISPR gene editing platform. Furthermore, the invention incorporates improvements of this Cas9 system: for example, truncating the single guide RNA sequences, and the packing of -Nme1Cas9 or Nme2Cas9 with its guide RNA in an adeno-associated viral vector that is compatible for in vivo administration. Furthermore, Type II-C Cas9 orthologs have been identified that target protospacer adjacent motif sequences limited to between one-four required nucleotides.

### I. Neisseria meningitidis Cas9 (Nme1Cas9)/CRISPR Gene Editing Accuracy

Previously, a hyper-accurate version of type II-C CRISPR-Cas9 systems called Neisseria meningitidis Cas9 (Nme1Cas9) was reported. In addition to being hyper-accurate, Nme1Cas9 is also smaller than the widely used Streptococcus pyogenes Cas9 (SpyCas9), allowing Nme1Cas9 to be delivered more readily via viral and messenger RNA (mRNA)-based methods. Genome editing with Nme1Cas9 typically has been accomplished using plasmid transfections. Zhang et al., “Processing-independent CRISPR RNAs limit natural transformation in Neisseria meningitidis” Mol Cell 50:488-503 (2013); Hou et al., “Efficient genome engineering in human pluripotent stem cells using Cas9 from Neisseria meningitidis” Procd Natl Acad Sci U.S.A. 110:15644-15649 (2013); Esvelt et al., “Orthogonal Cas9 proteins for RNA-guided gene regulation and editing” Nature Methods 10:1116-1121 (2013); Zhang et al., “DNase H activity of Neisseria meningitidis Cas9” Mol Cell 60:242-255 (2015); Lee et al., “The Neisseria meningitidis CRISPR-Cas9 system enables specific genome editing in mammalian cells” Molecular Therapy 24:645-654 (2016); Pawluk et al., “Naturally occurring off-switches for CRISPR-Cas9” Cell 167:1829-1838 (2016); and Amrani et al., “Nme1Cas9 is an intrinsically high-fidelity genome editing platform” biorxiv.org/content/early/2017/08/04/172650 (2017).

However, Nme1Cas9 viral, RNA- and ribonucleoproteins (RNP)-based delivery has not been extensively explored. RNA- and RNP-based delivery of Cas9 orthologs for genome engineering holds several advantages over other delivery methods. They not only result in faster editing since they bypass the expression issues related to DNA-based delivery of Cas9 and its sgRNA, but they also reduce off-target effects associated with Cas9-based editing. Reduced off-target activity results from finer control of the Cas9 RNA and RNP concentrations, and from relatively rapid Cas9 RNA and RNP degradation in cells. Prolonged presence of active Cas9 within the cell has been shown to be associated with higher off-target effects. Since Cas9 RNAs and RNPs are more rapidly degraded within cells, Cas9 delivered as RNA or RNP does not persist for long periods of time and consequently have reduced off-target effects.

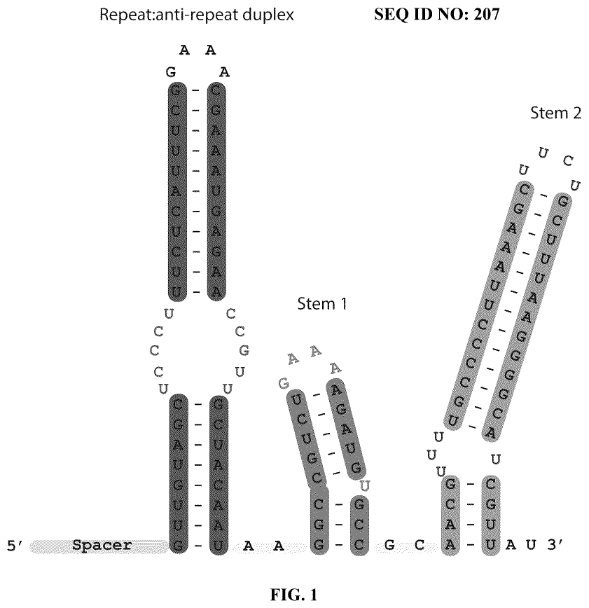

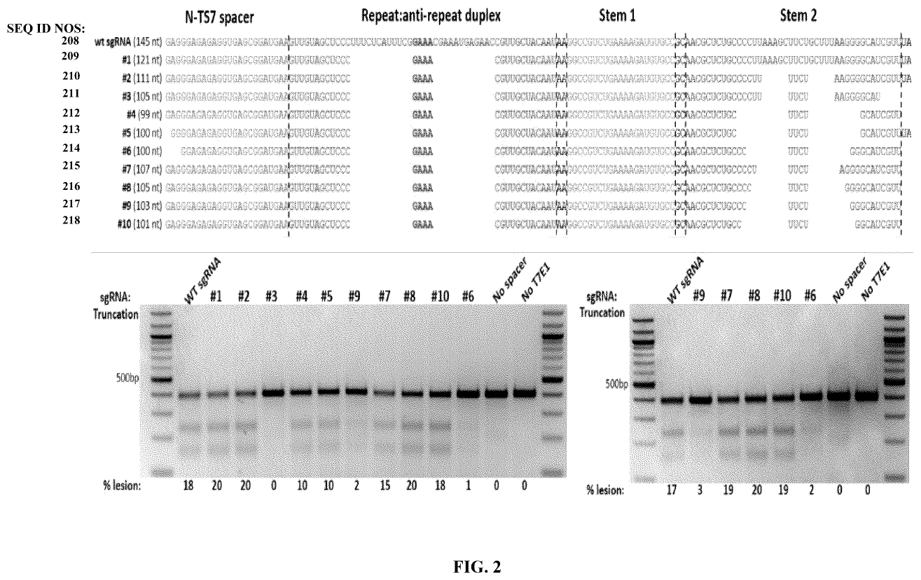

Conventionally used full-length 145 nt Nme1Cas9 sgRNA includes a 48 nucleotide (nt) crRNA, a 4 nt linker, and a 93 nt tracrRNA. The crRNA region of the sgRNA is composed of a first 24 nt spacer sequence, and a second 24 nt repeat sequence that pairs with a 24 nt tracrRNA anti-repeat 5′ region thereby forming a repeat:anti-repeat region. The remaining 69 nt tracrRNA region includes the Stem 1 region and Stem 2 region. FIG. 1.

This full-length Nme1Cas9 sgRNA has been successfully used for genome editing using plasmid-based methods. Furthermore, in vitro transcribed Nme1Cas9 sgRNA can be complexed with purified Nme1Cas9 and used for genome editing in human cells. While genome editing of human cells has been successful with in vitro transcribed sgRNAs, the editing efficiency of an Nme1Cas9 RNP is reduced in harder-to-transfect human cell lines such as PLB985.

It has previously been shown that the editing efficiency of Cas9 RNPs is proportional to the chemical stability their sgRNAs. Although it is not necessary to understand the mechanism of an invention, it is believed that several cellular mechanisms are employed to rapidly degrade RNAs. For this reason, Cas9 sgRNAs are routinely modified by chemical means. Some of the chemical modifications that confer increased stability to sgRNA include, but are not limited to, ribose 2′-O-methylation and/or phosphorothioate linkages. While chemically modified RNAs are options for improved genome editing by Cas9 RNPs, their effectiveness is limited by the fact that chemical synthesis of RNAs becomes increasingly difficult and expensive as the length of RNA increases. At 145 nt, Nme1Cas9 sgRNA synthesis is out of reach for routine genome editing applications that employ chemically synthesized sgRNAs.

### II. Truncated Nme1Cas9 sgRNA Sequences

Due to the above identified limitation that a full-length 145 nt Nme1Cas9 sgRNA is too large for routine chemical synthesis of sgRNAs for genome editing, one embodiment of the present invention contemplates a truncated Nme1Cas9 sgRNA. Although it is not necessary to understand the mechanism of an invention, it is believed that a truncated Nme1Cas-sgRNA does not compromise the function of an Nme1Cas9 RNP. Furthermore, sgRNAs for Nme1Cas9 and Nme2Cas9 are identical and interchangeable (FIG. 35B), so sgRNA truncations are equally applicable to both Nme1Cas9 and Nme2Cas9. Exemplary sequences of truncated sgRNAs and associated target sites are disclosed below, where variable sgRNA nts in guide regions are given as “N” residues. In the target sequences, the 24 nts recognized by the sgRNA guide region are underlined, and the protospacer adjacent motif (PAM) region is given in bold. Table 1.

TABLE 1
Exemplary Truncated sgRNA Sequences And Associated Genomic Targets

| SEQ ID | | |
|---|---|---|
| NO: | Description | Sequence |
| 1 | wt sgRNA | NNNNNNNNNNNNNNNNNNNNNNNNGUUGUAGCUCCCUUUCUCAUUUCGGAA |
| | | ACGAAAUGAGAACCGUUGCUACAAUAAGGCCGUCUGAAAAGAUGUGCCGCA |
| | | ACGCUCUGCCCCUUAAAGCUUCUGCUUUAAGGGGCAUCGUUUA |
| 2 | sgRNA #1 | NNNNNNNNNNNNNNNNNNNNNNNNGUUGUAGCUCCCGAAACGUUGCUACAA |
| | | UAAGGCCGUCUGAAAAGAUGUGCCGCAACGCUCUGCCCCUUAAAGCUUCUG |
| | | CUUUAAGGGGCAUCGUUUA |
| 3 | sgRNA #2 | NNNNNNNNNNNNNNNNNNNNNNNNGUUGUAGCUCCCGAAACGUUGCUACAA |
| | | UAAGGCCGUCUGAAAAGAUGUGCCGCAACGCUCUGCCCCUUUUCUAAGGGG |
| | | CAUCGUUUA |
| 4 | sgRNA #3 | NNNNNNNNNNNNNNNNNNNNNNNNGUUGUAGCUCCCGAAACGUUGCUACAA |
| | | UAAGGCCGUCUGAAAAGAUGUGCCGCAACGCUCUGCCCCUUUUCUAAGGGG |
| | | CAU |
| 5 | sgRNA #4 | NNNNNNNNNNNNNNNNNNNNNNNNGUUGUAGCUCCCGAAACGUUGCUACAA |
| | | UAAGGCCGUCUGAAAAGAUGUGCCGCAACGCUCUGCUUCUGCAUCGUU |
| 6 | sgRNA #5 | NNNNNNNNNNNNNNNNNNNNNNNGUUGUAGCUCCCGAAACGUUGCUACAAU |
| | | AAGGCCGUCUGAAAAGAUGUGCCGCAACGCUCUGCUUCUGCAUCGUUUA |
| 7 | sgRNA #6 | NNNNNNNNNNNNNNNNNNNNNGUUGUAGCUCCCGAAACGUUGCUACAAUAA |
| | | GGCCGUCUGAAAAGAUGUGCCGCAACGCUCUGCCCUUCUGGGCAUCGUU |
| 8 | sgRNA #7 | NNNNNNNNNNNNNNNNNNNNNNNNGUUGUAGCUCCCGAAACGUUGCUACAA |
| | | UAAGGCCGUCUGAAAAGAUGUGCCGCAACGCUCUGCCCCUUUCUAGGGGCA |
| | | UCGUU |
| 9 | sgRNA #8 | NNNNNNNNNNNNNNNNNNNNNNNNGUUGUAGCUCCCGAAACGUUGCUACAA |
| | | UAAGGCCGUCUGAAAAGAUGUGCCGCAACGCUCUGCCCCUUCUGGGGCAUC |
| | | GUU |
| 10 | sgRNA #9 | NNNNNNNNNNNNNNNNNNNNNNNNGUUGUAGCUCCCGAAACGUUGCUACAA |
| | | UAAGGCCGUCUGAAAAGAUGUGCCGCAACGCUCUGCCCUUCUGGGCAUCGUU |
| 11 | sgRNA #10 | NNNNNNNNNNNNNNNNNNNNNNNNGUUGUAGCUCCCGAAACGUUGCUACAA |
| | | UAAGGCCGUCUGAAAAGAUGUGCCGCAACGCUCUGCCUUCUGGCAUCGUU |
| 12 | sgRNA #11 | NNNNNNNNNNNNNNNNNNNNNNNGUUGUAGCUCCCGAAACGUUGCUACAAU |
| | | AAGGCCGUCUGAAAAGAUGUGCCGCAACGCUCUGCCUUCUGGCAUCGUU |
| 13 | N-TS7 Spacer (24 nt) | GAGGGAGAGAGGUGAGCGGAUGAA |
| 14 | N-TS7 Spacer (23 nt) | GGGGAGAGAGGUGAGCGGAUGAA |
| 15 | N-TS27 Spacer (24 nt) | GUUCUCCAAGCCCUCGGACCUCGU |
| 16 | N-TS27 Spacer (23 nt) | GUCUCCAAGCCCUCGGACCUCGU |
| 17 | N-TS55 Spacer (24 nt) | GCUGGAUUACUGUGUGGUAGAGGG |
| 18 | N-TS55 Spacer (23 nt) | GUGGAUUACUGUGUGGUAGAGGG |
| 19 | N-TS7 Genomic | AGCTTGAGCAAAGGGAGAGAGGTGAGCGGATGAAGGGAGATTGGTGAGTAT |
| | Target Site | C |
| 20 | N-TS27 Genomic | CGCTTCGCGGCTTCTCCAAGCCCTCGGACCTCGTGGGCGTCTTCTCCTGCG |
| | Target Site | T |
| 21 | N-TS55 Genomic | GAATTCACTAGCTGGATTACTGTGTGGTAGAGGGAGGTGATTAGCACCTGT |
| | Target Site | G |

As contemplated herein, a truncated Nme1Cas9 sgRNA would not only allow synthesis at a reasonable cost, but also facilitates use in virus-based delivery methods (e.g., for example adeno-associated viral delivery platforms) where the allowed length of DNA is limited. In one embodiment, the truncated sgRNA reduces off-target Nme1Cas9 editing effect. In one embodiment, the truncated Nme1Cas9 sgRNA comprises at least one chemical modification that increases Nme1Cas9 editing efficiency.

As discussed above, the full length 145 nt sgRNA of Nme1Cas9 includes a guide region, a repeat:anti-repeat duplex region, a Stem 1 region and a Stem 2 region. FIG. 1. However, because the length of the sgRNA is problematic for routine genomic editing, and it was highly desirable to develop a truncated sgRNA for Nme1Cas9. Currently, commercially available RNA synthesis methods require that RNA end product be not more than ˜100 nt.

In one embodiment, the present invention contemplates an Nme1Cas9 sgRNA comprising a truncated repeat:anti-repeat duplex. In one embodiment, the present invention contemplates an Nme1Cas9 sgRNA comprising a truncated stem 2. FIG. 2. Furthermore, it has previously been shown that a 5′ variable guide crRNA region (e.g., spacer region) of Nme1Cas9 can also be truncated by a few nucleotides without loss of function. Amrani et al., “Nme1Cas9 is an intrinsically high-fidelity genome editing platform” biorxiv.org/content/early/2017/08/04/172650(2017); and Lee et al., “The Neisseria meningitidis CRISPR-Cas9 system enables specific genome editing in mammalian cells” Molecular Therapy 24:645-654 (2016).

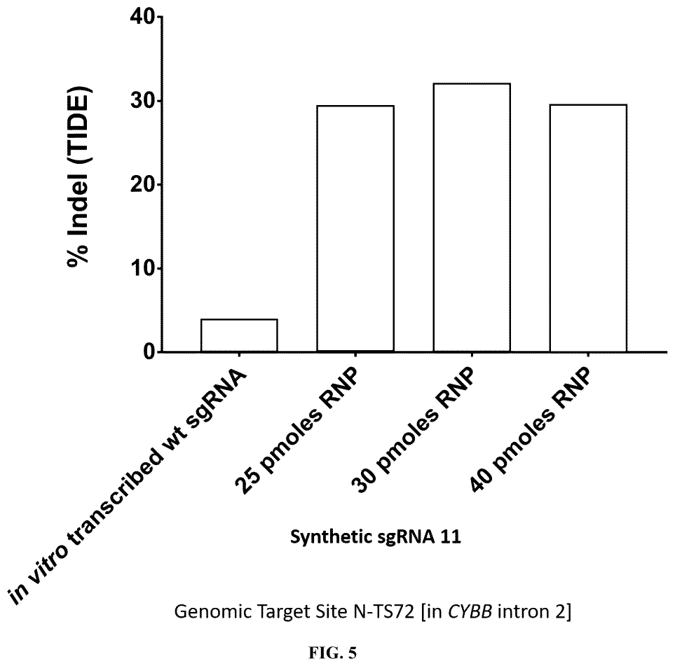

In one embodiment, the present invention contemplates a 100 nt Nme1Cas9-truncated sgRNA. FIG. 3, Construct #11. This 100 nt Nme1Cas9 truncated-sgRNA Construct #11 was tested on three different human genomic sites by transient transfections in HEK293T cells, and at all three sites they support Nme1Cas9 function at the same level as, if not better than, the full-length Nme1Cas9 sgRNA. FIG. 3, Bottom Panel. Moreover, sgRNA 11 and sgRNA 13 were also tested at several genomic target sites using RNP delivery and editing efficiency was similar or higher than the wt sgRNA. FIG. 4A-E. The synthetic version of construct #11 was also tested in PLB985 cells resulting in higher editing efficiency relative to in vitro transcribed wt sgRNA. FIG. 5.

### III. Associated-Adenovirus CRISPR Delivery Platforms

Compared to transcription activator-like effector nucleases (TALENs) and Zinc-finger nucleases (ZFNs), Cas9s are distinguished by their flexibility and versatility. Komor et al., “CRISPR-based technologies for the manipulation of eukaryotic genomes” Cell 2017; 168:20-36. Such characteristics make them ideal for driving the field of genome engineering forward. Over the past few years, CRISPR-Cas9 has been used to enhance products in agriculture, food, and industry, in addition to the promising applications in gene therapy and personalized medicine. Barrangou et al., “Applications of CRISPR technologies in research and beyond” Nat Biotechnol. 2016; 34:933-41. Despite the diversity of Class 2 CRISPR systems that have been described, only a handful of them have been developed and validated for genome editing in vivo. As shown herein, NmeCas9 is a compact, high-fidelity Cas9 that can be considered for future in vivo genome editing applications using all-in-one rAAV. NmeCas9's unique PAM enables editing at additional targets that are inaccessible to the other two compact, all-in-one rAAV-validated orthologs (SauCas9 and CjeCas9).

Genome editing using a bacterial CRISPR system has opened a new avenue for human gene therapy. Named for Clustered Regularly Interspaced Short Palindromic Repeats that capture snippets of invasive nucleic acids in bacteria, the CRISPR complex comprises a guide RNA (e.g., sgRNA) that directs a nuclease Cas9 (CRISPR-associated protein 9) to cleave complementary double-stranded DNA. Non-homologous repair of a Cas9-induced DNA break leads to small insertions or deletions (indels) that inactivate target genes, but breaks can also be repaired by homologous DNA templates resulting in gene replacement. Nelson et al., “In vivo genome editing improves muscle function in a mouse model of Duchenne muscular dystrophy” Science 351: 403-407 (2016); and Ran et al., “In vivo genome editing using Staphylococcus aureus Cas9” Nature 520:186-191 (2015); and Yin et al., “Genome editing with Cas9 in adult mice corrects a disease mutation and phenotype” Nature Biotechnology 32:551-553 (2014).

The current and widely-used Type II-A Streptococcus pyogenes (Spy) Cas9 as a flexible genome-editing tool demonstrates several disadvantages: i) inefficient delivery; ii) off-target cleavage; and iii) unregulated activity. These disadvantages strictly limit SpyCas9 as a potential gene therapy tool. As discussed herein a highly accurate and precise Nme1Cas9 or Nme2Cas9 complex can overcome these SpyCas9 limitations.

Nme1Cas9 and Nme2Cas9 have been shown herein to be an efficient genome-editing platform in mammalian cells and, as a smaller protein than SpyCas9, it is easier to engineer viral vectors for in vivo delivery. Furthermore, Nme1Cas9 and Nme2Cas9 have significantly lower off-target editing than SpyCas9 and anti-CRISPR proteins have been identified that allow control of Nme1Cas9 and Nme2Cas9 activity. Esvelt et al., “Orthogonal Cas9 proteins for RNA-guided gene regulation and editing” Nature Methods 10:1116-1121 (2013); Amrani et al., “Nme1Cas9 is an intrinsically high-fidelity genome editing platform” biorxiv.org/content/early/2017/08/04/172650 (2017); Lee et al., ‘The Neisseria meningitidis CRISPR-Cas9 System Enables Specific Genome Editing in Mammalian Cells” Molecular Therapy 24:645-654 (2016); Hou et al., “‘Efficient genome engineering in human pluripotent stem cells using Cas9 from Neisseria meningitidis” Procd Natl Acad Sci USA 110:15644-15649 (2013); and Pawluk et al., “Naturally Occurring Off-Switches for CRISPR-Cas9” Cell 167:1829-38 e9 (2016); and FIG. 21.

Adeno-Associated Virus (AAV) has been demonstrated as a delivery shuttle with minimal pathogenicity in pre-clinical and clinical settings, but it has a limited packaging capacity. Nme1Cas9, encoded by a ˜3.3 kb open reading frame, and its guide RNAs are within the packaging limit of AAV. Nme2Cas9 has similar advantages. Unlike SpyCas9, which requires delivery by separate vectors for the sgRNA and Cas9, Nme1Cas9, Nme2Cas9 and their sgRNA are small enough to be delivered with a single AAV vector.

Other Cas9 orthologs have been successfully delivered in vivo by AAV, such as Campylobacter jejuni Cas9 (CjeCas9) and Staphylococcus aureus (SauCas9). Kim et al., “In vivo genome editing with a small Cas9 orthologue derived from Campylobacter jejuni” Nat Commun 8:14500 (2017); and Ran et al., “In vivo genome editing using Staphylococcus aureus Cas9” Nature 520:186-191 (2015). Nme1Cas9 is usually associated with an N₄GATT PAM, which is unlike the CjeCas9 PAM (e.g., N₄RYAC), or the SauCas9 PAM (e.g., NNGRRT) (R=purine (A or G), Y=pyrimidine (C or T)).

Nme1Cas9 has been successfully delivered as a ribonucleoprotein (RNP) complex in human cells. FIG. 2 and FIG. 3. Further, the data presented herein show that an Nme1Cas9 nucleic acid sequence can be expressed in vivo in mice to target genes using an all-in-one sgRNA-Nme1Cas9-AAV vector subsequent to a tail vein injection.

The data presented herein demonstrates a targeting of a mouse Proprotein Convertase Subtilisin/Kexin type 9 (Pcsk9) gene. PCSK9 functions as an antagonist to the low-density lipoprotein (LDL) receptor and limits LDL cholesterol uptake. Detection of reduced cholesterol levels in the serum can thereby provide a direct functional readout of efficient Nme1Cas9 editing using a PCSK9-directed Cas9 platform.

In one embodiment, the present invention contemplates an adeno-associated viral vector comprising an Nme1Cas9-sgRNA complex or an Nme2Cas9-sgRNA complex. Although it is not necessary to understand the mechanism of an invention, it is believed that an AAV/Nme1Cas9-sgRNA complex or an AAV/Nme2Cas9-sgRNA complex are compatible with an in vivo delivery route in order to provide gene editing.

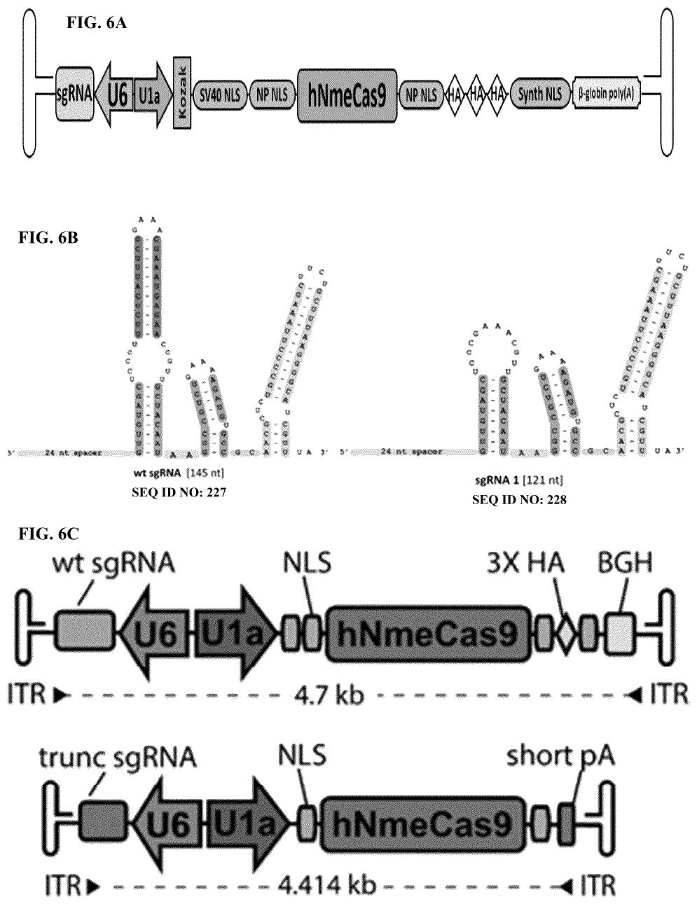

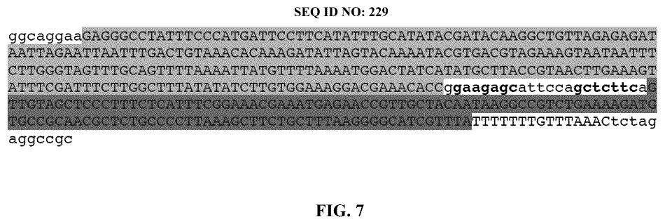

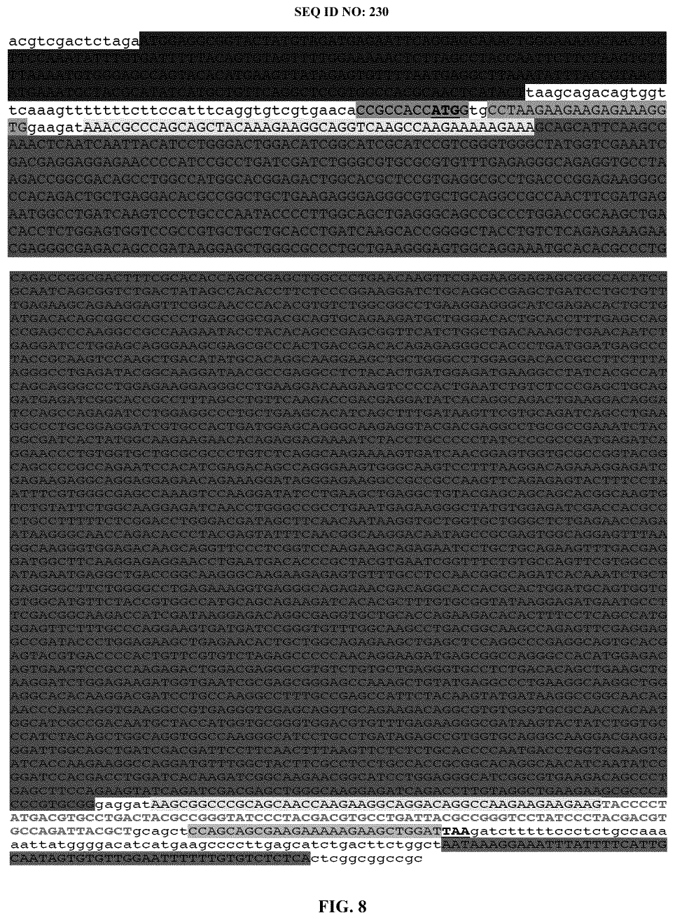

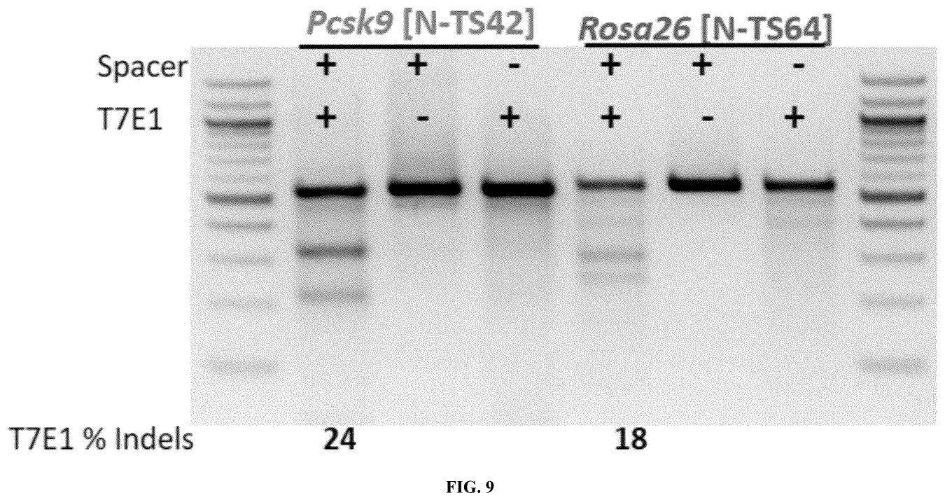

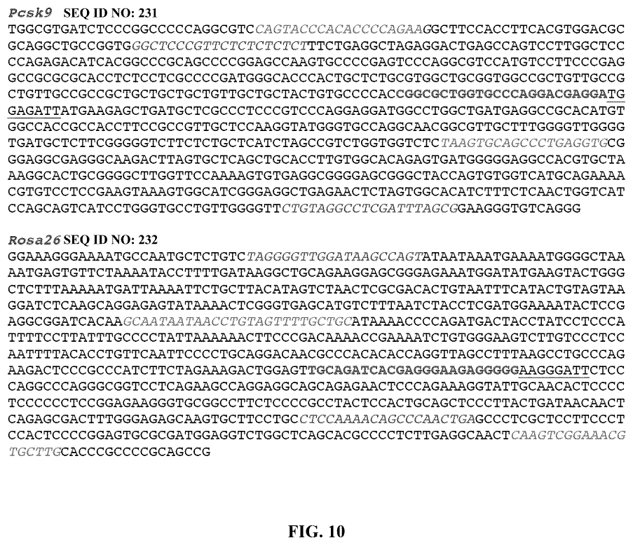

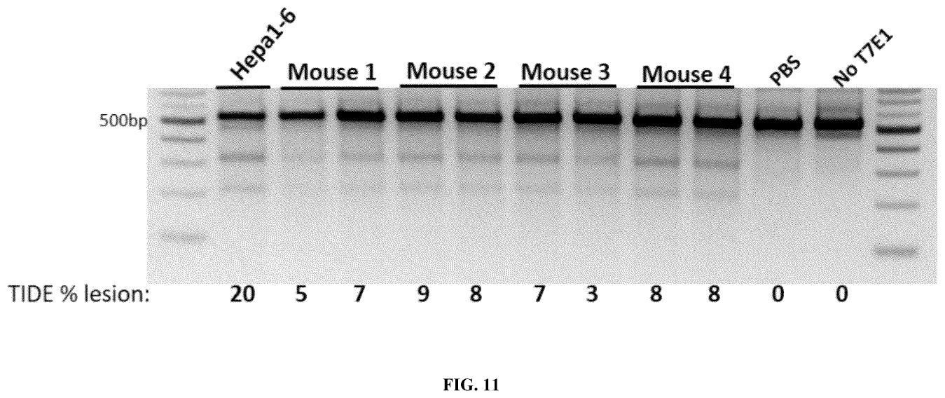

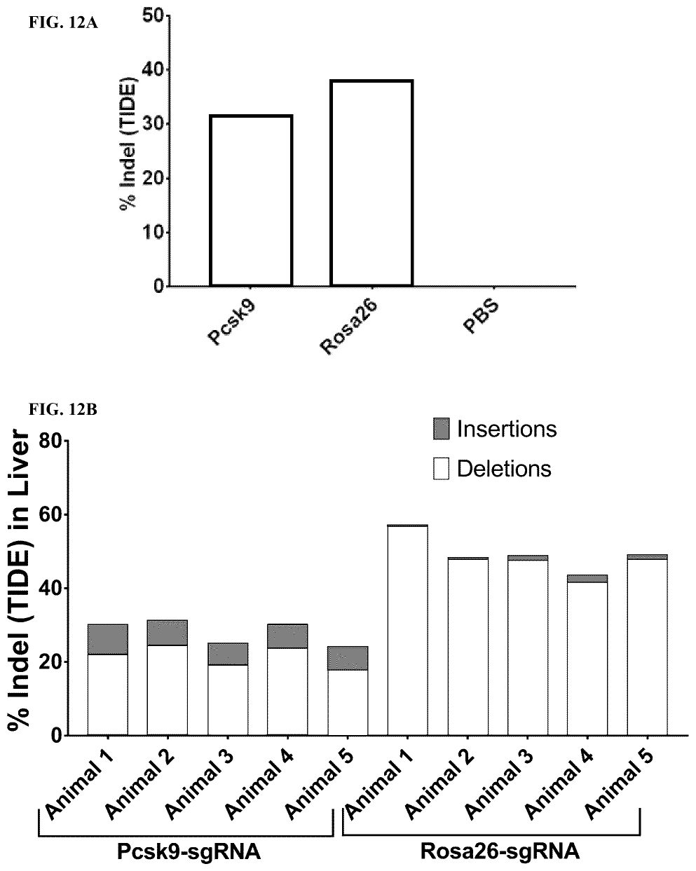

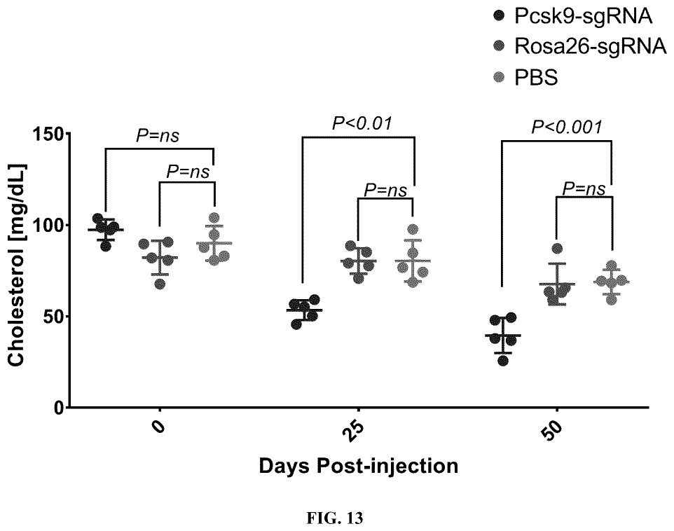

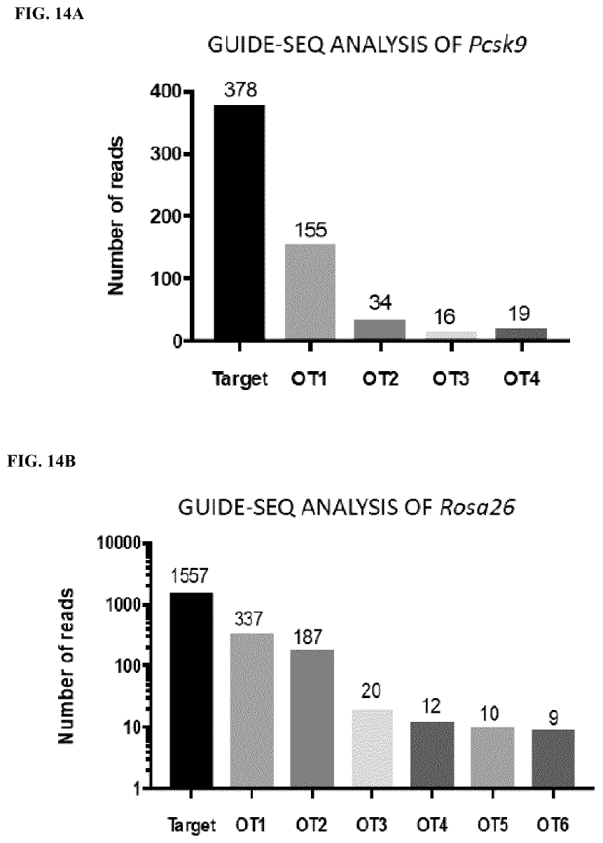

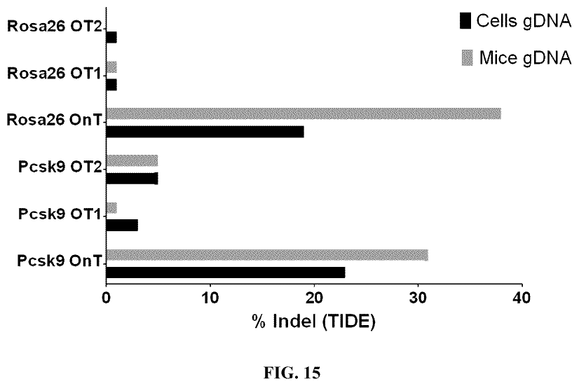

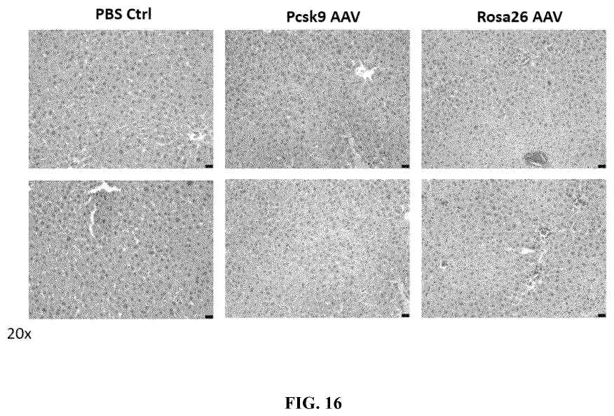

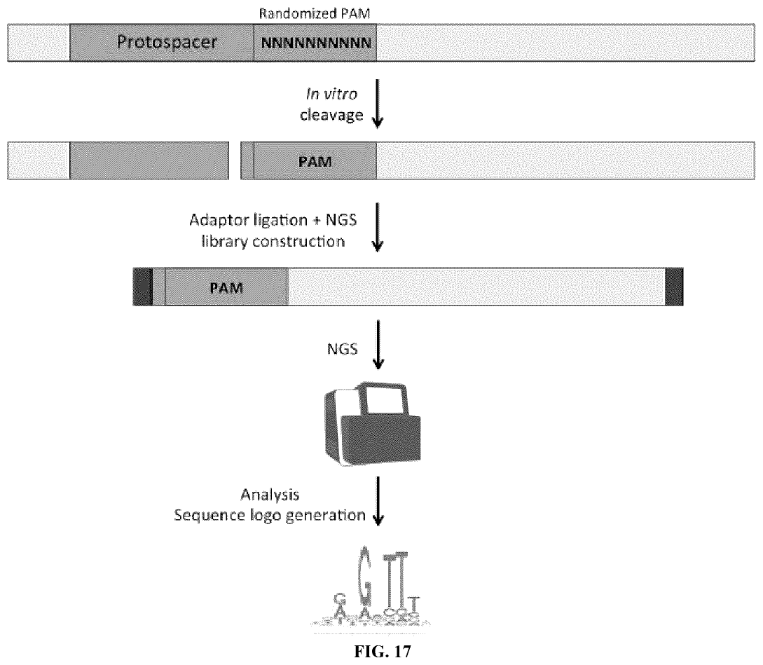

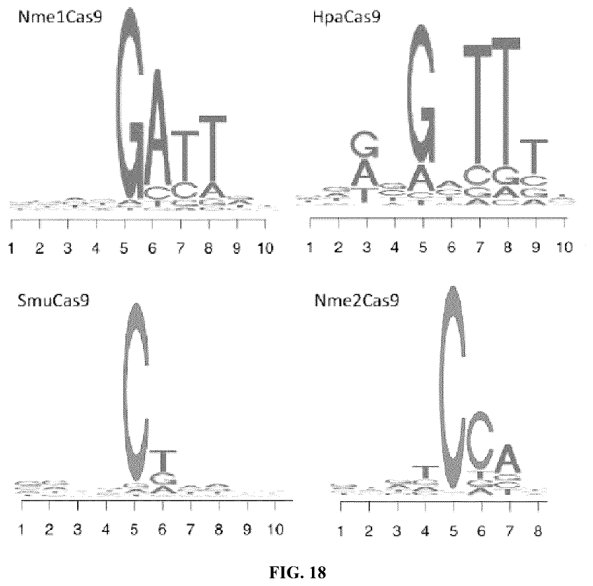

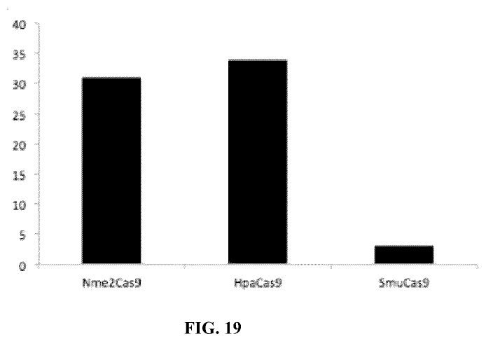

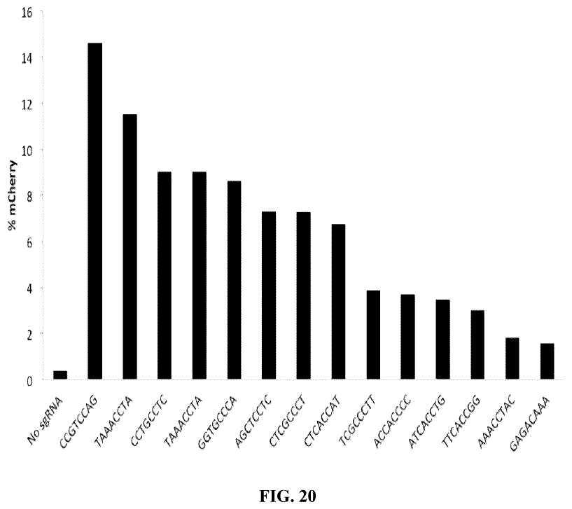

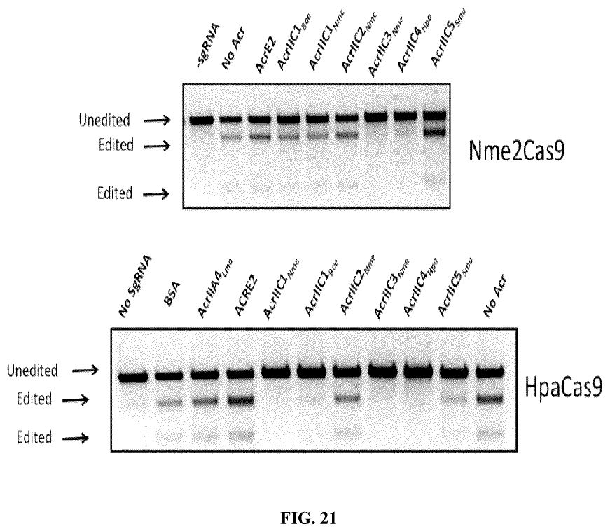

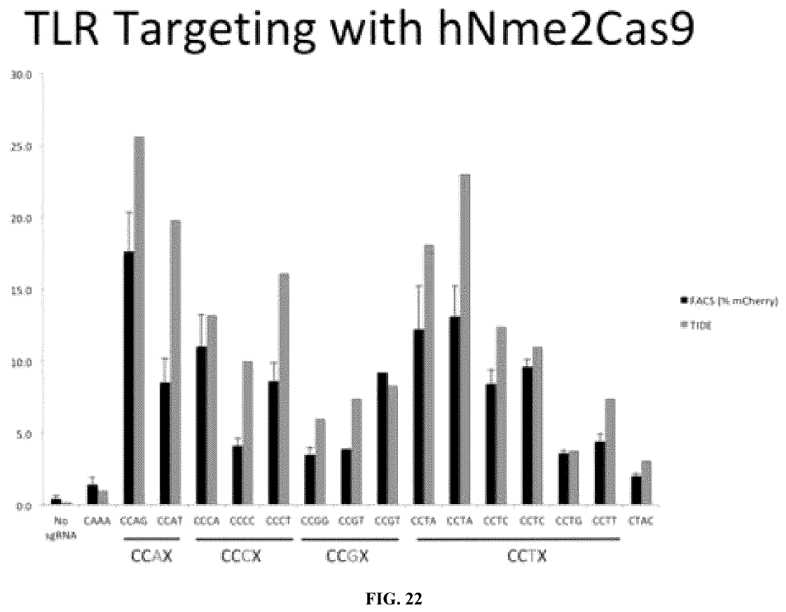

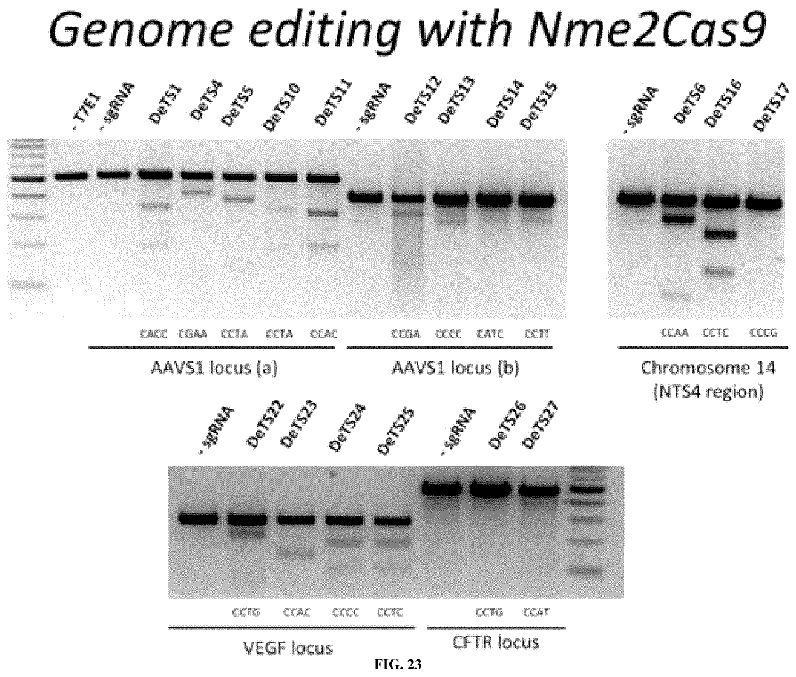

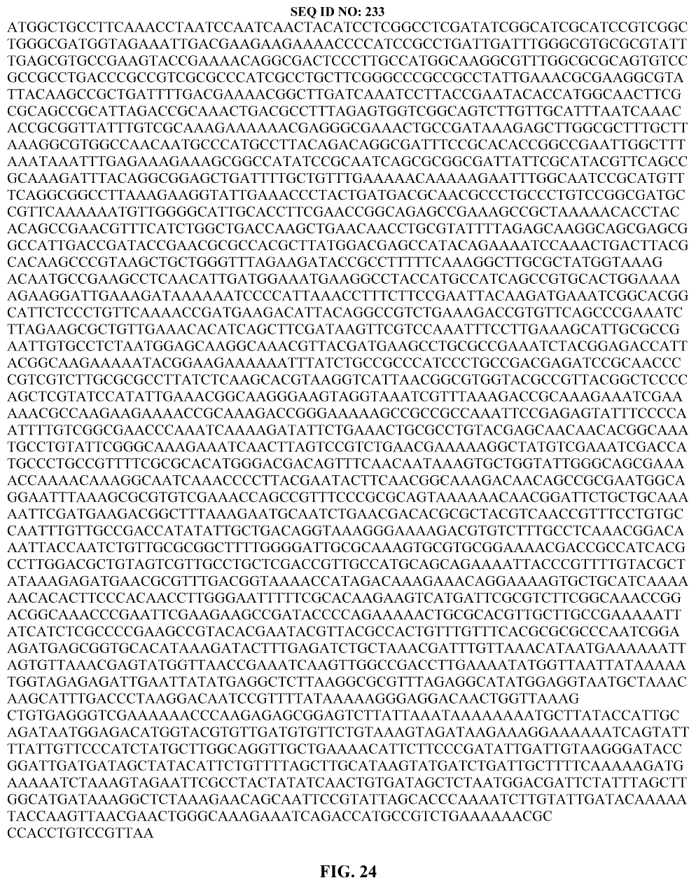

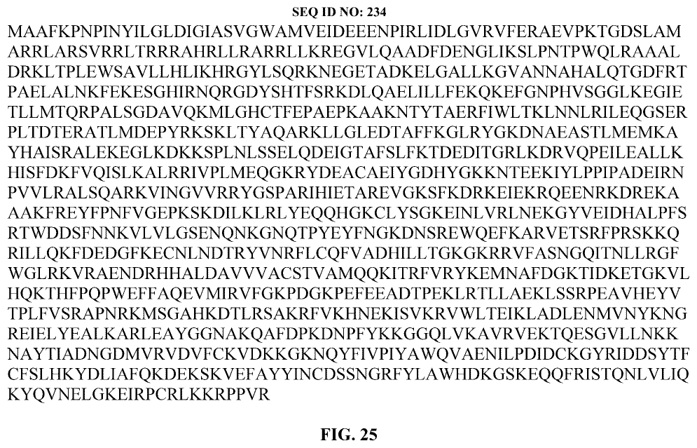

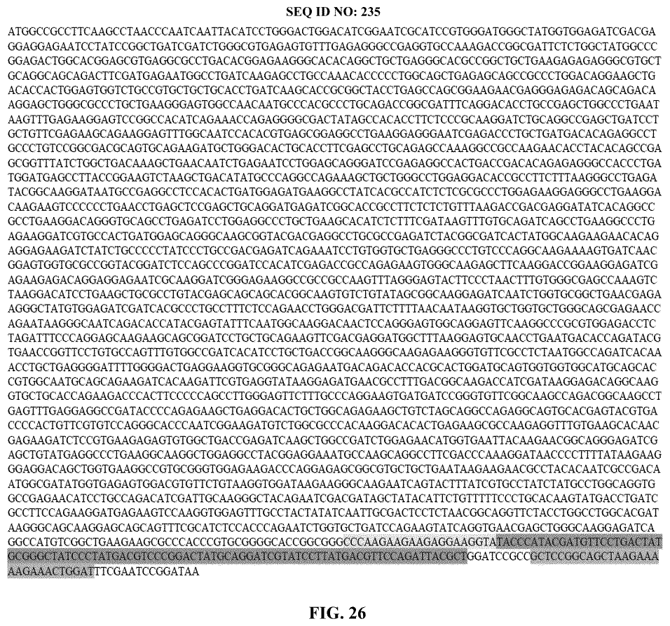

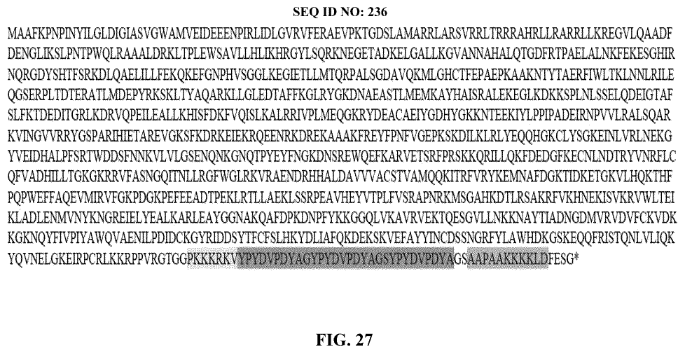

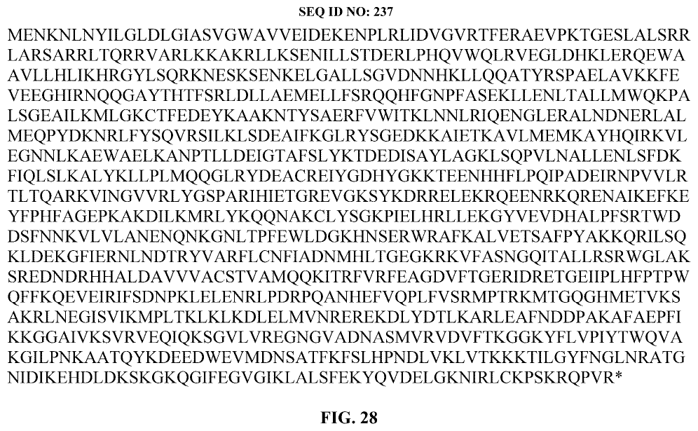

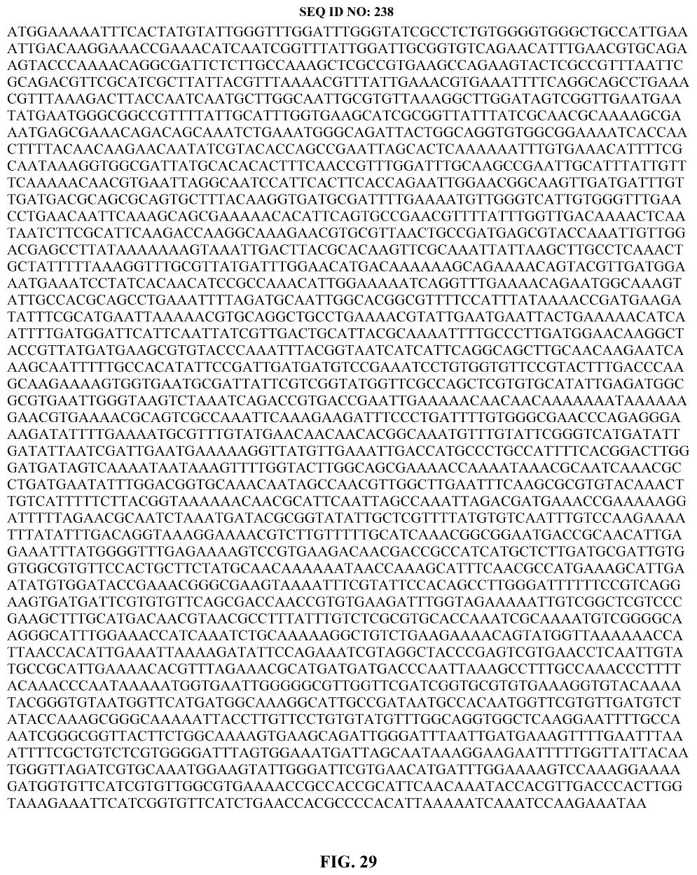

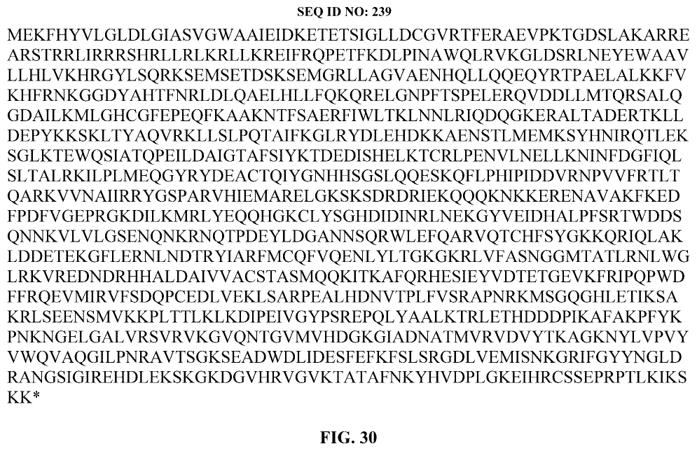

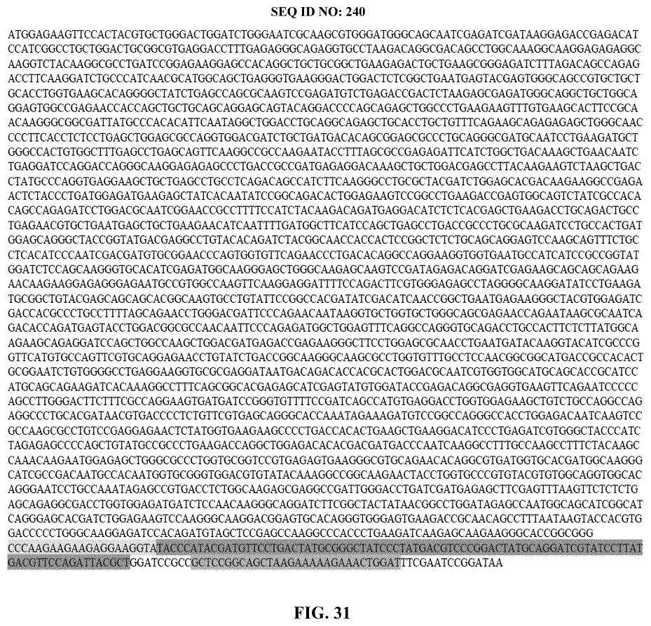

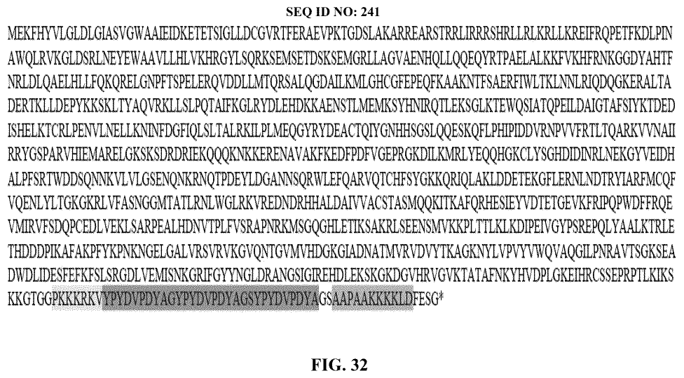

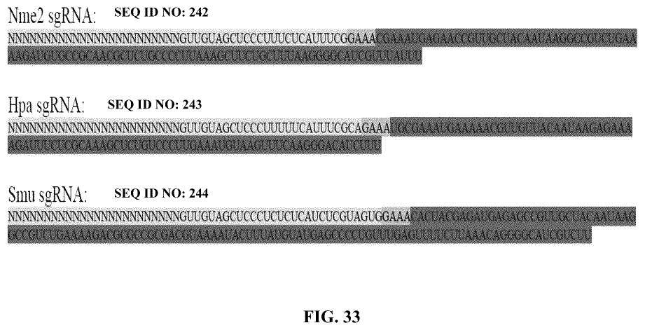

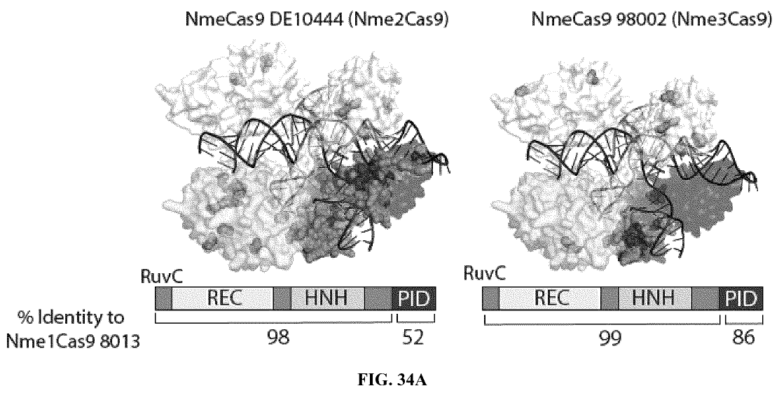

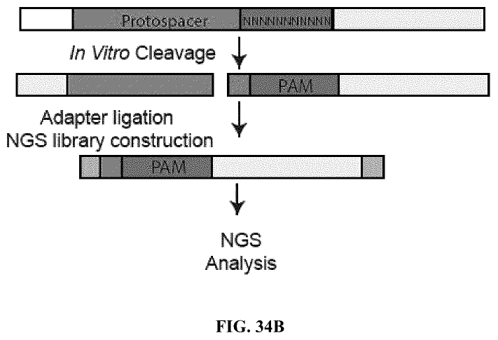

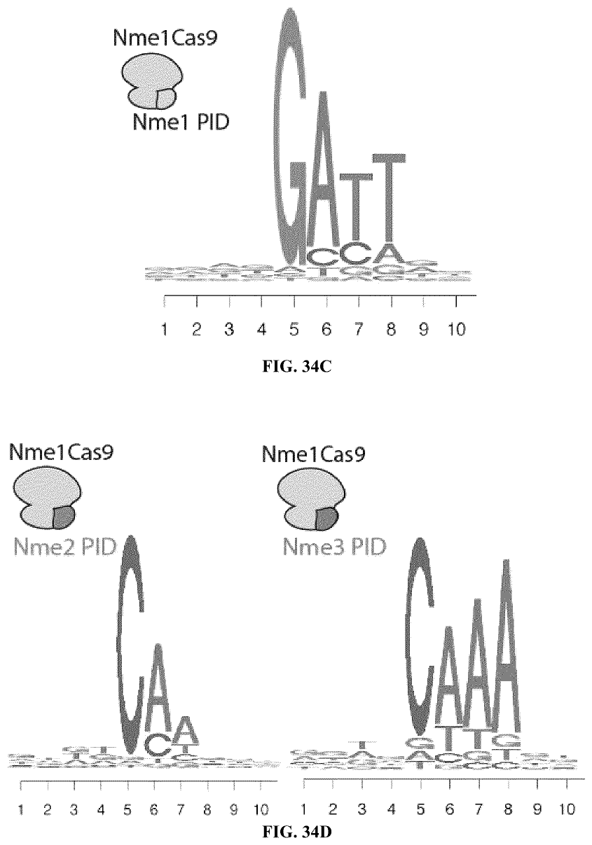

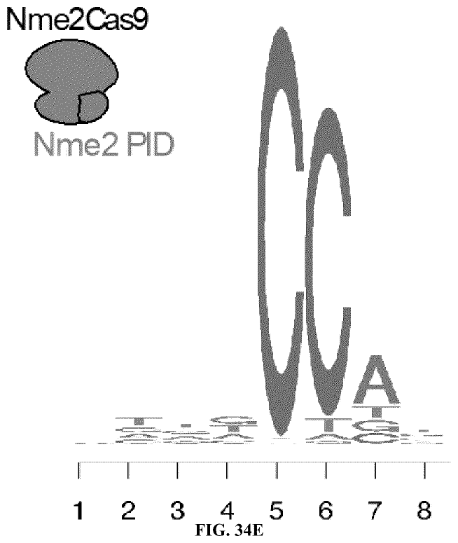

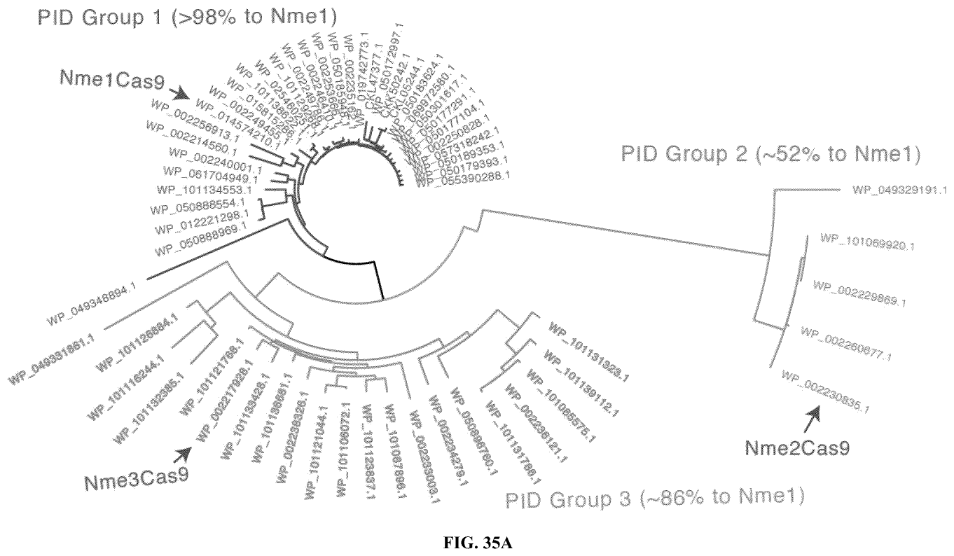

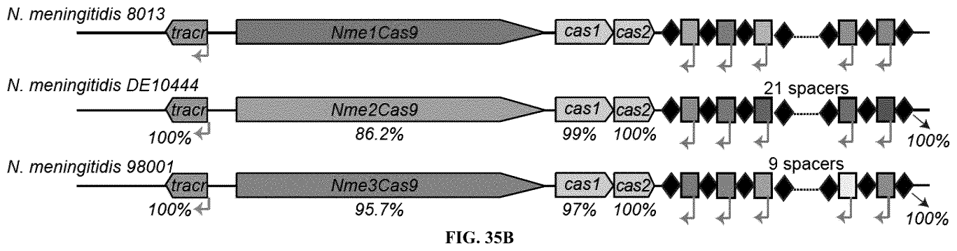

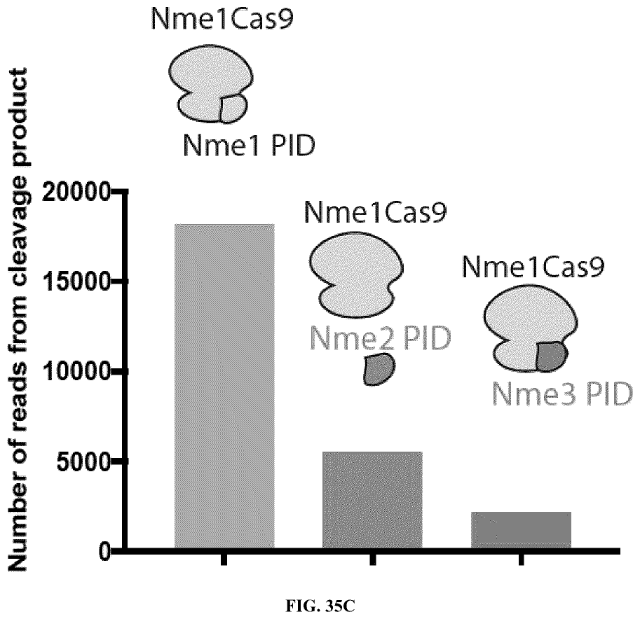

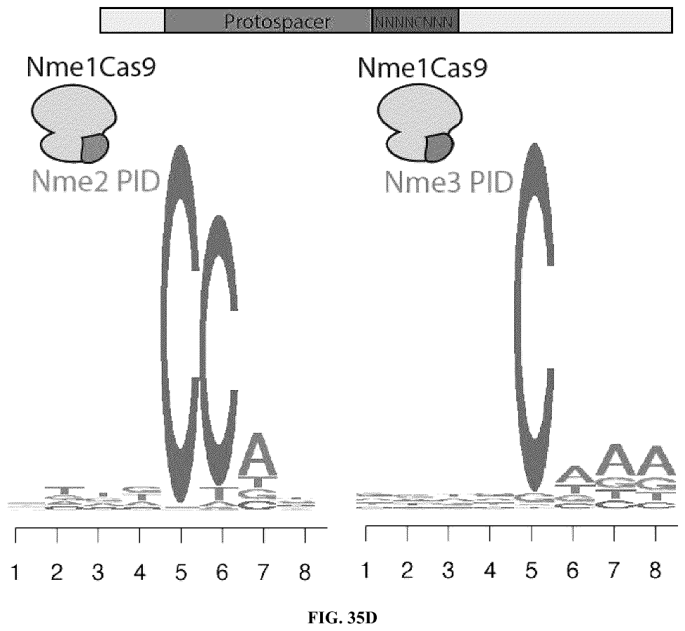

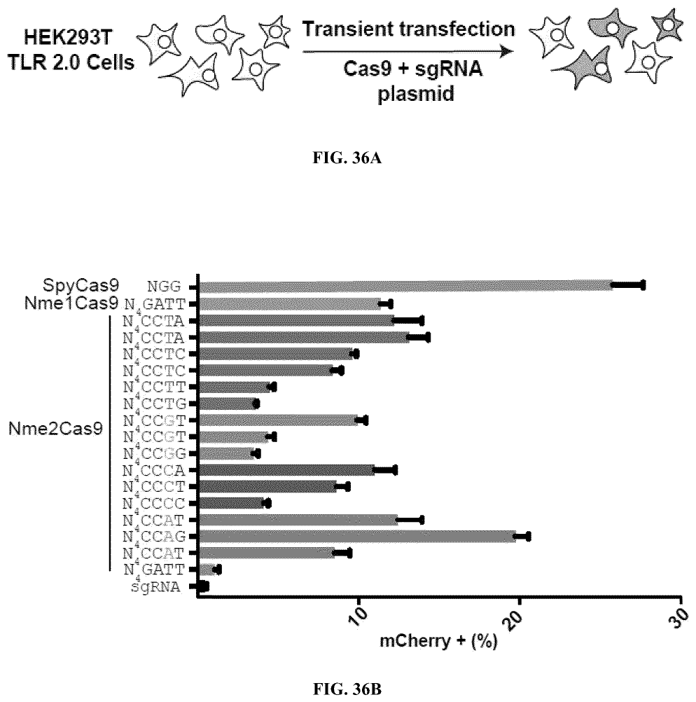

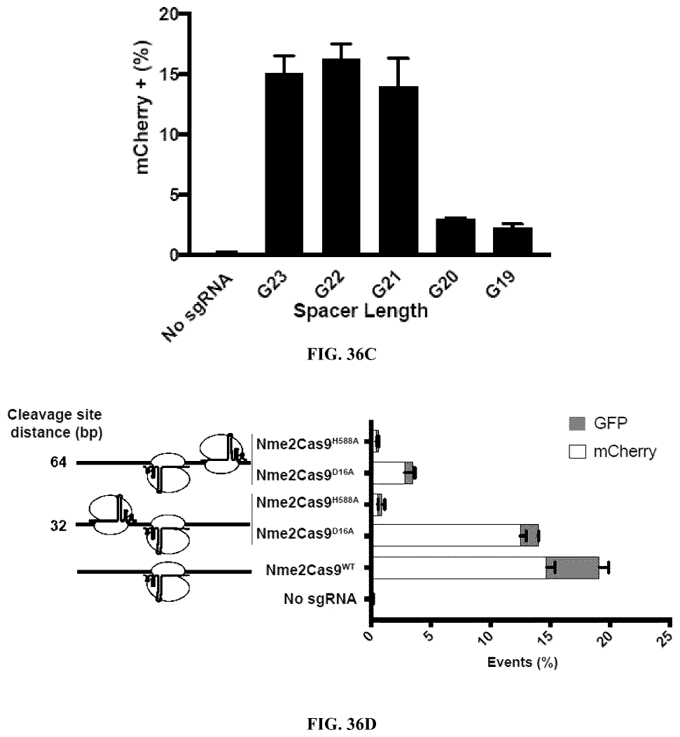

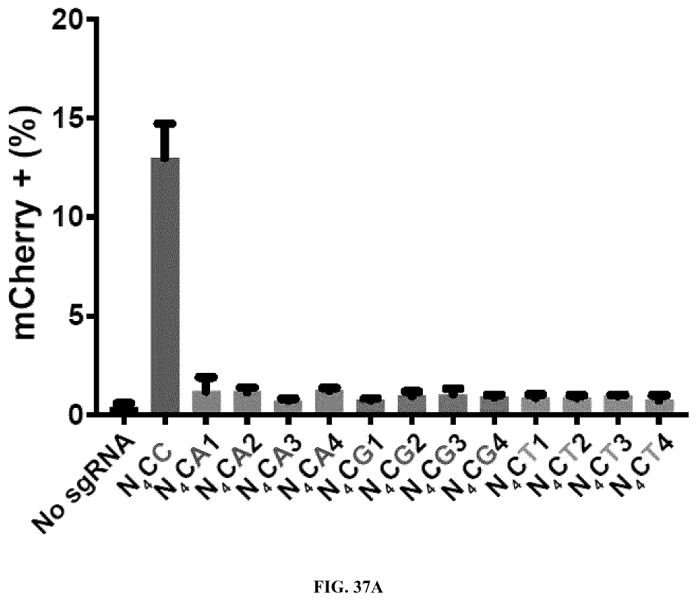

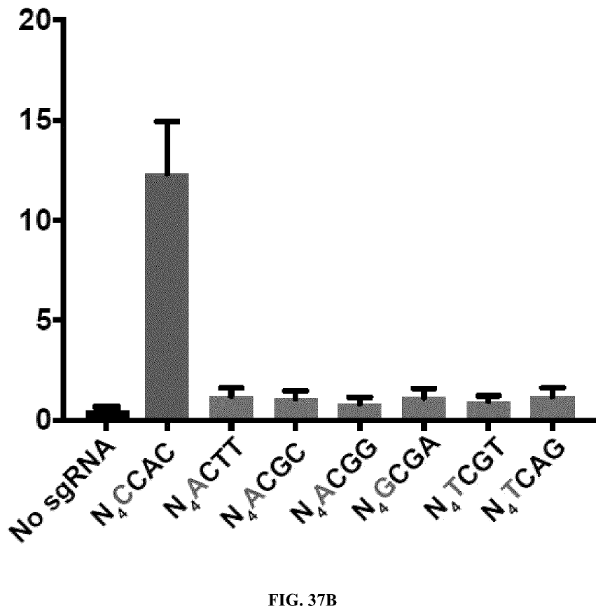

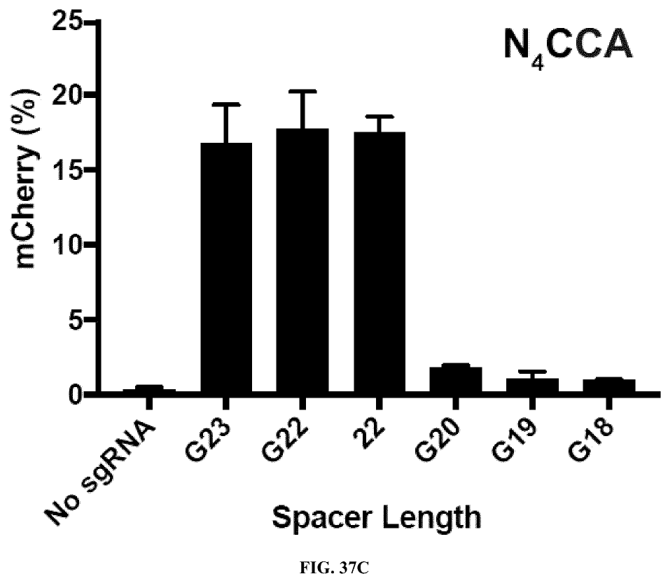

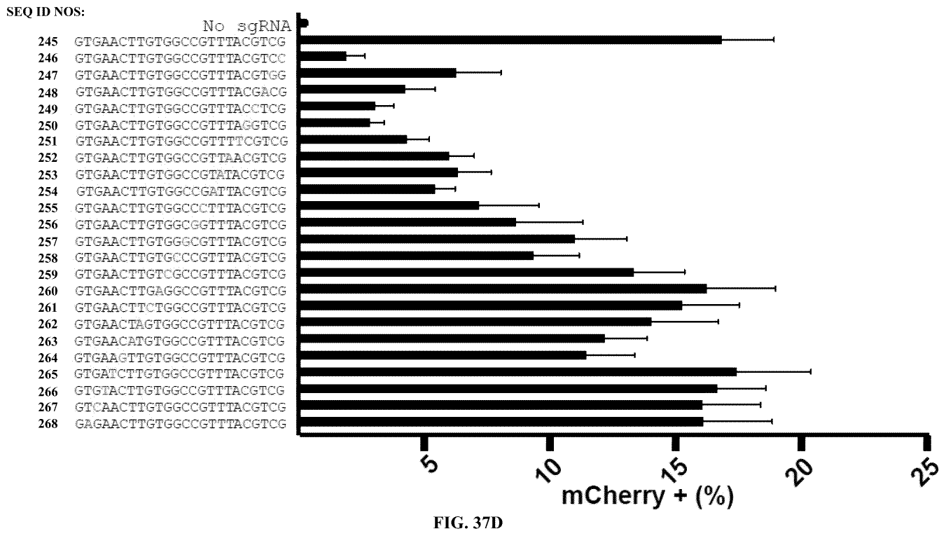

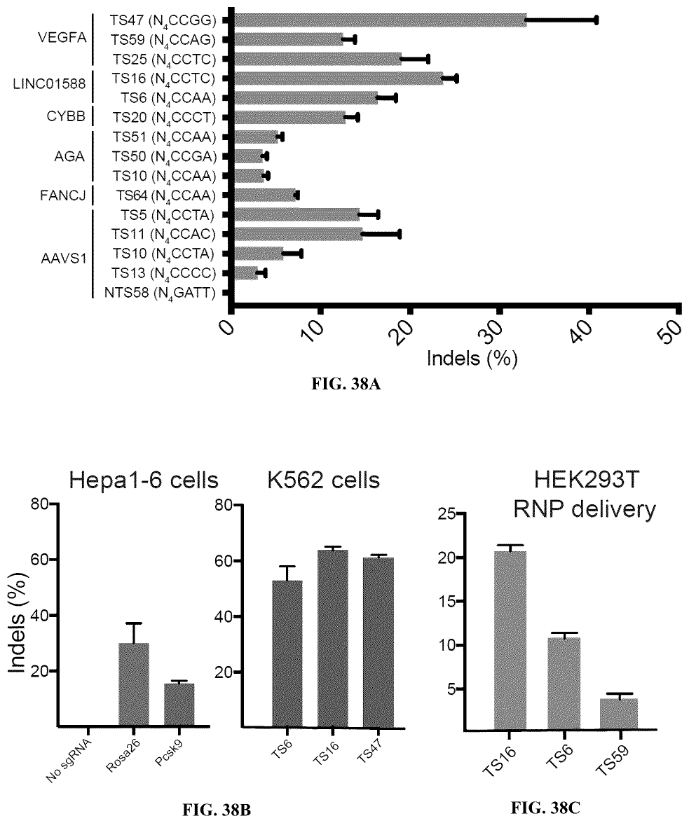

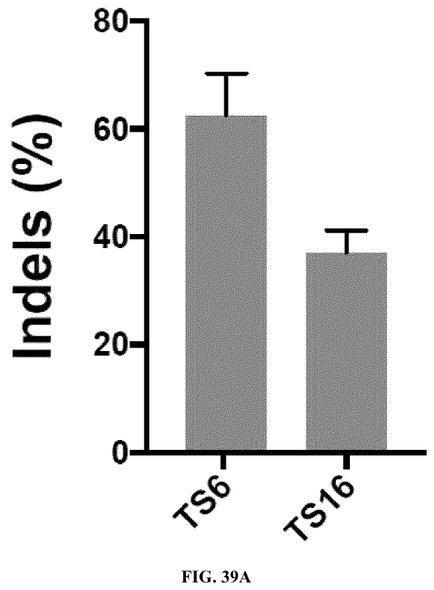

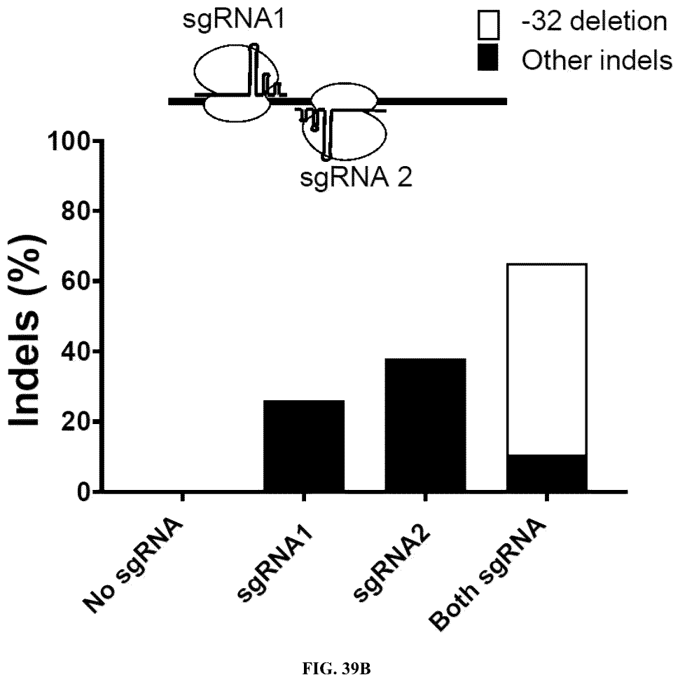

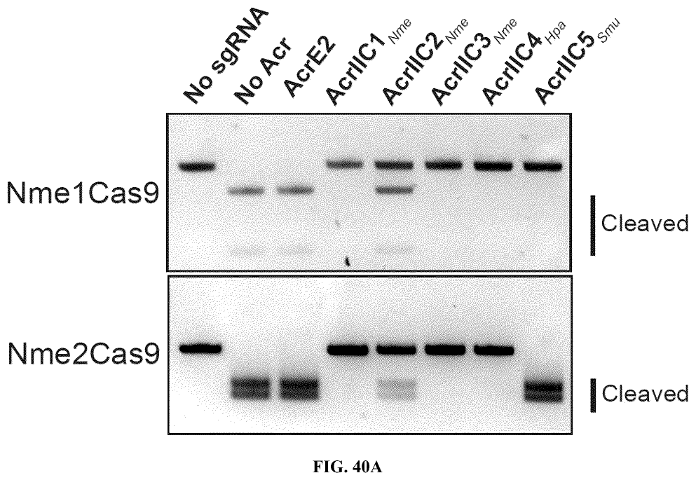

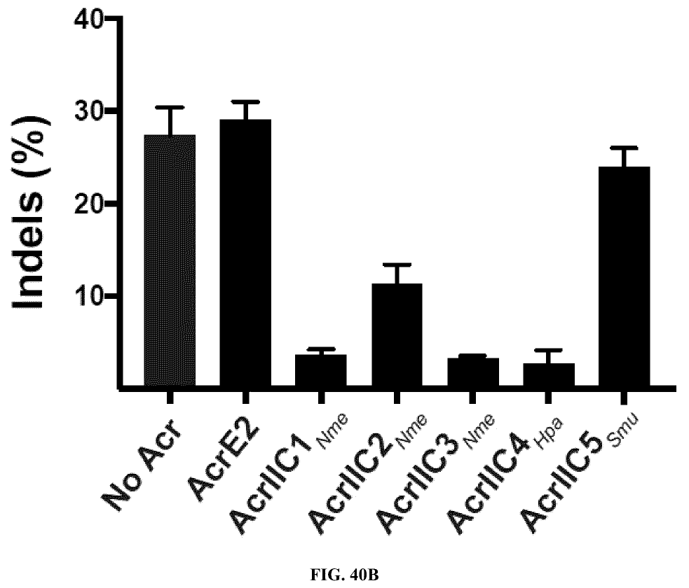

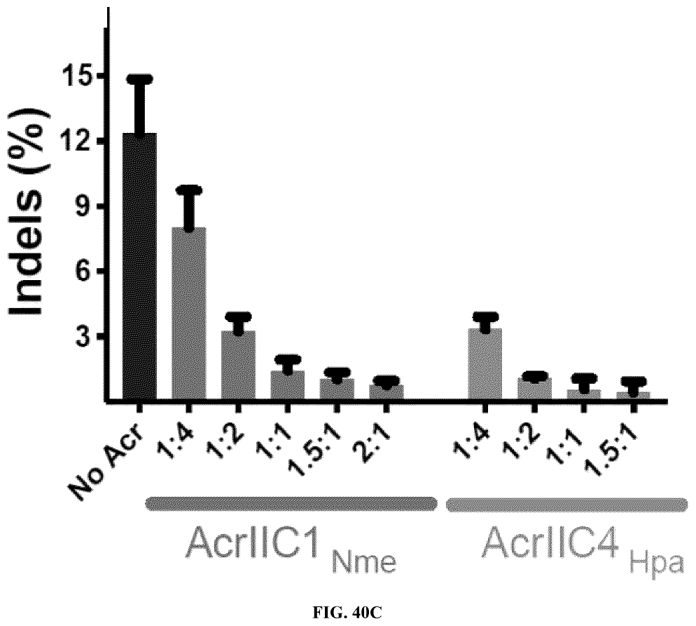

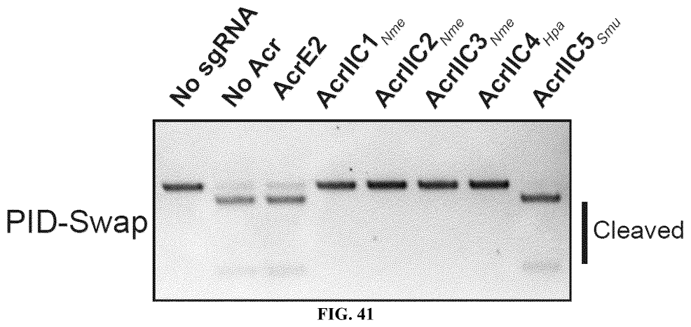

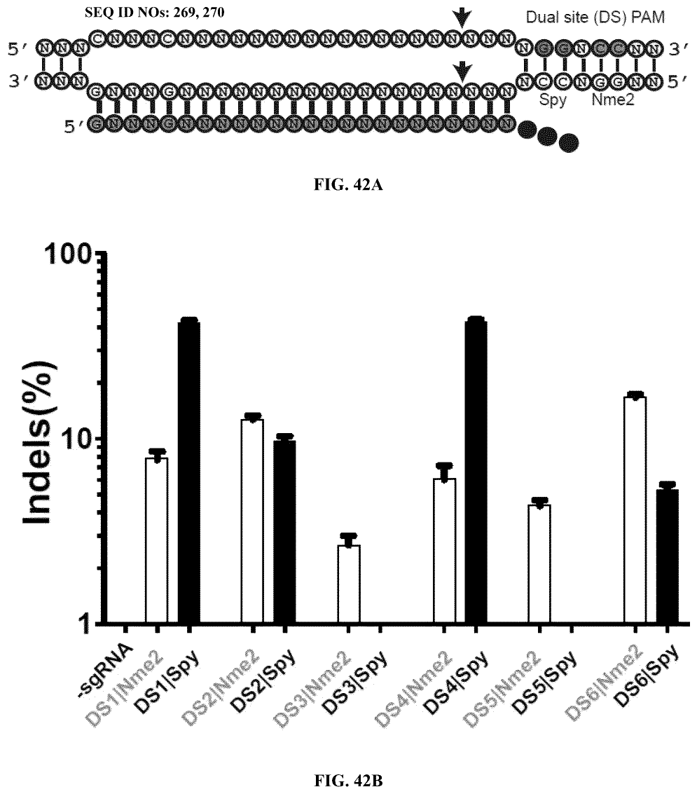

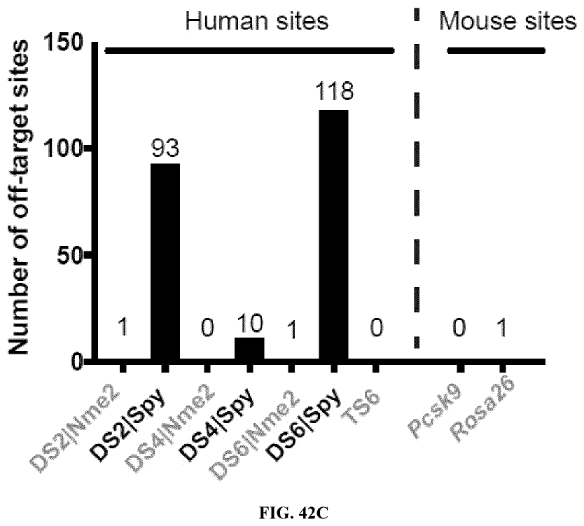

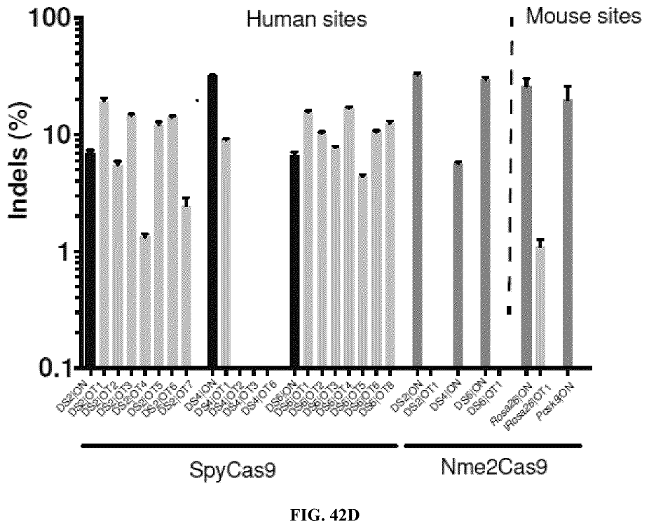

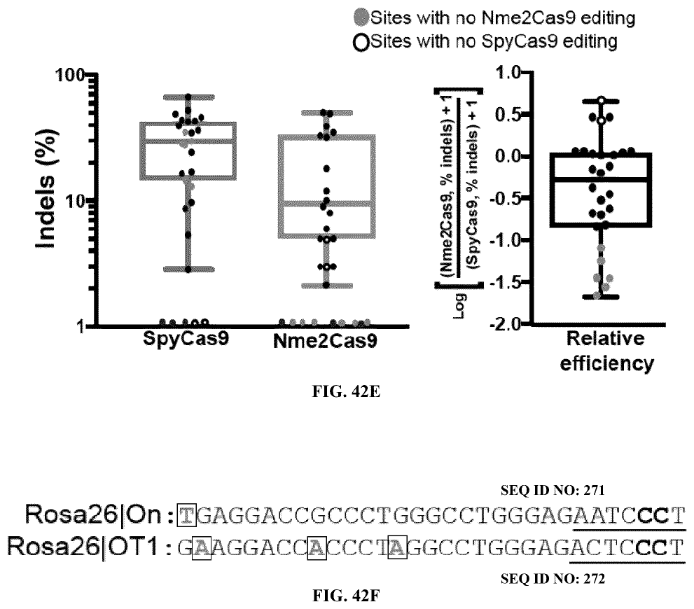

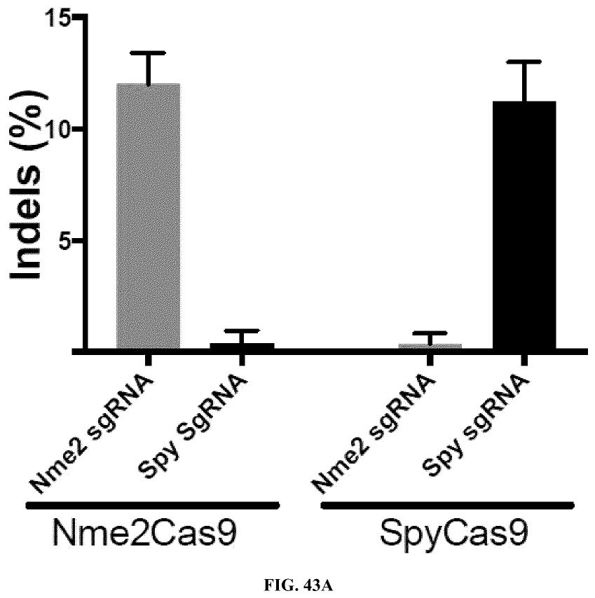

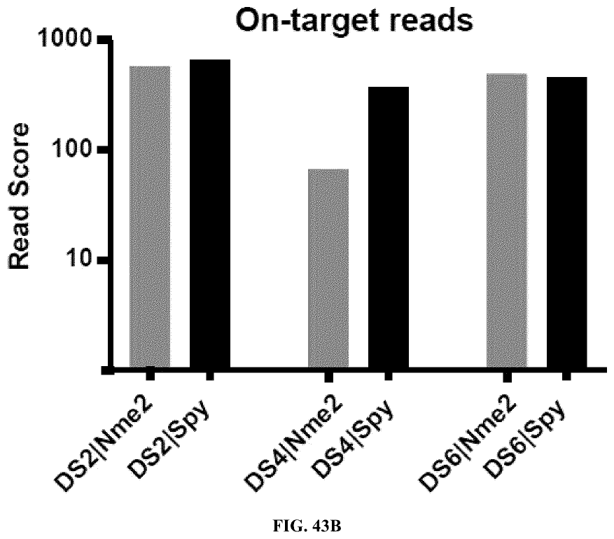

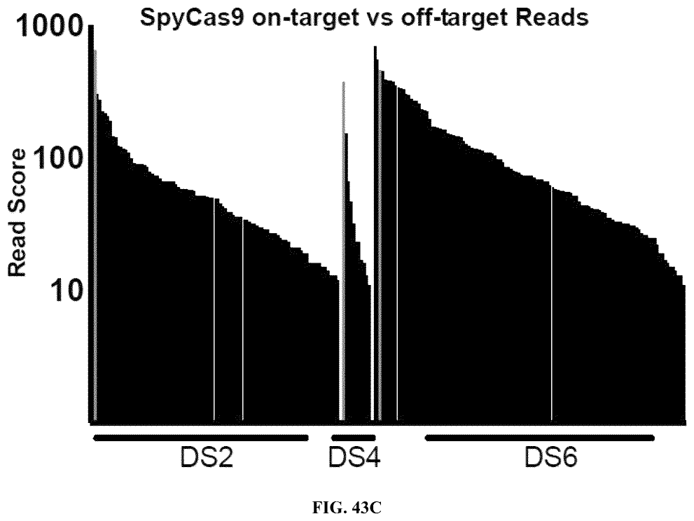

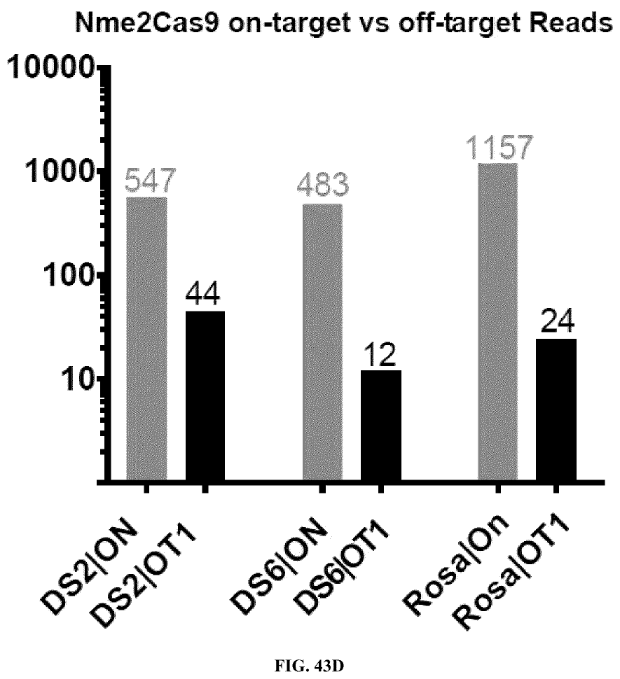

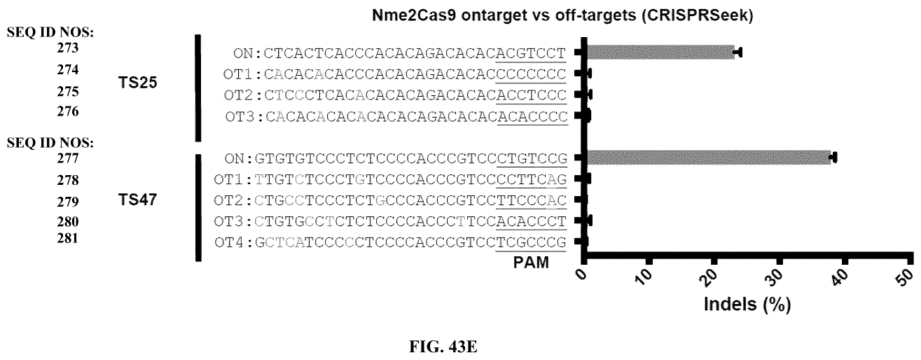

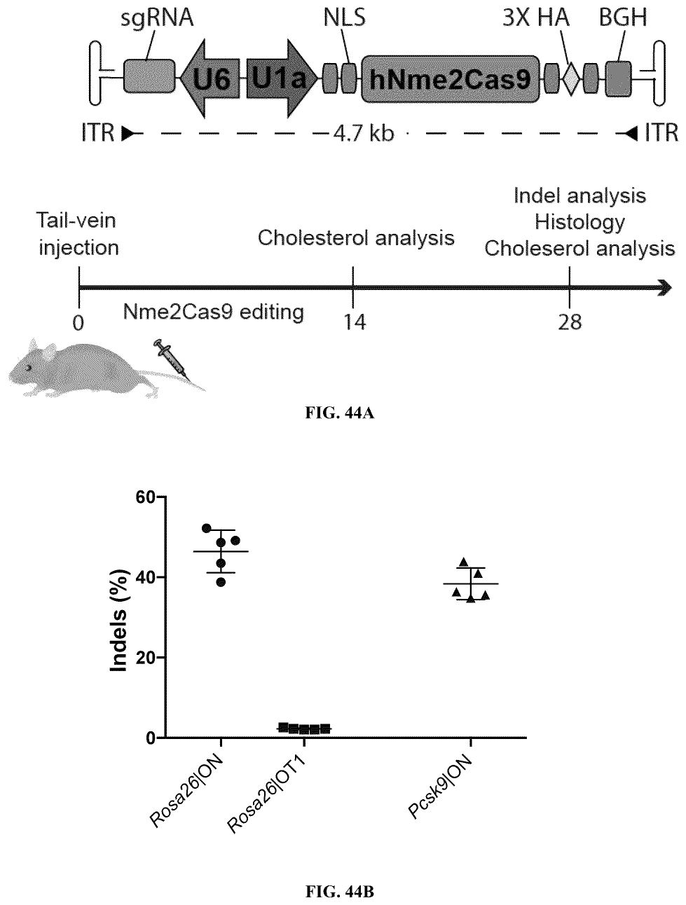

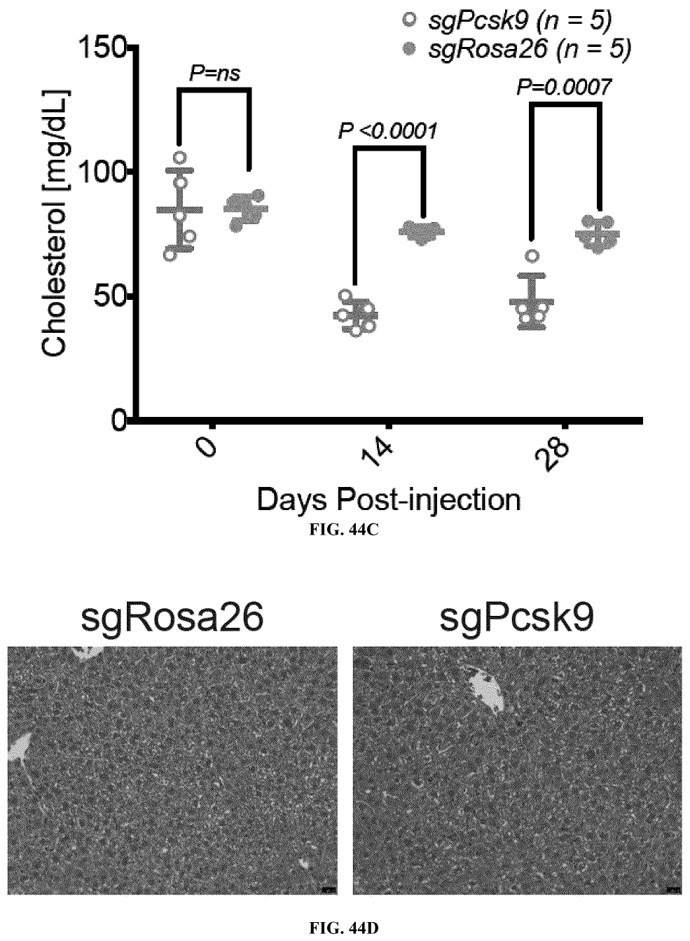

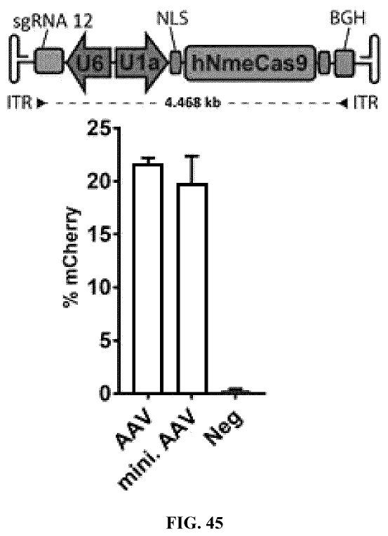

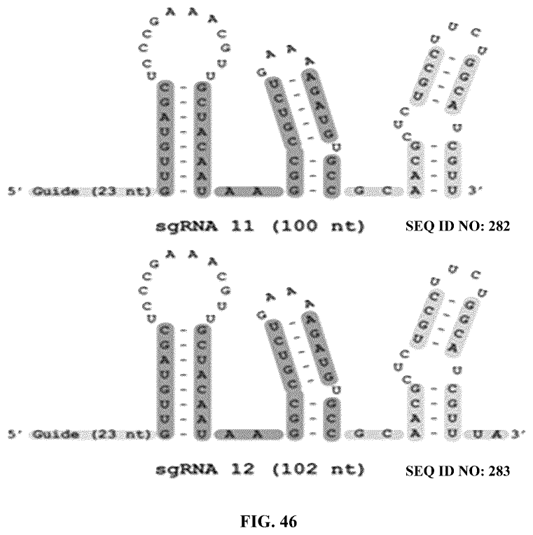

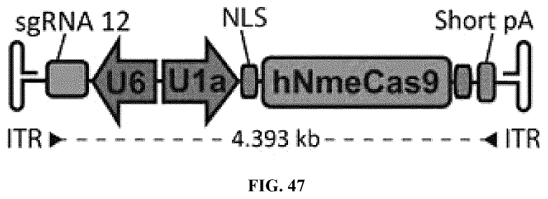

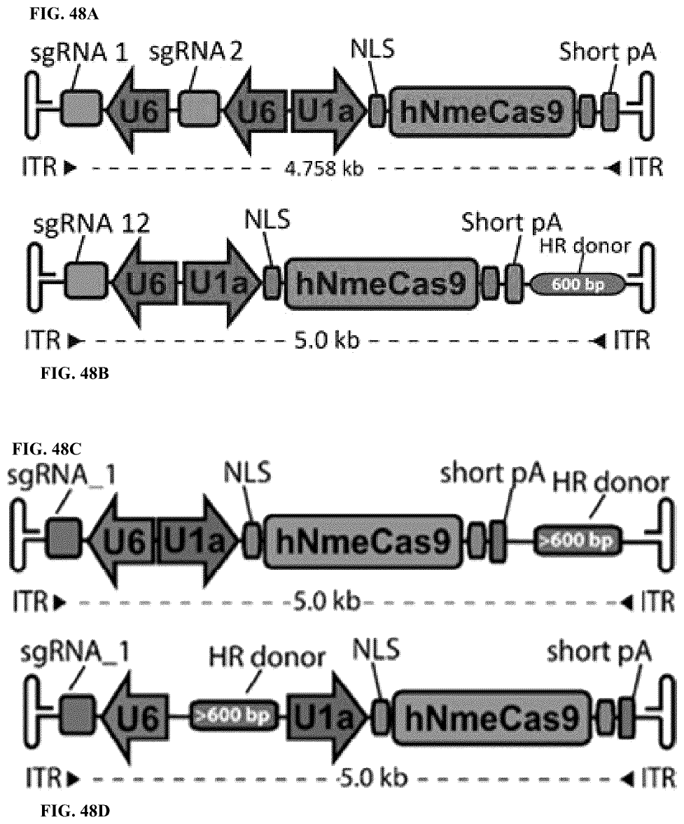

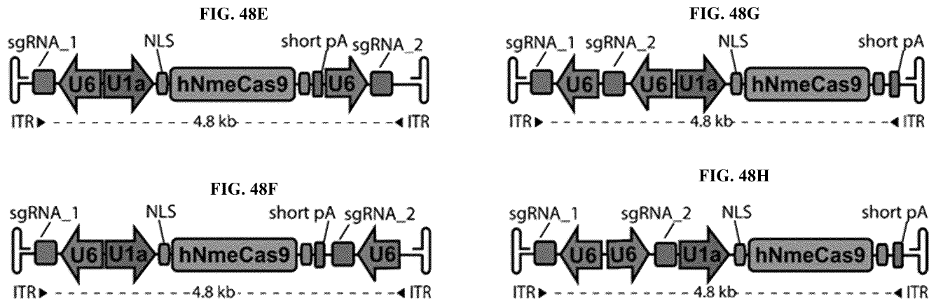

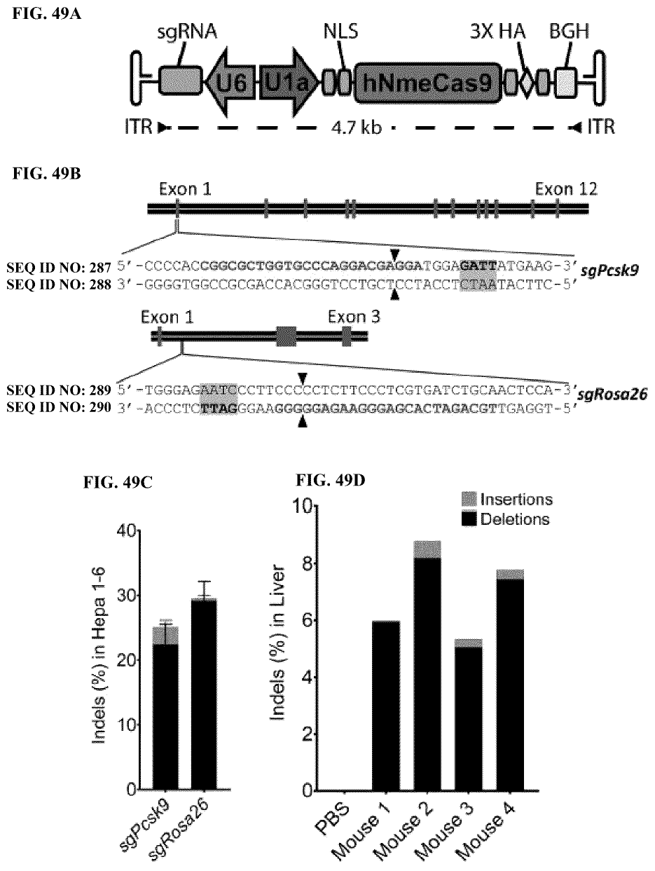

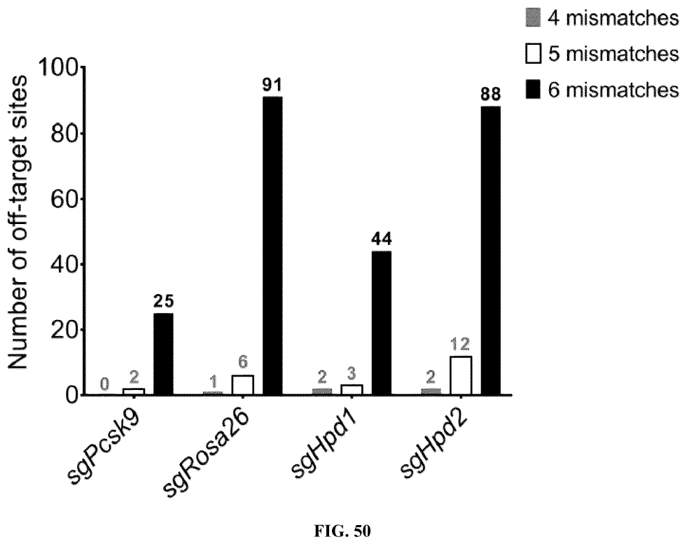

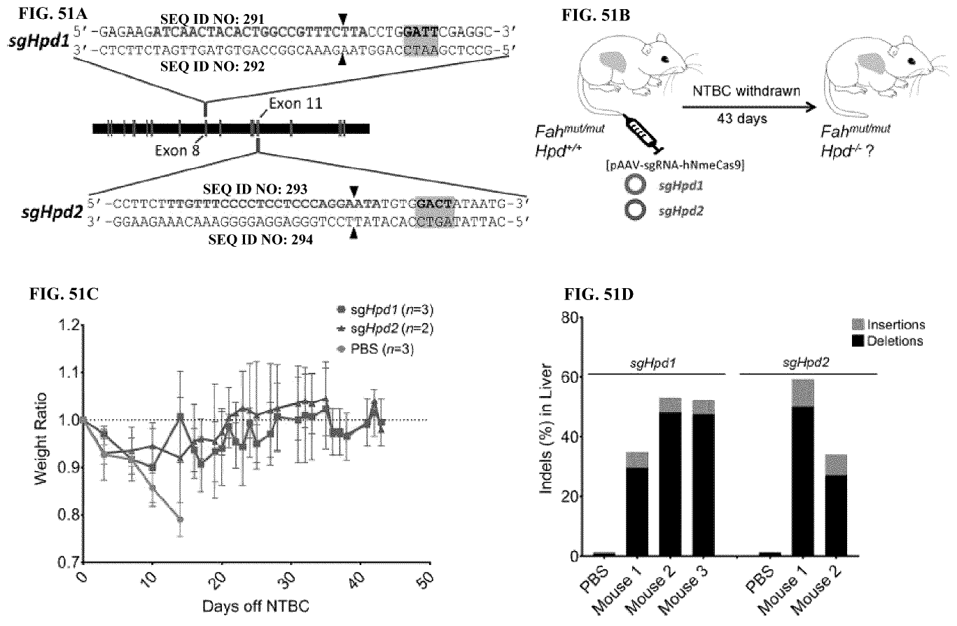

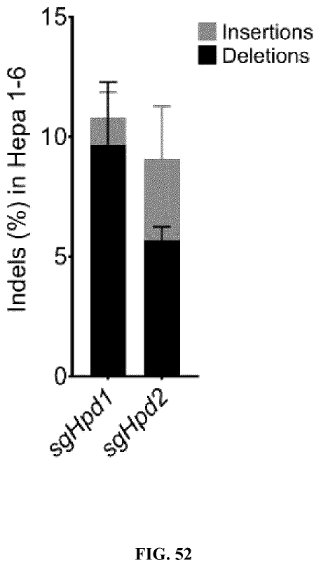

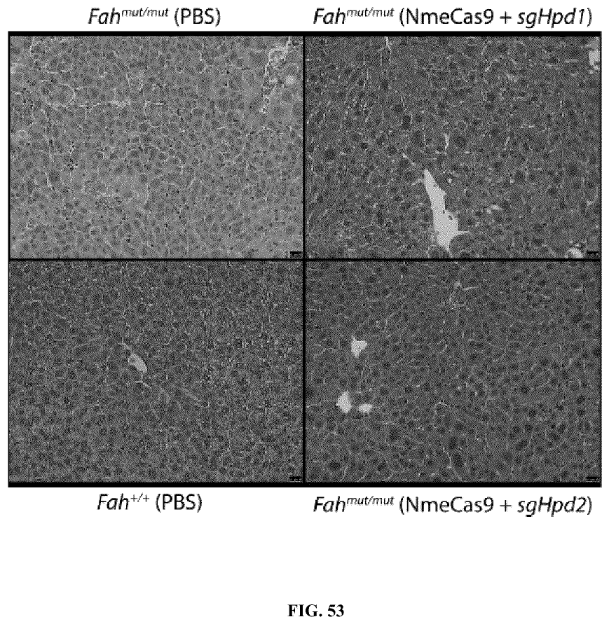

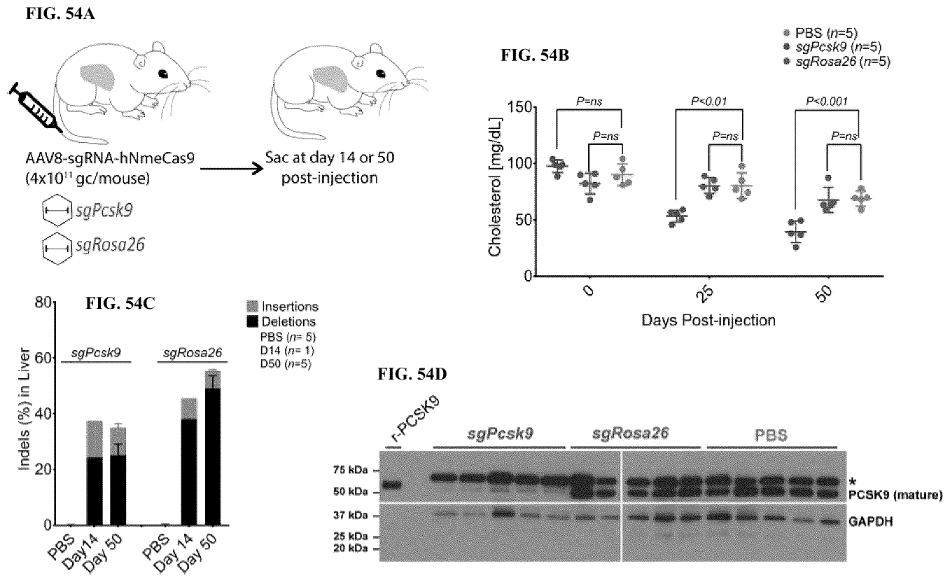

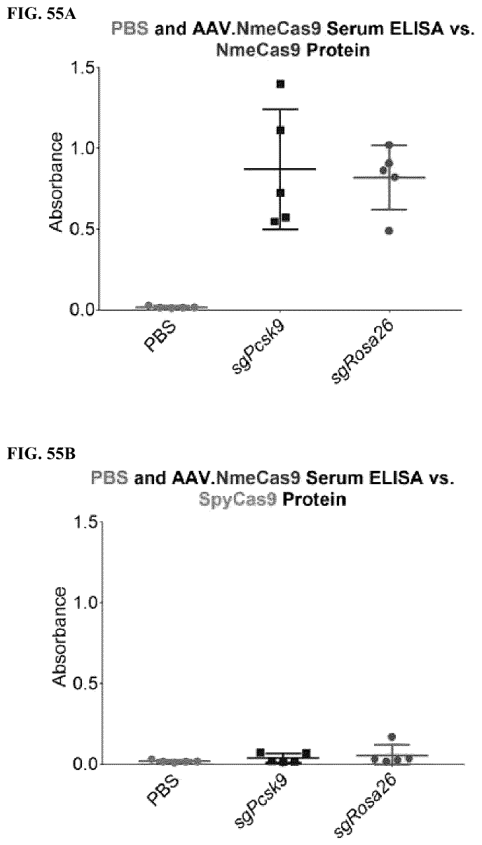

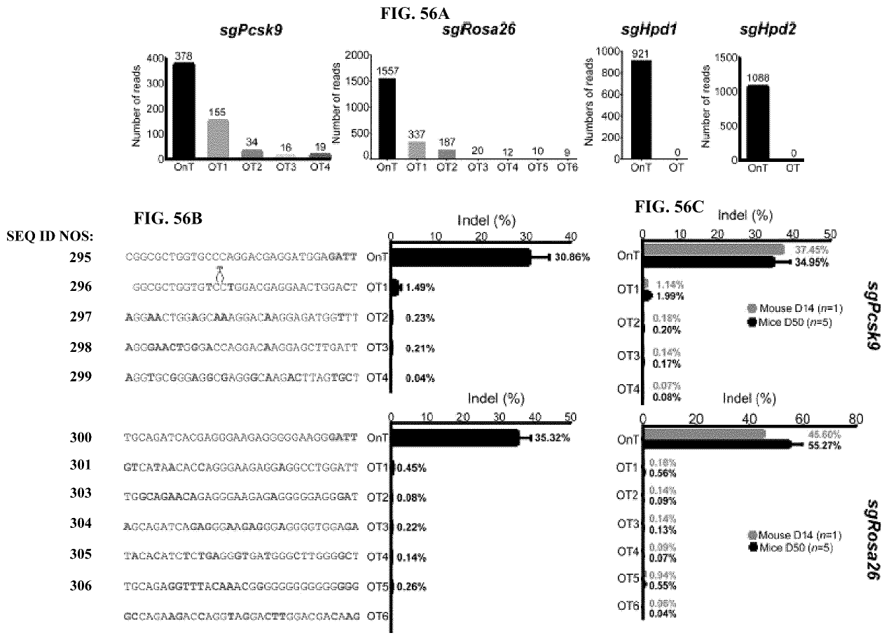

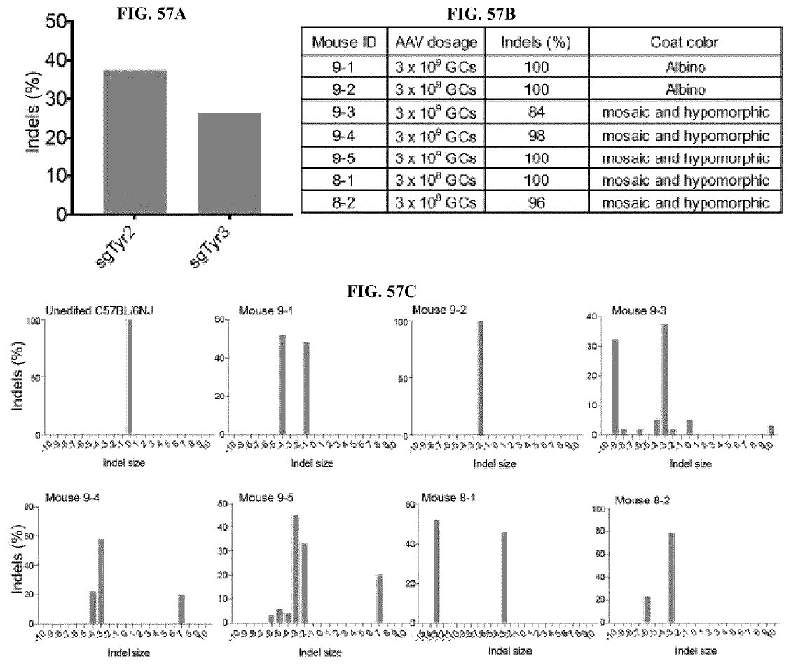

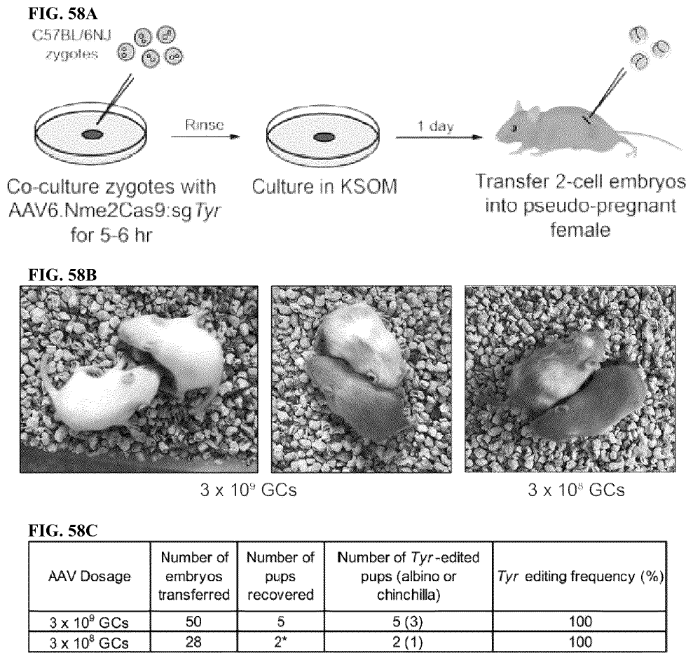

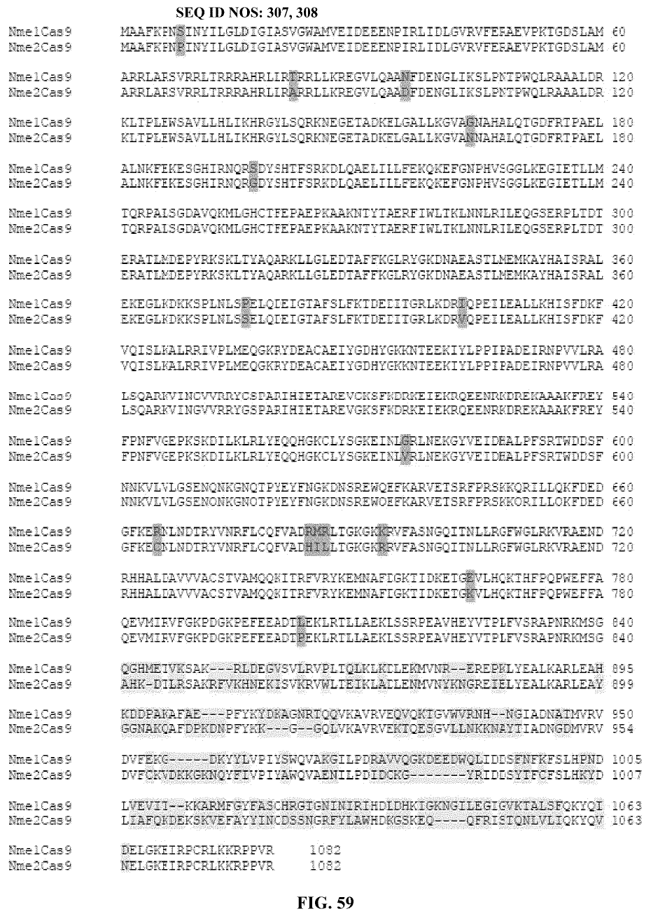

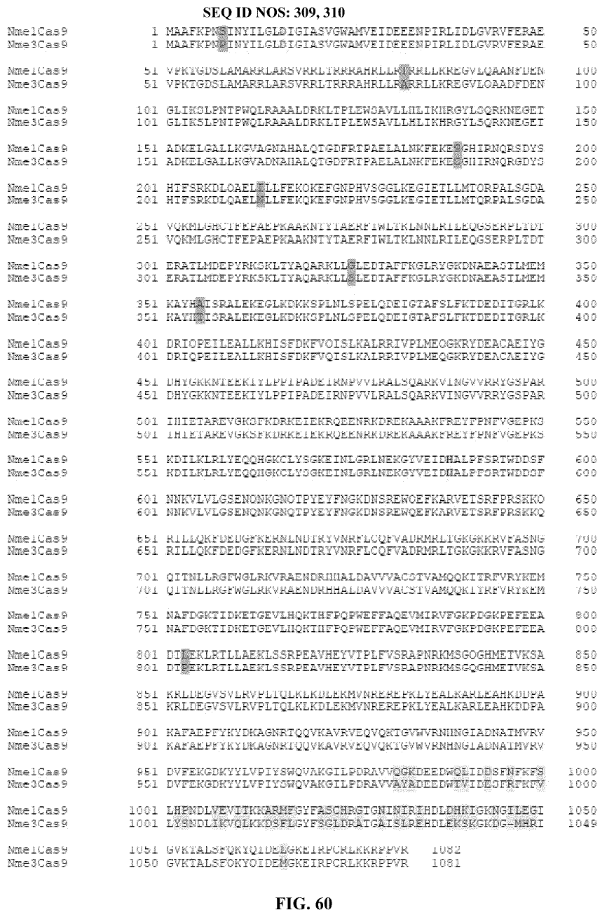

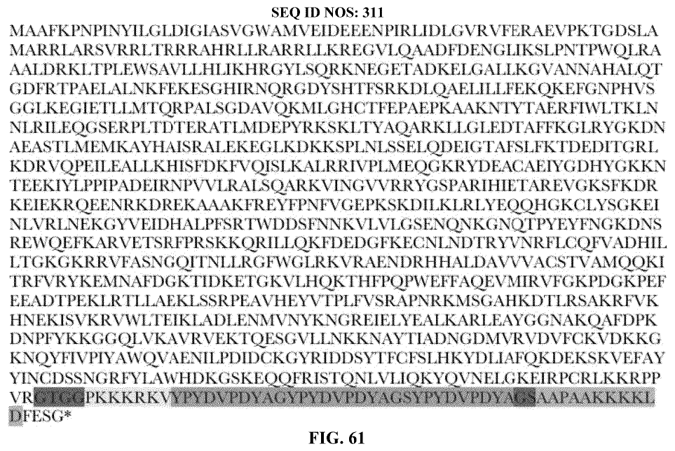

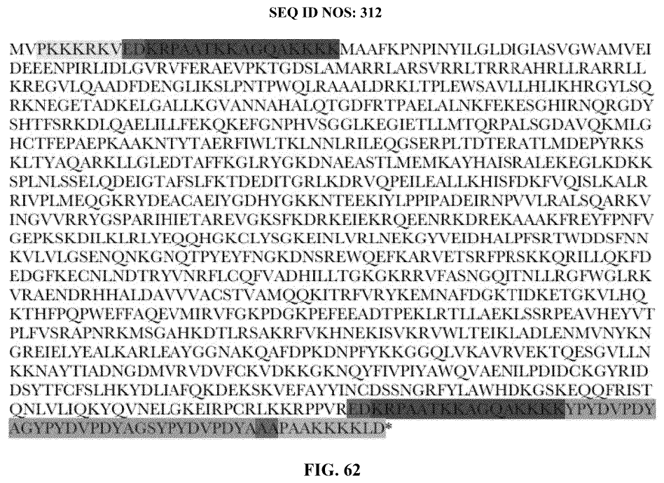

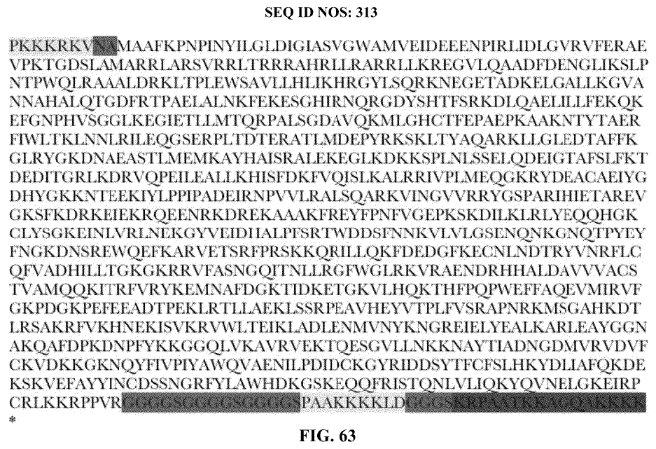

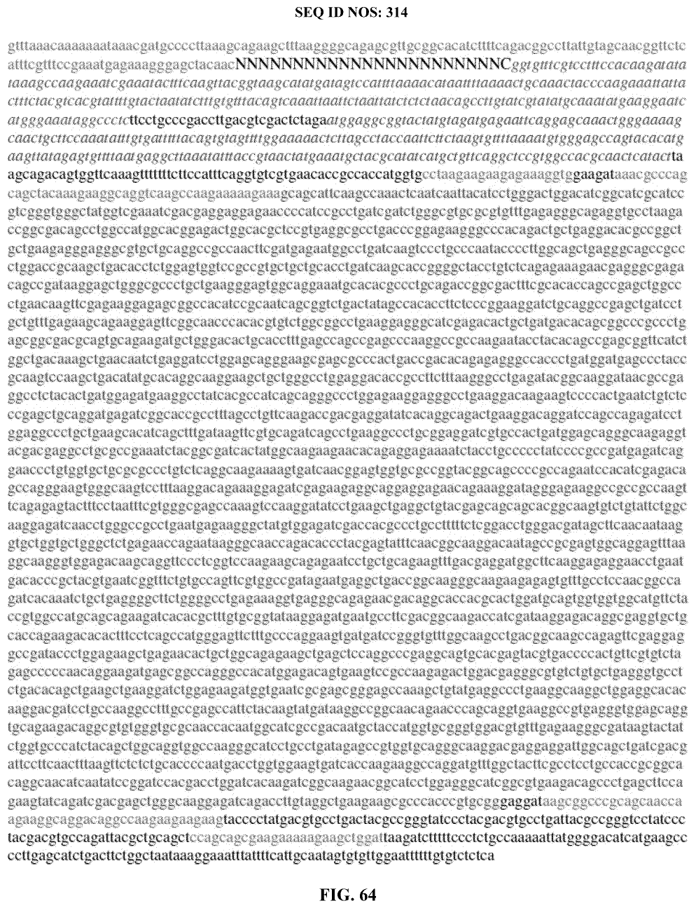

In one embodiment, the present invention contemplates an sgRNA-Nme1Cas9-AAV vector comprising an sgRNA sequence, an RNA Polymerase III U6 promoter sequence, a human codon-optimized Nme1Cas9 sequence, and an RNA Polymerase II U1a promoter sequence. FIG. 6. U1a is a ubiquitous promoter allowing versatile expression of Cas9 in various tissues of interest. Specific genes to be edited can be targeted by inserting a spacer sequence matching a target gene into an sgRNA cassette using conventional restriction sites (e.g., Sap1). Representative sequences of the various elements of the sgRNA-Nme1Cas9-AAV are shown by colored annotations. FIG. 7 and FIG. 8.

Editing efficiencies of several target sites using a Pcsk9-sgRNA-Nme1Cas9-AAV plasmid and a Rosa26-sgRNA-Nme1Cas9-AAV plasmid were estimated by an T7E1 assay following transient transfection into mouse Hepa1-6 hepatoma cells. FIG. 9. Representative target site sequences within a Pcsk9 gene and a Rosa26 gene complementary with a Pcsk9-sgRNA-Nme1Cas9-AAV plasmid and a Rosa26-sgRNA-Nme1Cas9-AAV plasmid are shown by colored annotations. FIG. 10.

The plasmid design was validated in vivo with mice by hydrodynamic injection of 30 μg of endotoxin-free sgRNA-Nme1Cas9-AAV plasmid targeting Pcsk9 via tail-vein. Significant gene editing was detected in mouse liver 10 days after injection as measured by Tracking of Indels by DEcomposition (TIDE), a sequencing-based method of evaluating indel efficiencies. FIG. 11.

The plasmid backbones targeting a Pcsk9 gene and a Rosa26 gene were packaged in hepatocyte-specific AAV8 serotype, and a dose of 4×10¹⁰ genomic copies (gc) per mouse was injected via tail-vein. Preliminary data show indel values from mice sacrificed at 14 days post-injection at a significant indel level in liver Pcsk9 and Rosa26 genes. FIG. 12A. Deep-sequencing data has also been collected at day 50 post-injection.

The three mice groups were sacrificed at day 50 post-injection, and liver gDNA was used to measure the indel values at Pcsk9 and Rosa26 using TIDE. FIG. 12B. Deep-sequencing analyses has also been performed to record accurate measurements of indel values.

PCSK9 protein “knock-down” may lead to significant lowering of cholesterol levels in mice. Serum cholesterol level was measured by Infinity™ colorimetric endpoint assay (Thermo-Scientific) in 3 mice groups injected with vectors targeting a Pcsk9 gene, a Rosa26 gene and a PBS control group. Results suggest that Nme1Cas9-induced indel formation has led to the interruption of the normal reading frame of the Pcsk9 gene, as showed by significantly reduced values of serum cholesterol at 25 and 50 days post-injection. FIG. 13. Western blot assay has also been performed to measure the level of PCSK9 protein in mice liver at day 50.

A genome-wide unbiased identification of double strand breaks (DSBs) enabled by a sequencing assay (e.g., GUIDE-Seq®, Illumina) searched for off-target editing sites subsequent to injection of vectors targeting a Pcsk9 gene and a Rosa26 gene. The data revealed four (4) potential off-target sites for Pcsk9 and six (6) potential off-target sites for Rosa26. FIG. 14A and FIG. 14B.

A targeted TIDE analyses revealed on-target genome editing in cells and in the mice at day 14 subsequent to injection of AAV vectors targeting a Pcsk9 gene and a Rosa26 gene. FIG. 15. Deep-sequencing analyses for off-target cleavage at these sites has also been performed at 50 days post-injection.

A hematoxylin and eosin stain assay did not show signs of massive immune cell infiltration in the liver sections of mice sacrificed at day 14 subsequent to injection of vectors targeting a Pcsk9 gene and a Rosa26 gene. FIG. 16. Specific immune-response assays will be performed at 50 day post-injection.

In one embodiment, the present invention contemplates a method for therapeutic in vivo genome editing by all-in-one AAV delivery of an Nme2Cas9. Although it is not necessary to understand the mechanism of an invention it is believed that the compactness, small PAM and high fidelity make Nme2Cas9 an ideal tool for in vivo genome editing using AAV. To this end, Nme2Cas9 was cloned with its cognate sgRNA and their respective promoters into a single AAV vector backbone. FIG. 44A; top. This all-in-one AAV.sgRNA.Nme2Cas9 was packaged in a hepatocyte-selective AAV8 capsid. Two genes were targeted: i) Rosa26, a commonly used locus as a negative control; and ii) the Proprotein convertase subtilisin/kexin type 9 (Pcsk9), a major regulator of circulating cholesterol homeostasis. Studies have shown that knocking out Pcsk9 using Cas9 results in reduced cholesterol levels (Ran et al).

Two groups of mice (n=5) were injected with packaged AAV8.sgRNA.Nme2Cas9 targeting either Pcsk9 or Rosa26. Serum was collected at 0, 14 and 28 days post vector injection for cholesterol level measurement. Mice were sacrificed at 28 days post-injection and liver tissues were harvested. (FIG. 44A, bottom. A deep sequencing analysis showed significantly high level of indels at Pcsk9 and Rosa26. FIG. 44B. These indel values were accompanied by significant reduction in blood cholesterol level in mice injected with sgPcsk9 after 14 and 28 days; where mice injected with sgRosa26 maintained normal level of cholesterol throughout the study. FIG. 44C. An H&E analyses showed no signs of toxicity or tissue damage at both groups after Nme2Cas9 expression. FIG. 44D. These data validate that Nme2Cas9 is highly functional in vivo, and it can be readily delivered by the favorable all-in-one AAV platform.

In one embodiment, the present invention contemplates a minimized AAV.hNmeCas9 construct. See, FIG. 44A. As discussed above, the present invention contemplates an engineered all-in-one AAV.sgRNA.hNme1Cas9 construct, which is packaged in AAV8 virions that successfully edited Pcsk9 and Rosa26 genes in mice liver.

In one embodiment, the present invention contemplates an AAV8 backbone comprising an Nme2Cas9 cassette. Similar to Nme1Cas9, Nme2Cas9 also showed robust editing at Pcsk9 and Rosa26 in mice (infra). The data presented herein shows that in vivo administration of AAV8-NmeCas9 to mice is accompanied by significant reduction in level of circulating cholesterol after 28 days post vector injection.

In order to increase the utility of this all-in-one AAV platform, various truncations were introduced to minimize the size of the cargo to make a space for additional features in the AAV capsid, such as dual sgRNAs or donor DNA segment.

In order to minimize the cargo of the all-in-one AAV backbone, the extra features (3×HA tags and 2×NLS sequences) were systematically removed without compromising the nuclease activity of the Cas9. Nme1Cas9, using the traffic light reporter (TLR) system, show that this minimized all-in-one AAV.sgRNA.hNme1Cas9 (4.468 kb) is as potent as the previous longer version with 4 NLS sequences. See, FIG. 45. Truncated sgRNAs were constructed to free more space using a new sgRNA12, which is similar to an sgRNA11 version, but with UA added at the 3′ end. See, FIG. 46.

Previously, it has been reported that a short poly A sequence may be useful for Cas9 constructs. Platt et. al. (2015). In one embodiment, the present invention contemplates an AAV-Nme2Cas9 construct comprising a BGH polyA. See, FIG. 47. Although it is not necessary to understand the mechanism of an invention, it is believed that this polyA sequence further reduces the size of the all-in-one AAV backbone.

It is further believed that this minimized (4.4 kb) all-in-one AAV backbone increases the utility of Nme1Cas9 and Nme2Cas9 by including another sgRNA for dual genes knockout or DNA fragment excision. See, FIG. 48A-J, top. This configuration also provides free space in the AAV capsid to include a donor template (˜600 base pairs) for homology-directed repair application. See, FIG. 48A-J, bottom. In some embodiments, dual sgRNA AAV constructs are packaged within a single AAV vector.

The relatively compact Nme1Cas9 is active in genome editing in a range of cell types. To exploit the small size of this Cas9 ortholog, an all-in-one AAV construct was generated with human-codon-optimized Nme1Cas9 under the expression of the mouse U1a promoter and with its sgRNA driven by the U6 promoter. See, FIG. 49A. Two sites in the mouse genome were selected initially to test the nuclease activity of Nme1Cas9 in vivo: the Rosa26 “safe-harbor” gene (targeted by sgRosa26); and the proprotein convertase subtilisin/kexin type 9 (Pcsk9) gene (targeted by sgPcsk9), a common therapeutic target for lowering circulating cholesterol and reducing the risk of cardiovascular disease. FIG. 49B. Genome-wide off-target predictions for these guides were determined computationally using the Bioconductor package CRISPRseek 1.9.1 with N₄GN₃ PAMs and up to six mismatches. Zhu et al., “CRISPRseek: a bioconductor package to identify target-specific guide RNAs for CRISPR-Cas9 genomeediting systems” PLoS One 2014; 9:e108424. Many N₄GN₃ PAMS are inactive, so these search parameters are nearly certain to cast a wider net than the true off-target profile. Despite the expansive nature of the search, an analyses revealed no off-target sites with fewer than four mismatches in the mouse genome. See, FIG. 50. On-target editing efficiencies at these target sites were evaluated in mouse Hepa1-6 hepatoma cells by plasmid transfections and indel quantification was performed by sequence trace decomposition using the Tracking of Indels by Decomposition (TIDE) web tool. Brinkman et al., “Easy quantitative assessment of genome editing by sequence trace decomposition” Nucleic Acids Res. 2014; 42:e168. The data show >25% indel values for the selected guides, the majority of which were deletions. See, FIG. 49C.

To evaluate the preliminary efficacy of the constructed all-in-one AAV-sgRNA-hNme1Cas9 vector, endotoxin-free sgPcsk9 plasmid was hydrodynamically administered into the C57Bl/6 mice via tail-vein injection. This method can deliver plasmid DNA to ˜40% of hepatocytes for transient expression. Liu et al., “Hydrodynamics-based transfection in animals by systemic administration of plasmid DNA” Gene Ther. 1999; 6:1258-66. Indel analyses by TIDE using DNA extracted from liver tissues revealed 5-9% indels 10 days after vector administration, comparable to the editing efficiencies obtained with analogous tests of SpyCas9. See, FIG. 49D; and Xue et al., “CRISPR-mediated direct mutation of cancer genes in the mouse liver” Nature 2014; 514:380-4. These results suggest that Nme1Cas9 is capable of editing liver cells in vivo.

Hereditary Tyrosinemia type I (HT-I) is a fatal genetic disease caused by autosomal recessive mutations in the Fah gene, which codes for the fumarylacetoacetate hydroxylase (FAH) enzyme. Patients with diminished FAH have a disrupted tyrosine catabolic pathway, have a disrupted tyrosine catabolic pathway, leading to the accumulation of toxic fumarylacetoacetate and succinyl acetoacetate, causing liver and kidney damage. Grompe M., “The pathophysiology and treatment of hereditary tyrosinemia type 1” Semin Liver Dis. 2001; 21:563-71. Over the past two decades, the disease has been controlled by 2-(2-nitro-4-trifluoromethylbenzoyl)-1,3-cyclohexanedione (NTBC), which inhibits 4-hydroxyphenylpyruvate dioxygenase upstream in the tyrosine degradation pathway, thus preventing the accumulation of the toxic metabolites. Lindstedt et al., “Treatment of hereditary tyrosinaemia type I by inhibition of 4-hydroxyphenylpyruvate Dioxygenase” Lancet 1992; 340:813-7. However, this treatment requires lifelong management of diet and medication and may eventually require liver transplantation. Das, A M., “Clinical utility of nitisinone for the treatment of hereditary tyrosinemia type-1 (HT-1)” Appl Clin Genet. 2017; 10:43-8.

Several gene therapy strategies have been tested to correct a defective Fah gene using site-directed mutagenesis or homology-directed repair by CRISPR-Cas9. Paulk et al., “Adenoassociated virus gene repair corrects a mouse model of hereditary tyrosinemia in vivo” Hepatology 2010; 51:1200-8; Yin et al., “Therapeutic genome editing by combined viral and non-viral delivery of CRISPR system components in vivo” Nat Biotechnol. 2016; 34:328-33; and Yin et al., “Genome editing with Cas9 in adult mice corrects a disease mutation and phenotype” Nat Biotechnol. 2014; 32:551-3. It has been reported that successful modification of only 1/10,000 of hepatocytes in the liver is sufficient to rescue the phenotypes of Fah^{mut/mut} mice. Recently, a metabolic pathway reprogramming approach has been suggested in which the function of the hydroxyphenylpyruvate dioxygenase (HPD) enzyme was disrupted by the deletion of exons 3 and 4 of the Hpd gene in the liver. Pankowicz et al., “Reprogramming metabolic pathways in vivo with CRISPR/Cas9 genome editing to treat hereditary tyrosinaemia” Nat Commun. 2016; 7:12642. This provides a context in which to test the efficacy of Nme1Cas9 editing, for example, by targeting Hpd and assessing rescue of the disease phenotype in Fah mutant mice. Grompe et al., “Loss of fumarylacetoacetate hydrolase is responsible for the neonatal hepatic dysfunction phenotype of lethal albino mice” Genes Dev. 1993; 7:2298-307. For this purpose, two target sites (one each in exon 8 [sgHpd1] and exon 11 [sgHpd2]) were screened and identified within the open reading frame of Hpd. See, FIG. 51A. These guides (e.g., sgRNAs) facilitated Nme1Cas9-induced average indel efficiencies of 10.8% and 9.1%, respectively, by plasmid transfections in Hepa1-6 cells. FIG. 52.

Three groups of mice were treated by hydrodynamic injection with either phosphate-buffered saline (PBS) or with one of the two sgHpd1 and sgHpd2 all-in-one AAV-sgRNA-hNme1Cas9 plasmids. One mouse in the sgHpd1 group and two in the sgHpd2 group were excluded from the follow-up study due to failed tail-vein injections. Mice were taken off NTBC-containing water seven days after injections and their weight was monitored for 43 days post injection. See, FIG. 51B. Mice injected with PBS suffered severe weight loss (a hallmark of HT-I) and were sacrificed after losing 20% of their body weight. Overall, all sgHpd1 and sgHpd2 mice successfully maintained their body weight for 43 days overall and for at least 21 days without NTBC. See, FIG. 51C.

NTBC treatment had to be resumed for 2-3 days for two mice that received sgHpd1 and one that received sgHpd2 to allow them to regain body weight during the third week after plasmid injection, perhaps due to low initial editing efficiencies, liver injury due to hydrodynamic injection, or both. Conversely, all other sgHpd1 and sgHpd2 treated mice achieved indels with frequencies in the range of 35-60%. See, FIG. 51D. This level of gene inactivation likely reflects not only the initial editing events but also the competitive expansion of edited cell lineages (after NTBC withdrawal) at the expense of their unedited counterparts. Liver histology revealed that liver damage is substantially less severe in the sgHpd1- and sgHpd2-treated mice compared to Fah^{mut/mut} mice injected with PBS, as indicated by the smaller numbers of multinucleated hepatocytes compared to PBS-injected mice. See, FIG. 53.

AAV vectors have recently been used for the generation of genome-edited mice, without the need for microinjection or electroporation, simply by soaking the zygotes in culture medium containing AAV vector(s), followed by reimplantation into pseudopregnant females. Editing was obtained previously with a dual-AAV system in which SpyCas9 and its sgRNA were delivered in separate vectors. Yoon et al., “Streamlined ex vivo and in vivo genome editing in mouse embryos using recombinant adeno-associated viruses” Nat. Commun. 9:412 (2018). To test whether Nme2Cas9 could enable accurate and efficient editing in mouse zygotes with an all-in-one AAV delivery system, the tyrosinase gene (Tyr) was targeted, where a bi-allelic inactivation of which disrupts melanin production, resulting in albino pups. Yokoyama et al., “Conserved cysteine to serine mutation in tyrosinase is responsible for the classical albino mutation in laboratory mice” Nucleic Acids Res. 18:7293-7298 (1990).

An efficient Tyr sgRNA (which cleaves the Tyr locus only 17 bp from the site of the classic albino mutation) was validated in Hepa1-6 cells by transient transfections. See, FIG. 57A-C. Next, C57BL/6NJ zygotes were incubated for 5-6 hours in culture medium containing 3×10⁹ or 3×10⁸ GCs of an all-in-one AAV6 vector expressing Nme2Cas9 along with the Tyr sgRNA. After overnight culture in fresh media, those zygotes that advanced to the two-cell stage were transferred to the oviduct of pseudopregnant recipients and allowed to develop to term. See, FIG. 58A. Coat color analysis of pups revealed mice that were albino, light grey (suggesting a hypomorphic allele of Tyr), or that had variegated coat color composed of albino and light grey spots but lacking black pigmentation. See, FIGS. 58B & 58C. These results suggest a high frequency of biallelic mutations since the presence of a single wild-type Tyr allele should render black pigmentation. A total of five pups (10%) were born from the 3×10⁹ GCs experiment. All of them carried indels; phenotypically, two were albino, one was light grey, and two had variegated pigmentation, indicating mosaicism. From the 3×10⁸ GCs experiment, four (4) pups (14%) were obtained, two of which died at birth, preventing coat color or genome analysis. Coat color analysis of the remaining two pups revealed one light grey and one mosaic pup. These results indicate that single-AAV delivery of Nme2Cas9 and its sgRNA can be used to generate mutations in mouse zygotes without microinjection or electroporation.

To measure on-target indel formation in the Tyr gene, DNA was isolated from the tails of each mouse, the locus was amplified and a TIDE analysis was performed. The data showed that all mice had high levels of on-target editing by Nme2Cas9, varying from 84% to 100%. See, FIG. 57B and FIG. 5C. Most lesions in albino mouse 9-1 were either a 1- or a 4-bp deletion, suggesting either mosaicism or trans-heterozygosity. Albino mouse 9-2 exhibited a uniform 2-bp deletion. See, FIG. 58C. Analysis of tail DNA from light grey mice revealed the presence of in-frame mutations that are potentially a cause of the light grey coat color. The limited mutational complexity suggests that editing occurred early during embryonic development in these mice. One female (mouse 9-2) was mated with a classical albino male, and all six of the resulting pups were albino, demonstrating that mutations generated by zygotic all-in-one AAV delivery of Nme2Cas9+sgRNA can be transmitted through the germline. These results provide a streamlined route toward mammalian mutagenesis through the application of a single AAV vector, in this case delivering both Nme2Cas9 and its sgRNA.

Patients with mutations in the Hpd gene are considered to have Type III Tyrosinemia and exhibit high level of tyrosine in blood, but otherwise appear to be largely asymptomatic. Szymanska et al., “Tyrosinemia type III in an asymptomatic girl. Mol Genet Metab Rep. 2015; 5:48-50; and Nakamura et al., “Animal models of tyrosinemia” J Nutr. 2007; 137:1556S-60S. HPD acts upstream of FAH in the tyrosine catabolism pathway and Hpd disruption ameliorates HT-I symptoms by preventing the toxic metabolite build-up that results from loss of FAH. Structural analyses of HPD reveal that the catalytic domain of the HPD enzyme is located at the C-terminus of the enzyme and is encoded by exon 13 and 14. Huang et al., “The different catalytic roles of the metal-binding ligands in human 4-hydroxyphenylpyruvate dioxygenase” Biochem J. 2016; 473:1179-89. Thus, frameshift-inducing indels upstream of exon 13 should render the enzyme inactive. This context was used to demonstrate that Hpd inactivation by hydrodynamic injection of Nme1Cas9 plasmid is a viable approach to rescue HT-I mice. Nme1Cas9 can edit sites carrying several different PAMs (N₄GATT [consensus], N₄GCTT, N₄GTTT, N₄GACT, N₄GATA, N₄GTCT, and N₄GACA). Hpd editing experiments confirmed one of the variant PAMs in vivo with the sgHpd2 guide, which targets a site with a N₄GACT PAM.

Although plasmid hydrodynamic injections can generate indels, therapeutic development may require less invasive delivery strategies, such as by using an rAAV. To this end, all-in-one AAV-sgRNA-hNme1Cas9 plasmids were packaged in hepatocyte-tropic AAV8 capsids to target Pcsk9 (sgPcsk9) and Rosa26 (sgRosa26). See, FIG. 49B; Gao et al., “Novel adenoassociated viruses from rhesus monkeys as vectors for human gene therapy” Proc Natl Acad Sci USA 2002; 99:11854-9; and Nakai et al., “Unrestricted hepatocyte transduction with adeno-associated virus serotype 8 vectors in mice” J Virol. 2005; 79:214-24. Pcsk9 and Rosa26 were used in part to enable Nme1Cas9 AAV delivery to be benchmarked with that of other Cas9 orthologs delivered similarly and targeted to the same loci. Ran et al., “In vivo genome editing using Staphylococcus aureus Cas9” Nature 2015; 520:186-91. Vectors were administered into C57BL/6 mice via tail vein. See, FIG. 54A. Cholesterol levels were monitored in the serum and measured PCSK9 protein and indel frequencies in the liver tissues 25 and 50 days post injection.

Using a colorimetric endpoint assay, it was determined that the circulating serum cholesterol level in the mice administered Nme1Cas9/sgPcsk9 decreased significantly (p<0.001) compared to the PBS and Nme1Cas9/sgRosa26 mice at 25 and 50 days post injection. See, FIG. 54B. Targeted deep-sequencing analyses at Pcsk9 and Rosa26 target sites revealed very efficient indels of 35% and 55%, respectively, at 50 days post vector administration. FIG. 54C. Additionally, one mouse of each group was euthanized at 14 days post injection and revealed on-target indel efficiencies of 37% and 46% at Pcsk9 and Rosa26, respectively. As expected, PCSK9 protein levels in the livers of Nme1Cas9/sgPcsk9 treated mice were substantially reduced compared to the mice injected with PBS and Nme1Cas9/sgRosa26. See, FIG. 54D. The efficient editing, PCSK9 reduction, and diminished serum cholesterol indicate the successful delivery and activity of Nme1Cas9 at the Pcsk9 locus.

SpyCas9 delivered by viral vectors is known to elicit host immune responses. Chew et al., “A multifunctional AAV-CRISPR-Cas9 and its host response” Nat Methods 2016; 13:868-74; and Wang et al., “Adenovirus-mediated somatic genome editing of Pten by CRISPR/Cas9 in mouse liver in spite of Cas9-specific immune responses” Hum Gene Ther. 2015; 26:432-42. To investigate if the mice injected with AAV8-sgRNA-hNme1Cas9 generate anti-Nme1Cas9 antibodies, sera was used from the treated animals to perform IgG1 ELISA. These results show that Nme1Cas9 elicits a humoral response in these animals. See, FIG. 55. Despite the presence of an immune response, Nme1Cas9 delivered by rAAV is highly functional in vivo, with no apparent signs of abnormalities or liver damage. See, FIG. 16.

A significant concern in therapeutic CRISPR/Cas9 genome editing is the possibility of activity at off-target edits. For example, it has been found that wild-type Nme1Cas9 is a naturally high-accuracy genome editing platform in cultured mammalian cells. Lee et al., “The Neisseria meningitidis CRISPR-Cas9 system enables specific genome editing in mammalian cells” Mol Ther. 2016; 24:645-54. To determine if Nme1Cas9 maintains its minimal off-targeting profile in mouse cells and in vivo, off-target sites were screened in the mouse genome using genome-wide, unbiased identification of DSBs enabled by sequencing (GUIDE-seq). Tsai et al., “Defining and improving the genome-wide specificities of CRISPR-Cas9 nucleases” Nat Rev Genet. 2016; 17:300-12. Hepa1-6 cells were transfected with sgPcsk9, sgRosa26, sgHpd1, and sgHpd2 all-in-one AAV-sgRNA-hNme1Cas9 plasmids and the resulting genomic DNA was subjected to GUIDE-seq analysis. Consistent with observations in human cells (data not shown), GUIDE-seq revealed very few off-target (OT) sites in the mouse genome. Four potential OT sites were identified for sgPcsk9 and another six for sgRosa26. Off-target edits with sgHpd1 and sgHpd2 were not detected. See, FIG. 56A. These data further validate that Nme1Cas9 is intrinsically hyper-accurate.

Several of the putative OT sites for sgPcsk9 and sgRosa26 lack the Nme1Cas9 PAM preferences (i.e., N₄GATT, N₄GCTT, N₄GTTT, N₄GACT, N₄GATA, N₄GTCT, and N₄GACA). See, FIG. 56B. To validate these OT sites, targeted deep sequencing was performed using genomic DNA from Hepa1-6 cells. By this more sensitive readout, indels were undetectable above background at all these OT sites except OT1 of Pcsk9, which had an indel frequency<2%. See, FIG. 56B. To validate Nme1Cas9's high fidelity in vivo, indel formation was measured at these OT sites in liver genomic DNA from the AAV8-Nme1Cas9-treated, sgPcsk9-targeted, and sgRosa26-targeted mice. Little or no detectable off-target editing was found in mice liver sacrificed at 14 days at all sites except sgPcsk9 OT1, which exhibited<2% lesion efficiency. More importantly, this level of OT editing stayed below<2% even after 50 days and also remained either undetectable or very low for all other candidate OT sites. These results suggested that extended (50 days) expression of Nme1Cas9 in vivo does not compromise its targeting fidelity. See, FIG. 56C.

To achieve targeted delivery of Nme1Cas9 to various tissues in vivo, rAAV vectors are a promising delivery platform due to the compact size of Nme1Cas9 transgene, which allows the delivery of Nme1Cas9 and its guide in an all-in-one format. The data presented herein validates this approach for the targeting of Pcsk9 and Rosa26 genes in adult mice, with efficient editing observed even at 14 days post injection. Nme1Cas9 is intrinsically accurate, even without the extensive engineering that was required to reduce off-targeting by SpyCas9. Lee et al., “The Neisseria meningitidis CRISPR-Cas9 system enables specific genome editing in mammalian cells” Mol Ther. 2016; 24:645-54; Bolukbasi et al., “Creating and evaluating accurate CRISPRCas9 scalpels for genomic surgery” Nat Methods 2016; 13:41-50; Tsai et al., “Defining and improving the genome-wide specificities of CRISPR-Cas9 nucleases” Nat Rev Genet. 2016; 17:300-12; and Tycko et al., “Methods for optimizing CRISPR-Cas9 genome editing specificity” Mol Cell. 2016; 63:355-70.

Side-by-side comparisons of Nme1Cas9 OT editing were performed in cultured cells and in vivo by targeted deep sequencing and found that off-targeting is minimal in both settings. Editing at the sgPcsk9 OT1 site (within an unannotated locus) was the highest detectable at ˜2%.

### IV. Small Cas9 Orthologs With Cytosine-Rich PAMs

As noted above, CRISPR systems may be classified into at least six (6) different types. Generally, Type II systems are categorized by the presence of a Cas9 nuclease protein. For example, a Cas9 nuclease protein is believed to be an RNA-guided nuclease that can be repurposed as a genome editing platform in almost all organisms, including humans. Reports have indicated that Cas9 genome editing has been used in medicine, agriculture, human gene therapy and many other applications.

Generally, targeting of a specific gene locus in the human genome may be accomplished by a Cas9 nuclease protein bound to a single guide RNA (sgRNA) that targets the locus via an interaction with a specific nucleic acid sequence (e.g., for example, a protospacer adjacent motif; PAM). sgRNA's usually comprise a 20-24 nucleotide segment that is complementary to a target nucleic acid sequence followed by a constant region that interacts (e.g., for example, binds) with the Cas9 protein. For the Cas9 nuclease protein to perform genome editing, the Cas9:sgRNA complex first recognizes a protospacer adjacent motif (PAM) sequence that is normally found downstream of the target site sequence. Although it is not necessary to understand the mechanism of an invention, it is believed that each Cas9 nuclease protein has affinity for a particular PAM (i.e., mediated by a protospacer adjacent motif recognition domain). In the absence of the PAM recognition domain binding to a downstream PAM target nucleic acid sequence double-stranded DNA (dsDNA) cannot be cleaved by the Cas9 nuclease.

Reports suggest that only a handful of Cas9 orthologs have been validated for human genome editing. Three of the reported CRISPR-Cas9 types include II-A, II-B and II-C. Type II-A Cas9 (e.g., Streptococcus pyogenes (SpyCas9)), is the most commonly used Cas9 to date. However, SpyCas9 (and most other type II-A orthologs) possesses several characteristics that may make it unsuitable for certain applications. First, SpyCas9 is relatively large, making this Cas9 unsuitable for efficient packaging into viral vectors. Second, SpyCas9 has a high rate of off-target activity (i.e. it cleaves DNA at unintended loci in the human genome), although higher-specificity variants have been engineered. Finally, SpyCas9's PAM (e.g., NGG) has limited use in some sites in the human genome, or for applications where a specific nucleotide is to be recognized during editing. To overcome these shortcomings, several groups have repurposed other Cas9 orthologs to function in humans and other organisms. As discussed above, type II-C Cas9 orthologs (e.g., Nme1Cas9) are small enough for all-in-one viral packaging (e.g., adeno-associated virus (AAV) vectors] that results in higher fidelity activity in mammalian cells. However, wild type Cas9 II-C PAMs are usually approximately four (4) nucleotides in length as opposed to an SpyCas9 PAM that is usually two (2) nucleotides in length. This additional PAM length can limit the number of loci that can be targeted by a wild type Cas9 II-C PAM. This creates a need in the art for the identification of more Cas9 orthologs for genome editing.

While there are thousands of Cas9 orthologs in the NCBI database to choose from, an empirical process is required to develop small type II-C Cas9 orthologs with less restrictive PAMs that provide improved functionality in mammalian cells. In one embodiment, the present invention contemplates an improved type II-C Cas9 ortholog that enables precise genome editing with a broader range of target sites. In one embodiment, the improved type II-C Cas9 ortholog has a compact size capable of efficient viral delivery. In one embodiment, the improved type II-C Cas9 ortholog includes, but is not limited to, Haemophilus parainfluenzae (HpaCas9), Simonsiella muelleri (SmuCas9) and Neisseria meningitidis strain De10444 (Nme2Cas9).

### A. Short PAMs Associated With Type II-C Cas9 Orthologs

The data presented herein shows the characterization of short PAM targets for several type II-C Cas9 orthologs. FIG. 17. For example, type II-C Cas9 orthologs may interact with short PAMs comprising between one-four required nucleotides. Although it is not necessary to understand the mechanism of an invention, it is believed that these short C-rich PAMs provide improved Cas9 genome editing of target sites previously not accessible even by the more compact Cas9 orthologs (e.g., Nme1Cas9). In one embodiment, an Nme2Cas9 PAM has a sequence of NNNNCc, wherein “c” is the only a partial preference. In one embodiment, an SmuCas9 PAM has a sequence of NNNNCT. FIG. 18.

It is currently believed that no Cas9 orthologs with short C-rich PAMs have been validated for genome editing and that Nme2Cas9 is particularly compelling as a potential candidate for highly efficient gene editing activity in human cells. In one embodiment, the present invention contemplates an Nme2Cas9 nuclease bound to a wild type Nme1Cas9 sgRNA (e.g., Neisseria meningitidis 8013 Cas9; previously referred to as NmeCas9). Nme1Cas9 has been previously described. Sontheimer et al., “RNA-Directed DNA Cleavage and Gene Editing by Cas9 Enzyme From Neisseria meningitidis” United States Patent Application Publication Number 2014/0349,405 (herein incorporated by reference). Although Nme1Cas9 can be useful for genome editing, its main limitation is its relatively long PAM, which restricts the number of editable sites in any given genomic locus.

In some embodiments, the present invention contemplates shorter and less stringent PAMs for type II-C Cas9 orthologs including, but not limited to, Nme2Cas9. Although it is not necessary to understand the mechanism of an invention, it is believed that short and less stringent PAMs partially relieve target restriction limitations, while still leaving many, if not most, of the advantages of Nme1Cas9 including, but not limited to, small size (e.g., compactness) for efficient all-in-one AAV delivery and improved target accuracy (e.g., reductions in off-target cleavages). In addition, minimized sgRNAs for Nme1Cas9 discussed above are also compatible with Nme2Cas9 constructs. Consequently, such truncated guide RNAs could likely be used for genome editing with Nme2Cas9 as well.

In one embodiment, the present invention contemplates an HpaCas9 PAM having a sequence of NNNNGNTTT. Despite the fact that the long PAM limits the number of targetable sites in the human genome it is believed that the HpaCas9 PAM may target sites with very high accuracy that is similar to the extreme accuracy Nme1Cas9 (supra).

The data presented herein demonstrates the ability of type II-C Cas9 nucleases targeted to short C-rich PAMs to perform genome editing in human (HEK293T) cells. Certo et al., “Tracking genome engineering outcome at individual DNA breakpoints” Nature Methods 8:671-676 (2011). For example, HpaCas9 and Nme2Cas9, were shown to provide efficient genome editing at specific loci demonstrating that they are active in mammalian cells. FIG. 19 and Table 2.

TABLE 2
Representative Type II-C Cas9 Orthologs Target Sequences in The Human Genome

| Cas9 | Spacer sequence | PAM | Chromosome | SEQ ID NOS: |
|---|---|---|---|---|
| Nme2 | GAATATCAGGAGACTAGGAAGGAG | GAGGCCTA | 19 | 22, 23 |
| Hpa | GGACAGGAGTCGCCAGAGGCCGGT | GGTGGATTT | 4 | 24, 25 |
| Smu | GCACCTGCCTCGTGGAATACGGT | AAACCTAC | Traffic Light | 26, 27 |
| | | | Reporter | |

These data show that both Nme2Cas9 and HpaCas9 performed genome editing at comparable levels to the previously validated Nme1Cas9 at the same genomic locus. For SmuCas9, the efficiency of editing is relatively low, though it is significant that the activity is not zero, and efficiency improvements are expected. Nme2Cas9 was then used to test fourteen (14) additional sites in the traffic light reporter (TLR) integrated into the genome of HEK293T cells. In these assays, each site conforms to a PAM template that a “C” is the fifth nucleotide of the PAM region (i.e., NNNNCNNN). Remarkably, all fourteen sites were edited by Nme2Cas9, indicating that this enzyme is consistently active with a variety of guides in mammalian cells. The most successful guide RNAs conform to the NNNNCCN PAM consensus. FIG. 20.

Type II-C Cas9 ortholog cleavage was tested for sensitivity to anti-CRISPR proteins. Anti-CRISPR proteins are naturally occurring proteins that can turn Cas9 off when Cas9 activity is no longer desired. The data show that all three Type II-C Cas9 orthologs are inhibited by certain anti-CRISPRs. FIG. 21. The controllability of these Cas9 orthologs by anti-CRISPRs could increase their potential utility in genome editing.

### B. Nme2Cas9 Gene Editing

The data presented herein shows gene editing using the Nme2Cas9-sgRNA complex. The data employs the traffic light reporter (TLR) system to demonstrate that any CC dinucleotide in a gene target sequence can function as a PAM, within the context of an NNNNCC sequence (supra). FIG. 22. Black bars are the % of cells that exhibit fluorescence, whereas gray bars indicate % editing more accurately based on sequencing (“TIDE analysis”). These data confirm that a dinucleotide is sufficient for Nme2Cas9 PAM binding as opposed to a requirement for a trinucleotide sequence (e.g, the “X” in the sequence NNNNCCX). Although it is not necessary to understand the mechanism of an invention, it is believed that this means that Nme2Cas9 editable genomic target sites are at least as frequent as SpyCas9 editable sites, and more frequent than with SauCas9, Nme1Cas9 or CjeCas9 and other current alternatives.

Furthermore, T7E1 assays were employed to analyze editing of native genomic sites (e.g., not an integrated, artificial fluorescent reporter). These data suggest that, in some situations, the second “C” might not even be required. See, FIG. 23. Note that target sites DeTS1 and DeTS4, both in the AAVS1 locus, enables editing at target sites with NNNNCA and NNNNCG candidate PAMs, respectively. Several of these Nme2Cas9 target sites are disclosed herein. See, Table 3.

TABLE 3
Representative PAM Target Sites For Nme2Cas9

| Target site | Target | | SEQ ID |
|---|---|---|---|
| name | locus | Target Sequence (Spacer-PAM) | NOS: |
| Nme2TS1 | AAVS1 | ATGTGGCTCTGGTTCTGGGTACTTTTATCTGTCCC | 28 |
| | | CTCCACCCCACAGTGGG | |
| Nme2TS4 | AAVS1 | CAGATAAGGAATCTGCCTAACAGGAGGTGGGGG | 29 |
| | | TTAGACGAATATCAGGAGA | |
| Nme2TS5 | AAVS1 | GGGGTTAGACGAATATCAGGAGACTAGGAAGGA | 30 |
| | | GGAGGCCTAAGGATGGGGG | |
| Nme2TS6 | Chr. 14 | CCCACCCGGCGGCGCCTCCCTGCAGGGCTGCTCC | 31 |
| | | CCAGCCCAAACCGCCGCG | |
| Nme2TS10 | AAVS1 | TCCGAGAGCTCAGCTAGTCTTCTTCCTCCAACCC | 32 |
| | | GGGCCCTATGTCCACTTC | |
| Nme2TS11 | AAVS1 | TGGGTACTTTTATCTGTCCCCTCCACCCCACAGTG | 33 |
| | | GGGCCACTAGGGACAGG | |
| Nme2TS12 | AAVS1 | GTAGGGGAGCTGCCCAAATGAAAGGAGTGAGAG | 34 |
| | | GTGACCCGAATCCACAGGA | |
| Nme2TS13 | AAVS1 | TAGCACCTCTCCATCCTCTTGCTTTCTTTGCCTGG | 35 |
| | | ACACCCCGTTCTCCTGT | |
| Nme2TS14 | AAVS1 | GTCTCCCTTGCGTCCCGCCTCCCCTTCTTGTAGGC | 36 |
| | | CTGCATCATCACCGTTT | |
| Nme2TS15 | AAVS1 | CCTCACCCAACCCCATGCCGTGTTCACTCGCTGG | 37 |
| | | GTTCCCTTTTCCTTCTCCT | |
| Nme2TS16 | Chr. 14 | GCGCAGGACAGGAGTCGCCAGAGGCCGGTGGTG | 38 |
| | | GATTTCCTCCCCGCATCTC | |
| Nme2TS17 | Chr. 14 | CGCGGGGACGCCCAGCGGCCGGATATCAGCTGC | 39 |
| | | CACGCCCGCGTGGGCGGA | |
| Nme2TS22 | VEGF | GATTCCAATAGATCTGTGTGTCCCTCTCCCCACCC | 40 |
| | | GTCCCTGTCCGGCTCTC | |
| Nme2TS23 | VEGF | TGACCCCTGGCCTTCTCCCCGCTCCAACGCCCTCA | 41 |
| | | ACCCCACACGCACACAC | |
| Nme2TS24 | VEGF | TCCCTCTCCCCACCCGTCCCTGTCCGGCTCTCCGC | 42 |
| | | CTTCCCCTGCCCCCTTC | |
| Nme2TS25 | VEGF | ACACGCACACACTCACTCACCCACACAGACACAC | 43 |
| | | ACGTCCTCACTCTCGAAG | |
| Nme2TS26 | Chr. 7 | TAAGCACAGTGGAAGAATTTCATTCTGTTCTCAG | 44 |
| | (CFTR) | TTTTCCTGGATTATGCCT | |
| Nme2TS27 | Chr. 7 | TTCATTCTGTTCTCAGTTTTCCTGGATTATGCCTG | 45 |
| | (CFTR) | GCACCATTAAAGAAAAT | |

Although it is not necessary to understand the mechanism of an invention, it is believed that these data suggest that there may be candidate editing sites in a genome at every 4-8 base pairs, on average. These data also suggest that most Cas9 sgRNAs have some functionality, consequently the need for sgRNA screening may be overemphasized in the art.

### C. Rapidly-Evolving PAM-Interacting Domains

In vivo applications of CRISPR-Cas9 have the potential to transform many areas of biotechnology and therapeutics. There are thousands of Cas9 orthologs in nature, only a handful of which have been validated for in vivo genome editing. The Cas9 from Streptococcus pyogenes (SpyCas9) has been widely used due to its high efficiency and non-restrictive NGG protospacer adjacent motif (PAM). However, the relatively large size of SpyCas9 restricts its use in in vivo therapeutic applications using delivery shuttles with limited packaging capacity such as adeno-associated virus (AAV). Several smaller Cas9 orthologs are known to be active in mammalian cells, but they possess more restrictive PAMs that limit target site density. The natural variation in the PAM Interacting Domains (PIDs) of closely related Cas9 orthologs may be taken advantage of to identify a genome editing enzyme that overcomes these limitations. In some embodiments, the present invention contemplates using an Nme2Cas9 complex which is compact, naturally hyper-accurate Cas9 with an N₄CC PAM. The data presented herein show that Nme2Cas9 is a high-fidelity mammalian genome editing platform that affords the same target site density as SpyCas9. Delivery of Nme2Cas9 with its guide RNA via an all-in-one AAV vector leads to efficient genome editing in adult mice, with Pcsk9 gene targeting in the liver inducing serum cholesterol reduction with no significant off-targeting (infra). Nme2Cas9 also provides a unique combination of all-in-one AAV compatibility, natural hyper-accuracy, and high target site density for in vivo genome editing in mammals.

In addition to target density, minimizing off-target activity (e.g., cleavage at undesired loci) of a Cas9 is highly desirable for its use as a safe therapeutic agent. Wild-type (wt) SpyCas9 possesses a high degree of off-target activity due to its unique hybridization kinetics. (Klein et al, 2018). In particular, questions remain regarding their on-target editing efficiency and these variants do not overcome the above discussed limitations regarding overall size. In contrast, it has been shown herein that embodiments of Nme1Cas9 and CjeCas9 comprise naturally accurate gene editing activity. Although it is not necessary to understand the mechanism of an invention, it is believed that no Cas9 ortholog has been previously reported that: i) is active in human cells; ii) exhibits the exceptionally high target-site density of SpyCas9; iii) is sufficiently compact for all-in-one AAV deliverability; and iv) is naturally hyper-accurate. In one embodiment, the present invention contemplates an Nme2Cas9 as a genome editing platform comprising all of the characteristics described above. For example, Nme2Cas9 comprises a binding site comprising a high affinity for an N₄CC PAM, is hyper-accurate and functions efficiently in mammalian cells. In one embodiment, Nme2Cas9 is packaged in an all-in-one AAV delivery platform for therapeutic genome editing.

### 1. Closely-Related Nme1Cas9 Orthologs With Rapidly-Evolving PIDs

It has previously been reported that Nme1Cas9 (from Neisseria meningitidis strain 8013) is a small, hyper-accurate Cas9 for in vivo genome editing (Amrani et al, 2018). However, Nme1Cas9 binds to a long PAM (N₄GMTT) which limits its use in certain contexts where a small window can be targeted. PAM recognition by Cas9 occurs predominantly through protein-DNA interaction between the PAM-Interacting Domain (PID) of Cas9 and the nucleotides adjacent to the PAM. PIDs are subject to high selection pressure by phages and other mobile genetic elements (MGEs). For example, anti-CRISPR proteins have been shown to interact with PIDs to inhibit Cas9 (infra). This may result in closely-related Cas9 orthologs having PIDs that recognize drastically different PAMs.

Recently, this principle was highlighted using two species of Geobacillus. G. sterothermpophilus's was determined to comprise a PID specific for a N₄CRAA PAM but when exchanged for a strain LC300 PID its affinity changed to a N₄GMAA PAM (Harrington et al, 2017). It was hypothesized that given that N. meninigitidis strains are highly sequenced, a closely related Cas9 ortholog could be found with rapidly-evolved PIDs that recognize different PAMs. Cas9 orthologs with high sequence identity (>80%) to NmeCas9 strain 8013 were investigated because this Cas9 has been fully characterized for genome editing, is small and hyper-accurate. Several Cas9 orthologs were identified which differed in their PID amino acid sequences a compared with strain 8013. FIG. 34A.

Three distinct groups of Cas9 orthologs were found with drastically different PIDs. FIG. 35A. One strain was selected from each PID group, for example, De11444 from group 2 and 98002 from group 3. These two CRISPR loci had intact Cas9 open reading frames and CRISPR arrays with several spacers, which suggest they are active loci. Interestingly, the crRNA and tracrRNA of these CRISPR loci were identical to that of 8013 and can utilize the same sgRNAs. FIG. 35B.

To test whether these Cas9 orthologs indeed had PIDs with affinity for different PAMs, because of the high sequence identity in the remainder of the protein from these orthologs, the 8013 PID was interchanged with the 98002 PID and the De11444 PID. To identify the PAMs, these protein “chimeras” were recombinantly expressed, purified and used for in vitro PAM identification as described previously. Briefly, a DNA fragment comprising a protospacer and a ten (10) nucleotide randomized sequence downstream was cleaved in vitro using recombinant Cas9 and an sgRNA targeting the protospacer. FIG. 34B. A G23 nucleotide spacer length was used for the sgRNA, consistent with Nme1Cas9 8013 and other type II-C systems studied. The PAM identification assay revealed that these different Cas9 chimeras had PIDs recognizing different PAMs. For example, by recognizing a C residue at position 5 instead of a G recognized by Nme1Cas9 8013 with its N₄GATT PAM. FIG. 34C.

However, the remaining nucleotides could not be confidently characterized due to the low cleavage efficiency of the chimeric proteins, which suggests that the few residues outside of the PID are likely involved for efficient activity. FIG. 35C. To further resolve the PAMs, an in vitro assay was performed on a library with a 7-nucleotide randomized PAM, with a C at position 5 (e.g., NNNNCNNN). The results suggested that NmeCas9-De11444 and NmeCas9-98002 recognized NNNNCC(A) and NNNNCAAA PAMs, respectively. FIG. 35D. NmeCas9-De11444 had a strong preference for the C at position 5, but less so for nucleotides 6 and 7. As used herein, the Cas9 De11444 ortholog is termed “Nme2Cas9”, and the Cas9 98002 ortholog is termed “Nme3Cas9”.

We also performed this assay using full-length (e.g., not PID-swapped) Nme2Cas9 and observed similar results. FIG. 34E. These results suggest that Nme2Cas9 and Nme3Cas9 have PIDs recognizing drastically different PAMs than that of Nme1Cas9.

### 2. Nme2Cas9 in Human Cells

Because the Nme2Cas9 PID binds with a small PAM sequence, this ortholog is useful for human genome editing, especially when high-targeting density is involved. To characterize the Nme2Cas9, a full-length (not PID-swapped) humanized Nme2Cas9 was cloned into a CMV-driven plasmid along with NLSs for mammalian expression. For characterization in human cells, a Traffic Light Reporter system was used similar to the one described previously (Certo et al., 2011)

Induction of +1 frameshift indels were created by imperfect repair via non-homologous end joining (NHEJ) in the TLR 2.0 locus. In the absence of a donor DNA an in-frame mCherry protein resulted, which can be quantified through flow cytometry. FIG. 36A. As an initial test, a Nme2Cas9 plasmid was transfected along with fifteen (15) sgRNA plasmids with spacers targeting protospacers with N₄CCX PAMs. As controls, SpyCas9 and Nme1Cas9 were used along with their cognate sgRNAs targeting NGG and N₄GATT protospacers, respectively. Cells were harvested after seventy-two (72) hours and the number of mCherry positive cells was quantified for each target site. SpyCas9 and Nme1Cas9 showed efficient editing at their respective targets (˜28% and 10% mCherry, respectively) FIG. 36B. For Nme2Cas9, all fifteen (15) targets with N₄CCX PAMs were functional to various degrees (ranging from 4% to 20% mCherry), while NmeCas9 treatments without accompanying sgRNA and/or N₄GATT controls yielded no mCherry cells. FIG. 36B. These data suggested that Nme2Cas9 recognizes an N₄CC PAM in human cells.

To further resolve Nme2Cas9 PAMs, target sites were also tested with N₅CX and N₄CD (D=A, T, G) in TLR reporter cells. No detectable editing was observed at target sites with N₅CX and N₄CD PAMs, suggesting that both C nucleotides at positions 5 and 6 are required for Nme2Cas9's activity based on the TLR 2.0 reporter. FIG. 37A and FIG. 37B. These results demonstrate that Nme2Cas9 comprises a PID that binds to an N₄CC PAM and is consistently functional in mammalian cells at the TLR 2.0 locus.

The length of the spacer portion of the crRNA differs between different Cas9 orthologs. SpyCas9's optimal spacer length is twenty (20) nucleotides, however, truncations down to seventeen (17) nucleotides are tolerated. Fu et al., Nature Biotechnology 32, 279 (2014). In contrast, Nme1Cas9 comprises sgRNAs with twenty-four (24) nucleotide spacers and tolerates truncations down to eighteen (18) nucleotides. (Amrani et al., 2018). To test the spacer length for Nme2Cas9, sgRNA plasmids were created that targeted the same locus, but with varying spacer lengths. FIG. 36C and FIG. 37B. Comparable activities were observed when G23, G22 and G21 spacers were used, with a significant decrease in activity when the guide was truncated to G20 and G19. FIG. 36C. These results suggest that Nme2Cas9's optimal spacer length is between 22-24 nucleotides, similar to that of Nme1Cas9, GeoCas9 and CjeCas9. Therefore, all experiments described below were performed with 23-24 nucleotide spacers.

Cas9 orthologs are believed to use their HNH and RuvC domains to induce a double stranded break in the complementary and non-complementary strands of the target DNA, respectively. Alternatively, Cas9 nickases have been used to improve genome editing specificity and homology-directed repair (HDR) by creating overhangs. (Ran et al, 2013). However, this approach has only been successful by use of SpyCas9 due to its high target density. To use Nme2Cas9 as a nickase, Nme2Cas9^{D16A} and Nme2Cas9^{H588A} were created which provide mutations in the catalytic residues of the RuvC and HNH domains, respectively. Since TLR 2.0 can also be used to study the efficiency of HDR, where a repaired locus expresses GFP when a donor is provided, a donor DNA sequence was included to test HDR with these Nme2Cas9 nickases. Target sites were selected within the TLR 2.0 gene to test the functionality of each nickase using guide RNAs that targeted cleavage sites spaced 32 bp and 64 bp apart. As a control, wild type Nme2Cas9 targeted to a single site showed efficient editing, accompanied by induction of both NHEJ and HDR repair pathways. For nickases, the cleavage sites spaced 32 bp and 64 bp apart showed editing using the Nme2Cas9^{D16A} (HNH nickase), but neither target was nicked using Nme2Cas9^{H588A}. FIG. 36D.

Cas9 orthologs comprise a seed sequence that usually hybridizes to a target sequence between eight to twelve (8-12) nucleotides proximal to the PAM. Mismatches (e.g., non-complementarity) between the seed sequence and the PAM can reduce Cas9 nuclease activity. A series of transient transfections were performed that targeted the same locus in the TLR 2.0 gene by walking single nucleotide mismatches along a twenty-three (23) nucleotide spacer. FIG. 37C. Similar to other Cas9 orthologs, the data suggest that Nme2Cas9 possesses a “seed sequence” in the first eight-to-nine (8-9) nucleotides that hybridize to a target sequence proximal to the PAM, as deduced from the decrease in the number of mCherry positive cells. Even though tolerance to mismatches is highly dependent on the sequence and the target locus of an sgRNA, these results suggest that Nme2Cas9 has very low tolerance for mismatches particularly in its seed sequence.

### 3. Nme2Cas9 Genome Editing Efficiency

Nme2Cas9 was used to target forty (40) different target sites throughout the human genome in HEK293T cells using transient transfections. Table 4.

TABLE 4
Representative HEK293T Cell Nme2Cas9 Target Sites

| SEQ ID NOS: | Number | Ste Name | Spacer Seq | P [symbol] | L [symbol] | [symbol] Cas |
|---|---|---|---|---|---|---|
| 46, 47, 48, 49 | 1 | T [symbol] | GGTT [symbol] TGGGTACTTTATCTGTC [symbol] | CCTCCA [symbol] C | AAV [symbol] 1 | [symbol] 2 |
| 50, 51, 52, 53 | 2 | T [symbol] | GTCTGCCTAACAGGAGG [symbol] GGGGGT | TAGA [symbol] A | AAV [symbol] | [symbol] 1 |
| 54, 55, 56, 47 | 3 | T [symbol] | GAATATCAGG [symbol] GGAAGGAG | G [symbol] [symbol] [symbol] | AAV [symbol] | 18 |
| 58, 59, 60, 61 | 4 | T [symbol] | [symbol] | C [symbol] [symbol] A | [symbol] | [symbol] |
| 62, 63, 64, 65 | 5 | [symbol] | [symbol] | [symbol] | A [symbol] | [symbol] |
| 66, 67, 68, 69 | 6 | [symbol] | [symbol] | [symbol] | [symbol] | [symbol] |
| 70, 71, 72, 73 | 7 | [symbol] | [symbol] | T [symbol] | [symbol] | [symbol] |
| 74, 75, 76, 77 | 8 | T [symbol] | [symbol] | [symbol] | [symbol] | [symbol] |
| 78, 79, 80, 81 | 9 | T [symbol] | [symbol] | ATTT [symbol] | [symbol] | [symbol] |
| 82, 83, 84, 85 | 10 | T [symbol] | [symbol] CCCA [symbol] | [symbol] | [symbol] | [symbol] |
| 86, 87, 88, 89 | 11 | T [symbol] | [symbol] | TTT [symbol] | [symbol] | 1 |
| 90, 91, 92, 93 | 12 | T [symbol] | [symbol] | CTAT [symbol] | [symbol] | [symbol] |
| 94, 95, 96, 97 | 13 | T [symbol] | GA [symbol] | CT [symbol] | C [symbol] | 11.2 |
| 98, 99, 100, 101 | 14 | T [symbol] | GT [symbol] | CA [symbol] | [symbol] | 1 |
| 102, 103, 104, 105 | 15 | T [symbol] | [symbol] V [symbol] | [symbol] | [symbol] | [symbol] |
| 106, 107, 108, 109 | 16 | T [symbol] | [symbol] | C [symbol] | [symbol] | [symbol] |
| 110, 111, 112, 113 | 17 | T [symbol] | [symbol] | GGCA [symbol] | [symbol] | [symbol] |
| 114, 115, 116, 117 | 18 | T [symbol] | G [symbol] | [symbol] | [symbol] | [symbol] |
| 118, 119, 120, 121 | 19 | T [symbol] | [symbol] | AGGG [symbol] | [symbol] | [symbol] |
| 122, 123, 124, 125 | 20 | T [symbol] | [symbol] | [symbol] | [symbol] | [symbol] |
| 126, 127, 128, 129 | 21 | T [symbol] | [symbol] | [symbol] | [symbol] | [symbol] |
| 130, 131, 132, 133 | 22 | T [symbol] | G [symbol] | [symbol] | [symbol] | [symbol] |
| 134, 135, 136, 137 | 23 | T [symbol] | GT [symbol] | [symbol] | [symbol] | [symbol] |
| 138, 139, 140, 141 | 24 | T [symbol] | [symbol] | CC [symbol] | AGA | [symbol] |
| 142, 143, 144, 145 | 25 | T [symbol] | GCT [symbol] | TCT [symbol] | VEGFA | [symbol] |
| 146, 147, 148, 149 | 26 | T [symbol] | GT [symbol] | [symbol] | VEGFA | 4 |
| 150, 151, 152, 153 | 27 | T [symbol] | GAT [symbol] | [symbol] | VEGFA | 8 |
| 154, 155, 156, 157 | 28 | T [symbol] | GTG [symbol] | CTG [symbol] | VEGFA | [symbol] |
| 158, 159, 160, 161 | 29 | T [symbol] | GTT [symbol] | GTCA [symbol] | VEGFA | 2 |
| 162, 163, 164, 165 | 30 | T [symbol] | GCGTTGGA [symbol] | [symbol] | VEGFA | 4 |
| 166, 167, 168, 169 | 31 | T [symbol] | GTACCCTCCAATAATTT [symbol] | AATT [symbol] | AGA | 8 |
| 170, 171, 172, 173 | 32 | T [symbol] | [symbol] AT [symbol] ATTT | CA [symbol] | AGA | [symbol] |
| 174, 175, 176, 177 | 33 | T [symbol] | [symbol] | GG [symbol] | VEGFA | [symbol] |
| 178, 179, 180, 181 | 34 | T [symbol] | [symbol] | [symbol] | VEGFA | [symbol] |
| 182, 183, 184, 185 | 35 | T [symbol] | [symbol] | C [symbol] | VEGFA | [symbol] |
| 186, 187, 188, 189 | 36 | T [symbol] | [symbol] | [symbol] | VEGFA | [symbol] |
| 190, 191, 192, 193 | 37 | T [symbol] | [symbol] | [symbol] | VEGFA | [symbol] |
| 194, 195, 196, 197 | 38 | T [symbol] | G [symbol] | [symbol] | VEGFA | 18 |
| 198, 199, 200, 201 | 39 | T684 | GAA [symbol] | [symbol] | F [symbol] | [symbol] |
| 202, 203, 204, 205 | 40 | T365 | [symbol] | [symbol] | F [symbol] | [symbol] |

| SEQ ID NOS: | [symbol] name | [symbol] primer | [symbol] primer |
|---|---|---|---|
| 46, 47, 48, 49 | AAVEE_MD [symbol] | T [symbol] CCAT | AGA [symbol] TTCTG [symbol] [symbol] [symbol] [symbol] [symbol] [symbol] [symbol] [symbol] [symbol] [symbol] |
| 50, 51, 52, 53 | AAV [symbol] _T [symbol] 1 | T [symbol] | [symbol] |
| 54, 55, 56, 47 | L [symbol] | A [symbol] | AG [symbol] |
| 58, 59, 60, 61 | [symbol] | [symbol] | [symbol] |
| 62, 63, 64, 65 | [symbol] | TG [symbol] | [symbol] |
| 66, 67, 68, 69 | AAV [symbol] _ [symbol] | TCC [symbol] | [symbol] |
| 70, 71, 72, 73 | A [symbol] | A [symbol] | TAGGAA [symbol] |
| 74, 75, 76, 77 | [symbol] | [symbol] | TAGGAA [symbol] |
| 78, 79, 80, 81 | L [symbol] _T | AG [symbol] A | ATGACAGAC [symbol] |
| 82, 83, 84, 85 | L [symbol] _TI | A [symbol] | AT [symbol] |
| 86, 87, 88, 89 | [symbol] | [symbol] | CCAAT [symbol] |
| 90, 91, 92, 93 | [symbol] | [symbol] | [symbol] CAATATTGCAT [symbol] GG |
| 94, 95, 96, 97 | [symbol] | [symbol] | CCAATATTGCATGGGATGG |
| 98, 99, 100, | [symbol] _ [symbol] | [symbol] CCA | AT [symbol] |
| 102, 103, 104, 105 | [symbol] _ [symbol] | TGGTGATT [symbol] | AT [symbol] |
| 106, 107, 108, 109 | VEGF_T [symbol] | [symbol] | ACCAT [symbol] |
| 110, 111, 112, 113 | W_T [symbol] | TGG [symbol] | ACCAT [symbol] |
| 114, 115, 116, 117 | VEGF_T [symbol] | [symbol] | [symbol] |
| 118, 119, 120, 121 | L [symbol] _ [symbol] | A [symbol] | AT [symbol] |
| 122, 123, 124, 125 | LN [symbol] | [symbol] | ATGA [symbol] |
| 126, 127, 128, 129 | LN [symbol] | [symbol] | ATGACAGAC [symbol] |
| 130, 131, 132, 133 | LN [symbol] | [symbol] | ATGAC [symbol] |
| 134, 135, 136, 137 | LN [symbol] | A [symbol] | ATGACAGAC [symbol] |
| 138, 139, 140, 141 | [symbol] | [symbol] | CAT [symbol] |
| 142, 143, 144, 145 | VEGF_T [symbol] | [symbol] | AT [symbol] |
| 146, 147, 148, 149 | VEGF_T [symbol] | [symbol] | AT [symbol] |
| 150, 151, 152, 153 | VEGF_T [symbol] | [symbol] | AT [symbol] |
| 154, 155, 156, 157 | VEGF_T [symbol] | G [symbol] | A [symbol] |
| 158, 159, 160, 161 | VEGF_T [symbol] | GT [symbol] | [symbol] |
| 162, 163, 164, 165 | VEGF_T [symbol] | [symbol] | [symbol] |
| 166, 167, 168, 169 | AGA_T [symbol] | [symbol] | CATGTCCTGAA [symbol] |
| 170, 171, 172, 173 | AGA_T [symbol] | G [symbol] | CATGTC [symbol] |
| 174, 175, 176, 177 | VEGF_T [symbol] | [symbol] | [symbol] |
| 178, 179, 180, 181 | VEGF_T [symbol] | [symbol] | [symbol] |
| 182, 183, 184, 185 | V [symbol] _ [symbol] | [symbol] | [symbol] |
| 186, 187, 188, 189 | V [symbol] GF_ [symbol] | [symbol] | [symbol] |
| 190, 191, 192, 193 | V [symbol] _T [symbol] | [symbol] | [symbol] |
| 194, 195, 196, 197 | V [symbol] _T [symbol] | [symbol] | [symbol] |
| 198, 199, 200, 201 | [symbol] _ [symbol] | [symbol] | CTT [symbol] |
| 202, 203, 204, 205 | [symbol] _ [symbol] | G [symbol] | CTT [symbol] |

[symbol]

indicates data missing or illegible when filed

72-hours post transfection, cells were harvested followed by gDNA extraction and selective amplification of the targeted locus. A Tracking of Indels by Decomposition (TIDE) analysis was used to measure indel rates at each locus. Efficient editing by Nme2Cas9 was observed, even though indel rates varied significantly depending on the target sequence and the locus. FIG. 38A. Moreover, Nme2Cas9's affinity for target sites near/at therapeutically-relevant loci such as CYBB (mutations cause x-linked chronic granulomatous disease) and AGA (mutations cause aspartylglycosaminuria) suggests Nme2Cas9 has therapeutic potential. In addition, editing efficiency could be increased by increasing the quantity of the Nme2Cas9 plasmid. FIG. 39A. Taken together, these results demonstrate that Nme2Cas9 can be constructed to selectively edit specific target genomic sites in HEK293T cells.

In addition to HEK293T cells, Nme2Cas9's gene editing efficiency was determined in several other mammalian cells, including human leukemia K562 cells, human osteosarcoma U2OS cells and mouse liver hepatoma Hepa1-6 cells. A lentiviral construct expressing Nme2Cas9 was created and transduced K562 cells to stably express Nme2Cas9 under the control of SFFV promoter. This stable cell line did not show any significant differences with respect to growth and morphology as compared to untreated cells, suggesting Nme2Cas9 is not toxic when stably expressed. These cells were transiently electroporated with plasmids expressing sgRNAs targeting several target sites and analyzed after seventy-two (72) hours for indel rates by TIDE. Efficient editing was observed at the three sites tested, demonstrating Nme2Cas9's ability to function in K562 cells. For Hepa1-6 cells, plasmids encoding Nme2Cas9 and sgRNA were co-transfected using techniques similar to HEK293T transduction described above. These data also show that Nme2Cas9 efficiently edited Pcsk9 and Rosa26 sites in this mouse cell line. FIG. 38B.

Previous work suggests that ribonucleoprotein (RNP) delivery of Cas9s, instead of plasmid transfection, may be an alternative choice for some genome editing applications. For example, off-target effects of SpyCas9 may be significantly reduced with RNP electroporations compared to plasmid delivery. Kim et al., Genome Research 24:1012-1019 (2014). To test whether Nme2Cas9 is functional by RNP delivery, a His-tagged Nme2Cas9 was cloned along with three (3) nuclear localization signals (NLSs) and a purified recombinant protein into a bacterial expression construct. sgRNAs targeting several validated target sites were generated by T7 in vitro transcription. Electroporation of a Nme2Cas9:sgRNA complex induced successful editing at the target sites, as detected by TIDE. FIG. 38C. These results suggest that Nme2Cas9 can be delivered as a plasmid, or as an RNP complex. Overall, these results demonstrate that Nme2Cas9 is functional in various cell types with different modes of delivery.

### 4. Anti-CRISPR Protein Inhibition

Five (5) anti-CRISPR (Acr) protein families against Nme1Cas9 from diverse bacterial species have been reported to inhibit Nme1Cas9 in vitro and in human cells. (Pawluk et al. 2016, Lee et al., mBio, in press). Considering the high sequence identity between Nme1Cas9 and Nme2Cas9, it seemed likely that at least some species within these Acr families might also inhibit Nme2Cas9. All five Acr families were recombinantly expressed, purified and Nme2Cas9's ability to cleave a target sequence in vitro was tested (10:1 Acr:Cas9 molar ratio). As a negative control, an inhibitor for the type I-E CRISPR system in E. coli (AcrE2) was used. As expected, all Arc families inhibited Nme1Cas9, while AcrE2 failed to do so. In particular, Acrs IIC1_{Nme}, -IIC2_{Nme}, -IIC3_{Nme} and -IIC4_{Hpa} inhibited Nme2Cas9 gene editing activity. FIG. 40A, top.

Strikingly, AcrIIC5_{Smu} did not inhibit Nme2Cas9 in vitro even at 10-fold excess, suggesting that it likely inhibits Nme1Cas9 by interacting with a PID. To further confirm this, the same in vitro cleavage assay was performed using a hybrid version of NmeCas9 (e.g., Nme1Cas9 with the PID of Nme2Cas9). Due to the reduced activity of this hybrid, higher concentration (˜30×) of Cas9 was used to achieve similar cleavage profile while maintaining the 10:1 Cas9:Acr molar ratio. Consistent with the initial results, no inhibition by AcrIIC5_{Smu} on this protein chimera was observed. FIG. 41. The inability of AcrIIC5_{Smu} to inhibit the hybrid protein further suggests that AcrIIC5_{Smu} likely interacts with the PID of Nme1Cas9.

The above in vitro data, suggested that Acrs-IIC1_{Nme}, -IIC2_{Nme}, -IIC3_{Nme} and -IIC4_{Hpa} could be used as off-switches for Nme2Cas9 genome editing. To test this, transfections were performed as described above in the presence or absence of plasmids encoding Acrs driven by mammalian promoters. Approximately 150 ng of each plasmid (e.g., having a 1:1:1 ratio of sgRNA:Cas9:Acr) was transfected, as most ACRs have been reported to inhibit Nme1Cas9 at those ratios. (Pawluk et al., 2016). As expected from the in vitro experiment, AcrIIC1_{Nme}, -IIC2_{Nme}, -IIC3_{Nme} and -IIC4_{Hpa} inhibited Nme2Cas9 genome editing, while AcrIIC5_{Smu} failed to do so. (FIG. 40B. Moreover, complete inhibition was observed to be below detection levels by Acr3Nme and Acr4Hpa, suggesting their high potency as compared to AcrsIIC1_{Nme} and AcrIIC2_{Nme}. To further compare the potency of AcrIIC1_{Nme} and AcrIIC4_{Hpa}, experiments were performed at various ratios of Acr to Cas9. FIG. 40C. Consequently, AcrIIC4_{Hpa} is a highly potent inhibitor against Nme2Cas9, with concentrations as low as 25 ng:100 ng Acr:Cas9 inhibiting Nme2Cas9 by 4 fold. Together, these data suggest that Acr proteins can be used as off-switches for Nme2Cas9-based applications.

### 5. Nme2Cas9 Hyper-Accuracy

Off-target effects could potentially confound therapeutic applications during ex vivo and in vivo human gene therapy by creating unintended mutations. Since wildtype SpyCas9 has a relatively high number of off-target sites in human cells, there have been several efforts to engineer high-fidelity SpyCas9 variants with variable success. In contrast, Nme1Cas9 is naturally hyper-accurate, demonstrating remarkable fidelity in cells and mouse models. Previous work shows that hybridization kinetics, which is not determined by the PID, may determine the fidelity of a Cas9, therefore suggesting that Nme2Cas9 may also be hyper-accurate.

To empirically assess NmeCas9 off-target profiles, Genome-Wide, Unbiased Identification of double-stranded breaks Enabled by Sequencing (GUIDE-Seq) techniques were used to determine potential off-target sites in an unbiased fashion. GUIDE-Seq relies on the incorporation of double-stranded oligodeoxynucleotides (dsODNs) into DNA double-stranded break sites throughout the genome. These cleavage sites are detected by amplification and high-throughput sequencing.

As a benchmark for GUIDE-Seq, wildtype SpyCas9 was used. In particular, SpyCas9 and Nme2Cas9 were able to be cloned into identical backbones driven by the same promoter, and used to target the same sites because of their non-overlapping PAMs. This technique allows side-by-side comparison the two nucleases. Six (6) dual sites (DS) were targeted in VEGFA with a NGGNCCN sequence. FIG. 42A. Seventy-two (72) hours after transfection, TIDE analysis was performed on the target sites. Nme2Cas9 induced indels at all six (6) sites, albeit at low efficiencies at two of them, while SpyCas9 induced indels at 4/6 sites. FIG. 42B. On two of those 4 sites (DS1 and DS4), SpyCas9 induced ˜7 fold more indels than Nme2Cas9, while Nme2Cas9 induced by ˜3 folds increase in indels at DS6. For GUIDE-seq, targets DS2, DS4 and DS6 were selected to determine off-target cleavage at sites where Nme2Cas9 is as efficient, less efficient or more efficient than SpyCas9, respectively.

In addition to the three dual target sites, a TS6 target site with a 30-50% indel rate (depending on the cell type) along with the mouse Pcsk9 and Rosa26 genes were subjected to GUIDE-Seq analysis. It was considered that the off-target profiles would be more prominent because the TS6 target is known to undergo highly efficient gene editing. In addition, testing of the mouse Pcsk9 and Rosa26 sites would then reveal the fidelity of Nme2Cas9 in a different cell line, and candidate loci for in vivo genome editing. Consequently, transfections were performed for each Cas9 along with their cognate sgRNAs and the dsODNs and GUIDE-Seq libraries were prepared. GUIDE-Seq analysis demonstrated efficient on-target editing with both Cas9 orthologs with similar patterns observed by TIDE. For off-target identification, the analysis revealed that while the three SpyCas9 sites had the expected high number of off-target sites (e.g., ranging between approximately between 10-1000). Nme2Cas9 had a strikingly clean off-target profile. Specifically, Nme2Cas9 targeting the same dual site showed, at most, one off-target site. See, FIG. 42C.

To validate the off-target sites detected by GUIDE-seq, targeted deep sequencing was performed to measure indel formation at the top off-target loci following GUIDE-seq-independent editing (i.e. without co-transfection of the dsODN). While SpyCas9 showed considerable editing at most off-target sites tested (in some instances, more efficient than that at the corresponding on-target site), Nme2Cas9 exhibited no detectable indels at the lone DS2 and DS6 candidate off-target sites. With the Rosa26 sgRNA, Nme2Cas9 induced ˜1% editing at the Rosa26-OT1 site in Hepa1-6 cells, compared to ˜30% on-target editing. FIG. 42D.

Next, to enable the use of SpyCas9 as a benchmark for GUIDE-seq, due to the fact that SpyCas9 and Nme2Cas9 have non-overlapping PAMs they can therefore potentially edit any dual site (DS) flanked by a 5′-NGGNCC-3′ sequence, which simultaneously fulfills the PAM requirements of both Cas9's binding properties. This enables side-by-side comparisons of off-targeting with sgRNAs that bind the exact same on-target site. Using matched plasmids expressing each Cas9 and their respective sgRNAs, twenty-eight (28) DSs were targeted at multiple loci throughout the human genome. Seventy-two (72) hours after plasmid delivery, a TIDE analysis was performed on the sites targeted by each nuclease. Nme2Cas9 induced indels at nineteen (19) target sites, albeit at low efficiencies (<5%) at four of them, while SpyCas9 induced indels at twenty-three (23) of the target sites, in one case with <5% efficiency. Three dual target sites were recalcitrant to editing by both nucleases. While SpyCas9 is clearly more efficient overall, both enzymes have similar efficiencies at many of the sites, and at two of the seventeen sites that were edited by both nucleases, Nme2Cas9 was more efficient under these conditions. See, FIG. 42E.

It is noteworthy that this off-target site has a consensus Nme2Cas9 PAM (ACTCCCT) with only 3 mismatches at the PAM-distal end of the guide-complementary region (i.e. outside of the seed). See, FIG. 42F. These data support and reinforce our GUIDE-seq results indicating a high degree of accuracy for Nme2Cas9 genome editing in mammalian cells.

On- vs. off-target on these sides were compared by targeted amplification of each locus followed by TIDE analysis. FIG. 43A. Interestingly, no indels could be detected at those off-target sites for either sgRNA by TIDE, while efficient on-target editing was observed. Furthermore, the read counts for these off-targets were negligible as compared to those observed in the case of SpyCas9 suggesting Nme2Cas9 is highly specific. (FIG. 43C, left versus right, respectively). To further corroborate these GUIDE-Seq results, CRISPRseek was used to computationally predict potential off-target sites for two of the most active sgRNAs with highly similar sites in the genome. (Zhu et al., 2014). These were performed with N₄CX PAMs and 2-5 mismatches, mostly in the PAM-distal region. FIG. 43D. Taken together, these data suggest that Nme2Cas9 is a high-fidelity nuclease in mammalian cells.

### 6. Clinical Applications

In one embodiment, the present invention contemplates an Nme2Cas9 complex as the first compact, hyper-accurate Cas9 with a small non-restrictive PAM for therapeutic genome editing by AAV delivery. Although small, previously reported hyper-accurate Cas9 orthologs have longer PAMs than those disclosed herein, thereby restricting their therapeutic use due to limited target sites in a given gene (and off-target profile in the case of SauCas9). This disadvantage is exacerbated in loci where only a specific window can be targeted, or a precise block deletion is required.

The all-in-one AAV delivery platform established herein can be used to target any gene in any tissue. Moreover, Nme2Cas9's hyper-accuracy enables precise editing of the target genes, therefore ameliorating safety concerns raised due to off-target activities previously observed. To this end, Nme2Cas9 has the potential to not only complement existing tools, but to become a preferred choice for therapeutic genome editing by viral delivery.

Furthermore, inhibition of Nme2Cas9 by various Acrs suggest a possible evolutionary pressure imposed on Cas9 to rapidly evolve a particular domain. Specifically, the lack of inhibition of Nme2Cas9 by AcrIIC5_{Smu} raises the possibility that its mechanism of inhibition is through a PID. Considering that AcrIIC5_{Smu} is the most potent inhibitor of Nme1Cas9 to date, it is contemplated herein where AcrIIC5_{Smu} can be used to robustly turn off Nme1Cas9 but not Nme2Cas9. This is of particular interest in cellular contexts where multiplexing would be enhanced by the ability to control a specific ortholog.

Finally, while there are thousands of Cas9 orthologs in the public database, only a handful of which have been characterized. Some embodiments contemplated herein take advantage of the natural variation in closely-related Cas9 orthologs to create two novel Cas9 nucleases, namely Nme2Cas9 and Nme3Cas9, with N4CC and N4CAAA PAMs, respectively. The data presented herein demonstrate that even closely related orthologs can have vastly different properties. For example, these orthologs use the exact same sgRNA as Nme1Cas9, which circumvent the difficulties in the prediction of tracrRNAs and determining the right spacer length for each ortholog. Furthermore, it is likely that shorter and more stable sgRNAs (such as chemical modifications) can be engineered to expand to all three nucleases. These characteristics may ease genome editing efforts and reduce the costs associated with protein and RNA engineering.

It should be apparent to one of skill in the art that the embodiments described herein are not restricted to Cas9s and can be applied to other Cas proteins such as Cas12 and Cas13. It should also be appreciated that Cas9's hyper-variability is not restricted to PIDs. It is considered herein that strains exist which share high degree of homology with a given Cas9 but differ in other domains due to other types of selective pressure. Taken together, Nme2Cas9 is a novel nuclease which improves the current CRISPR platforms for therapeutic genome editing.

### V. Nucleotide Delivery Platforms

Aside from the above described AAV nucleotide delivery systems, the present invention contemplates several delivery systems compatible with nucleic acids that provide for roughly uniform distribution and have controllable rates of release. Some embodiments of the present invention contemplate nucleic acid delivery systems encoding Type II-C Cas9-sgRNA complexes as described herein.

A variety of different media are described below that are useful in creating nucleic acid delivery systems. It is not intended that any one medium or carrier is limiting to the present invention. Note that any medium or carrier may be combined with another medium or carrier; for example, in one embodiment a polymer microparticle carrier attached to a compound may be combined with a gel medium.

Carriers or mediums contemplated by this invention comprise a material selected from the group comprising gelatin, collagen, cellulose esters, dextran sulfate, pentosan polysulfate, chitin, saccharides, albumin, fibrin sealants, synthetic polyvinyl pyrrolidone, polyethylene oxide, polypropylene oxide, block polymers of polyethylene oxide and polypropylene oxide, polyethylene glycol, acrylates, acrylamides, methacrylates including, but not limited to, 2-hydroxyethyl methacrylate, poly(ortho esters), cyanoacrylates, gelatin-resorcin-aldehyde type bioadhesives, polyacrylic acid and copolymers and block copolymers thereof.

### Microparticles

One embodiment of the present invention contemplates a nucleic acid delivery system comprising a microparticle. Preferably, microparticles comprise liposomes, nanoparticles, microspheres, nanospheres, microcapsules, and nanocapsules. Preferably, some microparticles contemplated by the present invention comprise poly(lactide-co-glycolide), aliphatic polyesters including, but not limited to, poly-glycolic acid and poly-lactic acid, hyaluronic acid, modified polysacchrides, chitosan, cellulose, dextran, polyurethanes, polyacrylic acids, psuedo-poly(amino acids), polyhydroxybutrate-related copolymers, polyanhydrides, polymethylmethacrylate, poly(ethylene oxide), lecithin and phospholipids.

### Liposomes

One embodiment of the present invention contemplates liposomes capable of attaching and releasing nucleic acids as described herein. Liposomes are microscopic spherical lipid bilayers surrounding an aqueous core that are made from amphiphilic molecules such as phospholipids. For example, a liposome may trap a nucleic acid between the hydrophobic tails of the phospholipid micelle. Water soluble agents can be entrapped in the core and lipid-soluble agents can be dissolved in the shell-like bilayer. Liposomes have a special characteristic in that they enable water soluble and water insoluble chemicals to be used together in a medium without the use of surfactants or other emulsifiers. Liposomes can form spontaneously by forcefully mixing phospholipids in aqueous media. Water soluble compounds are dissolved in an aqueous solution capable of hydrating phospholipids. Upon formation of the liposomes, therefore, these compounds are trapped within the aqueous liposomal center. The liposome wall, being a phospholipid membrane, holds fat soluble materials such as oils. Liposomes provide controlled release of incorporated compounds. In addition, liposomes can be coated with water soluble polymers, such as polyethylene glycol to increase the pharmacokinetic half-life. One embodiment of the present invention contemplates an ultra high-shear technology to refine liposome production, resulting in stable, unilamellar (single layer) liposomes having specifically designed structural characteristics. These unique properties of liposomes, allow the simultaneous storage of normally immiscible compounds and the capability of their controlled release.

In some embodiments, the present invention contemplates cationic and anionic liposomes, as well as liposomes having neutral lipids. Preferably, cationic liposomes comprise negatively-charged materials by mixing the materials and fatty acid liposomal components and allowing them to charge-associate. Clearly, the choice of a cationic or anionic liposome depends upon the desired pH of the final liposome mixture. Examples of cationic liposomes include lipofectin, lipofectamine, and lipofectace.

One embodiment of the present invention contemplates a nucleic acid delivery system comprising liposomes that provides controlled release of at least one nucleic acid. Preferably, liposomes that are capable of controlled release: i) are biodegradable and non-toxic; ii) carry both water and oil soluble compounds; iii) solubilize recalcitrant compounds; iv) prevent compound oxidation; v) promote protein stabilization; vi) control hydration; vii) control compound release by variations in bilayer composition such as, but not limited to, fatty acid chain length, fatty acid lipid composition, relative amounts of saturated and unsaturated fatty acids, and physical configuration; viii) have solvent dependency; iv) have pH-dependency and v) have temperature dependency.

The compositions of liposomes are broadly categorized into two classifications. Conventional liposomes are generally mixtures of stabilized natural lecithin (PC) that may comprise synthetic identical-chain phospholipids that may or may not contain glycolipids. Special liposomes may comprise: i) bipolar fatty acids; ii) the ability to attach antibodies for tissue-targeted therapies; iii) coated with materials such as, but not limited to lipoprotein and carbohydrate; iv) multiple encapsulation and v) emulsion compatibility.

Liposomes may be easily made in the laboratory by methods such as, but not limited to, sonication and vibration. Alternatively, compound-delivery liposomes are commercially available. For example, Collaborative Laboratories, Inc. are known to manufacture custom designed liposomes for specific delivery requirements.

### Microspheres, Microparticles and Microcapsules

Microspheres and microcapsules are useful due to their ability to maintain a generally uniform distribution, provide stable controlled compound release and are economical to produce and dispense. Preferably, an associated delivery gel or the compound-impregnated gel is clear or, alternatively, said gel is colored for easy visualization by medical personnel.

Microspheres are obtainable commercially (Prolease®, Alkerme's: Cambridge, Mass.). For example, a freeze dried medium comprising at least one therapeutic agent is homogenized in a suitable solvent and sprayed to manufacture microspheres in the range of 20 to 90 μm. Techniques are then followed that maintain sustained release integrity during phases of purification, encapsulation and storage. Scott et al., Improving Protein Therapeutics With Sustained Release Formulations, Nature Biotechnology, Volume 16:153-157 (1998).

Modification of the microsphere composition by the use of biodegradable polymers can provide an ability to control the rate of nucleic acid release. Miller et al., Degradation Rates of Oral Resorbable Implants {Polylactates and Polyglycolates: Rate Modification and Changes in PLA/PGA Copolymer Ratios, J. Biomed. Mater. Res., Vol. II:711-719 (1977).

Alternatively, a sustained or controlled release microsphere preparation is prepared using an in-water drying method, where an organic solvent solution of a biodegradable polymer metal salt is first prepared. Subsequently, a dissolved or dispersed medium of a nucleic acid is added to the biodegradable polymer metal salt solution. The weight ratio of a nucleic acid to the biodegradable polymer metal salt may for example be about 1:100000 to about 1:1, preferably about 1:20000 to about 1:500 and more preferably about 1:10000 to about 1:500. Next, the organic solvent solution containing the biodegradable polymer metal salt and nucleic acid is poured into an aqueous phase to prepare an oil/water emulsion. The solvent in the oil phase is then evaporated off to provide microspheres. Finally, these microspheres are then recovered, washed and lyophilized. Thereafter, the microspheres may be heated under reduced pressure to remove the residual water and organic solvent.

Other methods useful in producing microspheres that are compatible with a biodegradable polymer metal salt and nucleic acid mixture are: i) phase separation during a gradual addition of a coacervating agent; ii) an in-water drying method or phase separation method, where an antiflocculant is added to prevent particle agglomeration and iii) by a spray-drying method.

In one embodiment, the present invention contemplates a medium comprising a microsphere or microcapsule capable of delivering a controlled release of a nucleic acid for a duration of approximately between 1 day and 6 months. In one embodiment, the microsphere or microparticle may be colored to allow the medical practitioner the ability to see the medium clearly as it is dispensed. In another embodiment, the microsphere or microcapsule may be clear. In another embodiment, the microsphere or microparticle is impregnated with a radio-opaque fluoroscopic dye.

Controlled release microcapsules may be produced by using known encapsulation techniques such as centrifugal extrusion, pan coating and air suspension. Such microspheres and/or microcapsules can be engineered to achieve desired release rates. For example, Oliosphere® (Macromed) is a controlled release microsphere system. These particular microsphere's are available in uniform sizes ranging between 5-500 μm and composed of biocompatible and biodegradable polymers. Specific polymer compositions of a microsphere can control the nucleic acid release rate such that custom-designed microspheres are possible, including effective management of the burst effect. ProMaxx® (Epic Therapeutics, Inc.) is a protein-matrix delivery system. The system is aqueous in nature and is adaptable to standard pharmaceutical delivery models. In particular, ProMaxx® are bioerodible protein microspheres that deliver both small and macromolecular drugs, and may be customized regarding both microsphere size and desired release characteristics.

In one embodiment, a microsphere or microparticle comprises a pH sensitive encapsulation material that is stable at a pH less than the pH of the internal mesentery. The typical range in the internal mesentery is pH 7.6 to pH 7.2. Consequently, the microcapsules should be maintained at a pH of less than 7. However, if pH variability is expected, the pH sensitive material can be selected based on the different pH criteria needed for the dissolution of the microcapsules. The encapsulated nucleic acid, therefore, will be selected for the pH environment in which dissolution is desired and stored in a pH preselected to maintain stability. Examples of pH sensitive material useful as encapsulants are Eudragit® L-100 or S-100 (Rohm GMBH), hydroxypropyl methylcellulose phthalate, hydroxypropyl methylcellulose acetate succinate, polyvinyl acetate phthalate, cellulose acetate phthalate, and cellulose acetate trimellitate. In one embodiment, lipids comprise the inner coating of the microcapsules. In these compositions, these lipids may be, but are not limited to, partial esters of fatty acids and hexitiol anhydrides, and edible fats such as triglycerides. Lew C. W., Controlled-Release pH Sensitive Capsule And Adhesive System And Method. U.S. Pat. No. 5,364,634 (herein incorporated by reference).

In one embodiment, the present invention contemplates a microparticle comprising a gelatin, or other polymeric cation having a similar charge density to gelatin (i.e., poly-L-lysine) and is used as a complex to form a primary microparticle. A primary microparticle is produced as a mixture of the following composition: i) Gelatin (60 bloom, type A from porcine skin), ii) chondroitin 4-sulfate (0.005%-0.1%), iii) glutaraldehyde (25%, grade 1), and iv) 1-ethyl-3-(3-dimethylaminopropyl)-carbodiimide hydrochloride (EDC hydrochloride), and ultra-pure sucrose (Sigma Chemical Co., St. Louis, Mo.). The source of gelatin is not thought to be critical; it can be from bovine, porcine, human, or other animal source. Typically, the polymeric cation is between 19,000-30,000 daltons. Chondroitin sulfate is then added to the complex with sodium sulfate, or ethanol as a coacervation agent.

Following the formation of a microparticle, a nucleic acid is directly bound to the surface of the microparticle or is indirectly attached using a “bridge” or “spacer”. The amino groups of the gelatin lysine groups are easily derivatized to provide sites for direct coupling of a compound. Alternatively, spacers (i.e., linking molecules and derivatizing moieties on targeting ligands) such as avidin-biotin are also useful to indirectly couple targeting ligands to the microparticles. Stability of the microparticle is controlled by the amount of glutaraldehyde-spacer crosslinking induced by the EDC hydrochloride. A controlled release medium is also empirically determined by the final density of glutaraldehyde-spacer crosslinks.

In one embodiment, the present invention contemplates microparticles formed by spray-drying a composition comprising fibrinogen or thrombin with a nucleic acid. Preferably, these microparticles are soluble and the selected protein (i.e., fibrinogen or thrombin) creates the walls of the microparticles. Consequently, the nucleic acids are incorporated within, and between, the protein walls of the microparticle. Heath et al., Microparticles And Their Use In Wound Therapy. U.S. Pat. No. 6,113,948 (herein incorporated by reference). Following the application of the microparticles to living tissue, the subsequent reaction between the fibrinogen and thrombin creates a tissue sealant thereby releasing the incorporated compound into the immediate surrounding area.

One having skill in the art will understand that the shape of the microspheres need not be exactly spherical; only as very small particles capable of being sprayed or spread into or onto a surgical site (i.e., either open or closed). In one embodiment, microparticles are comprised of a biocompatible and/or biodegradable material selected from the group consisting of polylactide, polyglycolide and copolymers of lactide/glycolide (PLGA), hyaluronic acid, modified polysaccharides and any other well known material.

### EXPERIMENTAL

### Example I

### Construction of All-in-One sgRNA-Nme1Cas9-AAV Vector Plasmid

Bacterial Nme1Cas9 gene has been codon-optimized for expression in humans, and cloned into an AAV2 plasmid under U1a ubiquitous promoter. Guide RNA is under U6 promoter. The cas9 gene contains four nuclear localization signals and three HA tag sequences in tandem. Spacer sequences were inserted into the crRNA cassette by digesting the plasmid with SapI restriction enzyme using annealed synthetic oligonucleotides to generate a duplex with overhangs compatible with those generated by SapI digested backbone.

The human-codon optimized Nme1Cas9 gene under the control of the U1a promoter and a sgRNA cassette driven by the U6 promoter were cloned into an AAV2 plasmid backbone. The NmeCas9 ORF was flanked by four nuclear localization signals—two on each terminus—in addition to a triple-HA epitope tag. This plasmid is available through Addgene (plasmid ID 112139). See, FIG. 64. Oligonucleotides with spacer sequences targeting Hpd, Pcsk9, and Rosa26 were inserted into the sgRNA cassette by ligation into a SapI cloning site.

AAV vector production was performed at the Horae Gene Therapy Center at the University of Massachusetts Medical School. Briefly, plasmids were packaged in AAV8 capsids by triple-plasmid transfection in HEK293 cells and purified by sedimentation as previously described. Gao et al., “Introducing genes into mammalian cells: viral vectors” In: Green M R, Sambrook J, editors. Molecular cloning: a laboratory manual. Volume 2. 4th ed. New York: Cold Spring Harbor Laboratory Press; 2012. p. 1209-13. The off-target profiles of these spacers were predicted computationally using the Bioconductor package CRISPRseek. Search parameters were adapted to Nme1Cas9 settings: gRNA.size=24, PAM=“NNNNGATT,” PAM.-size=8, RNA.PAM.pattern=“NNNNGNNN$,” weights=c(0, 0, 0, 0, 0, 0, 0.014, 0, 0, 0.395, 0.317, 0, 0.389, 0.079, 0.445, 0.508, 0.613, 0.851, 0.732, 0.828, 0.615, 0.804, 0.685, 0.583), max.mismatch=6, allowed.mismatch.PAM=7, topN=10,000, min.score=0.

### Example II

### Cell Culture and Transfection

Mouse Hepa1-6 hepatoma cells were cultured in DMEM with 10% FBS and 1% Penicillin/Streptomycin (Gibco) in a 37° C. incubator with 5% CO₂. Human HEK293T cells and PLB985 cells were cultured in DMEM and RPMI media respectively. Both were supplemented with 10% FBS and 1% Penicillin/Streptomycin (Gibco). Transient transfections of Hepa 1-6 cells were performed using Lipofectamine LTX whereas Polyfect transfection reagent (Qiagen) was used for HEK293T cells. For transient transfection, approximately 1×10⁵ cells per well were cultured in 24-well plate 24 hours before transfection. Each well was transfected with 500 ng all-in-one sgRNA-Nme1Cas9-AAV plasmids, using Lipofectamine LTX with Plus Reagent (ThermoFisher) according to the manufacturer's protocol. HEK293T cells were transfected with 400 ng of all-in-one plasmid expressing Nme1Cas9 and sgRNA in 24-well plate according to manufacturer's guidelines (e.g., Psck9 & Rosa26).

All cell lines were maintained in a 37° C. incubator with 5% CO₂. Mouse Hepa1-6 hepatoma and HEK293T cells were cultured in DMEM with 10% FBS and 1% Penicillin/Streptomycin (Gibco). K562 cells were grown in the same conditions but using IMDM. IMR-90 cells were cultured in EMEM and 10% FBS. Finally, HDFa cells were grown in DMEM and 20% FBS.

### Example III

### Expression and Purification of Nme1Cas9

Nme1Cas9 was cloned into a pMCSG7 vector containing a T7 promoter followed by 6×His-tag and then a tobacco etch virus (TEV) protease cleavage site. This construct was transformed into Rosetta2 DE3 strain of E. coli and Nme1Cas9 was expressed. Briefly, bacterial culture was grown at 37° C. until OD600 of 0.6 was reached. At this point the temperature was lowered to 18° C. followed by addition of 1 mM Isopropyl β-D-1-thiogalactopyranoside (IPTG). Cells were grown overnight, and then harvested for purification.

Purification of Nme1Cas9 was performed in three steps: Nickel affinity chromatography, cation exchange chromatography, and then size exclusion chromatography. The detailed protocols for these can be found in previous publications (Jinek et al., Science 337, 816-821, 2012).

### Example IV

### Ribonucleoprotein (RNP) Delivery of Nme1Cas9

RNP delivery of Nme1Cas9 was performed using the Neon transfection system (ThermoFisher). Approximately 20 picomoles of Nme1Cas9 and 25 picomoles of sgRNA were mixed in buffer R and incubated at room temperature for 20-30 minutes. This preassembled complex was then mixed with 50,000-100,000 cells, and electroporated using 10 μL Neon tips. After electroporation, cells were plated in 24-well plates containing the appropriate culture media without antibiotics.

### Example V

### DNA Isolation From Cells and Tissue

Genomic DNA was isolated 72 hours post-transfection from cells via DNeasy® Blood and Tissue kit (Qiagen) according to the manufacturer's protocol. Mice were sacrificed and liver tissue was harvested 10 days post-hydrodynamic injection or 50 days post-tail vein vector administration, and genomic DNA was isolated with a DNeasy® Blood and Tissue kit (Qiagen) according to the manufacturer's protocol.

### Example VI

### Indel Analysis

50 ng of genomic DNA was used for PCR amplification with genomic site-specific primers and High Fidelity® 2×PCR Master Mix (New England Biolabs). For TIDE analysis, 30 μl of PCR product was purified using QIAquick® PCR Purification Kit (Qiagen), and subjected to Sanger sequencing. Indel values were obtained using the TIDE web tool (tide-calculator.nki.nl/) as described previously. Brinkman et al., Nucl. Acids Res. (2014).

For the T7 Endonuclease I (T7EI) assay, 10 μl of the PCR product was hybridized and treated with 0.5 μl T7 Endonuclease I (New England Biolabs) in 1×NEB Buffer 2 for 1 hour. The samples were run on a 2.5% agarose gel and quantified with ImageMaster-TotalLab® program. Indel percentages were calculated as previously described. Guschin et al., Engineered Zinc Finger Proteins: Methods and Protocols (2010).

### Example VII

### GUIDE-Seq for Off-Target Analysis

GUIDE-seq analysis was performed as previously described. Tsai et al., Nature Biotechnology (2014), Bolukbasi et al., Nature Methods (2015a); Amrani et al., biorxiv.org/content/early/2017/08/04/172650 (2017).

Briefly, Hepa1-6 cells were transfected with 500 ng of all-in-one sgRNA-Nme1Cas9-AAV plasmids and 7.5 pmol of annealed GUIDE-seq oligonucleotide using Lipofectamine LTX® with Plus® Reagent (ThermoFisher), for the two spacers targeting Pcsk9 and Rosa26 genes. Genomic DNA was extracted with a DNeasy® Blood and Tissue kit (Qiagen) at 72 hours after transfection following the manufacturer protocol. Library preparations, deep sequencing, and reads analysis were performed as previously described. Tsai et al., Nature Biotechnology (2014), Bolukbasi et al., Nature Methods (2015a); Amrani et al., biorxiv.org/content/early/2017/08/04/172650 (2017).

### Example IX

### AAV Vector Production

Plasmids were packaged in AAV8 by triple-plasmid transfection in HEK 293 cells and purified by sedimentation as previously described at the Horae Gene Therapy Center at the University of Massachusetts Medical School. Gao G P, Sena-Esteves M. Introducing Genes into Mammalian Cells: Viral Vectors. In: Green M R, Sambrook J, eds. Molecular Cloning, Volume 2: A Laboratory Manual. New York: Cold Spring Harbor Laboratory Press; 2012:1209-1313.

### Example X

### Animals, AAV Vector Injections, and Liver Tissue Processing

All animal experiments were approved under the guidelines of the University of Massachusetts Medical School Institutional Animal Care and Use Committee. For hydrodynamic injections, 2.5 mL of 30 μg of endotoxin-free sgRNA-Nme1Cas9-AAV plasmid targeting Pcsk9, or PBS as a control, were injected via tail vein into 9-18 weeks old female C57BL/6 mice. For the AAV8 vector injections, 9-18 weeks old female C57BL/6 mice were injected with 4×10¹¹ genome copies per mouse via tail vein. 8-week-old female C57BL/6NJ mice were used for genome editing experiments in vivo. For ex vivo experiments, embryos that were advanced to two-cell stage were transferred into the oviduct of E0.5 pseudo-pregnant female mice.

Mice were euthanized by CO₂ and liver was collected. Tissues were fixed in 4% paraformaldehyde overnight, and embedded in paraffin, sectioned and stained with hematoxylin and eosin (H&E).

### Example XI

### Serum Analysis

Blood (˜200 μL) was drawn from the facial vein at 0, 25, and 50 days post vector administration. Serum was isolated using a serum separator (BD, Cat. No. 365967) and stored under −80° C. until assay. Serum cholesterol levels were measured by Infinity™ colorimetric endpoint assay (Thermo-Scientific) following the manufacturer's protocol. Briefly, serial dilutions of Data-Cal™ Chemistry Calibrator were prepared in PBS. In a 96-well plate, 2 μL of mice sera or calibrator dilution was mixed with 200 μL of Infinity™ cholesterol liquid reagent, then incubated at 37° C. for 5 min. The absorbance was measured at 500 nm using a BioTek Synergy® HT microplate reader.

### Example XII

### Discovery of Cas9 Orthologs With Hyper-Evolved PIDs

Nme1Cas9 sequence was blasted to find all Cas9 orthologs in Neisseria species. Orthologs with >80% identity to Nme1Cas9 were selected for the remainder of this analysis. The PIDs of each was then aligned using ClustalW2 with that of Nme1Cas9 (from 820^{th} amino acid to 1082^{nd}) and those with clusters of mutations in the PID were selected.

Nme1Cas9 peptide sequence was used as a query in BLAST searches to find all Cas9 orthologs in Neisseria meningitidis strains. Orthologs with >80% identity to Nme1Cas9 were selected for study. The PIDs were then aligned with that of Nme1Cas9 (residues 820-1082) using ClustalW2® and those with clusters of mutations in the PID were selected for further analysis. An unrooted phylogenetic tree of NmeCas9 orthologs was constructed using FigTree (tree.bio.ed.ac.uk/software/figtree).

### Example XIII

### Cloning and Purification of Nme2 and Nme3 Cas9 and Acr Orthologs

The PIDs of Nme2Cas9 and Nme3Cas9 were ordered as gBlocks (IDT) to replace the PID of Nme1Cas9 using Gibson Assembly (NEB) in a bacterial expression plasmid pMSCG7 with 6×His-tag. The construct was transformed into E. coli, expressed and purified as previously described.

Briefly, Rosetta (DE3) cells containing the respective Cas9 plasmids were grown at 37° C. to an optical density of 0.6 and protein expression was induced by 1 mM IPTG for 16 hr at 18° C. Cells were harvested and lysed by sonication in lysis buffer (50 mM Tris pH 7.5, 500 mM NaCl, 5 mM imidazole, 1 mM DTT) supplemented with Lysozyme and protease inhibitor cocktail (Sigma).

The lysate was then run through a Ni-NTA agarose column (Qiagen), the bound protein was eluted with 300 mM imidazole and dialyzed into storage buffer (20 mM HEPES pH 7.5, 250 mM NaCl, 1 mM DTT). For Acr proteins, 6×His tagged proteins were expressed in E. coli strain BL21 Rosetta (DE3). Cells were grown at 37° C. to an optical density (OD_{600 nm}) of 0.6 in a shaking incubator. The bacterial cultures were cooled to 18° C., and protein expression was induced by adding 1 mM IPTG for overnight expression. The next day, cells were harvested and resuspended in lysis buffer (50 mM Tris pH 7.5, 500 mM NaCl, 5 mM imidazole, 1 mM DTT) supplemented with 1 mg/mL Lysozyme and protease inhibitor cocktail (Sigma) and protein was purified using the same protocol as for Cas9. The 6×His tag was removed by incubation with Tobacco Etch Virus (TEV) protease overnight at 4° C. to isolate successfully cleaved, untagged Acrs.

### Example IVX

### In Vitro PAM Discovery Assay

A library of protospacers with randomized PAM sequences was generated using overlapping PCRs, with the forward primer containing the 10-nucleotide randomized PAM.

The library was gel purified and subjected to in vitro cleavage reaction by purified Cas9 along with in vitro transcribed sgRNAs. 300 nM Cas9:sgRNA complex was used to cleave 300 nM of the target fragment in 1×NE Buffer 3.1 (NEB) at 37° C. for 1 hr. The reaction was then treated with proteinase K at 50° C. for 10 minutes and run on a 4% agarose gel with 1×TAE. The cleavage product was purified and subjected to library preparation. The library was sequenced using the Illumina NextSeq500® sequencing platform and analyzed. Sequence logos were generated using R.

### Example XV

### Transfections and Mammalian Genome Editing

Humanized Nme2Cas9 was cloned into pCDest2 plasmid previously used for Nme1Cas9 and SpyCas9 expression using Gibson Assembly. Transfection of HEK293T and HEK293T-TLR cells was performed as previously described (Amrani et al. 2018). For Hepa1-6 transfections, Lipofectamine LTX was used to transfect 500 ng of all-in-one AAV.sgRNA.Nme2Cas9 plasmid in approximately 1×10⁵ cells per well that had been cultured in 24-well plates 24 hours before transfection. For K562 cells stably expressing Nme2Cas9, 50,000-150,000 cells were electroporated with 500 sgRNA plasmid using 10 μL Neon tips.

To measure indels in all cells, 72 hr after transfections, cells were harvested, and genomic DNA was extracted using the DNeasy® Blood and Tissue kit (Qiagen). The targeted locus was amplified by PCR, Sanger sequenced (Genewiz®) and analyzed by TIDE (Brinkman et al. 2014).

### Example XVI

### Lentiviral Transduction of K562 Cells to Stably Express Nme2Cas9

K562 cells stably expressing Nme2Cas9 were generated as previously described. For lentivirus production, the lentiviral vector was co-transfected into HEK293T cells along with the packaging plasmids (Addgene 12260 & 12259) in 6-well plates using TransIT-LT1 transfection reagent (Mirus Bio) as recommended by the manufacturer. After 24 hours, the medium was aspirated from the transfected cells and replaced with fresh 1 mL of fresh DMEM media.

The next day, the supernatant containing the virus from the transfected cells was collected and filtered through 0.45 μm filter. 10 uL of the undiluted supernatant along with 2.5 μg of Polybrene was used to transduce ˜1 million K562 cells in 6-well plates. The transduced cells were selected using 2.5 μg/mL of Puromycin containing media.

### Example XVII

### RNP Delivery for Mammalian Genome Editing

For RNP experiments, a Neon electroporation system was used. 40 picomoles of 3×NLS Nme2Cas9 along with 50 picomoles of in vitro transcribed sgRNA was assembled in buffer R, and electroporated using 10 μL Neon tips. After electroporation, cells were plated in pre-warmed 24-well plates containing the appropriate culture media without antibiotics. Electroporation parameters (voltage, width, number of pulses) were 1150 v, 20 ms, 2 pulses for HEK293T cells; 1000 v, 50 ms, 1 pulse for K562 cells.

### Example XVIII

### GUIDE-Seq

GUIDE-Seq experiments were performed as described previously with minor modifications (Amrani et al., 2018).

Briefly, HEK293T cells were transfected with 200 ng of Cas9, 200 ng of sgRNA, and 7.5 pmol of annealed GUIDE-seq oligonucleotide using Polyfect (Qiagen) for guides targeting dual sites with SpyCas9 or Nme2Cas9. Hepa1-6 cells were transfected as described above.

Genomic DNA was extracted with a DNeasy® Blood and Tissue kit (Qiagen) 72 h after transfection according to the manufacturer protocol. Library preparation and sequencing were performed exactly as described previously.

For analysis, sites that matched a sequence with ten mismatches with the target site were considered potential off-target sites. Data were analyzed using the Bioconductor package GUIDEseq version 1.1.17 (Zhu et al., 2017).

### Example XIX

### Targeted Deep Sequencing and Analysis

Targeted deep sequencing was used to confirm the results of GUIDE-Seq and more quantitatively measure indel rates. A two-step PCR amplification was used to produce DNA fragments for each on- and off-target site. For SpyCas9, the top off-target locations were selected.

In the first step, locus-specific primers bearing universal overhangs with complementary ends to the adapters were mixed with 2× Phusion® PCR master mix (NEB) to generate fragments bearing the overhangs. In the second step, the purified PCR products were amplified with a universal forward primer and and indexed reverse primers.

Full-size products (˜250 bp in length) were gel-extracted and sequenced using a paired-end MiSeq run. MiSeq data analysis was performed exactly as previously described (Amrani 2018).

### Example XX

### Off-Target Analysis Using CRISPRseek

Global off-target analyses for TS25 and TS47 were performed using the Bioconductor package CRISPRseek.

Minor changes were made to accommodate for characteristics of Nme2Cas9 not shared with SpyCas9. Specifically, the following changes were used: gRNA.size=24, PAM=“NNNNCC”, PAM.size=6, RNA.PAM.pattern=“NNNNCN”, off-target sites with less than 6 mismatches were collected. The top potential off-target sites based on the number and position of mismatches were selected. gDNA from cells targeted by each respective sgRNA was used to amplify each off-target locus and analyzed by TIDE.

### Example XXI

### In Vivo AAV8.Nme2Cas9 Delivery and Liver Tissue Processing

All animal procedures were reviewed and approved by The Institutional Animal Care and Use Committee (IACUC) at University of Massachusetts Medical School.

For the AAV8 vector injections, 8 weeks old female C57BL/6 mice were injected with 4×10¹¹ genome copies per mouse via tail vein targeting Pcsk9 or Rosa26. Mice were sacrificed 28 days after vector administration and liver tissues were collected for analysis. Liver tissues were fixed in 4% formalin overnight, and embedded in paraffin, sectioned and stained with hematoxylin and eosin (H&E). Blood was drawn from facial vein at 0, 14 and 28 days post injection, and serum was isolated using a serum separator (BD, Cat. No. 365967) and stored at −80° C. until assay. Serum cholesterol level was measured using the Infinity™ colorimetric endpoint assay (Thermo-Scientific) following manufacturer's protocol and as previously described (Ibraheim et al, 2018).

### Example XXII

### Animals and Liver Tissue Processing

For hydrodynamic injections, 2.5 mL of 30 μg of endotoxin-free AAV-sgRNA-hNme1Cas9 plasmid targeting Pcsk9 or 2.5 mL PBS was injected by tail vein into 9- to 18-week-old female C57BL/6 mice. Mice were euthanized 10 days later and liver tissue was harvested. For the AAV8 vector injections, 12- to 16-week-old female C57BL/6 mice were injected with 4×10¹¹ genome copies per mouse via tail vein, using vectors targeting Pcsk9 or Rosa26. Mice were sacrificed 14 and 50 days after vector administration and liver tissues were collected for analysis. For Hpd targeting, 2 mL PBS or 2 mL of 30 μg of endotoxin-free AAV-sgRNA-hNme1Cas9 plasmid was administered into 15- to 21-week-old Type 1 Tyrosinemia Fah knockout mice (Fahneo) via tail vein. The encoded sgRNAs targeted sites in exon 8 (sgHpd1) or exon 11 (sgHpd2). The HT1 mice were fed with 10 mg/L NTBC (2-(2-nitro-4-trifluoromethylbenzoyl)-1,3-cyclohexanedione) (Sigma-Aldrich, Cat. No. PHR1731-1G) in drinking water when indicated. Both sexes were used in these experiments. Mice were maintained on NTBC water for seven days post injection and then switched to normal water. Body weight was monitored every 1-3 days. The PBS-injected control mice were sacrificed when they became moribund after losing 20% of their body weight after removal from NTBC treatment.

Mice were euthanized according to our protocol and liver tissue was sliced and fragments stored at −80° C. Some liver tissues were fixed in 4% formalin overnight, embedded in paraffin, sectioned and stained with hematoxylin and eosin (H&E).

### XXIII

### Western Blot

Liver tissue fractions were ground and resuspended in 150 μL of RIPA lysis buffer. Total protein content was estimated by Pierce™ BCA Protein Assay Kit (Thermo-Scientific) following the manufacturer's protocol. A total of 20 μg of protein from tissue or 2 ng of Recombinant Mouse Proprotein Convertase 9/PCSK9 Protein (R&D Systems, 9258-SE-020) were loaded onto a 4-20% Mini-Rotean® TGX™ Precast Gel (Bio-Rad). The separated bands were transferred onto PVDF membrane and blocked with 5% Blocking-Grade Blocker solution (Bio-Rad) for 2 h at room temperature. Membranes were incubated with rabbit anti-GAPDH (Abcam ab9485, 1:2000) or goat anti-PCSK9 (R&D Systems AF3985, 1:400) antibodies overnight at 4° C. Membranes were washed five times in TBST and incubated with horseradish peroxidase (HRP)-conjugated goat anti-rabbit (Bio-Rad 1,706,515, 1:4000) and donkey anti-goat (R&D Systems HAF109, 1:2000) secondary antibodies for 2 h at room temperature. The membranes were washed five times in TBST and visualized with Clarity™ western ECL substrate (Bio-Rad) using an M35A X-OMAT Processor (Kodak).

### Example XXIV

### Humoral Immune Response

Humoral IgG1 immune response to Nme1Cas9 was measured by ELISA (Bethyl; Mouse IgG1 ELISA Kit, E99-105) following manufacturer's protocol with a few modifications. Briefly, expression and three-step purification of Nme1Cas9 and SpyCas9 was performed. A total of 0.5 μg of recombinant Nme1Cas9 or SpyCas9 proteins suspended in 1× coating buffer (Bethyl) were used to coat 96-well plates (Corning) and incubated for 12 h at 4° C. with shaking. The wells were washed three times while shaking for 5 min using 1× Wash Buffer. Plates were blocked with 1×BSA Blocking Solution (Bethyl) for 2 h at room temperature, then washed three times. Serum samples were diluted 1:40 using PBS and added to each well in duplicate. After incubating the samples at 4° C. for 5 h, the plates were washed 3 times for 5 min and 100 μL of biotinylated anti-mouse IgG1 antibody (Bethyl; 1:100,000 in 1×BSA Blocking Solution) was added to each well. After incubating for 1 h at room temperature, the plates were washed four times and 100 μL of TMB Substrate was added to each well. The plates were allowed to develop in the dark for 20 min at room temperature and 100 μL of ELISA Stop Solution was then added per well. Following the development of the yellow solution, absorbance was recorded at 450 nm using a BioTek Synergy® HT microplate reader.

### Example XXV

### Zygote Incubation and Transfection

### Mouse Strains and Embryo Collection

All animal experiments were conducted under the guidance of the Institutional Animal Care and Use Committee (IACUC) of the University of Massachusetts Medical School. C57BL/6NJ (Stock No. 005304) mice were obtained from The Jackson Laboratory. All animals were maintained in a 12 h light cycle. The middle of the light cycle of the day when a mating plug was observed was considered embryonic day 0.5 (E0.5) of gestation. Zygotes were collected at E0.5 by tearing the ampulla with forceps and incubation in M2 medium containing hyaluronidase to remove cumulus cells.

### In Vivo AAV8.Nme2Cas9+sgRNA Delivery and Liver Tissue Processing

For the AAV8 vector injections, 8-week-old female C57BL/6NJ mice were injected with 4×10¹¹ genome copies per mouse via tail vein, with the sgRNA targeting a validated site in either Pcsk9 or Rosa26. Mice were sacrificed 28 days after vector administration and liver tissues were collected for analysis. Liver tissues were fixed in 4% formalin overnight, embedded in paraffin, sectioned and stained with hematoxylin and eosin (H&E). Blood was drawn from the facial vein at 0, 14 and 28 days post injection, and serum was isolated using a serum separator (BD, Cat. No. 365967) and stored at −80° C. until assay. Serum cholesterol level was measured using the Infinity™ colorimetric endpoint assay (Thermo-Scientific) following the manufacturer's protocol and as previously described. Ibraheim et al., “All-in-One Adeno-associated Virus Delivery and Genome Editing by Neisseria meningitidis Cas9 in vivo” Genome Biology 19:137 (2018).

For an anti-PCSK9 Western blot, 40 μg of protein from tissue or 2 ng of Recombinant Mouse PCSK9 Protein (R&D Systems, 9258-SE-020) were loaded onto a MiniProtean® TGX™ Precast Gel (Bio-Rad). The separated bands were transferred onto a PVDF membrane and blocked with 5% Blocking-Grade Blocker® solution (Bio-Rad) for 2 hours at room temperature. Next, the membrane was incubated with rabbit anti-GAPDH (Abcam ab9485, 1:2,000) or goat anti-PCSK9 (R&D Systems AF3985, 1:400) antibodies overnight. Membranes were washed in TBST and incubated with horseradish peroxidase (HRP)-conjugated goat anti-rabbit (Bio-Rad 1706515, 1:4,000), and donkey anti-goat (R&D Systems HAF109, 1:2,000) secondary antibodies for 2 hours at room temperature. The membranes were washed again in TBST and visualized using Clarity™ western ECL substrate (Bio-Rad) using an M35A XOMAT Processor (Kodak).

### Ex Vivo AAV6.Nme2Cas9 Delivery in Mouse Zygotes

Zygotes were incubated in 15 μl drops of KSOM (Potassium-Supplemented Simplex Optimized Medium, Millipore, Cat. No. MR-106-D) containing 3×10⁹ or 3×10⁸ GCs of AAV6.Nme2Cas9.sgTyr vector for 5-6 h (4 zygotes in each drop). After incubation, zygotes were rinsed in M2 and transferred to fresh KSOM for overnight culture. The next day, the embryos that advanced to 2-cell stage were transferred into the oviduct of pseudopregnant recipients and allowed to develop to term.

### Example XXVI

### Quantification and Statistical Analyses

An analysis of in vitro PAM discovery data was performed using R. GraphPad Prism 6® for all statistical analyses. For mammalian cell experiments using Nme2Cas9, 3 independent replicates were performed and indel percentages were calculated using TIDE software, with error bars depicting s.e.m. The TIDE parameters were set to quantify indels <20 nucleotides for all figures. For side-by-side comparisons of Nme2Cas9 and SpyCas9, average indel percentages were calculated using Microsoft Excel. For in vivo experiments in mice, n=5 for control and test subjects. P values were calculated by unpaired two-tailed t-test.

We claim:

1. A Neisseria meningitidis single guide ribonucleic acid (Nme sgRNA) comprising a Stem 2 region that is 24 nucleotides in length.

2. The Nme sgRNA of claim 1, wherein the Stem 2 region comprises residues 78 to 101 of SEQ ID NO: 220.

3. The Nme sgRNA of claim 1, wherein the length of the Nme sgRNA is 101 nucleotides.

4. The Nme sgRNA of claim 1, wherein the Nme sgRNA is an Nme1Cas9 sgRNA.

5. The Nme sgRNA of claim 1, wherein the Nme sgRNA is an Nme2Cas9 sgRNA.

6. The Nme sgRNA of claim 1, further comprising a truncated repeat:anti-repeat region relative to a repeat:anti-repeat region of a full-length Nme sgRNA, wherein the repeat:anti-repeat region of the full-length Nme sgRNA corresponds to residues 25-76 of SEQ ID NO: 219.

7. The Nme sgRNA of claim 6, wherein the truncated repeat:anti-repeat region comprises a first nucleotide sequence corresponding to residues 25-36 of SEQ ID NO: 219, a second nucleotide sequence corresponding to residues 49-52 of SEQ ID NO: 219, and a third nucleotide sequence corresponding to residues 65-76 of SEQ ID NO: 219.

8. The Nme sgRNA of claim 6, wherein the truncated repeat:anti-repeat region is less than 52 nucleotides in length.

9. The Nme sgRNA of claim 1, further comprising a spacer that is 24 nucleotides in length.

10. The Nme sgRNA of claim 1, wherein the Nme sgRNA comprises at least one chemical modification.

11. The Nme sgRNA of claim 10, wherein the at least one chemical modification comprises a ribose 2′-O-methylation.

12. The Nme sgRNA of claim 10, wherein the at least one chemical modification comprises a phosphorothioate linkage.


SEQUENCE LISTING (314 sequences)

SEQ ID NO: 1 - RNA, 145 nt, synthetic construct
  source 1..145: mol_type = other RNA
nnnnnnnnnnnnnnnnnnnnnnnngttgtagctccctttctcatttcggaaacgaaatgagaaccgttgctacaataaggccgtctgaaaagatgtgccgcaacgctctgccccttaaagcttctgctttaaggggcatcgttta

SEQ ID NO: 2 - RNA, 121 nt, synthetic construct
  source 1..121: mol_type = other RNA
nnnnnnnnnnnnnnnnnnnnnnnngttgtagctcccgaaacgttgctacaataaggccgtctgaaaagatgtgccgcaacgctctgccccttaaagcttctgctttaaggggcatcgttta

SEQ ID NO: 3 - RNA, 111 nt, synthetic construct
  source 1..111: mol_type = other RNA
nnnnnnnnnnnnnnnnnnnnnnnngttgtagctcccgaaacgttgctacaataaggccgtctgaaaagatgtgccgcaacgctctgccccttttctaaggggcatcgttta

SEQ ID NO: 4 - RNA, 105 nt, synthetic construct
  source 1..105: mol_type = other RNA
nnnnnnnnnnnnnnnnnnnnnnnngttgtagctcccgaaacgttgctacaataaggccgtctgaaaagatgtgccgcaacgctctgccccttttctaaggggcat

SEQ ID NO: 5 - RNA, 99 nt, synthetic construct
  source 1..99: mol_type = other RNA
nnnnnnnnnnnnnnnnnnnnnnnngttgtagctcccgaaacgttgctacaataaggccgtctgaaaagatgtgccgcaacgctctgcttctgcatcgtt

SEQ ID NO: 6 - RNA, 100 nt, synthetic construct
  source 1..100: mol_type = other RNA
nnnnnnnnnnnnnnnnnnnnnnngttgtagctcccgaaacgttgctacaataaggccgtctgaaaagatgtgccgcaacgctctgcttctgcatcgttta

SEQ ID NO: 7 - RNA, 100 nt, synthetic construct
  source 1..100: mol_type = other RNA
nnnnnnnnnnnnnnnnnnnnngttgtagctcccgaaacgttgctacaataaggccgtctgaaaagatgtgccgcaacgctctgcccttctgggcatcgtt

SEQ ID NO: 8 - RNA, 107 nt, synthetic construct
  source 1..107: mol_type = other RNA
nnnnnnnnnnnnnnnnnnnnnnnngttgtagctcccgaaacgttgctacaataaggccgtctgaaaagatgtgccgcaacgctctgcccctttctaggggcatcgtt

SEQ ID NO: 9 - RNA, 105 nt, synthetic construct
  source 1..105: mol_type = other RNA
nnnnnnnnnnnnnnnnnnnnnnnngttgtagctcccgaaacgttgctacaataaggccgtctgaaaagatgtgccgcaacgctctgccccttctggggcatcgtt

SEQ ID NO: 10 - RNA, 103 nt, synthetic construct
  source 1..103: mol_type = other RNA
nnnnnnnnnnnnnnnnnnnnnnnngttgtagctcccgaaacgttgctacaataaggccgtctgaaaagatgtgccgcaacgctctgcccttctgggcatcgtt

SEQ ID NO: 11 - RNA, 101 nt, synthetic construct
  source 1..101: mol_type = other RNA
nnnnnnnnnnnnnnnnnnnnnnnngttgtagctcccgaaacgttgctacaataaggccgtctgaaaagatgtgccgcaacgctctgccttctggcatcgtt

SEQ ID NO: 12 - RNA, 100 nt, synthetic construct
  source 1..100: mol_type = other RNA
nnnnnnnnnnnnnnnnnnnnnnngttgtagctcccgaaacgttgctacaataaggccgtctgaaaagatgtgccgcaacgctctgccttctggcatcgtt

SEQ ID NO: 13 - RNA, 24 nt, synthetic construct
  source 1..24: mol_type = other RNA
gagggagagaggtgagcggatgaa

SEQ ID NO: 14 - RNA, 23 nt, synthetic construct
  source 1..23: mol_type = other RNA
ggggagagaggtgagcggatgaa

SEQ ID NO: 15 - RNA, 24 nt, synthetic construct
  source 1..24: mol_type = other RNA
gttctccaagccctcggacctcgt

SEQ ID NO: 16 - RNA, 23 nt, synthetic construct
  source 1..23: mol_type = other RNA
gtctccaagccctcggacctcgt

SEQ ID NO: 17 - RNA, 24 nt, synthetic construct
  source 1..24: mol_type = other RNA
gctggattactgtgtggtagaggg

SEQ ID NO: 18 - RNA, 23 nt, synthetic construct
  source 1..23: mol_type = other RNA
gtggattactgtgtggtagaggg

SEQ ID NO: 19 - DNA, 52 bp, synthetic construct
  misc_feature 1..52: note = Synthetic
  source 1..52: mol_type = other DNA
agcttgagcaaagggagagaggtgagcggatgaagggagattggtgagtatc

SEQ ID NO: 20 - DNA, 52 bp, synthetic construct
  misc_feature 1..52: note = Synthetic
  source 1..52: mol_type = other DNA
cgcttcgcggcttctccaagccctcggacctcgtgggcgtcttctcctgcgt

SEQ ID NO: 21 - DNA, 52 bp, synthetic construct
  misc_feature 1..52: note = Synthetic
  source 1..52: mol_type = other DNA
gaattcactagctggattactgtgtggtagagggaggtgattagcacctgtg

SEQ ID NO: 22 - DNA, 24 bp, synthetic construct
  misc_feature 1..24: note = Synthetic
  source 1..24: mol_type = other DNA
gaatatcaggagactaggaaggag

SEQ ID NO: 23 - skipped

SEQ ID NO: 24 - DNA, 24 bp, synthetic construct
  misc_feature 1..24: note = Synthetic
  source 1..24: mol_type = other DNA
ggacaggagtcgccagaggccggt

SEQ ID NO: 25 - skipped

SEQ ID NO: 26 - DNA, 23 bp, synthetic construct
  misc_feature 1..23: note = Synthetic
  source 1..23: mol_type = other DNA
gcacctgcctcgtggaatacggt

SEQ ID NO: 27 - skipped

SEQ ID NO: 28 - DNA, 52 bp, synthetic construct
  misc_feature 1..52: note = Synthetic
  source 1..52: mol_type = other DNA
atgtggctctggttctgggtacttttatctgtcccctccaccccacagtggg

SEQ ID NO: 29 - DNA, 52 bp, synthetic construct
  misc_feature 1..52: note = Synthetic
  source 1..52: mol_type = other DNA
cagataaggaatctgcctaacaggaggtgggggttagacgaatatcaggaga

SEQ ID NO: 30 - DNA, 52 bp, synthetic construct
  misc_feature 1..52: note = Synthetic
  source 1..52: mol_type = other DNA
ggggttagacgaatatcaggagactaggaaggaggaggcctaaggatggggg

SEQ ID NO: 31 - DNA, 52 bp, synthetic construct
  misc_feature 1..52: note = Synthetic
  source 1..52: mol_type = other DNA
cccacccggcggcgcctccctgcagggctgctccccagcccaaaccgccgcg

SEQ ID NO: 32 - DNA, 52 bp, synthetic construct
  misc_feature 1..52: note = Synthetic
  source 1..52: mol_type = other DNA
tccgagagctcagctagtcttcttcctccaacccgggccctatgtccacttc

SEQ ID NO: 33 - DNA, 52 bp, synthetic construct
  misc_feature 1..52: note = Synthetic
  source 1..52: mol_type = other DNA
tgggtacttttatctgtcccctccaccccacagtggggccactagggacagg

SEQ ID NO: 34 - DNA, 52 bp, synthetic construct
  misc_feature 1..52: note = Synthetic
  source 1..52: mol_type = other DNA
gtaggggagctgcccaaatgaaaggagtgagaggtgacccgaatccacagga

SEQ ID NO: 35 - DNA, 52 bp, synthetic construct
  misc_feature 1..52: note = Synthetic
  source 1..52: mol_type = other DNA
tagcacctctccatcctcttgctttctttgcctggacaccccgttctcctgt

SEQ ID NO: 36 - DNA, 52 bp, synthetic construct
  misc_feature 1..52: note = Synthetic
  source 1..52: mol_type = other DNA
gtctcccttgcgtcccgcctccccttcttgtaggcctgcatcatcaccgttt

SEQ ID NO: 37 - DNA, 53 bp, synthetic construct
  misc_feature 1..53: note = Synthetic
  source 1..53: mol_type = other DNA
cctcacccaaccccatgccgtgttcactcgctgggttcccttttccttctcct

SEQ ID NO: 38 - DNA, 52 bp, synthetic construct
  misc_feature 1..52: note = Synthetic
  source 1..52: mol_type = other DNA
gcgcaggacaggagtcgccagaggccggtggtggatttcctccccgcatctc

SEQ ID NO: 39 - DNA, 51 bp, synthetic construct
  misc_feature 1..51: note = Synthetic
  source 1..51: mol_type = other DNA
cgcggggacgcccagcggccggatatcagctgccacgcccgcgtgggcgga

SEQ ID NO: 40 - DNA, 52 bp, synthetic construct
  misc_feature 1..52: note = Synthetic
  source 1..52: mol_type = other DNA
gattccaatagatctgtgtgtccctctccccacccgtccctgtccggctctc

SEQ ID NO: 41 - DNA, 52 bp, synthetic construct
  misc_feature 1..52: note = Synthetic
  source 1..52: mol_type = other DNA
tgacccctggccttctccccgctccaacgccctcaaccccacacgcacacac

SEQ ID NO: 42 - DNA, 52 bp, synthetic construct
  misc_feature 1..52: note = Synthetic
  source 1..52: mol_type = other DNA
tccctctccccacccgtccctgtccggctctccgccttcccctgcccccttc

SEQ ID NO: 43 - DNA, 52 bp, synthetic construct
  misc_feature 1..52: note = Synthetic
  source 1..52: mol_type = other DNA
acacgcacacactcactcacccacacagacacacacgtcctcactctcgaag

SEQ ID NO: 44 - DNA, 52 bp, synthetic construct
  misc_feature 1..52: note = Synthetic
  source 1..52: mol_type = other DNA
taagcacagtggaagaatttcattctgttctcagttttcctggattatgcct

SEQ ID NO: 45 - DNA, 52 bp, synthetic construct
  misc_feature 1..52: note = Synthetic
  source 1..52: mol_type = other DNA
ttcattctgttctcagttttcctggattatgcctggcaccattaaagaaaat

SEQ ID NO: 46 - DNA, 24 bp, synthetic construct
  misc_feature 1..24: note = Synthetic
  source 1..24: mol_type = other DNA
ggttctgggtacttttatctgtcc

SEQ ID NO: 47 - skipped

SEQ ID NO: 48 - DNA, 19 bp, synthetic construct
  misc_feature 1..19: note = Synthetic
  source 1..19: mol_type = other DNA
tggcttagcacctctccat

SEQ ID NO: 49 - DNA, 24 bp, synthetic construct
  misc_feature 1..24: note = Synthetic
  source 1..24: mol_type = other DNA
agaactcaggaccaacttattctg

SEQ ID NO: 50 - DNA, 24 bp, synthetic construct
  misc_feature 1..24: note = Synthetic
  source 1..24: mol_type = other DNA
gtctgcctaacaggaggtgggggt

SEQ ID NO: 51 - skipped

SEQ ID NO: 52 - DNA, 19 bp, synthetic construct
  misc_feature 1..19: note = Synthetic
  source 1..19: mol_type = other DNA
tggcttagcacctctccat

SEQ ID NO: 53 - DNA, 24 bp, synthetic construct
  misc_feature 1..24: note = Synthetic
  source 1..24: mol_type = other DNA
agaactcaggaccaacttattctg

SEQ ID NO: 54 - DNA, 24 bp, synthetic construct
  misc_feature 1..24: note = Synthetic
  source 1..24: mol_type = other DNA
gaatatcaggagactaggaaggag

SEQ ID NO: 55 - skipped

SEQ ID NO: 56 - DNA, 19 bp, synthetic construct
  misc_feature 1..19: note = Synthetic
  source 1..19: mol_type = other DNA
tggcttagcacctctccat

SEQ ID NO: 57 - DNA, 24 bp, synthetic construct
  misc_feature 1..24: note = Synthetic
  source 1..24: mol_type = other DNA
agaactcaggaccaacttattctg

SEQ ID NO: 58 - DNA, 22 bp, synthetic construct
  misc_feature 1..22: note = Synthetic
  source 1..22: mol_type = other DNA
gcctccctgcagggctgctccc

SEQ ID NO: 59 - skipped

SEQ ID NO: 60 - DNA, 25 bp, synthetic construct
  misc_feature 1..25: note = Synthetic
  source 1..25: mol_type = other DNA
agaggagccttctgactgctgcaga

SEQ ID NO: 61 - DNA, 23 bp, synthetic construct
  misc_feature 1..23: note = Synthetic
  source 1..23: mol_type = other DNA
atgacagacacaaccagagggca

SEQ ID NO: 62 - DNA, 24 bp, synthetic construct
  misc_feature 1..24: note = Synthetic
  source 1..24: mol_type = other DNA
gagctagtcttcttcctccaaccc

SEQ ID NO: 63 - skipped

SEQ ID NO: 64 - DNA, 19 bp, synthetic construct
  misc_feature 1..19: note = Synthetic
  source 1..19: mol_type = other DNA
tggcttagcacctctccat

SEQ ID NO: 65 - DNA, 24 bp, synthetic construct
  misc_feature 1..24: note = Synthetic
  source 1..24: mol_type = other DNA
agaactcaggaccaacttattctg

SEQ ID NO: 66 - DNA, 24 bp, synthetic construct
  misc_feature 1..24: note = Synthetic
  source 1..24: mol_type = other DNA
gatctgtcccctccaccccacagt

SEQ ID NO: 67 - skipped

SEQ ID NO: 68 - DNA, 19 bp, synthetic construct
  misc_feature 1..19: note = Synthetic
  source 1..19: mol_type = other DNA
tggcttagcacctctccat

SEQ ID NO: 69 - DNA, 24 bp, synthetic construct
  misc_feature 1..24: note = Synthetic
  source 1..24: mol_type = other DNA
agaactcaggaccaacttattctg

SEQ ID NO: 70 - DNA, 24 bp, synthetic construct
  misc_feature 1..24: note = Synthetic
  source 1..24: mol_type = other DNA
ggcccaaatgaaaggagtgagagg

SEQ ID NO: 71 - skipped

SEQ ID NO: 72 - DNA, 18 bp, synthetic construct
  misc_feature 1..18: note = Synthetic
  source 1..18: mol_type = other DNA
tccgtcttcctccactcc

SEQ ID NO: 73 - DNA, 20 bp, synthetic construct
  misc_feature 1..20: note = Synthetic
  source 1..20: mol_type = other DNA
taggaaggaggaggcctaag

SEQ ID NO: 74 - DNA, 24 bp, synthetic construct
  misc_feature 1..24: note = Synthetic
  source 1..24: mol_type = other DNA
gcatcctcttgctttctttgcctg

SEQ ID NO: 75 - skipped

SEQ ID NO: 76 - DNA, 18 bp, synthetic construct
  misc_feature 1..18: note = Synthetic
  source 1..18: mol_type = other DNA
tccgtcttcctccactcc

SEQ ID NO: 77 - DNA, 20 bp, synthetic construct
  misc_feature 1..20: note = Synthetic
  source 1..20: mol_type = other DNA
taggaaggaggaggcctaag

SEQ ID NO: 78 - DNA, 24 bp, synthetic construct
  misc_feature 1..24: note = Synthetic
  source 1..24: mol_type = other DNA
ggagtcgccagaggccggtggtgg

SEQ ID NO: 79 - skipped

SEQ ID NO: 80 - DNA, 25 bp, synthetic construct
  misc_feature 1..25: note = Synthetic
  source 1..25: mol_type = other DNA
agaggagccttctgactgctgcaga

SEQ ID NO: 81 - DNA, 23 bp, synthetic construct
  misc_feature 1..23: note = Synthetic
  source 1..23: mol_type = other DNA
atgacagacacaaccagagggca

SEQ ID NO: 82 - DNA, 24 bp, synthetic construct
  misc_feature 1..24: note = Synthetic
  source 1..24: mol_type = other DNA
gcccagcggccggatatcagctgc

SEQ ID NO: 83 - skipped

SEQ ID NO: 84 - DNA, 25 bp, synthetic construct
  misc_feature 1..25: note = Synthetic
  source 1..25: mol_type = other DNA
agaggagccttctgactgctgcaga

SEQ ID NO: 85 - DNA, 23 bp, synthetic construct
  misc_feature 1..23: note = Synthetic
  source 1..23: mol_type = other DNA
atgacagacacaaccagagggca

SEQ ID NO: 86 - DNA, 23 bp, synthetic construct
  misc_feature 1..23: note = Synthetic
  source 1..23: mol_type = other DNA
ggaagggaacatattactattgc

SEQ ID NO: 87 - skipped

SEQ ID NO: 88 - DNA, 19 bp, synthetic construct
  misc_feature 1..19: note = Synthetic
  source 1..19: mol_type = other DNA
tagagaactgggtagtgtg

SEQ ID NO: 89 - DNA, 19 bp, synthetic construct
  misc_feature 1..19: note = Synthetic
  source 1..19: mol_type = other DNA
ccaatattgcatgggatgg

SEQ ID NO: 90 - DNA, 24 bp, synthetic construct
  misc_feature 1..24: note = Synthetic
  source 1..24: mol_type = other DNA
gtggagtggcctgctatcagctac

SEQ ID NO: 91 - skipped

SEQ ID NO: 92 - DNA, 19 bp, synthetic construct
  misc_feature 1..19: note = Synthetic
  source 1..19: mol_type = other DNA
tagagaactgggtagtgtg

SEQ ID NO: 93 - DNA, 19 bp, synthetic construct
  misc_feature 1..19: note = Synthetic
  source 1..19: mol_type = other DNA
ccaatattgcatgggatgg

SEQ ID NO: 94 - DNA, 24 bp, synthetic construct
  misc_feature 1..24: note = Synthetic
  source 1..24: mol_type = other DNA
gaggaagggaacatattactattg

SEQ ID NO: 95 - skipped

SEQ ID NO: 96 - DNA, 19 bp, synthetic construct
  misc_feature 1..19: note = Synthetic
  source 1..19: mol_type = other DNA
tagagaactgggtagtgtg

SEQ ID NO: 97 - DNA, 19 bp, synthetic construct
  misc_feature 1..19: note = Synthetic
  source 1..19: mol_type = other DNA
ccaatattgcatgggatgg

SEQ ID NO: 98 - DNA, 24 bp, synthetic construct
  misc_feature 1..24: note = Synthetic
  source 1..24: mol_type = other DNA
gtgaattctcatcagctaaaatgc

SEQ ID NO: 99 - skipped

SEQ ID NO: 100 - DNA, 19 bp, synthetic construct
  misc_feature 1..19: note = Synthetic
  source 1..19: mol_type = other DNA
tagagaactgggtagtgtg

SEQ ID NO: 101 - DNA, 19 bp, synthetic construct
  misc_feature 1..19: note = Synthetic
  source 1..19: mol_type = other DNA
ccaatattgcatgggatgg

SEQ ID NO: 102 - DNA, 24 bp, synthetic construct
  misc_feature 1..24: note = Synthetic
  source 1..24: mol_type = other DNA
gctcactcacccacacagacacac

SEQ ID NO: 103 - skipped

SEQ ID NO: 104 - DNA, 24 bp, synthetic construct
  misc_feature 1..24: note = Synthetic
  source 1..24: mol_type = other DNA
gtacatgaagcaactccagtccca

SEQ ID NO: 105 - DNA, 22 bp, synthetic construct
  misc_feature 1..22: note = Synthetic
  source 1..22: mol_type = other DNA
atcaaattccagcaccgagcgc

SEQ ID NO: 106 - DNA, 24 bp, synthetic construct
  misc_feature 1..24: note = Synthetic
  source 1..24: mol_type = other DNA
ggaagaatttcattctgttctcag

SEQ ID NO: 107 - skipped

SEQ ID NO: 108 - DNA, 24 bp, synthetic construct
  misc_feature 1..24: note = Synthetic
  source 1..24: mol_type = other DNA
tggtgattatgggagaactggagc

SEQ ID NO: 109 - DNA, 23 bp, synthetic construct
  misc_feature 1..23: note = Synthetic
  source 1..23: mol_type = other DNA
accattgaggacgtttgtctcac

SEQ ID NO: 110 - DNA, 24 bp, synthetic construct
  misc_feature 1..24: note = Synthetic
  source 1..24: mol_type = other DNA
gctcagttttcctggattatgcct

SEQ ID NO: 111 - skipped

SEQ ID NO: 112 - DNA, 24 bp, synthetic construct
  misc_feature 1..24: note = Synthetic
  source 1..24: mol_type = other DNA
tggtgattatgggagaactggagc

SEQ ID NO: 113 - DNA, 23 bp, synthetic construct
  misc_feature 1..23: note = Synthetic
  source 1..23: mol_type = other DNA
accattgaggacgtttgtctcac

SEQ ID NO: 114 - DNA, 24 bp, synthetic construct
  misc_feature 1..24: note = Synthetic
  source 1..24: mol_type = other DNA
gcgttggagcggggagaaggccag

SEQ ID NO: 115 - skipped

SEQ ID NO: 116 - DNA, 24 bp, synthetic construct
  misc_feature 1..24: note = Synthetic
  source 1..24: mol_type = other DNA
gtacatgaagcaactccagtccca

SEQ ID NO: 117 - DNA, 22 bp, synthetic construct
  misc_feature 1..22: note = Synthetic
  source 1..22: mol_type = other DNA
atcaaattccagcaccgagcgc

SEQ ID NO: 118 - DNA, 24 bp, synthetic construct
  misc_feature 1..24: note = Synthetic
  source 1..24: mol_type = other DNA
gggccgcggagatagctgcagggc

SEQ ID NO: 119 - skipped

SEQ ID NO: 120 - DNA, 25 bp, synthetic construct
  misc_feature 1..25: note = Synthetic
  source 1..25: mol_type = other DNA
agaggagccttctgactgctgcaga

SEQ ID NO: 121 - DNA, 23 bp, synthetic construct
  misc_feature 1..23: note = Synthetic
  source 1..23: mol_type = other DNA
atgacagacacaaccagagggca

SEQ ID NO: 122 - DNA, 24 bp, synthetic construct
  misc_feature 1..24: note = Synthetic
  source 1..24: mol_type = other DNA
gcccacccggcggcgcctccctgc

SEQ ID NO: 123 - skipped

SEQ ID NO: 124 - DNA, 25 bp, synthetic construct
  misc_feature 1..25: note = Synthetic
  source 1..25: mol_type = other DNA
agaggagccttctgactgctgcaga

SEQ ID NO: 125 - DNA, 23 bp, synthetic construct
  misc_feature 1..23: note = Synthetic
  source 1..23: mol_type = other DNA
atgacagacacaaccagagggca

SEQ ID NO: 126 - DNA, 24 bp, synthetic construct
  misc_feature 1..24: note = Synthetic
  source 1..24: mol_type = other DNA
gcgtggcagctgatatccggccgc

SEQ ID NO: 127 - skipped

SEQ ID NO: 128 - DNA, 25 bp, synthetic construct
  misc_feature 1..25: note = Synthetic
  source 1..25: mol_type = other DNA
agaggagccttctgactgctgcaga

SEQ ID NO: 129 - DNA, 23 bp, synthetic construct
  misc_feature 1..23: note = Synthetic
  source 1..23: mol_type = other DNA
atgacagacacaaccagagggca

SEQ ID NO: 130 - DNA, 24 bp, synthetic construct
  misc_feature 1..24: note = Synthetic
  source 1..24: mol_type = other DNA
gccgcggcgcgacgtggagccagc

SEQ ID NO: 131 - skipped

SEQ ID NO: 132 - DNA, 25 bp, synthetic construct
  misc_feature 1..25: note = Synthetic
  source 1..25: mol_type = other DNA
agaggagccttctgactgctgcaga

SEQ ID NO: 133 - DNA, 23 bp, synthetic construct
  misc_feature 1..23: note = Synthetic
  source 1..23: mol_type = other DNA
atgacagacacaaccagagggca

SEQ ID NO: 134 - DNA, 24 bp, synthetic construct
  misc_feature 1..24: note = Synthetic
  source 1..24: mol_type = other DNA
gtgctccccagcccaaaccgccgc

SEQ ID NO: 135 - skipped

SEQ ID NO: 136 - DNA, 25 bp, synthetic construct
  misc_feature 1..25: note = Synthetic
  source 1..25: mol_type = other DNA
agaggagccttctgactgctgcaga

SEQ ID NO: 137 - DNA, 23 bp, synthetic construct
  misc_feature 1..23: note = Synthetic
  source 1..23: mol_type = other DNA
atgacagacacaaccagagggca

SEQ ID NO: 138 - DNA, 24 bp, synthetic construct
  misc_feature 1..24: note = Synthetic
  source 1..24: mol_type = other DNA
gtcagattggcttgctcggaattg

SEQ ID NO: 139 - skipped

SEQ ID NO: 140 - DNA, 21 bp, synthetic construct
  misc_feature 1..21: note = Synthetic
  source 1..21: mol_type = other DNA
ggcataaggaaatcgaaggtc

SEQ ID NO: 141 - DNA, 23 bp, synthetic construct
  misc_feature 1..23: note = Synthetic
  source 1..23: mol_type = other DNA
catgtcctcaagtcaagaacaag

SEQ ID NO: 142 - DNA, 24 bp, synthetic construct
  misc_feature 1..24: note = Synthetic
  source 1..24: mol_type = other DNA
gctgggtgaatggagcgagcagcg

SEQ ID NO: 143 - skipped

SEQ ID NO: 144 - DNA, 24 bp, synthetic construct
  misc_feature 1..24: note = Synthetic
  source 1..24: mol_type = other DNA
gtacatgaagcaactccagtccca

SEQ ID NO: 145 - DNA, 22 bp, synthetic construct
  misc_feature 1..22: note = Synthetic
  source 1..22: mol_type = other DNA
atcaaattccagcaccgagcgc

SEQ ID NO: 146 - DNA, 24 bp, synthetic construct
  misc_feature 1..24: note = Synthetic
  source 1..24: mol_type = other DNA
gtcctggagtgacccctggccttc

SEQ ID NO: 147 - skipped

SEQ ID NO: 148 - DNA, 24 bp, synthetic construct
  misc_feature 1..24: note = Synthetic
  source 1..24: mol_type = other DNA
gtacatgaagcaactccagtccca

SEQ ID NO: 149 - DNA, 22 bp, synthetic construct
  misc_feature 1..22: note = Synthetic
  source 1..22: mol_type = other DNA
atcaaattccagcaccgagcgc

SEQ ID NO: 150 - DNA, 24 bp, synthetic construct
  misc_feature 1..24: note = Synthetic
  source 1..24: mol_type = other DNA
gatcctggagtgacccctggcctt

SEQ ID NO: 151 - skipped

SEQ ID NO: 152 - DNA, 24 bp, synthetic construct
  misc_feature 1..24: note = Synthetic
  source 1..24: mol_type = other DNA
gtacatgaagcaactccagtccca

SEQ ID NO: 153 - DNA, 22 bp, synthetic construct
  misc_feature 1..22: note = Synthetic
  source 1..22: mol_type = other DNA
atcaaattccagcaccgagcgc

SEQ ID NO: 154 - DNA, 24 bp, synthetic construct
  misc_feature 1..24: note = Synthetic
  source 1..24: mol_type = other DNA
gtgtgtccctctccccacccgtcc

SEQ ID NO: 155 - skipped

SEQ ID NO: 156 - DNA, 24 bp, synthetic construct
  misc_feature 1..24: note = Synthetic
  source 1..24: mol_type = other DNA
gtacatgaagcaactccagtccca

SEQ ID NO: 157 - DNA, 22 bp, synthetic construct
  misc_feature 1..22: note = Synthetic
  source 1..22: mol_type = other DNA
atcaaattccagcaccgagcgc

SEQ ID NO: 158 - DNA, 24 bp, synthetic construct
  misc_feature 1..24: note = Synthetic
  source 1..24: mol_type = other DNA
gttggagcggggagaaggccaggg

SEQ ID NO: 159 - skipped

SEQ ID NO: 160 - DNA, 24 bp, synthetic construct
  misc_feature 1..24: note = Synthetic
  source 1..24: mol_type = other DNA
gtacatgaagcaactccagtccca

SEQ ID NO: 161 - DNA, 22 bp, synthetic construct
  misc_feature 1..22: note = Synthetic
  source 1..22: mol_type = other DNA
atcaaattccagcaccgagcgc

SEQ ID NO: 162 - DNA, 24 bp, synthetic construct
  misc_feature 1..24: note = Synthetic
  source 1..24: mol_type = other DNA
gcgttggagcggggagaaggccag

SEQ ID NO: 163 - skipped

SEQ ID NO: 164 - DNA, 24 bp, synthetic construct
  misc_feature 1..24: note = Synthetic
  source 1..24: mol_type = other DNA
gtacatgaagcaactccagtccca

SEQ ID NO: 165 - DNA, 22 bp, synthetic construct
  misc_feature 1..22: note = Synthetic
  source 1..22: mol_type = other DNA
atcaaattccagcaccgagcgc

SEQ ID NO: 166 - DNA, 24 bp, synthetic construct
  misc_feature 1..24: note = Synthetic
  source 1..24: mol_type = other DNA
gtaccctccaataatttggctggc

SEQ ID NO: 167 - skipped

SEQ ID NO: 168 - DNA, 21 bp, synthetic construct
  misc_feature 1..21: note = Synthetic
  source 1..21: mol_type = other DNA
ggcataaggaaatcgaaggtc

SEQ ID NO: 169 - DNA, 23 bp, synthetic construct
  misc_feature 1..23: note = Synthetic
  source 1..23: mol_type = other DNA
catgtcctcaagtcaagaacaag

SEQ ID NO: 170 - DNA, 24 bp, synthetic construct
  misc_feature 1..24: note = Synthetic
  source 1..24: mol_type = other DNA
gataatttggctggcaattccgag

SEQ ID NO: 171 - skipped

SEQ ID NO: 172 - DNA, 21 bp, synthetic construct
  misc_feature 1..21: note = Synthetic
  source 1..21: mol_type = other DNA
ggcataaggaaatcgaaggtc

SEQ ID NO: 173 - DNA, 23 bp, synthetic construct
  misc_feature 1..23: note = Synthetic
  source 1..23: mol_type = other DNA
catgtcctcaagtcaagaacaag

SEQ ID NO: 174 - DNA, 24 bp, synthetic construct
  misc_feature 1..24: note = Synthetic
  source 1..24: mol_type = other DNA
gcaggggccaggtgtccttctctg

SEQ ID NO: 175 - skipped

SEQ ID NO: 176 - DNA, 23 bp, synthetic construct
  misc_feature 1..23: note = Synthetic
  source 1..23: mol_type = other DNA
acacgggcagcatgggaatagtc

SEQ ID NO: 177 - DNA, 24 bp, synthetic construct
  misc_feature 1..24: note = Synthetic
  source 1..24: mol_type = other DNA
gctaggggagagtcccactgtcca

SEQ ID NO: 178 - DNA, 24 bp, synthetic construct
  misc_feature 1..24: note = Synthetic
  source 1..24: mol_type = other DNA
gaatggcaggcggaggttgtactg

SEQ ID NO: 179 - skipped

SEQ ID NO: 180 - DNA, 23 bp, synthetic construct
  misc_feature 1..23: note = Synthetic
  source 1..23: mol_type = other DNA
cctgtgtggctttgctttggtcg

SEQ ID NO: 181 - DNA, 24 bp, synthetic construct
  misc_feature 1..24: note = Synthetic
  source 1..24: mol_type = other DNA
gtagggtgtgatgggaggctaagc

SEQ ID NO: 182 - DNA, 24 bp, synthetic construct
  misc_feature 1..24: note = Synthetic
  source 1..24: mol_type = other DNA
gagtgagagagtgagagagagaca

SEQ ID NO: 183 - skipped

SEQ ID NO: 184 - DNA, 23 bp, synthetic construct
  misc_feature 1..23: note = Synthetic
  source 1..23: mol_type = other DNA
cctgtgtggctttgctttggtcg

SEQ ID NO: 185 - DNA, 24 bp, synthetic construct
  misc_feature 1..24: note = Synthetic
  source 1..24: mol_type = other DNA
gtagggtgtgatgggaggctaagc

SEQ ID NO: 186 - DNA, 24 bp, synthetic construct
  misc_feature 1..24: note = Synthetic
  source 1..24: mol_type = other DNA
gtgagcaggcacctgtgccaacat

SEQ ID NO: 187 - skipped

SEQ ID NO: 188 - DNA, 23 bp, synthetic construct
  misc_feature 1..23: note = Synthetic
  source 1..23: mol_type = other DNA
cctgtgtggctttgctttggtcg

SEQ ID NO: 189 - DNA, 24 bp, synthetic construct
  misc_feature 1..24: note = Synthetic
  source 1..24: mol_type = other DNA
gtagggtgtgatgggaggctaagc

SEQ ID NO: 190 - DNA, 24 bp, synthetic construct
  misc_feature 1..24: note = Synthetic
  source 1..24: mol_type = other DNA
gcgtgggggctccgtgccccacgc

SEQ ID NO: 191 - skipped

SEQ ID NO: 192 - DNA, 23 bp, synthetic construct
  misc_feature 1..23: note = Synthetic
  source 1..23: mol_type = other DNA
ggaggaagagtagctcgccgagg

SEQ ID NO: 193 - DNA, 24 bp, synthetic construct
  misc_feature 1..24: note = Synthetic
  source 1..24: mol_type = other DNA
agaccgagtggcagtgacagcaag

SEQ ID NO: 194 - DNA, 24 bp, synthetic construct
  misc_feature 1..24: note = Synthetic
  source 1..24: mol_type = other DNA
gcatgggcaggggctggggtgcac

SEQ ID NO: 195 - skipped

SEQ ID NO: 196 - DNA, 24 bp, synthetic construct
  misc_feature 1..24: note = Synthetic
  source 1..24: mol_type = other DNA
agggagagggaagtgtggggaagg

SEQ ID NO: 197 - DNA, 24 bp, synthetic construct
  misc_feature 1..24: note = Synthetic
  source 1..24: mol_type = other DNA
gtcttcctgctctgtgcgcacgac

SEQ ID NO: 198 - DNA, 24 bp, synthetic construct
  misc_feature 1..24: note = Synthetic
  source 1..24: mol_type = other DNA
gaaaattgtgatttccagatccac

SEQ ID NO: 199 - skipped

SEQ ID NO: 200 - DNA, 23 bp, synthetic construct
  misc_feature 1..23: note = Synthetic
  source 1..23: mol_type = other DNA
gttgggggctctaagttatgtat

SEQ ID NO: 201 - DNA, 23 bp, synthetic construct
  misc_feature 1..23: note = Synthetic
  source 1..23: mol_type = other DNA
cttcatctgtatcttcaggatca

SEQ ID NO: 202 - DNA, 23 bp, synthetic construct
  misc_feature 1..23: note = Synthetic
  source 1..23: mol_type = other DNA
gagcagaaaaaattgtgatttcc

SEQ ID NO: 203 - skipped

SEQ ID NO: 204 - DNA, 23 bp, synthetic construct
  misc_feature 1..23: note = Synthetic
  source 1..23: mol_type = other DNA
gttgggggctctaagttatgtat

SEQ ID NO: 205 - DNA, 23 bp, synthetic construct
  misc_feature 1..23: note = Synthetic
  source 1..23: mol_type = other DNA
cttcatctgtatcttcaggatca

SEQ ID NO: 206 - skipped

SEQ ID NO: 207 - RNA, 121 nt, synthetic construct
  source 1..121: mol_type = other RNA
gttgtagctccctttctcatttcggaaacgaaatgagaaccgttgctacaataaggccgtctgaaaagatgtgccgcaacgctctgccccttaaagcttctgctttaaggggcatcgttat

SEQ ID NO: 208 - RNA, 144 nt, synthetic construct
  source 1..144: mol_type = other RNA
gagggagagaggtgagcggatgaagttgtagctccctttctcatttcggaaacgaaatgagaaccgttgctacaataaggcgctctgaaaagatgtgccgcaacgctctgcccttaaagcttctgctttaaggggcatcgttta

SEQ ID NO: 209 - RNA, 121 nt, synthetic construct
  source 1..121: mol_type = other RNA
gagggagagaggtgagcggatgaagttgtagctcccgaaacgttgctacaataaggccgtctgaaaagatgtgccgcaacgctctgccccttaaagcttctgctttaaggggcatcgttta

SEQ ID NO: 210 - RNA, 111 nt, synthetic construct
  source 1..111: mol_type = other RNA
gagggagagaggtgagcggatgaagttgtagctcccgaaacgttgctacaataaggccgtctgaaaagatgtgccgcaacgctctgccccttttctaaggggcatcgttta

SEQ ID NO: 211 - RNA, 105 nt, synthetic construct
  source 1..105: mol_type = other RNA
gagggagagaggtgagcggatgaagttgtagctcccgaaacgttgctacaataaggccgtctgaaaagatgtgccgcaacgctctgccccttttctaaggggcat

SEQ ID NO: 212 - RNA, 99 nt, synthetic construct
  source 1..99: mol_type = other RNA
gagggagagaggtgagcggatgaagttgtagctcccgaaacgttgctacaataaggccgtctgaaaagatgtgccgcaacgctctgcttctgcatcgtt

SEQ ID NO: 213 - RNA, 99 nt, synthetic construct
  source 1..99: mol_type = other RNA
gggagagaggtgagcggatgaagttgtagctcccgaaacgttgctacaataaggccgtctgaaaagatgtgccgcaacgctctgcttctgcatcgttta

SEQ ID NO: 214 - RNA, 100 nt, synthetic construct
  source 1..100: mol_type = other RNA
ggagagaggtgagcggatgaagttgtagctcccgaaacgttgctacaataaggccgtctgaaaagatgtgccgcaacgctctgcccttctgggcatcgtt

SEQ ID NO: 215 - RNA, 107 nt, synthetic construct
  source 1..107: mol_type = other RNA
gagggagagaggtgagcggatgaagttgtagctcccgaaacgttgctacaataaggccgtctgaaaagatgtgccgcaacgctctgcccctttctaggggcatcgtt

SEQ ID NO: 216 - RNA, 105 nt, synthetic construct
  source 1..105: mol_type = other RNA
gagggagagaggtgagcggatgaagttgtagctcccgaaacgttgctacaataaggccgtctgaaaagatgtgccgcaacgctctgccccttctggggcatcgtt

SEQ ID NO: 217 - RNA, 96 nt, synthetic construct
  source 1..96: mol_type = other RNA
gagggagagaggtgagcggatgaagttgtagctcccgaaacgttgctacaataaggccgtctgaaaagatgtgccgcaacgctctgcccttctggg

SEQ ID NO: 218 - RNA, 101 nt, synthetic construct
  source 1..101: mol_type = other RNA
gagggagagaggtgagcggatgaagttgtagctcccgaaacgttgctacaataaggccgtctgaaaagatgtgccgcaacgctctgccttctggcatcgtt

SEQ ID NO: 219 - RNA, 145 nt, synthetic construct; identical to SEQ ID NO: 1
  source 1..145: mol_type = other RNA

SEQ ID NO: 220 - RNA, 101 nt, synthetic construct; identical to SEQ ID NO: 11
  source 1..101: mol_type = other RNA

SEQ ID NO: 221 - RNA, 100 nt, synthetic construct; identical to SEQ ID NO: 12
  source 1..100: mol_type = other RNA

SEQ ID NO: 222 - RNA, 121 nt, synthetic construct
  source 1..121: mol_type = other RNA
gttgtagctccctttctcatttcggaaacgaaatgagaaccgttgctacaataaggccgtctgaaaagatgtgccgcaacgctctgccccttaaagcttctgctttaaggggcatcgttta

SEQ ID NO: 223 - RNA, 77 nt, synthetic construct
  source 1..77: mol_type = other RNA
gttgtagctcccgaaacgttgctacaataaggccgtctgaaaagatgtgccgcaacgctctgccttctggcatcgtt

SEQ ID NO: 224 - RNA, 77 nt, synthetic construct
  source 1..77: mol_type = other RNA
gttgtagctcccgaaacgttgctacaataaggccgttgaaaagtgtgccgcaacgctctgccttctggcatcgttta

SEQ ID NO: 225 - RNA, 121 nt, synthetic construct; identical to SEQ ID NO: 222
  source 1..121: mol_type = other RNA

SEQ ID NO: 226 - RNA, 79 nt, synthetic construct
  source 1..79: mol_type = other RNA
gttgtagctcccgaaacgttgctacaataaggccgtctgaaaagatgtgccgcaacgctctgccttctggcatcgttta

SEQ ID NO: 227 - RNA, 121 nt, synthetic construct; identical to SEQ ID NO: 222
  source 1..121: mol_type = other RNA

SEQ ID NO: 228 - RNA, 97 nt, synthetic construct
  source 1..97: mol_type = other RNA
gttgtagctcccgaaacgttgctacaataaggccgtctgaaaagatgtgccgcaacgctctgccccttaaagcttctgctttaaggggcatcgttta

SEQ ID NO: 229 - DNA, 427 bp, Neisseria meningitidis
  source 1..427: mol_type = other DNA
ggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggaagagcattccagctcttcagttgtagctccctttctcatttcggaaacgaaatgagaaccgttgctacaataaggccgtctgaaaagatgtgccgcaacgctctgccccttaaagcttctgctttaaggggcatcgtttatttttttgtttaaactctagaggccgc

SEQ ID NO: 230 - DNA, 3963 bp, Neisseria meningitidis
  source 1..3963: mol_type = other DNA
acgtcgactctagaatggaggcggtactatgtagatgagaattcaggagcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcatacttaagcagacagtggttcaaagtttttttcttccatttcaggtgtcgtgaacaccgccaccatggtgcctaagaagaagagaaaggtggaagataaacgcccagcagctacaaagaaggcaggtcaagccaagaaaaagaaagcagcattcaagccaaactcaatcaattacatcctgggactggacatcggcatcgcatccgtcgggtgggctatggtcgaaatcgacgaggaggagaaccccatccgcctgatcgatctgggcgtgcgcgtgtttgagagggcagaggtgcctaagaccggcgacagcctggccatggcacggagactggcacgctccgtgaggcgcctgacccggagaagggcccacagactgctgaggacacgccggctgctgaagagggagggcgtgctgcaggccgccaacttcgatgagaatggcctgatcaagtccctgcccaataccccttggcagctgagggcagccgccctggaccgcaagctgacacctctggagtggtccgccgtgctgctgcacctgatcaagcaccggggctacctgtctcagagaaagaacgagggcgagacagccgataaggagctgggcgccctgctgaagggagtggcaggaaatgcacacgccctgcagaccggcgactttcgcacaccagccgagctggccctgaacaagttcgagaaggagagcggccacatccgcaatcagcggtctgactatagccacaccttctcccggaaggatctgcaggccgagctgatcctgctgtttgagaagcagaaggagttcggcaacccacacgtgtctggcggcctgaaggagggcatcgagacactgctgatgacacagcggcccgccctgagcggcgacgcagtgcagaagatgctgggacactgcacctttgagccagccgagcccaaggccgccaagaatacctacacagccgagcggttcatctggctgacaaagctgaacaatctgaggatcctggagcagggaagcgagcgcccactgaccgacacagagagggccaccctgatggatgagccctaccgcaagtccaagctgacatatgcacaggcaaggaagctgctgggcctggaggacaccgccttctttaagggcctgagatacggcaaggataacgccgaggcctctacactgatggagatgaaggcctatcacgccatcagcagggccctggagaaggagggcctgaaggacaagaagtccccactgaatctgtctcccgagctgcaggatgagatcggcaccgcctttagcctgttcaagaccgacgaggatatcacaggcagactgaaggacaggatccagccagagatcctggaggccctgctgaagcacatcagctttgataagttcgtgcagatcagcctgaaggccctgcggaggatcgtgccactgatggagcagggcaagaggtacgacgaggcctgcgccgaaatctacggcgatcactatggcaagaagaacacagaggagaaaatctacctgccccctatccccgccgatgagatcaggaaccctgtggtgctgcgcgccctgtctcaggcaagaaaagtgatcaacggagtggtgcgccggtacggcagccccgccagaatccacatcgagacagccagggaagtgggcaagtcctttaaggacagaaaggagatcgagaagaggcaggaggagaacagaaaggatagggagaaggccgccgccaagttcagagagtactttcctaatttcgtgggcgagccaaagtccaaggatatcctgaagctgaggctgtacgagcagcagcacggcaagtgtctgtattctggcaaggagatcaacctgggccgcctgaatgagaagggctatgtggagatcgaccacgccctgcctttttctcggacctgggacgatagcttcaacaataaggtgctggtgctgggctctgagaaccagaataagggcaaccagacaccctacgagtatttcaacggcaaggacaatagccgcgagtggcaggagtttaaggcaagggtggagacaagcaggttccctcggtccaagaagcagagaatcctgctgcagaagtttgacgaggatggcttcaaggagaggaacctgaatgacacccgctacgtgaatcggtttctgtgccagttcgtggccgatagaatgaggctgaccggcaagggcaagaagagagtgtttgcctccaacggccagatcacaaatctgctgaggggcttctggggcctgagaaaggtgagggcagagaacgacaggcaccacgcactggatgcagtggtggtggcatgttctaccgtggccatgcagcagaagatcacacgctttgtgcggtataaggagatgaatgccttcgacggcaagaccatcgataaggagacaggcgaggtgctgcaccagaagacacactttcctcagccatgggagttctttgcccaggaagtgatgatccgggtgtttggcaagcctgacggcaagccagagttcgaggaggccgataccctggagaagctgagaacactgctggcagagaagctgagctccaggcccgaggcagtgcaccagtacgtgaccccactgttcgtgtctagagcccccaacaggaagatgagcggccagggccacatggagacagtgaagtccgccaagagactggacgagggcgtgtctgtgctgagggtgcctctgacacagctgaagctgaaggatctggagaagatggtgaatcgcgagcgggagccaaagctgtatgaggccctgaaggcaaggctggaggcacacaaggacgatcctgccaaggcctttgccgagccattctacaagtatgataaggccggcaacagaacccagcaggtgaaggccgtgagggtggagcaggtgcagaagacaggcgtgtgggtgcgcaaccacaatggcatcgccgacaatgctaccatggtgcgggtggacgtgtttgagaagggcgataagtactatctggtgcccatctacagctggcaggtggccaagggcatcctgcctgatagagccgtggtgcagggcaaggacgaggaggattggcagctgatcgacgattccttcaactttaagttctctctgcaccccaatgacctggtggaagtgatcaccaagaaggccaggatgtttggctacttcgcctcctgccaccgcggcacaggcaacatcaatatccggatccacgacctggatcacaagatcggcaagaacggcatcctggagggcatcggcgtgaagacagccctgagcttccagaagtatcagatcgacgagctgggcaaggagatcagaccttgtaggctgaagaagcgcccacccgtgcgggaggataagcggcccgcagcaaccaagaaggcaggacaggccaagaagaagaagtacccctatgacgtgcctgactacgccgggtatccctacgacgtgcctgattacgccgggtcctatccctacgacgtgccagattacgctgcagctccagcagcgaagaaaagaagctggattaagatctttttccctctgccaaaaattatggggacatcatgaagccccttgagcatctgacttctggctaataaaggaaatttattttcattgcaatagtgtgttggaattttttgtgtctctcactcggcggccgc

SEQ ID NO: 231 - DNA, 835 bp, synthetic construct
  misc_feature 1..835: note = Synthetic
  source 1..835: mol_type = other DNA
tggcgtgatctcccggcccccaggcgtccagtacccacaccccagaaggcttccaccttcacgtggacgcgcaggctgccggtgggctcccgttctctctctctttctgaggctagaggactgagccagtccttggctccccagagacatcacggcccgcagccccggagccaagtgccccgagtcccaggcgtccatgtccttcccgaggccgcgcgcacctctcctcgccccgatgggcacccactgctctgcgtggctgcggtggccgctgttgccgctgttgccgccgctgctgctgctgttgctgctactgtgccccaccggcgctggtgcccaggacgaggatggagattatgaagagctgatgctcgccctcccgtcccaggaggatggcctggctgatgaggccgcacatgtggccaccgccaccttccgccgttgctccaaggtatgggtgccaggcaacggcgttgctttggggttggggtgatgctcttcgggggtcttctctgctcatctagccgtctggtggtctctaagtgcagccctgaggtgcgggaggcgagggcaagacttagtgctcagctgcaccttgtggcacagagtgatgggggaggccacgtgctaaaggcactgcggggcttggttccaaaagtgtgaggcggggagcgggctaccagtgtggtcatgcagaaaacgtgtcctccgaagtaaagtggcatcgggaggctgagaactctagtggcacatctttctcaactggtcatccagcagtcatcctgggtgcctgttggggttctgtaggcctcgatttagcggaagggtgtcaggg

SEQ ID NO: 232 - DNA, 863 bp, synthetic construct
  misc_feature 1..863: note = Synthetic
  source 1..863: mol_type = other DNA
ggaaagggaaaatgccaatgctctgtctaggggttggataagccagtataataaatgaaaatggggctaaaatgagtgttctaaaataccttttgataaggctgcagaaggagcgggagaaatggatatgaagtactgggctctttaaaaatgattaaaattctgcttacatagtctaactcgcgacactgtaatttcatactgtagtaaggatctcaagcaggagagtataaaactcgggtgagcatgtctttaatctacctcgatggaaaatactccgaggcggatcacaagcaataataacctgtagttttgctgcataaaaccccagatgactacctatcctcccattttccttatttgcccctattaaaaaacttcccgacaaaaccgaaaatctgtgggaagtcttgtccctccaattttacacctgttcaattcccctgcaggacaacgcccacacaccaggttagcctttaagcctgcccagaagactcccgcccatcttctagaaagactggagttgcagatcacgagggaagagggggaagggattctcccaggcccagggcggtcctcagaagccaggaggcagcagagaactcccagaaaggtattgcaacactcccctcccccctccggagaagggtgcggccttctccccgcctactccactgcagctcccttactgataacaactcagagcgactttgggagagcaagtgcttcctgcctccaaaacagcccaactgagccctcgctccttccctccactccccggagtgcgcgatggaggtctggctcagcacgcccctcttgaggcaactcaagtcggaaacgtgcttgcacccgccccgcagccg

SEQ ID NO: 233 - DNA, 3249 bp, Neisseria meningitidis
  source 1..3249: mol_type = other DNA
atggctgccttcaaacctaatccaatcaactacatcctcggcctcgatatcggcatcgcatccgtcggctgggcgatggtagaaattgacgaagaagaaaaccccatccgcctgattgatttgggcgtgcgcgtatttgagcgtgccgaagtaccgaaaacaggcgactcccttgccatggcaaggcgtttggcgcgcagtgtccgccgcctgacccgccgtcgcgcccatcgcctgcttcgggcccgccgcctattgaaacgcgaaggcgtattacaagccgctgattttgacgaaaacggcttgatcaaatccttaccgaatacaccatggcaacttcgcgcagccgcattagaccgcaaactgacgcctttagagtggtcggcagtcttgttgcatttaatcaaacaccgcggttatttgtcgcaaagaaaaaacgagggcgaaactgccgataaagagcttggcgctttgcttaaaggcgtggccaacaatgcccatgccttacagacaggcgatttccgcacaccggccgaattggctttaaataaatttgagaaagaaagcggccatatccgcaatcagcgcggcgattattcgcatacgttcagccgcaaagatttacaggcggagctgattttgctgtttgaaaaacaaaaagaatttggcaatccgcatgtttcaggcggccttaaagaaggtattgaaaccctactgatgacgcaacgccctgccctgtccggcgatgccgttcaaaaaatgttggggcattgcaccttcgaaccggcagagccgaaagccgctaaaaacacctacacagccgaacgtttcatctggctgaccaagctgaacaacctgcgtattttagagcaaggcagcgagcggccattgaccgataccgaacgcgccacgcttatggacgagccatacagaaaatccaaactgacttacgcacaagcccgtaagctgctgggtttagaagataccgcctttttcaaaggcttgcgctatggtaaagacaatgccgaagcctcaacattgatggaaatgaaggcctaccatgccatcagccgtgcactggaaaaagaaggattgaaagataaaaaatccccattaaacctttcttccgaattacaagatgaaatcggcacggcattctccctgttcaaaaccgatgaagacattacaggccgtctgaaagaccgtgttcagcccgaaatcttagaagcgctgttgaaacacatcagcttcgataagttcgtccaaatttccttgaaagcattgcgccgaattgtgcctctaatggagcaaggcaaacgttacgatgaagcctgcgccgaaatctacggagaccattacggcaagaaaaatacggaagaaaaaatttatctgccgcccatccctgccgacgagatccgcaaccccgtcgtcttgcgcgccttatctcaagcacgtaaggtcattaacggcgtggtacgccgttacggctccccagctcgtatccatattgaaacggcaagggaagtaggtaaatcgtttaaagaccgcaaagaaatcgaaaaacgccaagaagaaaaccgcaaagaccgggaaaaagccgccgccaaattccgagagtatttccccaattttgtcggcgaacccaaatcaaaagatattctgaaactgcgcctgtacgagcaacaacacggcaaatgcctgtattcgggcaaagaaatcaacttagtccgtctgaacgaaaaaggctatgtcgaaatcgaccatgccctgccgttttcgcgcacatgggacgacagtttcaacaataaagtgctggtattgggcagcgaaaaccaaaacaaaggcaatcaaaccccttacgaatacttcaacggcaaagacaacagccgcgaatggcaggaatttaaagcgcgtgtcgaaaccagccgtttcccgcgcagtaaaaaacaacggattctgctgcaaaaattcgatgaagacggctttaaagaatgcaatctgaacgacacgcgctacgtcaaccgtttcctgtgccaatttgttgccgaccatatattgctgacaggtaaagggaaaagacgtgtctttgcctcaaacggacaaattaccaatctgttgcgcggcttttggggattgcgcaaagtgcgtgcggaaaacgaccgccatcacgccttggacgctgtagtcgttgcctgctcgaccgttgccatgcagcagaaaattacccgttttgtacgctataaagagatgaacgcgtttgacggtaaaaccatagacaaagaaacaggaaaagtgctgcatcaaaaaacacacttcccacaaccttgggaatttttcgcacaagaagtcatgattcgcgtcttcggcaaaccggacggcaaacccgaattcgaagaagccgataccccagaaaaactgcgcacgttgcttgccgaaaaattatcatctcgccccgaagccgtacacgaatacgttacgccactgtttgtttcacgcgcgcccaatcggaagatgagcggtgcacataaagatactttgagatctgctaaacgatttgttaaacataatgaaaaaattagtgttaaacgagtatggttaaccgaaatcaagttggccgaccttgaaaatatggttaattataaaaatggtagagagattgaattatatgaggctcttaaggcgcgtttagaggcatatggaggtaatgctaaacaagcatttgaccctaaggacaatccgttttataaaaagggaggacaactggttaaagctgtgagggtcgaaaaaacccaagagagcggagtcttattaaataaaaaaaatgcttataccattgcagataatggagacatggtacgtgttgatgtgttctgtaaagtagataagaaaggaaaaaatcagtattttattgttcccatctatgcttggcaggttgctgaaaacattcttcccgatattgattgtaagggataccggattgatgatagctatacattctgttttagcttgcataagtatgatctgattgcttttcaaaaagatgaaaaatctaaagtagaattcgcctactatatcaactgtgatagctctaatggacgattctatttagcttggcatgataaaggctctaaagaacagcaattccgtattagcacccaaaatcttgtattgatacaaaaataccaagttaacgaactgggcaaagaaatcagaccatgccgtctgaaaaaacgcccacctgtccgttaa

SEQ ID NO: 234 - protein, 1082 aa, Neisseria meningitidis
MAAFKPNPINYILGLDIGIASVGWAMVEIDEEENPIRLIDLGVRVFERAEVPKTGDSLAMARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVANNAHALQTGDFRTPAELALNKFEKESGHIRNQRGDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSSELQDEIGTAFSLFKTDEDITGRLKDRVQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLVRLNEKGYVEIDHALPFSRTWDDSFNNKVLVLGSENQNKGNQTPYEYFNGKDNSREWQEFKARVETSRFPRSKKQRILLQKFDEDGFKECNLNDTRYVNRFLCQFVADHILLTGKGKRRVFASNGQITNLLRGFWGLRKVRAENDRHHALDAVVVACSTVAMQQKITRFVRYKEMNAFDGKTIDKETGKVLHQKTHFPQPWEFFAQEVMIRVFGKPDGKPEFEEADTPEKLRTLLAEKLSSRPEAVHEYVTPLFVSRAPNRKMSGAHKDTLRSAKRFVKHNEKISVKRVWLTEIKLADLENMVNYKNGREIELYEALKARLEAYGGNAKQAFDPKDNPFYKKGGQLVKAVRVEKTQESGVLLNKKNAYTIADNGDMVRVDVFCKVDKKGKNQYFIVPIYAWQVAENILPDIDCKGYRIDDSYTFCFSLHKYDLIAFQKDEKSKVEFAYYINCDSSNGRFYLAWHDKGSKEQQFRISTQNLVLIQKYQVNELGKEIRPCRLKKRPPVR

SEQ ID NO: 235 - DNA, 3423 bp, synthetic construct
  misc_feature 1..3423: note = Synthetic
  source 1..3423: mol_type = other DNA
atggccgccttcaagcctaacccaatcaattacatcctgggactggacatcggaatcgcatccgtgggatgggctatggtggagatcgacgaggaggagaatcctatccggctgatcgatctgggcgtgagagtgtttgagagggccgaggtgccaaagaccggcgattctctggctatggcccggagactggcacggagcgtgaggcgcctgacacggagaagggcacacaggctgctgagggcacgccggctgctgaagagagagggcgtgctgcaggcagcagacttcgatgagaatggcctgatcaagagcctgccaaacaccccctggcagctgagagcagccgccctggacaggaagctgacaccactggagtggtctgccgtgctgctgcacctgatcaagcaccgcggctacctgagccagcggaagaacgagggagagacagcagacaaggagctgggcgccctgctgaagggagtggccaacaatgcccacgccctgcagaccggcgatttcaggacacctgccgagctggccctgaataagtttgagaaggagtccggccacatcagaaaccagaggggcgactatagccacaccttctcccgcaaggatctgcaggccgagctgatcctgctgttcgagaagcagaaggagtttggcaatccacacgtgagcggaggcctgaaggagggaatcgagaccctgctgatgacacagaggcctgccctgtccggcgacgcagtgcagaagatgctgggacactgcaccttcgagcctgcagagccaaaggccgccaagaacacctacacagccgagcggtttatctggctgacaaagctgaacaatctgagaatcctggagcagggatccgagaggccactgaccgacacagagagggccaccctgatggatgagccttaccggaagtctaagctgacatatgcccaggccagaaagctgctgggcctggaggacaccgccttctttaagggcctgagatacggcaaggataatgccgaggcctccacactgatggagatgaaggcctatcacgccatctctcgcgccctggagaaggagggcctgaaggacaagaagtcccccctgaacctgagctccgagctgcaggatgagatcggcaccgccttctctctgtttaagaccgacgaggatatcacaggccgcctgaaggacagggtgcagcctgagatcctggaggccctgctgaagcacatctctttcgataagtttgtgcagatcagcctgaaggccctgagaaggatcgtgccactgatggagcagggcaagcggtacgacgaggcctgcgccgagatctacggcgatcactatggcaagaagaacacagaggagaagatctatctgccccctatccctgccgacgagatcagaaatcctgtggtgctgagggccctgtcccaggcaagaaaagtgatcaacggagtggtgcgccggtacggatctccagcccggatccacatcgagaccgccagagaagtgggcaagagcttcaaggaccggaaggagatcgagaagagacaggaggagaatcgcaaggatcgggagaaggccgccgccaagtttagggagtacttccctaactttgtgggcgagccaaagtctaaggacatcctgaagctgcgcctgtacgagcagcagcacggcaagtgtctgtatagcggcaaggagatcaatctggtgcggctgaacgagaagggctatgtggagatcgatcacgccctgcctttctccagaacctgggacgattcttttaacaataaggtgctggtgctgggcagcgagaaccagaataagggcaatcagacaccatacgagtatttcaatggcaaggacaactccagggagtggcaggagttcaaggcccgcgtggagacctctagatttcccaggagcaagaagcagcggatcctgctgcagaagttcgacgaggatggctttaaggagtgcaacctgaatgacaccagatacgtgaaccggttcctgtgccagtttgtggccgatcacatcctgctgaccggcaagggcaagagaagggtgttcgcctctaatggccagatcacaaacctgctgaggggattttggggactgaggaaggtgcgggcagagaatgacagacaccacgcactggatgcagtggtggtggcatgcagcaccgtggcaatgcagcagaagatcacaagattcgtgaggtataaggagatgaacgcctttgacggcaagaccatcgataaggagacaggcaaggtgctgcaccagaagacccacttcccccagccttgggagttctttgcccaggaagtgatgatccgggtgttcggcaagccagacggcaagcctgagtttgaggaggccgataccccagagaagctgaggacactgctggcagagaagctgtctagcaggccagaggcagtgcacgagtacgtgaccccactgttcgtgtccagggcacccaatcggaagatgtctggcgcccacaaggacacactgagaagcgccaagaggtttgtgaagcacaacgagaagatctccgtgaagagagtgtggctgaccgagatcaagctggccgatctggagaacatggtgaattacaagaacggcagggagatcgagctgtatgaggccctgaaggcaaggctggaggcctacggaggaaatgccaagcaggccttcgacccaaaggataaccccttttataagaagggaggacagctggtgaaggccgtgcgggtggagaagacccaggagagcggcgtgctgctgaataagaagaacgcctacacaatcgccgacaatggcgatatggtgagagtggacgtgttctgtaaggtggataagaagggcaagaatcagtactttatcgtgcctatctatgcctggcaggtggccgagaacatcctgccagacatcgattgcaagggctacagaatcgacgatagctatacattctgtttttccctgcacaagtatgacctgatcgccttccagaaggatgagaagtccaaggtggagtttgcctactatatcaattgcgactcctctaacggcaggttctacctggcctggcacgataagggcagcaaggagcagcagtttcgcatctccacccagaatctggtgctgatccagaagtatcaggtgaacgagctgggcaaggagatcaggccatgtcggctgaagaagcgcccacccgtgcggggcaccggcgggcccaagaagaagaggaaggtatacccatacgatgttcctgactatgcgggctatccctatgacgtcccggactatgcaggatcgtatccttatgacgttccagattacgctggatccgccgctccggcagctaagaaaaagaaactggatttcgaatccggataa

SEQ ID NO: 236 - protein, 1141 aa, synthetic construct
  REGION 1..1141: note = Synthetic
MAAFKPNPINYILGLDIGIASVGWAMVEIDEEENPIRLIDLGVRVFERAEVPKTGDSLAMARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVANNAHALQTGDFRTPAELALNKFEKESGIILRNQRGDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSSELQDEIGTAFSLFKTDEDITGRLKDRVQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLVRLNEKGYVEIDHALPFSRTWDDSFNNKVLVLGSENQNKGNQTPYEYFNGKDNSREWQEFKARVETSRFPRSKKQRILLQKFDEDGFKECNLNDTRYVNRFLCQFVADHILLTGKGKRRVFASNGQITNLLRGFWGLRKVRAENDRHHALDAVVVACSTVAMQQKITRFVRYKEMNAFDGKTIDKETGKVLHQKTHFPQPWEFFAQEVMIRVFGKPDGKPEFEEADTPEKLRTLLAEKLSSRPEAVHEYVFPLFVSRAPNRKMSGAHKDTLRSAKRFVKHNEKISVKRVWLTEIKLADLENMVNYKNGREIELYEALKARLEAYGGNAKQAFDPKDNPFYKKGGQLVKAVRVEKTQESGVLLNKKNAYTIADNGDMVRVDVFCKVDKKGKNQYFIVPIYAWQVAENILPDIDCKGYRIDDSYTFCFSLHKYDLIAFQKDEKSKVEFAYYINCDSSNGRFYLAWHDKGSKEQQFRISTQNLVLIQKYQVNELGKEIRPCRLKKRPPVRGTGGPKKKRKVYPYDVPDYAGYPYDVPDYAGSYPYDVPDYAGSAAPAAKKKKLDFESG

SEQ ID NO: 237 - protein, 1054 aa, Haemophilus parainfluenzae
MENKNLNYILGLDLGIASVGWAVVEIDEKENPLRLIDVGVRTFERAEVPKTGESLALSRRLARSARRLTQRRVARLKKAKRLLKSENILLSTDERLPHQVWQLRVEGLDHKLERQEWAAVLLHLIKHRGYLSQRKNESKSENKELGALLSGVDNNHKLLQQATYRSPAELAVKKFEVEEGHIRNQQGAYTHTFSRLDLLAEMELLFSRQQHFGNPFASEKLLENLTALLMWQKPALSGEAILKMLGKCTFEDEYKAAKNTYSAERFVWITKLNNLRIQENGLERALNDNERLALMEQPYDKNRLFYSQVRSILKLSDEAIFKGLRYSGEDKKAIETKAVLMEMKAYHQIRKVLEGNNLKAEWAELKANPTLLDEIGTAFSLYKTDEDISAYLAGKLSQPVLNALLENLSFDKFIQLSLKALYKLLPLMQQGLRYDEACREIYGDHYGKKTEENHHFLPQIPADEIRNPVVLRTLTQARKVINGVVRLYGSPARIHIETGREVGKSYKDRRELEKRQEENRKQRENAIKEFKEYFPHFAGEPKAKDILKMRLYKQQNAKCLYSGKPIELHRLLEKGYVEVDHALPFSRTWDDSFNNKVLVLANENQNKGNLTPFEWLDGKHNSERWRAFKALVETSAFPYAKKQRILSQKLDEKGFIERNLNDTRYVARFLCNFIADNMHLTGEGKRKVFASNGQITALLRSRWGLAKSREDNDRHHALDAVVVACSTVAMQQKITRFVRFEAGDVFTGERIDRETGEIIPLHFPTPWQFFKQEVEIRIFSDNPKLELENRLPDRPQANHEFVQPLFVSRMPTRKMTGQGHMETVKSAKRLNEGISVIKMPLTKLKLKDLELMVNREREKDLYDTLKARLEAFNDDPAKAFAEPFIKKGGAIVKSVRVEQIQKSGVLVREGNGVADNASMVRVDVFTKGGKYFLVPIYTWQVAKGILPNKAATQYKDEEDWEVMDNSATFKFSLHPNDLVKLVTKKKTILGYFNGLNRATGNIDIKEHDLDKSKGKQGIFEGVGIKLALSFEKYQVDELGKNIRLCKPSKRQPVR

SEQ ID NO: 238 - DNA, 3192 bp, synthetic construct
  source 1..3192: mol_type = other DNA
atggaaaaatttcactatgtattgggtttggatttgggtatcgcctctgtggggtgggctgccattgaaattgacaaggaaaccgaaacatcaatcggtttattggattgcggtgtcagaacatttgaacgtgcagaagtacccaaaacaggcgattctcttgccaaagctcgccgtgaagccagaagtactcgccgtttaattcgcagacgttcgcatcgcttattacgtttaaaacgtttattgaaacgtgaaattttcaggcagcctgaaacgtttaaagacttaccaatcaatgcttggcaattgcgtgttaaaggcttggatagtcggttgaatgaatatgaatgggcggccgttttattgcatttggtgaagcatcgcggttatttatcgcaacgcaaaagcgaaatgagcgaaacagacagcaaatctgaaatgggcagattactggcaggtgtggcggaaaatcaccaacttttacaacaagaacaatatcgtacaccagccgaattagcactcaaaaaatttgtgaaacattttcgcaataaaggtggcgattatgcacacactttcaaccgtttggatttgcaagccgaattgcatttattgtttcaaaaacaacgtgaattaggcaatccattcacttcaccagaattggaacggcaagttgatgatttgttgatgacgcagcgcagtgctttacaaggtgatgcgattttgaaaatgttgggtcattgtgggtttgaacctgaacaattcaaagcagcgaaaaacacattcagtgccgaacgttttatttggttgacaaaactcaataatcttcgcattcaagaccaaggcaaagaacgtgcgttaactgccgatgagcgtaccaaattgttggacgagccttataaaaaaagtaaattgacttacgcacaagttcgcaaattattaagcttgcctcaaactgctatttttaaaggtttgcgttatgatttggaacatgacaaaaaagcagaaaacagtacgttgatggaaatgaaatcctatcacaacatccgccaaacattggaaaaatcaggtttgaaaacagaatggcaaagtattgccacgcagcctgaaattttagatgcaattggcacggcgttttccatttataaaaccgatgaagatatttcgcatgaattaaaaacgtgcaggctgcctgaaaacgtattgaatgaattactgaaaaacatcaattttgatggattcattcaattatcgttgactgcattacgcaaaattttgcccttgatggaacaaggctaccgttatgatgaagcgtgtacccaaatttacggtaatcatcattcaggcagcttgcaacaagaatcaaagcaatttttgccacatattccgattgatgatgtccgaaatcctgtggtgttccgtactttgacccaagcaagaaaagtggtgaatgcgattattcgtcggtatggttcgccagctcgtgtgcatattgagatggcgcgtgaattgggtaagtctaaatcagaccgtgaccgaattgaaaaacaacaacaaaaaaataaaaaagaacgtgaaaacgcagtcgccaaattcaaagaagatttccctgattttgtgggcgaacccagagggaaagatattttgaaaatgcgtttgtatgaacaacaacacggcaaatgtttgtattcgggtcatgatattgatattaatcgattgaatgaaaaaggttatgttgaaattgaccatgccctgccattttcacggacttgggatgatagtcaaaataataaagttttggtacttggcagcgaaaaccaaaataaacgcaatcaaacgcctgatgaatatttggacggtgcaaacaatagccaacgttggcttgaatttcaagcgcgtgtacaaacttgtcatttttcttacggtaaaaaacaacgcattcaattagccaaattagacgatgaaaccgaaaaaggatttttagaacgcaatctaaatgatacgcggtatattgctcgttttatgtgtcaatttgtccaagaaaatttatatttgacaggtaaaggaaaacgtcttgtttttgcatcaaacggcggaatgaccgcaacattgagaaatttatggggtttgagaaaagtccgtgaagacaacgaccgccatcatgctcttgatgcgattgtggtggcgtgttccactgcttctatgcaacaaaaaataaccaaagcatttcaacgccatgaaagcattgaatatgtggataccgaaacgggcgaagtaaaatttcgtattccacagccttgggattttttccgtcaggaagtgatgattcgtgtgttcagcgaccaaccgtgtgaagatttggtagaaaaattgtcggctcgtcccgaagctttgcatgacaacgtaacgcctttatttgtctcgcgtgcaccaaatcgcaaaatgtcggggcaagggcatttggaaaccatcaaatctgcaaaaaggctgtctgaagaaaacagtatggttaaaaaaccattaaccacattgaaattaaaagatattccagaaatcgtaggctacccgagtcgtgaacctcaattgtatgccgcattgaaaacacgtttagaaacgcatgatgatgacccaattaaagcctttgccaaacccttttacaaacccaataaaaatggtgaattgggggcgttggttcgatcggtgcgtgtgaaaggtgtacaaaatacgggtgtaatggttcatgatggcaaaggcattgccgataatgccacaatggttcgtgttgatgtctataccaaagcgggcaaaaattaccttgttcctgtgtatgtttggcaggtggctcaaggaattttgccaaatcgggcggttacttctggcaaaagtgaagcagattgggatttaattgatgaaagttttgaatttaaattttcgctgtctcgtggggatttagtggaaatgattagcaataaaggaagaatttttggttattacaatgggttagatcgtgcaaatggaagtattgggattcgtgaacatgatttggaaaagtccaaaggaaaagatggtgttcatcgtgttggcgtgaaaaccgccaccgcattcaacaaataccacgttgacccacttggtaaagaaattcatcggtgttcatctgaaccacgccccacattaaaaatcaaatccaagaaataa

SEQ ID NO: 239 - protein, 1063 aa, synthetic construct
MEKFHYVLGLDLGIASVGWAAIEIDKETETSIGLLDCGVRTFERAEVPKTGDSLAKARREARSTRRLIRRRSHRLLRLKRLLKREIFRQPETFKDLPINAWQLRVKGLDSRLNEYEWAAVLLHLVKHRGYLSQRKSEMSETDSKSEMGRLLAGVAENHQLLQQEQYRTPAELALKKFVKHFRNKGGDYAHTFNRLDLQAELHLLFQKQRELGNPFTSPELERQVDDLLMTQRSALQGDAILKMLGHCGFEPEQFKAAKNTFSAERFIWLTKLNNLRIQDQGKERALTADERTKLLDEPYKKSKLTYAQVRKLLSLPQTAIFKGLRYDLEHDKKAENSTLMEMKSYHNIRQTLEKSGLKTEWQSIATQPEILDAIGTAFSIYKTDEDISHELKTCRLPENVLNELLKNINFDGFIQLSLTALRKILPLMEQGYRYDEACTQIYGNHHSGSLQQESKQFLPHIPIDDVRNPVVFRTLTQARKVVNAIIRRYGSPARVHIEMARELGKSKSDRDRIEKQQQKNKKERENAVAKFKEDFPDFVGEPRGKDILKMRLYEQQHGKCLYSGHDIDINRLNEKGYVEIDHALPFSRTWDDSQNNKVLVLGSENQNKRNQTPDEYLDGANNSQRWLEFQARVQTCHFSYGKKQRIQLAKLDDETEKGFLERNLNDTRYIARFMCQFVQENLYLTGKGKRLVFASNGGMTATLRNLWGLRKVREDNDRHHALDAIVVACSTASMQQKITKAFQRHESIEYVDTETGEVKFRIPQPWDFFRQEVMIRVFSDQPCEDLVEKLSARPEALHDNVTPLFVSRAPNRKMSGQGHLETIKSAKRLSEENSMVKKPLTTLKLKDIPEIVGYPSREPQLYAALKTRLETHDDDPIKAFAKPFYKPNKNGELGALVRSVRVKGVQNTGVMVHDGKGIADNATMVRVDVYTKAGKNYLVPVYVWQVAQGILPNRAVTSGKSEADWDLIDESFEFKFSLSRGDLVEMISNKGRIFGYYNGLDRANGSIGIREHDLEKSKGKDGVHRVGVKTATAFNKYHVDPLGKEIHRCSSEPRPTLKIKSKK

SEQ ID NO: 240 - DNA, 3293 bp, synthetic construct
  misc_feature 1..3293: note = Synthetic
  source 1..3293: mol_type = other DNA
atggagaagttccactacgtgctgggactggatctgggaatcgcaagcgtgggatgggcagcaatcgagatcgataaggagaccgagacatccatcggcctgctggactgcggcgtgaggacctttgagagggcagaggtgcctaagacaggcgacagcctggcaaaggcaaggagagaggcaaggtctacaaggcgcctgatccggagaaggagccacaggctgctgcggctgaagagactgctgaagcgggagatctttagacagccagagaccttcaaggatctgcccatcaacgcatggcagctgagggtgaagggactggactctcggctgaatgagtacgagtgggcagccgtgctgctgcacctggtgaagcacaggggctatctgagccagcgcaagtccgagatgtctgagaccgactctaagagcgagatgggcaggctgctggcaggagtggccgagaaccaccagctgctgcagcaggagcagtacaggaccccagcagagctggccctgaagaagtttgtgaagcacttccgcaacaagggcggcgattatgcccacacattcaataggctggacctgcaggcagagctgcacctgctgtttcagaagcagagagagctgggcaaccccttcacctctcctgagctggagcgccaggtggacgatctgctgatgacacagcggagcgccctgcagggcgatgcaatcctgaagatgctgggccactgtggctttgagcctgagcagttcaaggccgccaagaatacctttagcgccgagagattcatctggctgacaaagctgaacaatctgaggatccaggaccagggcaaggagagagccctgaccgccgatgagaggacaaagctgctggacgagccttacaagaagtctaagctgacctatgcccaggtgaggaagctgctgagcctgcctcagacagccatcttcaagggcctgcgctacgatctggagcacgacaagaaggccgagaactctaccctgatggagatgaagagctatcacaatatccggcagacactggagaagtccggcctgaagaccgagtggcagtctatcgccacacagccagagatcctggacgcaatcggaaccgccttttccatctacaagacagatgaggacatctctcacgagctgaagacctgcagactgcctgagaacgtgctgaatgagctgctgaagaacatcaattttgatggcttcatccagctgagcctgaccgccctgcgcaagatcctgccactgatggagcagggctaccggtatgacgaggcctgtacacagatctacggcaaccaccactccggctctctgcagcaggagtccaagcagtttctgcctcacatcccaatcgacgatgtgcggaacccagtggtgttcagaaccctgacacaggccaggaaggtggtgaatgccatcatccgccggtatggatctccagcaagggtgcacatcgagatggcaagggagctgggcaagagcaagtccgatagagacaggatcgagaagcagcagcagaagaacaagaaggagagggagaatgccgtggccaagttcaaggaggattttccagacttcgtgggagagcctaggggcaaggatatcctgaagatgcggctgtacgagcagcagcacggcaagtgcctgtattccggccacgatatcgacatcaaccggctgaatgagaagggctacgtggagatcgaccacgccctgccttttagcagaacctgggacgattcccagaacaataaggtgctggtgctgggcagcgagaaccagaataagcgcaatcagacaccagatgagtacctggacggcgccaacaattcccagagatggctggagtttcaggccagggtgcagacctgccacttctcttatggcaagaagcagaggatccagctggccaagctggacgatgagaccgagaagggcttcctggagcgcaacctgaatgatacaaggtacatcgcccggttcatgtgccagttcgtgcaggagaacctgtatctgaccggcaagggcaagcgcctggtgtttgcctccaacggcggcatgaccgccacactgcggaatctgtggggcctgaggaaggtgcgcgaggataatgacagacaccacgcactggacgcaatcgtggtggcatgcagcaccgcatccatgcagcagaagatcacaaaggcctttcagcggcacgagagcatcgagtatgtggataccgagacaggcgaggtgaagttcagaatcccccagccttgggacttctttcgccaggaagtgatgatccgggtgttttccgatcagccatgtgaggacctggtggagaagctgtctgccaggccagaggccctgcacgataacgtgacccctctgttcgtgagcagggcaccaaatagaaagatgtccggccagggccacctggagacaatcaagtccgccaagcgcctgtccgaggagaactctatggtgaagaagcccctgaccacactgaagctgaaggacatccctgagatcgtgggctacccatctagagagccccagctgtatgccgccctgaagaccaggctggagacacacgacgatgacccaatcaaggcctttgccaagcctttctacaagccaaacaagaatggagagctgggcgccctggtgcggtccgtgagagtgaagggcgtgcagaacacaggcgtgatggtgcacgatggcaagggcatcgccgacaatgccacaatggtgcgggtggacgtgtatacaaaggccggcaagaactacctggtgcccgtgtacgtgtggcaggtggcacagggaatcctgccaaatagagccgtgacctctggcaagagcgaggccgattgggacctgatcgatgagagcttcgagtttaagttctctctgagcagaggcgacctggtggagatgatctccaacaagggcaggatcttcggctactataacggcctggatagagccaatggcagcatcggcatcagggagcacgatctggagaagtccaagggcaaggacggagtgcacagggtgggagtgaagaccgcaacagcctttaataagtaccacgtggaccccctgggcaaggagatccacagatgtagctccgagccaaggcccaccctgaagatcaagagcaagaagggcaccggcgggcccaagaagaagaggaggtatacccatacgatgttcctgactatgcgggctatccctatgacgtcccggactatgcaggatcgtatccttat

SEQ ID NO: 241 - protein, 1121 aa, synthetic construct
  REGION 1..1121: note = Synthetic
MEKFHYVLGLDLGIASVGWAAIEIDKETETSIGLLDCGVRTFERAEVPKTGDSLAKARREARSTRRLIRRRSHRLLRLKRLLKREIFRQPETFKDLPINAWQLRVKGLDSRLNEYEWAAVLLHLVKHRGYLSQRKSEMSETDSKSEMGRLLAGVAENHQLLQQEQYRTPAELALKKFVKHFRNKGGDYAHTFNRLDLQAELHLLFQKQRELGNPFTSPELERQVDDLLMTQRSALQGDAILKMLGHCGFEPEQFKAAKNTFSAERFIWLTKLNNLRIQDQGKERALTADERTKLLDEPYKKSKLTYAQVRKLLSLPQTAIFKGLRYDLEHDKKAENSTLMEMKSYHNIRQTLEKSGLKTEWQSIATQPEILDAIGTAFSIYKTDEDISHELKTCRLPENVLNELLKNINFDGFIQLSLTALRKILPLMEQGYRYDEACTQIYGNHHSGSLQQESKQFLPHIPIDDVRNPVVFRTLTQARKVVNAIIRRYGSPARVHIEMARELGKSKSDRDRIEKQQQKNKKERENAVAKFKEDFPDFVGEPRGKDILKMRLYEQQHGKCLYSGHDIDINRLNEKGYVEIDHALPFSRTWDDSQNNKVLVLGSENQNKRNQTPDEYLDGANNSQRWLEFQARVQTCHFSYGKKQRIQLAKLDDETEKGFLERNLNDTRYIARFMCQFVQENLYLTGKGKRLVFASNGGMTATLRNLWGLRKVREDNDRHHALDAIVVACSTASMQQKITKAFQRHESIEYVDTETGEVKFRIPQPWDFFRQEVMIRVFSDQPCEDLVEKLSARPEALHDNVTPLFVSRAPNRKMSGQGHLETIKSAKRLSEENSMVKKPLTILKLKDIPEIVGYPSREPQLYAALKTRLETHDDDPIKAFAKPFYKPNKNGELGALVRSVRVKGVQNTGVMVHDGKGIADNATMVRVDVYTKAGKNYLVPVYVWQVAQGILPNRAVTSGKSEADWDLIDESFEFKFSLSRGDLVEMISNKGRIFGYYNGLDRANGSIGIREHDLEKSKGKDGVHRVGVKTATAFNKYHVDPLGKEIHRCSSEPRPTLKIKSKKGTGGPKKKRKVYPYDVPDYAGYPYDVPDYAGSYPYDVPDYAGSAAPAAKKKKLDFESG

SEQ ID NO: 242 - RNA, 148 nt, synthetic construct
  source 1..148: mol_type = other RNA
nnnnnnnnnnnnnnnnnnnnnnnngttgtagctccctttctcatttcggaaacgaaatgagaaccgttgctacaataaggccgtctgaaaagatgtgccgcaacgctctgccccttaaagcttctgctttaaggggcatcgtttattt

SEQ ID NO: 243 - RNA, 141 nt, synthetic construct
  source 1..141: mol_type = other RNA
nnnnnnnnnnnnnnnnnnnnnnnngttgtagctccctttttcatttcgcagaaatgcgaaatgaaaaacgttgttacaataagagaaaagatttctcgcaaagctctgtcccttgaaatgtaagtttcaagggacatcttt

SEQ ID NO: 244 - RNA, 172 nt, synthetic construct
  source 1..172: mol_type = other RNA
nnnnnnnnnnnnnnnnnnnnnnnngttgtagctccctctctcatctcgtagtggaaacactacgagatgagagccgttgctacaataaggccgtctgaaaagacgcgccgcgacgtaaaatactttatgtatgagcccctgtttgagttttcttaaacaggggcatcgtctt

SEQ ID NO: 245 - DNA, 24 bp, synthetic construct
  misc_feature 1..24: note = Synthetic
  source 1..24: mol_type = other DNA
gtgaacttgtggccgtttacgtcg

SEQ ID NO: 246 - DNA, 24 bp, synthetic construct
  misc_feature 1..24: note = Synthetic
  source 1..24: mol_type = other DNA
gtgaacttgtggccgtttacgtcc

SEQ ID NO: 247 - DNA, 24 bp, synthetic construct
  misc_feature 1..24: note = Synthetic
  source 1..24: mol_type = other DNA
gtgaacttgtggccgtttacgtgg

SEQ ID NO: 248 - DNA, 24 bp, synthetic construct
  misc_feature 1..24: note = Synthetic
  source 1..24: mol_type = other DNA
gtgaacttgtggccgtttacgacg

SEQ ID NO: 249 - DNA, 24 bp, synthetic construct
  misc_feature 1..24: note = Synthetic
  source 1..24: mol_type = other DNA
gtgaacttgtggccgtttacctcg

SEQ ID NO: 250 - DNA, 24 bp, synthetic construct
  misc_feature 1..24: note = Synthetic
  source 1..24: mol_type = other DNA
gtgaacttgtggccgtttaggtcg

SEQ ID NO: 251 - DNA, 24 bp, synthetic construct
  misc_feature 1..24: note = Synthetic
  source 1..24: mol_type = other DNA
gtgaacttgtggccgttttcgtcg

SEQ ID NO: 252 - DNA, 24 bp, synthetic construct
  misc_feature 1..24: note = Synthetic
  source 1..24: mol_type = other DNA
gtgaacttgtggccgttaacgtcg

SEQ ID NO: 253 - DNA, 24 bp, synthetic construct
  misc_feature 1..24: note = Synthetic
  source 1..24: mol_type = other DNA
gtgaacttgtggccgtatacgtcg

SEQ ID NO: 254 - DNA, 24 bp, synthetic construct
  misc_feature 1..24: note = Synthetic
  source 1..24: mol_type = other DNA
gtgaacttgtggccgattacgtcg

SEQ ID NO: 255 - DNA, 24 bp, synthetic construct
  misc_feature 1..24: note = Synthetic
  source 1..24: mol_type = other DNA
gtgaacttgtggccctttacgtcg

SEQ ID NO: 256 - DNA, 24 bp, synthetic construct
  misc_feature 1..24: note = Synthetic
  source 1..24: mol_type = other DNA
gtgaacttgtggcggtttacgtcg

SEQ ID NO: 257 - DNA, 24 bp, synthetic construct
  misc_feature 1..24: note = Synthetic
  source 1..24: mol_type = other DNA
gtgaacttgtgggcgtttacgtcg

SEQ ID NO: 258 - DNA, 24 bp, synthetic construct
  misc_feature 1..24: note = Synthetic
  source 1..24: mol_type = other DNA
gtgaacttgtgcccgtttacgtcg

SEQ ID NO: 259 - DNA, 24 bp, synthetic construct
  misc_feature 1..24: note = Synthetic
  source 1..24: mol_type = other DNA
gtgaacttgtcgccgtttacgtcg

SEQ ID NO: 260 - DNA, 24 bp, synthetic construct
  misc_feature 1..24: note = Synthetic
  source 1..24: mol_type = other DNA
gtgaacttgaggccgtttacgtcg

SEQ ID NO: 261 - DNA, 24 bp, synthetic construct
  misc_feature 1..24: note = Synthetic
  source 1..24: mol_type = other DNA
gtgaacttctggccgtttacgtcg

SEQ ID NO: 262 - DNA, 24 bp, synthetic construct
  misc_feature 1..24: note = Synthetic
  source 1..24: mol_type = other DNA
gtgaactagtggccgtttacgtcg

SEQ ID NO: 263 - DNA, 24 bp, synthetic construct
  misc_feature 1..24: note = Synthetic
  source 1..24: mol_type = other DNA
gtgaacatgtggccgtttacgtcg

SEQ ID NO: 264 - DNA, 24 bp, synthetic construct
  misc_feature 1..24: note = Synthetic
  source 1..24: mol_type = other DNA
gtgaagttgtggccgtttacgtcg

SEQ ID NO: 265 - DNA, 24 bp, synthetic construct
  misc_feature 1..24: note = Synthetic
  source 1..24: mol_type = other DNA
gtgatcttgtggccgtttacgtcg

SEQ ID NO: 266 - DNA, 24 bp, synthetic construct
  misc_feature 1..24: note = Synthetic
  source 1..24: mol_type = other DNA
gtgtacttgtggccgtttacgtcg

SEQ ID NO: 267 - DNA, 24 bp, synthetic construct
  misc_feature 1..24: note = Synthetic
  source 1..24: mol_type = other DNA
gtcaacttgtggccgtttacgtcg

SEQ ID NO: 268 - DNA, 24 bp, synthetic construct
  misc_feature 1..24: note = Synthetic
  source 1..24: mol_type = other DNA
gagaacttgtggccgtttacgtcg

SEQ ID NO: 269-270 - skipped (2 entries)

SEQ ID NO: 271 - DNA, 31 bp, synthetic construct
  misc_feature 1..31: note = Synthetic
  source 1..31: mol_type = other DNA
tgaggaccgccctgggcctgggagaatccct

SEQ ID NO: 272 - DNA, 31 bp, synthetic construct
  misc_feature 1..31: note = Synthetic
  source 1..31: mol_type = other DNA
gaaggaccaccctaggcctgggagactccct

SEQ ID NO: 273 - DNA, 30 bp, synthetic construct
  misc_feature 1..30: note = Synthetic
  source 1..30: mol_type = other DNA
ctcactcacccacacagacacacacgtcct

SEQ ID NO: 274 - DNA, 30 bp, synthetic construct
  misc_feature 1..30: note = Synthetic
  source 1..30: mol_type = other DNA
cacacacacccacacagacacacccccccc

SEQ ID NO: 275 - DNA, 30 bp, synthetic construct
  misc_feature 1..30: note = Synthetic
  source 1..30: mol_type = other DNA
ctccctcacacacacagacacacacctccc

SEQ ID NO: 276 - DNA, 30 bp, synthetic construct
  misc_feature 1..30: note = Synthetic
  source 1..30: mol_type = other DNA
cacacacacacacacagacacacacacccc

SEQ ID NO: 277 - DNA, 31 bp, synthetic construct
  misc_feature 1..31: note = Synthetic
  source 1..31: mol_type = other DNA
gtgtgtccctctccccacccgtccctgtccg

SEQ ID NO: 278 - DNA, 31 bp, synthetic construct
  misc_feature 1..31: note = Synthetic
  source 1..31: mol_type = other DNA
ttgtctccctgtccccacccgtccccttcag

SEQ ID NO: 279 - DNA, 31 bp, synthetic construct
  misc_feature 1..31: note = Synthetic
  source 1..31: mol_type = other DNA
ctgcctccctctgcccacccgtccttcccac

SEQ ID NO: 280 - DNA, 31 bp, synthetic construct
  misc_feature 1..31: note = Synthetic
  source 1..31: mol_type = other DNA
ctgtgcctctctccccacccttccacaccct

SEQ ID NO: 281 - DNA, 31 bp, synthetic construct
  misc_feature 1..31: note = Synthetic
  source 1..31: mol_type = other DNA
gctcatccccctccccacccgtcctcgcccg

SEQ ID NO: 282 - RNA, 77 nt, synthetic construct; identical to SEQ ID NO: 223
  source 1..77: mol_type = other RNA

SEQ ID NO: 283 - DNA, 58 bp, synthetic construct
  misc_feature 1..58: note = Synthetic
  source 1..58: mol_type = other DNA
ggagccccgaaacggcacaaaaggccgcgaaaagaggccgcaacgccgcccggcacga

SEQ ID NO: 284 - DNA, 109 bp, synthetic construct
  misc_feature 1..109: note = Synthetic
  source 1..109: mol_type = other DNA
atcctggtcgagctggacggcgacgtaaacggccacaagttcagcgtgtccggctttggcgagacaaatcacctgcctgctggaatacggtaaacctacggcaagctga

SEQ ID NO: 285 - DNA, 109 bp, synthetic construct
  misc_feature 1..109: note = Synthetic
  source 1..109: mol_type = other DNA
tcagcttgccgtaggtttaccgtattccacgaggcaggtgatttgtctcgccaaagccggacacgctgaacttgtggccgtttacgtcgccgtccagctcgaccaggat

SEQ ID NO: 286 - protein, 35 aa, synthetic construct
  REGION 1..35: note = Synthetic
ILVELDGDVNGHKFSVSGFGETNHLPRGIRTYGKL

SEQ ID NO: 287 - DNA, 44 bp, synthetic construct
  misc_feature 1..44: note = Synthetic
  source 1..44: mol_type = other DNA
ccccaccggcgctggtgcccaggacgaggatggagattatgaag

SEQ ID NO: 288 - DNA, 44 bp, synthetic construct
  misc_feature 1..44: note = Synthetic
  source 1..44: mol_type = other DNA
cttcataatctccatcctcgtcctgggcaccagcgccggtgggg

SEQ ID NO: 289 - DNA, 44 bp, synthetic construct
  misc_feature 1..44: note = Synthetic
  source 1..44: mol_type = other DNA
tgggagaatcccttccccctcttccctcgtgatctgcaactcca

SEQ ID NO: 290 - DNA, 44 bp, synthetic construct
  misc_feature 1..44: note = Synthetic
  source 1..44: mol_type = other DNA
tggagttgcagatcacgagggaagagggggaagggattctccca

SEQ ID NO: 291 - DNA, 44 bp, synthetic construct
  misc_feature 1..44: note = Synthetic
  source 1..44: mol_type = other DNA
gagaagatcaactacactggccgtttcttacctggattcgaggc

SEQ ID NO: 292 - DNA, 44 bp, synthetic construct
  misc_feature 1..44: note = Synthetic
  source 1..44: mol_type = other DNA
gcctcgaatccaggtaagaaacggccagtgtagttgatcttctc

SEQ ID NO: 293 - DNA, 44 bp, synthetic construct
  misc_feature 1..44: note = Synthetic
  source 1..44: mol_type = other DNA
ccttctttgtttcccctcctcccaggaatatgtggactataatg

SEQ ID NO: 294 - DNA, 44 bp, synthetic construct
  misc_feature 1..44: note = Synthetic
  source 1..44: mol_type = other DNA
cattatagtccacatattcctgggaggaggggaaacaaagaagg

SEQ ID NO: 295 - DNA, 32 bp, synthetic construct
  misc_feature 1..32: note = Synthetic
  source 1..32: mol_type = other DNA
cggcgctggtgcccaggacgaggatggagatt

SEQ ID NO: 296 - DNA, 31 bp, synthetic construct
  misc_feature 1..31: note = Synthetic
  source 1..31: mol_type = other DNA
ggcgctggtgtcctggacgaggaactggact

SEQ ID NO: 297 - DNA, 32 bp, synthetic construct
  misc_feature 1..32: note = Synthetic
  source 1..32: mol_type = other DNA
aggaactggagcaaaggacaaggagatggttt

SEQ ID NO: 298 - DNA, 32 bp, synthetic construct
  misc_feature 1..32: note = Synthetic
  source 1..32: mol_type = other DNA
agggaactgggaccaggacaaggagcttgatt

SEQ ID NO: 299 - DNA, 32 bp, synthetic construct
  misc_feature 1..32: note = Synthetic
  source 1..32: mol_type = other DNA
aggtgcgggaggcgagggcaagacttagtgct

SEQ ID NO: 300 - DNA, 32 bp, synthetic construct
  misc_feature 1..32: note = Synthetic
  source 1..32: mol_type = other DNA
tgcagatcacgagggaagagggggaagggatt

SEQ ID NO: 301 - DNA, 32 bp, synthetic construct
  misc_feature 1..32: note = Synthetic
  source 1..32: mol_type = other DNA
gtcataacaccagggaagaggaggcctggatt

SEQ ID NO: 302 - DNA, 32 bp, synthetic construct
  misc_feature 1..32: note = Synthetic
  source 1..32: mol_type = other DNA
tggcagaacagagggaagagagggggagggat

SEQ ID NO: 303 - DNA, 32 bp, synthetic construct
  misc_feature 1..32: note = Synthetic
  source 1..32: mol_type = other DNA
agcagatcagagggaagagggaggggtggaga

SEQ ID NO: 304 - DNA, 32 bp, synthetic construct
  misc_feature 1..32: note = Synthetic
  source 1..32: mol_type = other DNA
tacacatctctgagggtgatgggcttggggct

SEQ ID NO: 305 - DNA, 32 bp, synthetic construct
  misc_feature 1..32: note = Synthetic
  source 1..32: mol_type = other DNA
tgcagaggtttacaaacggggggggggggggg

SEQ ID NO: 306 - DNA, 32 bp, synthetic construct
  misc_feature 1..32: note = Synthetic
  source 1..32: mol_type = other DNA
gccagaagaccaggtaggacttggacgacaag

SEQ ID NO: 307 - protein, 1082 aa, synthetic construct
  REGION 1..1082: note = Synthetic
MAAFKPNSINYILGLDIGIASVGWAMVEIDEEENPIRLIDLGVRVFERAEVPKTGDSLAMARRLARSVRRLTRRRAHRLLRTRRLLKREGVLQAANFDENGLIKSLPNTPWQLRAAALDRKLTPLEWSAVLLHLIKHRGYLSQRKNEGETADKELGALLKGVAGNAHALQTGDFRTPAELALNKFEKESGHIRNQRSDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSPELQDEIGTAFSLFKTDEDITGRLKDRIQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLGRLNEKGYVEIDHALPFSRTWDDSFNNKVLVLGSENQNKGNQTPYEYFNGKDNSREWQEFKARVETSRFPRSKKQRILLQKFDEDGFKERNLNDTRYVNRFLCQFVADRMRLTGKGKKRVFASNGQITNLLRGFWGLRKVRAENDRHHALDAVVVACSTVAMQQKITRFVRYKEMNAFDGKTIDKETGEVLHQKTHFPQPWEFFAQEVMIRVFGKPDGKPEFEEADTLEKLRTLLAEKLSSRPEAVHEYVTPLFVSRAPNRKMSGQGHMETVKSAKRLDEGVSVLRVPLTQLKLKDLEKMVNREREPKLYEALKARLEAHKDDPAKAFAEPFYKYDKAGNRTQQVKAVRVEQVQKTGVWVRNHNGIADNATMVRVDVFEKGDKYYLVPIYSWQVAKGILPDRAVVQGKDEEDWQLIDDSFNFKFSLHPNDLVEVITKKARMFGYFASCHRGTGNINIRIHDLDHKIGKNGILEGIGVKTALSFQKYQIDELGKEIRPCRLKKRPPVR

SEQ ID NO: 308 - protein, 1082 aa, synthetic construct
  REGION 1..1082: note = Synthetic
MAAFKPNPINYILGLDIGIASVGWAMVEIDEEENPIRLIDLGVRVFERAEVPKTGDSLAMARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVANNAHALQTGDFRTPAELALNKFEKESGHIRNQRDDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSSELQDEIGTAFSLFKTDEDITGRLKDRVQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLVRLNEKGYVEIDHALPFSRTWDDSFNNKVLVLGSENQNKGNQTPYEYFNGKDNSREWQEFKARVETSRFPRSKKQRILLQKFDEDGFKECNLNDTRYVNRFLCQFVADHILLTGKGKRRVFASNGQITNLLRGFWGLRKVRAENDRHHALDAVVVACSTVAMQQKITRFVRYKEMNAFDGKTIDKETGKVLHQKTHFPQPWEFFAQEVMIRVFGKPDGKPEFEEADTPEKLRTLLAEKLSSRPEAVHEYVTPLFVSRAPNRKMSGAHKDTLRSAKRFVKHNEKISVKRVWLTEIKLADLENMVNYKNGREIELYEALKARLEAYGGNAKQAFDPKDNPFYKKGGQLVKAVRVEKTQESGVLLNKKNAYTIADNGDMVRVDVFCKVDKKGKNQYFIVPIYAWQVAENILPDIDCKGYRIDDSYTFCFSLHKYDLIAFQKDEKSKVEFAYYINCDSSNGRFYLAWHDKGSKEQQFRISTQNLVLIQKYQVNELGKEIRPCRLKKRPPVR

SEQ ID NO: 309 - protein, 1082 aa, synthetic construct; identical to SEQ ID NO: 307
  REGION 1..1082: note = Synthetic

SEQ ID NO: 310 - protein, 1081 aa, synthetic construct
  REGION 1..1081: note = Synthetic
MAAFKPNPINYILGLDIGIASVGWAMVEIDEEENPIRLIDLGVRVFERAEVPKTGDSLAMARRLARSVRRLTRRRAHRLLRARRLLKREGVLQAANFDENGLIKSLPNTPWQLRAAALDRKLTPLEWSAVLLHLIKHRGYLSQRKNEGETADKELGALLKGVAGNAHALQTGDFRTPAELALNKFEKECGHIRNQRSDYSHTFSRKDLQAELNLLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLSLEDTAFFKGLRYGKDNAEASTLMEMKAYHTISRALEKEGLKDKKSPLNLSPELQDEIGTAFSLFKTDEDITGRLKDRIQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLGRLNEKGYVEIDHALPFSRTWDDSFNNKVLVLGSENQNKGNQTPYEYFNGKDNSREWQEFKARVETSRFPRSKKQRILLQKFDEDGFKERNLNDTRYVNRFLCQFVADRMRLTGKGKKRVFASNGQITNLLRGFWGLRKVRAENDRHHALDAVVVACSTVAMQQKITRFVRYKEMNAFDGKTIDKETGEVLHQKTHFPQPWEFFAQEVMIRVFGKPDGKPEFEEADTPEKLRTLLAEKLSSRPEAVHEYVTPLFVSRAPNRKMSGQGHMETVKSAKRLDEGVSVLRVPLTQLKLKDLEKMVNREREPKLYEALKARLEAHKDDPAKAFAEPFYKYDKAGNRTQQVKAVRVEQVQKTGVWVRNHNGIADNATMVRVDVFEKGDKYYLVPIYSWQVAKGILPDRAVVAYADEEDWTVIDESFRFKFVLYSNDLIKVQLKKDSFLGYFSGLDRATGAISLREHDLEKSKGKDGMHRIGVKTALSFQKYQIDEMGKEIRPCRLKKRPPVR

SEQ ID NO: 311 - protein, 1140 aa, synthetic construct
  REGION 1..1140: note = Synthetic
MAAFKPNPINYILGLDIGIASVGWAMVEIDEEENPIRLIDLGVRVFERAEVPKTGDSLAMARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVANNAHALQTGDFRTPAELALNKFEKESGHIRNQRGDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSSELQDEIGTAFSLFKTDEDITGRLKDRVQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLVRLNEKGYVEIDHALPFSRTWDDSFNNKVLVLGSENQNKGNQTPYEYFNGKDNSREWQEFKARVETSRFPRSKKQRILLQKFDEDGFKECNLNDTRYVNRFLCQFVADHILLTGKGKRRVFASNGQITNLLRGFWGLRKVRAENDRHHALDAVVVACSTVAMQQKITRFVRYKEMNAFDGKTIDKETGKVLHQKTHFPQPWEFFAQEVMIRVFGKPDGKPEFEEADTPEKLRTLLAEKLSSRPEAVHEYVTPLFVSRAPNRKMSGAHKDTLRSAKRFVKHNEKISVKRVWLTEIKLADLENMVNYKNGREIELYEALKARLEAYGGNAKQAFDPKDNPFYKKGGQLVKAVRVEKTQESGVLLNKKNAYTIADNGDMVRVDVFCKVDKKGKNQYFIVPIYAWQVAENILPDIDCKGYRIDDSYTFCFSLHKYDLIAFQKDEKSKVEFAYYINCDSSNGRFYLAWHDKGSKEQQFRISTQNLVLIQKYQVNELGKEIRPCRLKKRPPVRGTGGPKKKRKVYPYDVPDYAGYPYDVPDYAGSYPYDVPDYAGSAAPAAKKKKLDFESG

SEQ ID NO: 312 - protein, 1168 aa, synthetic construct
  REGION 1..1168: note = Synthetic
MVPKKKRKVEDKRPAATKKAGQAKKKKMAAFKPNPINYILGLDIGIASVGWAMVEIDEEENPIRLIDLGVRVFERAEVPKTGDSLAMARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVANNAHALQTGDFRTPAELALNKFEKESGHIRNQRGDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSSELQDEIGTAFSLFKTDEDITGRLKDRVQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLVRLNEKGYVEIDHALPFSRTWDDSFNNKVLVLGSENQNKGNQTPYEYFNGKDNSREWQEFKARVETSRFPRSKKQRILLQKFDEDGFKECNLNDTRYVNRFLCQFVADHILLTGKGKRRVFASNGQITNLLRGFWGLRKVRAENDRHHALDAVVVACSTVAMQQKITRFVRYKEMNAFDGKTIDKETGKVLHQKTHFPQPWEFFAQEVMIRVFGKPDGKPEFEEADTPEKLRTLLAEKLSSRPEAVHEYVTPLFVSRAPNRKMSGAHKDTLRSAKRFVKHNEKISVKRVWLTEIKLADLENMVNYKNGREIELYEALKARLEAYGGNAKQAFDPKDNPFYKKGGQLVKAVRVEKTQESGVLLNKKNAYTIADNGDMVRVDVFCKVDKKGKNQYFIVPIYAWQVAENILPDIDCKGYRIDDSYTFCFSLHKYDLIAFQKDEKSKVEFAYYINCDSSNGRFYLAWHDKGSKEQQFRISTQNLVLIQKYQVNELGKEIRPCRLKKRPPVREDKRPAATKKAGQAKKKKYPYDVPDYAGYPYDVPDYAGSYPYDVPDYAAAPAAKKKKLD

SEQ ID NO: 313 - protein, 1135 aa, synthetic construct
  REGION 1..1135: note = Synthetic
PKKKRKVNAMAAFKPNPINYILGLDIGIASVGWAMVEIDEEENPIRLIDLGVRVFERAEVPKTGDSLAMARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVANNAHALQTGDFRTPAELALNKFEKESGHIRNQRGDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSSELQDEIGTAFSLFKTDEDITGRLKDRVQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLVRLNEKGYVEIDHALPFSRTWDDSFNNKVLVLGSENQNKGNQTPYEYFNGKDNSREWQEFKARVETSRFPRSKKQRILLQKFDEDGFKECNLNDTRYVNRFLCQFVADHILLTGKGKRRVFASNGQITNLLRGFWGLRKVRAENDRHHALDAVVVACSTVAMQQKITRFVRYKEMNAFDGKTIDKETGKVLHQKTHFPQPWEFFAQEVMIRVFGKPDGKPEFEEADTPEKLRTLLAEKLSSRPEAVHEYVTPLFVSRAPNRKMSGAHKDTLRSAKRFVKHNEKISVKRVWLTEIKLADLENMVNYKNGREIELYEALKARLEAYGGNAKQAFDPKDNPFYKKGGQLVKAVRVEKTQESGVLLNKKNAYTIADNGDMVRVDVFCKVDKKGKNQYFIVPIYAWQVAENILPDIDCKGYRIDDSYTFCFSLHKYDLIAFQKDEKSKVEFAYYINCDSSNGRFYLAWHDKGSKEQQFRISTQNLVLIQKYQVNELGKEIRPCRLKKRPPVRGGGGSGGGGSGGGGSPAAKKKKLDGGGSKRPAATKKAGQAKKKK

SEQ ID NO: 314 - DNA, 4377 bp, synthetic construct
  misc_feature 1..4377: note = Synthetic
  variation 137..159: note = n is a, c, g, or t
  source 1..4377: mol_type = other DNA
gtttaaacaaaaaaataaacgatgccccttaaagcagaagctttaaggggcagagcgttgcggcacatcttttcagacggccttattgtagcaacggttctcatttcgtttccgaaatgagaaagggagctacaacnnnnnnnnnnnnnnnnnnnnnnncggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattctaattatctctctaacagccttgtatcgtatatgcaaatatgaaggaatcatgggaaataggccctcttcctgcccgaccttgacgtcgactctagaatggaggcggtactatgtagatgagaattcaggagcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcatacttaagcagacagtggttcaaagtttttttcttccatttcaggtgtcgtgaacaccgccaccatggtgcctaagaagaagagaaaggtggaagataaacgcccagcagctacaaagaaggcaggtcaagccaagaaaaagaaagcagcattcaagccaaactcaatcaattacatcctgggactggacatcggcatcgcatccgtcgggtgggctatggtcgaaatcgacgaggaggagaaccccatccgcctgatcgatctgggcgtgcgcgtgtttgagagggcagaggtgcctaagaccggcgacagcctggccatggcacggagactggcacgctccgtgaggcgcctgacccggagaagggcccacagactgctgaggacacgccggctgctgaagagggagggcgtgctgcaggccgccaacttcgatgagaatggcctgatcaagtccctgcccaataccccttggcagctgagggcagccgccctggaccgcaagctgacacctctggagtggtccgccgtgctgctgcacctgatcaagcaccggggctacctgtctcagagaaagaacgagggcgagacagccgataaggagctgggcgccctgctgaagggagtggcaggaaatgcacacgccctgcagaccggcgactttcgcacaccagccgagctggccctgaacaagttcgagaaggagagcggccacatccgcaatcagcggtctgactatagccacaccttctcccggaaggatctgcaggccgagctgatcctgctgtttgagaagcagaaggagttcggcaacccacacgtgtctggcggcctgaaggagggcatcgagacactgctgatgacacagcggcccgccctgagcggcgacgcagtgcagaagatgctgggacactgcacctttgagccagccgagcccaaggccgccaagaatacctacacagccgagcggttcatctggctgacaaagctgaacaatctgaggatcctggagcagggaagcgagcgcccactgaccgacacagagagggccaccctgatggatgagccctaccgcaagtccaagctgacatatgcacaggcaaggaagctgctgggcctggaggacaccgccttctttaagggcctgagatacggcaaggataacgccgaggcctctacactgatggagatgaaggcctatcacgccatcagcagggccctggagaaggagggcctgaaggacaagaagtccccactgaatctgtctcccgagctgcaggatgagatcggcaccgcctttagcctgttcaagaccgacgaggatatcacaggcagactgaaggacaggatccagccagagatcctggaggccctgctgaagcacatcagctttgataagttcgtgcagatcagcctgaaggccctgcggaggatcgtgccactgatggagcagggcaagaggtacgacgaggcctgcgccgaaatctacggcgatcactatggcaagaagaacacagaggagaaaatctacctgccccctatccccgccgatgagatcaggaaccctgtggtgctgcgcgccctgtctcaggcaagaaaagtgatcaacggagtggtgcgccggtacggcagccccgccagaatccacatcgagacagccagggaagtgggcaagtcctttaaggacagaaaggagatcgagaagaggcaggaggagaacagaaaggatagggagaaggccgccgccaagttcagagagtactttcctaatttcgtgggcgagccaaagtccaaggatatcctgaagctgaggctgtacgagcagcagcacggcaagtgtctgtattctggcaaggagatcaacctgggccgcctgaatgagaagggctatgtggagatcgaccacgccctgcctttttctcggacctgggacgatagcttcaacaataaggtgctggtgctgggctctgagaaccagaataagggcaaccagacaccctacgagtatttcaacggcaaggacaatagccgcgagtggcaggagtttaaggcaagggtggagacaagcaggttccctcggtccaagaagcagagaatcctgctgcagaagtttgacgaggatggcttcaaggagaggaacctgaatgacacccgctacgtgaatcggtttctgtgccagttcgtggccgatagaatgaggctgaccggcaagggcaagaagagagtgtttgcctccaacggccagatcacaaatctgctgaggggcttctggggcctgagaaaggtgagggcagagaacgacaggcaccacgcactggatgcagtggtggtggcatgttctaccgtggccatgcagcagaagatcacacgctttgtgcggtataaggagatgaatgccttcgacggcaagaccatcgataaggagacaggcgaggtgctgcaccagaagacacactttcctcagccatgggagttctttgcccaggaagtgatgatccgggtgtttggcaagcctgacggcaagccagagttcgaggaggccgataccctggagaagctgagaacactgctggcagagaagctgagctccaggcccgaggcagtgcacgagtacgtgaccccactgttcgtgtctagagcccccaacaggaagatgagcggccagggccacatggagacagtgaagtccgccaagagactggacgagggcgtgtctgtgctgagggtgcctctgacacagctgaagctgaaggatctggagaagatggtgaatcgcgagcgggagccaaagctgtatgaggccctgaaggcaaggctggaggcacacaaggacgatcctgccaaggcctttgccgagccattctacaagtatgataaggccggcaacagaacccagcaggtgaaggccgtgagggtggagcaggtgcagaagacaggcgtgtgggtgcgcaaccacaatggcatcgccgacaatgctaccatggtgcgggtggacgtgtttgagaagggcgataagtactatctggtgcccatctacagctggcaggtggccaagggcatcctgcctgatagagccgtggtgcagggcaaggacgaggaggattggcagctgatcgacgattccttcaactttaagttctctctgcaccccaatgacctggtggaagtgatcaccaagaaggccaggatgtttggctacttcgcctcctgccaccgcggcacaggcaacatcaatatccggatccacgacctggatcacaagatcggcaagaacggcatcctggagggcatcggcgtgaagacagccctgagcttccagaagtatcagatcgacgagctgggcaaggagatcagaccttgtaggctgaagaagcgcccacccgtgcgggaggataagcggcccgcagcaaccaagaaggcaggacaggccaagaagaagaagtacccctatgacgtgcctgactacgccgggtatccctacgacgtgcctgattacgccgggtcctatccctacgacgtgccagattacgctgcagctccagcagcgaagaaaaagaagctggattaagatctttttccctctgccaaaaattatggggacatcatgaagccccttgagcatctgacttctggctaataaaggaaatttattttcattgcaatagtgtgttggaattttttgtgtctctca